SEQUENCE LISTING (154 sequences)

SEQ ID NO: 1 - DNA, 1653 bp, Artificial Sequence
  Synthetic Polynucleotide
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

SEQ ID NO: 2 - protein, 550 aa, Artificial Sequence
  Synthetic Polypeptide
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 3 - DNA, 516 bp, Artificial Sequence
  Synthetic Polynucleotide
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 4 - protein, 171 aa, Artificial Sequence
  Synthetic Polypeptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 5 - DNA, 1407 bp, Artificial Sequence
  Synthetic Polynucleotide
atgccaaaaaataacaaagaagaagaagttaaagaagtaaaccttaattcagtacaagaggatgcgttaaagtcctttacaactggttatggtatcacacctgatacacaaacagatgcaggggcactaagacgtgagttcctagacgaccaaatctcaatgcttacttggacagaaaatgatttaacattctacaaagacatcgctaaaaaaccagctacatctacagtagcaaaatacgatgtgtacatgcaacacggtaaagtaggtcatactagatttactcgtgagattggggtagcaccagtaagtgaccctaacatccgtcaaaaaacagtaaacatgaaatttgcttctgatactaaaaatattagtatcgcagcaggtctagtaaacaacattcaagaccctatgcaaattttgactgatgatgctatcgtaaatatcgctaaaacaattgagtgggcttcattctttggagattctgacttatcagatagcccagaaccacaagcaggattagaatttgatggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaa

SEQ ID NO: 6 - protein, 468 aa, Artificial Sequence
  Synthetic Polypeptide
MPKNNKEEEVKEVNLNSVQEDALKSFTTGYGITPDTQTDAGALRREFLDDQISMLTWTENDLTFYKDIAKKPATSTVAKYDVYMQHGKVGHTRFTREIGVAPVSDPNIRQKTVNMKFASDTKNISIAAGLVNNIQDPMQILTDDAIVNIAKTIEWASFFGDSDLSDSPEPQAGLEFDGLAKLINQDNVHDARGASLTESLLNQAAVMISKGYGTPTDAYMPVGVQADFVNQQLSKQTQLVRDNGNNVSVGFNIQGFHSARGFIKLHGSTVMENEQILDERILALPTAPQPAKVTATQEAGKKGQFRAEDLAAHEYKVVVSSDDAESIASEVATATVTAKDDGVKLEIELAPMYSSRPQFVSIYRKGAETGLFYLIARVPASKAENNVITFYDLNDSIPETVDVFVGEMSANVVHLFELLPMMRLPLAQINASVTFAVLWYGALALRAPKKWVRIRNVKYIPVKNVHSN

SEQ ID NO: 7 - DNA, 1407 bp, Artificial Sequence
  Synthetic Polynucleotide
atgccaaaaaataacaaagaagaagaagttaaagaagtaaaccttaattcagtacaagaggacgcgttaaagtcctttacaactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaacatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaa

SEQ ID NO: 8 - protein, 468 aa, Artificial Sequence; identical to SEQ ID NO: 6
  Synthetic Polypeptide

SEQ ID NO: 9 - DNA, 1407 bp, Artificial Sequence; identical to SEQ ID NO: 7
  Synthetic Polynucleotide

SEQ ID NO: 10 - protein, 468 aa, Artificial Sequence; identical to SEQ ID NO: 6
  Synthetic Polypeptide

SEQ ID NO: 11 - DNA, 1407 bp, Artificial Sequence; identical to SEQ ID NO: 7
  Synthetic Polynucleotide

SEQ ID NO: 12 - protein, 468 aa, Artificial Sequence; identical to SEQ ID NO: 6
  Synthetic Polypeptide

SEQ ID NO: 13 - DNA, 1407 bp, Artificial Sequence; identical to SEQ ID NO: 7
  Synthetic Polynucleotide

SEQ ID NO: 14 - protein, 468 aa, Artificial Sequence; identical to SEQ ID NO: 6
  Synthetic Polypeptide

SEQ ID NO: 15 - DNA, 1407 bp, Artificial Sequence
  Synthetic Polynucleotide
atgccaaaaaataacaaagaagaagaagttaaagaagtaaaccttaattcagtacaagaggacgcgttaaagtcctttacaactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaacatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgtgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaa

SEQ ID NO: 16 - protein, 468 aa, Artificial Sequence; identical to SEQ ID NO: 6
  Synthetic Polypeptide

SEQ ID NO: 17 - DNA, 1404 bp, Artificial Sequence
  Synthetic Polynucleotide
atgccaaaaaataacaaagaagaagttaaagaagtaaaccttaattcagtacaagaggatgcgttaaagtcctttacgactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaatatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaa

SEQ ID NO: 18 - protein, 467 aa, Artificial Sequence
  Synthetic Polypeptide
MPKNNKEEVKEVNLNSVQEDALKSFTTGYGITPDTQTDAGALRREFLDDQISMLTWTENDLTFYKDIAKKPATSTVAKYDVYMQHGKVGHTRFTREIGVAPVSDPNIRQKTVNMKFASDTKNISIAAGLVNNIQDPMQILTDDAIVNIAKTIEWASFFGDSDLSDSPEPQAGLEFDGLAKLINQDNVHDARGASLTESLLNQAAVMISKGYGTPTDAYMPVGVQADFVNQQLSKQTQLVRDNGNNVSVGFNIQGFHSARGFIKLHGSTVMENEQILDERILALPTAPQPAKVTATQEAGKKGQFRAEDLAAHEYKVVVSSDDAESIASEVATATVTAKDDGVKLEIELAPMYSSRPQFVSIYRKGAETGLFYLIARVPASKAENNVITFYDLNDSIPETVDVFVGEMSANVVHLFELLPMMRLPLAQINASVTFAVLWYGALALRAPKKWVRIRNVKYIPVKNVHSN

SEQ ID NO: 19 - DNA, 1404 bp, Artificial Sequence; identical to SEQ ID NO: 17
  Synthetic Polynucleotide

SEQ ID NO: 20 - protein, 467 aa, Artificial Sequence; identical to SEQ ID NO: 18
  Synthetic Polypeptide

SEQ ID NO: 21 - DNA, 1407 bp, Artificial Sequence
  Synthetic Polynucleotide
atgccaaaaaataacaaagaagaagaagttaaagaagtaaaccttaattcagtacaagaggacgcgttaaagtcctttacaactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaatatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacagcttgttcgtgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagacttagcagcacacgaatacaaagttgttgtaagttctgacgatgcagagtctattgcaagtgaagtggctacagctacagttactgcaaaagatgacggcgttaaactagaaatcgagttagctccaatgtacagctcccgtccacaattcgtttcaatctatagaaaaggtgcagaaacaggtttattctacctaatcgctcgtgtacctgctagcaaagcagagaacaacgtaatcactttctatgacttaaacgactctattcctgaaacagtagacgtattcgttggtgaaatgtctgctaacgtagtacacttgtttgaattactaccaatgatgagattacctctagctcaaattaacgcatctgttacatttgcagttttatggtatggagcattagctctaagagcacctaagaaatgggtacgtattagaaacgttaaatatattcctgtaaaaaacgttcatagcaactaa

SEQ ID NO: 22 - protein, 468 aa, Artificial Sequence; identical to SEQ ID NO: 6
  Synthetic Polypeptide

SEQ ID NO: 23 - DNA, 3729 bp, Artificial Sequence
  Synthetic Polynucleotide
atgccaaaaaataacaaagaagaagaagttaaagaagtaaaccttaattcagtacaagaggacgcgttaaagtcctttacaactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaacatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaagaggaggtaaatatatatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttccagccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaataattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaatgatataattgaagaactaaagagagggtaatgtataatgtatggaggttatgaaggacaagattcttacgaatacccttactcacatgggaaccctaagcatgtagagccagaaaaagttgacgaatatgttctttctgattatggttggactgcggaaacaattaaagcatacatgtatggtgttcgtgtagtagaccctgaaacaggagaggaaatgggagacaccttctacaatcatattatagaggttgccgttgataaggc

SEQ ID NO: 24 - DNA, 3789 bp, Artificial Sequence
  Synthetic Polynucleotide
atgccaaaaaataacaaagaagaagaagttaaagaagtaaaccttaattcagtacaagaggacgcgttaaagtcctttacaactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaacatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaagaggaggtaaatatatatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaataattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaagaacctaagaaagaagaacctaagaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaatgatataattgaagaactaaagagagggtaatgtataatgtatggaggttatgaaggacaagattcttacgaatacccttactcacatgggaaccctaagcatgtagagccagaaaaagttgacgaatatgttctttctgattatggttggactgcggaaacaattaaagcatacatgtatggtgttcgtgtagtagaccctgaaacaggagaggaaatgggagacaccttctacaatcatattatagaggttgccgttgataaggc

SEQ ID NO: 25 - DNA, 3789 bp, Artificial Sequence; identical to SEQ ID NO: 24
  Synthetic Polynucleotide

SEQ ID NO: 26 - DNA, 3789 bp, Artificial Sequence; identical to SEQ ID NO: 24
  Synthetic Polynucleotide

SEQ ID NO: 27 - DNA, 3729 bp, Artificial Sequence
  Synthetic Polynucleotide
atgccaaaaaataacaaagaagaagaagttaaagaagtaaaccttaattcagtacaagaggacgcgttaaagtcctttacaactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaacatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgtgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaagaggaggtaaatatatatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttccagccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaataattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaatgatataattgaagaactaaagagagggtaatgtataatgtatggaggttatgaaggacaagattcttacgaatacccttactcacatgggaaccctaagcatgtagagccagaaaaagttgacgaatatgttctttctgattatggttggactgcggaaacaattaaagcatacatgtatggtgttcgtgtagtagaccctgaaacaggagaggaaatgggagacaccttctacaatcatattatagaggttgccgttgataaggc

SEQ ID NO: 28 - DNA, 3786 bp, Artificial Sequence
  Synthetic Polynucleotide
atgccaaaaaataacaaagaagaagttaaagaagtaaaccttaattcagtacaagaggatgcgttaaagtcctttacgactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaatatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaagaggaggtaaatatatatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaataattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaagaacctaagaaagaagaacctaagaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaatgatataattgaagaactaaagagagggtaatgtataatgtatggaggttatgaaggacaagattcttacgaatacccttactcacatgggaaccctaagcatgtagagccagaaaaagttgacgaatatgttctttctgattatggttggactgcggaaacaattaaagcatacatgtatggtgttcgtgtagtagaccctgaaacaggagaggaaatgggagacaccttctacaatcatattatagaggttgccgttgataaggc

SEQ ID NO: 29 - DNA, 3786 bp, Artificial Sequence; identical to SEQ ID NO: 28
  Synthetic Polynucleotide

SEQ ID NO: 30 - DNA, 3729 bp, Artificial Sequence
  Synthetic Polynucleotide
atgccaaaaaataacaaagaagaagaagttaaagaagtaaaccttaattcagtacaagaggacgcgttaaagtcctttacaactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaatatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacagcttgttcgtgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagacttagcagcacacgaatacaaagttgttgtaagttctgacgatgcagagtctattgcaagtgaagtggctacagctacagttactgcaaaagatgacggcgttaaactagaaatcgagttagctccaatgtacagctcccgtccacaattcgtttcaatctatagaaaaggtgcagaaacaggtttattctacctaatcgctcgtgtacctgctagcaaagcagagaacaacgtaatcactttctatgacttaaacgactctattcctgaaacagtagacgtattcgttggtgaaatgtctgctaacgtagtacacttgtttgaattactaccaatgatgagattacctctagctcaaattaacgcatctgttacatttgcagttttatggtatggagcattagctctaagagcacctaagaaatgggtacgtattagaaacgttaaatatattcctgtaaaaaacgttcatagcaactaagaggaggtaaatatatatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaataattataggataattgaataaaaacagtatagagagcagataaatactgctctctattttactaataaggaggatttaaattgctaaaaaatacaaacttagctaattataaaaaagtgaatacacgatttggaaatcttagttttgatgataaaggtatttctaatgacctaacggaagagcagcaaaaagaattaggtaagcttagaggattcgaatatattaagacagaacagaaaacgaaagaagaacctaagaaagaagaacctaagaaagaaagtacagaaaatgaattagacagcttcttagctaaagaaccttcaatcaaagaattaaaagaatttgcgagtaaaaaaggcattaaaattgaaaaaactaagaaaaatgatataattgaagaactaaagagagggtaatgtacaatgtatggaggttatgaaggacaagattcttacgaatacccttactcacacgggaaccctaagcatgtagagccagaaaaagttgacgaatatgttctttctgattatggctggactgcggaaacaattaaagcatacatgtatggtgttcgtgtagtagaccctgaaacaggagaggaaatgggagacaccttctacaatcatattatagaggttgccgttgataaggc

SEQ ID NO: 31 - DNA, 2658 bp, Artificial Sequence
  Synthetic Polynucleotide
atgccaaaaaataacaaagaagaagaagttaaagaagtaaaccttaattcagtacaagaggacgcgttaaagtcctttacaactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaacatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaataataagaggaggtaaatatatatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaagaacctaagaaagaagaacctaagaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaatgatataattgaagaactaaagagagggtaatgtataatgtatggaggttatgaaggacaagattcttacgaatacccttactcacatgggaaccctaagcatgtagagccagaaaaagttgacgaatatgttctttctgattatggttggactgcggaaacaattaaagcatacatgtatggtgttcgtgtagtagaccctgaaacaggagaggaaatgggagacaccttctacaatcatattatagaggttgccgttgataaggc

SEQ ID NO: 32 - DNA, 2598 bp, Artificial Sequence
  Synthetic Polynucleotide
atgccaaaaaataacaaagaagaagaagttaaagaagtaaaccttaattcagtacaagaggacgcgttaaagtcctttacaactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaacatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgtgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaataataagaggaggtaaatatatatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaatgatataattgaagaactaaagagagggtaatgtataatgtatggaggttatgaaggacaagattcttacgaatacccttactcacatgggaaccctaagcatgtagagccagaaaaagttgacgaatatgttctttctgattatggttggactgcggaaacaattaaagcatacatgtatggtgttcgtgtagtagaccctgaaacaggagaggaaatgggagacaccttctacaatcatattatagaggttgccgttgataaggc

SEQ ID NO: 33 - DNA, 2649 bp, Artificial Sequence
  Synthetic Polynucleotide
atgccaaaaaataacaaagaagaagttaaagaagtaaaccttaattcagtacaagaggatgcgttaaagtcctttacgactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaatatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaagaggaggtaaatatatatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtaatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaagaacctaagaaagaagaacctaagaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaatgatataattgaagaactaaagagagggtaatgtataatgtatggaggttatgaaggacaagattcttacgaatacccttactcacatgggaaccctaagcatgtagagccagaaaaagttgacgaatatgttctttctgattatggttggactgcggaaacaattaaagcatacatgtatggtgttcgtgtagtagaccctgaaacaggagaggaaatgggagacaccttctacaatcatattatagaggttgccgttgataaggc

SEQ ID NO: 34 - DNA, 2592 bp, Artificial Sequence
  Synthetic Polynucleotide
atgccaaaaaataacaaagaagaagaagttaaagaagtaaaccttaattcagtacaagaggacgcgttaaagtcctttacaactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaatatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacagcttgttcgtgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagacttagcagcacacgaatacaaagttgttgtaagttctgacgatgcagagtctattgcaagtgaagtggctacagctacagttactgcaaaagatgacggcgttaaactagaaatcgagttagctccaatgtacagctcccgtccacaattcgtttcaatctatagaaaaggtgcagaaacaggtttattctacctaatcgctcgtgtacctgctagcaaagcagagaacaacgtaatcactttctatgacttaaacgactctattcctgaaacagtagacgtattcgttggtgaaatgtctgctaacgtagtacacttgtttgaattactaccaatgatgagattacctctagctcaaattaacgcatctgttacatttgcagttttatggtatggagcattagctctaagagcacctaagaaatgggtacgtattagaaacgttaaatatattcctgtaaaaaacgttcatagcaactaagaggaggtaaatatatatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataattataggataattgaataaaaacagtatagagagcagataaatactgctctctattttactaataaggaggatttaaattgctaaaaaatacaaacttagctaattataaaaaagtgaatacacgatttggaaatcttagttttgatgataaaggtatttctaatgacctaacggaagagcagcaaaaagaattaggtaagcttagaggattcgaatatattaagacagaacagaaaacgaaagaagaacctaagaaagaagaacctaagaaagaaagtacagaaaatgaattagacagcttcttagctaaagaaccttcaatcaaagaattaaaagaatttgcgagtaaaaaaggcattaaaattgaaaaaactaagaaaaatgatataattgaagaactaaagagagggtaatgtacaatgtatggaggttatgaaggacaagattcttacgaatacccttactcacacgggaaccctaagcatgtagagccagaaaaagttgacgaatatgttctttctgattatggctggactgcggaaacaattaaagcatacatgtatggtgttcgtgtagtagaccctgaaacaggagaggaaatgggagacaccttctacaatcatattatagaggttgccgttgataaggc

SEQ ID NO: 35 - DNA, 2613 bp, Artificial Sequence
  Synthetic Polynucleotide
atgccaaaaaataacaaagaagaagaagttaaagaagtaaaccttaattcagtacaagaggatgcgttaaagtcctttacaactggttatggtatcacacctgatacacaaacagatgcaggggcactaagacgtgagttcctagacgaccaaatctcaatgcttacttggacagaaaatgatttaacattctacaaagacatcgctaaaaaaccagctacatctacagtagcaaaatacgatgtgtacatgcaacacggtaaagtaggtcatactagatttactcgtgagattggggtagcaccagtaagtgaccctaacatccgtcaaaaaacagtaaacatgaaatttgcttctgatactaaaaatattagtatcgcagcaggtctagtaaacaacattcaagaccctatgcaaattttgactgatgatgctatcgtaaatatcgctaaaacaattgagtgggcttcattctttggagattctgacttatcagatagcccagaaccacaagcaggattagaatttgatggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaataataagaggaggtaaatatatatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaataattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaacgatataattgaagaactaaagagagggtaatgtataatgtatggaggttatgaaggacaagattcttacgaatacccttactcacatgggaaccctaagcatgtagagccagaaaaagttgacgaatatgttctttctgattatggttggactgcggaaacaattaaagcatacatgtatggtgttcgtgtagtagaccctgaaacaggagaggaaatgggagacaccttctacaatcatattatagaggttgccgttgataaggc

SEQ ID NO: 36 - DNA, 532 bp, Artificial Sequence
  Synthetic Polynucleotide
gaggaggtaaatatatatggtattcacattagaagattttgtaggggattggcgacaaacagcgggatataacttagatcaagttttggaacagggtggagtctcaagcctctttcaaaatcttggagtgagtgttactcctattcaaagaattgtactatctggtgaaaatggcttaaagattgatatacatgttatcattccatacgaaggcttatcgggtgatcaaatgggtcaaattgagaaaatctttaaagtagtgtatcctgtagacgatcatcatttcaaagttattcttcactatggtacgcttgtgatagacggggttacaccaaatatgattgattactttggtcggccgtatgaaggcattgctgtttttgacgggaaaaaaatcaccgtcactggaactttatggaatggtaacaaaatcattgatgaacgtttgataaatccagatggatccttacttttccgcgtgacaatcaacggagtaacgggctggagattatgtgaacgtattctagcataa

SEQ ID NO: 37 - protein, 516 aa, Artificial Sequence
  Synthetic Polypeptide
ATGGTATTCACATTGGAAGATTTTGTGGGGGATTGGAGACAGACAGCTGGATATAACTTAGACCAAGTATTAGAACAGGGTGGAGTGTCAAGCTTATTTCAAAACTTAGGTGTGTCAGTGACTCCAATTCAACGTATTGTGTTAAGTGGAGAAAACGGTTTAAAAATAGACATTCATGTGATTATTCCGTACGAAGGCCTCAGTGGTGACCAAATGGGACAAATAGAGAAAATCTTTAAAGTAGTGTACCCTGTGGACGACCATCACTTTAAAGTAATCTTACACTATGGTACGTTAGTAATTGATGGCGTAACGCCAAACATGATAGACTACTTTGGGCGTCCTTATGAAGGCATTGCCGTGTTTGACGGCAAAAAGATCACCGTGACAGGTACTCTATGGAATGGAAACAAAATCATTGACGAGCGTTTAATCAACCCAGACGGCTCTTTACTATTTCGGGTAACAATTAACGGCGTGACCGGATGGCGATTATGCGAGCGCATTTTAGCCTAA

SEQ ID NO: 38 - DNA, 2442 bp, Artificial Sequence
  Synthetic Polynucleotide
atgccaaaaaataacaaagaagaagttaaagaagtaaaccttaattcagtacaagaggatgcgttaaagtcctttacgactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaatatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaataataagaggaggtaaatatatatggtattcacattagaagattttgtaggggattggcgacaaacagcgggatataacttagatcaagttttggaacagggtggagtctcaagcctctttcaaaatcttggagtgagtgttactcctattcaaagaattgtactatctggtgaaaatggcttaaagattgatatacatgttatcattccatacgaaggcttatcgggtgatcaaatgggtcaaattgagaaaatctttaaagtagtgtatcctgtagacgatcatcatttcaaagttattcttcactatggtacgcttgtgatagacggggttacaccaaatatgattgattactttggtcggccgtatgaaggcattgctgtttttgacgggaaaaaaatcaccgtcactggaactttatggaatggtaacaaaatcattgatgaacgtttgataaatccagatggatccttacttttccgcgtgacaatcaacggagtaacgggctggagattatgtgaacgtattctagcataataattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaagaacctaagaaagaagaacctaagaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaatgatataattgaagaactaaagagagggtaatgtataatgtatggaggttatga

SEQ ID NO: 39 - DNA, 2445 bp, Artificial Sequence
  Synthetic Polynucleotide
atgccaaaaaataacaaagaagaagaagttaaagaagtaaaccttaattcagtacaagaggacgcgttaaagtcctttacaactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaacatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaataataagaggaggtaaatatatatggtattcacattagaagattttgtaggggattggcgacaaacagcgggatataacttagatcaagttttggaacagggtggagtctcaagcctctttcaaaatcttggagtgagtgttactcctattcaaagaattgtactatctggtgaaaatggcttaaagattgatatacatgttatcattccatacgaaggcttatcgggtgatcaaatgggtcaaattgagaaaatctttaaagtagtgtatcctgtagacgatcatcatttcaaagttattcttcactatggtacgcttgtgatagacggggttacaccaaatatgattgattactttggtcggccgtatgaaggcattgctgtttttgacgggaaaaaaatcaccgtcactggaactttatggaatggtaacaaaatcattgatgaacgtttgataaatccagatggatccttacttttccgcgtgacaatcaacggagtaacgggctggagattatgtgaacgtattctagcataataattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaagaacctaagaaagaagaacctaagaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaatgatataattgaagaactaaagagagggtaatgtataatgtatggaggttatga

SEQ ID NO: 40 - DNA, 2445 bp, Artificial Sequence
  Synthetic Polynucleotide
atgccaaaaaataacaaagaagaagaagttaaagaagtaaaccttaattcagtacaagaggatgcgttaaagtcctttacaactggttatggtatcacacctgatacacaaacagatgcaggggcactaagacgtgagttcctagacgaccaaatctcaatgcttacttggacagaaaatgatttaacattctacaaagacatcgctaaaaaaccagctacatctacagtagcaaaatacgatgtgtacatgcaacacggtaaagtaggtcatactagatttactcgtgagattggggtagcaccagtaagtgaccctaacatccgtcaaaaaacagtaaacatgaaatttgcttctgatactaaaaatattagtatcgcagcaggtctagtaaacaacattcaagaccctatgcaaattttgactgatgatgctatcgtaaatatcgctaaaacaattgagtgggcttcattctttggagattctgacttatcagatagcccagaaccacaagcaggattagaatttgatggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaataataagaggaggtaaatatatatggtattcacattagaagattttgtaggggattggcgacaaacagcgggatataacttagatcaagttttggaacagggtggagtctcaagcctctttcaaaatcttggagtgagtgttactcctattcaaagaattgtactatctggtgaaaatggcttaaagattgatatacatgttatcattccatacgaaggcttatcgggtgatcaaatgggtcaaattgagaaaatctttaaagtagtgtatcctgtagacgatcatcatttcaaagttattcttcactatggtacgcttgtgatagacggggttacaccaaatatgattgattactttggtcggccgtatgaaggcattgctgtttttgacgggaaaaaaatcaccgtcactggaactttatggaatggtaacaaaatcattgatgaacgtttgataaatccagatggatccttacttttccgcgtgacaatcaacggagtaacgggctggagattatgtgaacgtattctagcataataattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaacgatataattgaagaactaaagagagggtaatgtataatgtatggaggttatgaaggacaagattcttacgaatacccttactcacatgggaaccctaa

SEQ ID NO: 41 - DNA, 532 bp, Artificial Sequence; identical to SEQ ID NO: 36
  Synthetic Polynucleotide

SEQ ID NO: 42 - DNA, 2442 bp, Artificial Sequence
  Synthetic Polynucleotide
atgccaaaaaataacaaagaagaagttaaagaagtaaaccttaattcagtacaagaggatgcgttaaagtcctttacgactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaatatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaataataagaggaggtaaatatatatggtattcacattggaagattttgtgggggattggagacagacagctggatataacttagaccaagtattagaacagggtggagtgtcaagcttatttcaaaacttaggtgtgtcagtgactccaattcaacgtattgtgttaagtggagaaaacggtttaaaaatagacattcatgtgattattccgtacgaaggcctcagtggtgaccaaatgggacaaatagagaaaatctttaaagtagtgtaccctgtggacgaccatcactttaaagtaatcttacactatggtacgttagtaattgatggcgtaacgccaaacatgatagactactttgggcgtccttatgaaggcattgccgtgtttgacggcaaaaagatcaccgtgacaggtactctatggaatggaaacaaaatcattgacgagcgtttaatcaacccagacggctctttactatttcgggtaacaattaacggcgtgaccggatggcgattatgcgagcgcattttagcctaataattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaagaacctaagaaagaagaacctaagaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaatgatataattgaagaactaaagagagggtaatgtataatgtatggaggttatga

SEQ ID NO: 43 - DNA, 2445 bp, Artificial Sequence
  Synthetic Polynucleotide
atgccaaaaaataacaaagaagaagaagttaaagaagtaaaccttaattcagtacaagaggacgcgttaaagtcctttacaactggttatggtatcacacctgatacacaaacagatgcaggagcattaagacgtgagttcctagacgaccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaacatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaataataagaggaggtaaatatatatggtattcacattggaagattttgtgggggattggagacagacagctggatataacttagaccaagtattagaacagggtggagtgtcaagcttatttcaaaacttaggtgtgtcagtgactccaattcaacgtattgtgttaagtggagaaaacggtttaaaaatagacattcatgtgattattccgtacgaaggcctcagtggtgaccaaatgggacaaatagagaaaatctttaaagtagtgtaccctgtggacgaccatcactttaaagtaatcttacactatggtacgttagtaattgatggcgtaacgccaaacatgatagactactttgggcgtccttatgaaggcattgccgtgtttgacggcaaaaagatcaccgtgacaggtactctatggaatggaaacaaaatcattgacgagcgtttaatcaacccagacggctctttactatttcgggtaacaattaacggcgtgaccggatggcgattatgcgagcgcattttagcctaataattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaagaacctaagaaagaagaacctaagaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaatgatataattgaagaactaaagagagggtaatgtataatgtatggaggttatga

SEQ ID NO: 44 - DNA, 2445 bp, Artificial Sequence
  Synthetic Polynucleotide
atgccaaaaaataacaaagaagaagaagttaaagaagtaaaccttaattcagtacaagaggatgcgttaaagtcctttacaactggttatggtatcacacctgatacacaaacagatgcaggggcactaagacgtgagttcctagacgaccaaatctcaatgcttacttggacagaaaatgatttaacattctacaaagacatcgctaaaaaaccagctacatctacagtagcaaaatacgatgtgtacatgcaacacggtaaagtaggtcatactagatttactcgtgagattggggtagcaccagtaagtgaccctaacatccgtcaaaaaacagtaaacatgaaatttgcttctgatactaaaaatattagtatcgcagcaggtctagtaaacaacattcaagaccctatgcaaattttgactgatgatgctatcgtaaatatcgctaaaacaattgagtgggcttcattctttggagattctgacttatcagatagcccagaaccacaagcaggattagaatttgatggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaataataagaggaggtaaatatatatggtattcacattggaagattttgtgggggattggagacagacagctggatataacttagaccaagtattagaacagggtggagtgtcaagcttatttcaaaacttaggtgtgtcagtgactccaattcaacgtattgtgttaagtggagaaaacggtttaaaaatagacattcatgtgattattccgtacgaaggcctcagtggtgaccaaatgggacaaatagagaaaatctttaaagtagtgtaccctgtggacgaccatcactttaaagtaatcttacactatggtacgttagtaattgatggcgtaacgccaaacatgatagactactttgggcgtccttatgaaggcattgccgtgtttgacggcaaaaagatcaccgtgacaggtactctatggaatggaaacaaaatcattgacgagcgtttaatcaacccagacggctctttactatttcgggtaacaattaacggcgtgaccggatggcgattatgcgagcgcattttagcctaataattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaacgatataattgaagaactaaagagagggtaatgtataatgtatggaggttatgaaggacaagattcttacgaatacccttactcacatgggaaccctaa

SEQ ID NO: 45 - DNA, 16 bp, Artificial Sequence
  Synthetic Polynucleotide
gaggaggtaaatatat

SEQ ID NO: 46 - DNA, 35 bp, Artificial Sequence
  Synthetic Polynucleotide
ataattttgattaactttagaggaggtaaatatat

SEQ ID NO: 47 - DNA, 16 bp, Artificial Sequence
  Synthetic Polynucleotide
aaggagataaatatat

SEQ ID NO: 48 - DNA, 13 bp, Artificial Sequence
  Synthetic Polynucleotide
gaggaggtaaata

SEQ ID NO: 49 - DNA, 13 bp, Artificial Sequence
  Synthetic Polynucleotide
aaggagataaata

SEQ ID NO: 50 - DNA, 32 bp, Artificial Sequence
  Synthetic Polynucleotide
ataattttgattaactttagaggaggtaaata

SEQ ID NO: 51 - DNA, 35 bp, Artificial Sequence
  Synthetic Polynucleotide
ataattttgattaactttaaaggagataaatatat

SEQ ID NO: 52 - DNA, 32 bp, Artificial Sequence
  Synthetic Polynucleotide
ataattttgattaactttaaaggagataaata

SEQ ID NO: 53 - DNA, 519 bp, Artificial Sequence
  Synthetic Polynucleotide
atggtttttacactagaggattttgtcggggattggcgtcaaactgccggatacaacttagatcaagtgttagaacagggtggagtaagtagtcttttccaaaacttaggtgtgtcagtaactcctattcaacggattgttttatctggagagaacggtttgaaaattgatattcacgtgataattccgtacgaaggattaagcggagatcagatggggcaaattgagaaaatctttaaagtagtatacccagttgatgaccatcatttcaaagtgattttacattacggaactctagtaattgacggtgtgaccccaaatatgattgactattttggccgtccatacgaaggaatagctgtctttgacggtaaaaaaattacagtaactggaacattatggaacggaaacaaaatcattgacgagcgtttaatcaatccggatggctctttactctttcgcgtgacgattaacggagtgacaggttggcgtttgtgtgagcgtattcttgcctaatga

SEQ ID NO: 54 - DNA, 22 bp, Artificial Sequence
  Synthetic Polynucleotide
taataagaggaggtaaatatat

SEQ ID NO: 55 - DNA, 40 bp, Artificial Sequence
  Synthetic Polynucleotide
ttacgccaagcttggctgcaacgtgagttcctagacgacc

SEQ ID NO: 56 - DNA, 47 bp, Artificial Sequence
  Synthetic Polynucleotide
atatttacctcctcttattattagttgctatgaacgttttttacagg

SEQ ID NO: 57 - DNA, 45 bp, Artificial Sequence
  Synthetic Polynucleotide
ataagaggaggtaaatatatatggtattcacattagaagattttg

SEQ ID NO: 58 - DNA, 44 bp, Artificial Sequence
  Synthetic Polynucleotide
attcaattatcctataattattatgctagaatacgttcacataa

SEQ ID NO: 59 - DNA, 46 bp, Artificial Sequence
  Synthetic Polynucleotide
gtgaacgtattctagcataataattataggataattgaataaaaac

SEQ ID NO: 60 - DNA, 40 bp, Artificial Sequence
  Synthetic Polynucleotide
acgacggccagtgaattccctcgtggtgttctgactcccg

SEQ ID NO: 61 - DNA, 500 bp, Artificial Sequence
  Synthetic Polynucleotide
taattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaagaacctaagaaagaagaacctaagaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaatgatataattgaagaactaaagagagggtaatgtataatgtatggaggttatga

SEQ ID NO: 62 - DNA, 500 bp, Artificial Sequence; identical to SEQ ID NO: 61
  Synthetic Polynucleotide

SEQ ID NO: 63 - DNA, 500 bp, Artificial Sequence
  Synthetic Polynucleotide
taattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttcgaggattcgaatatattaagacagaacagaaaacaaaagaagaacctaagaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaacgatataattgaagaactaaagagagggtaatgtataatgtatggaggttatgaaggacaagattcttacgaatacccttactcacatgggaaccctaa

SEQ ID NO: 64 - DNA, 42 bp, Artificial Sequence
  Synthetic Polynucleotide
taataagaggaggtaaatatatatggtattcacattggaaga

SEQ ID NO: 65 - DNA, 40 bp, Artificial Sequence
  Synthetic Polynucleotide
attcaattatcctataattattaggctaaaatgcgctcgc

SEQ ID NO: 66 - DNA, 40 bp, Artificial Sequence
  Synthetic Polynucleotide
gcgagcgcattttagcctaataattataggataattgaat

SEQ ID NO: 67 - DNA, 16 bp, Artificial Sequence
  Synthetic Polynucleotide
gaggaggtaaatatat

SEQ ID NO: 68 - DNA, 56 bp, Artificial Sequence
  Synthetic Polynucleotide
atgtttttggcgtcttccatatatatttacctcctcttagttgctatgaacgtttt

SEQ ID NO: 69 - DNA, 56 bp, Artificial Sequence
  Synthetic Polynucleotide
aaaacgttcatagcaactaagaggaggtaaatatatatggaagacgccaaaaacat

SEQ ID NO: 70 - DNA, 40 bp, Artificial Sequence
  Synthetic Polynucleotide
attcaattatcctataattattacaatttggactttccgc

SEQ ID NO: 71 - DNA, 40 bp, Artificial Sequence
  Synthetic Polynucleotide
gcggaaagtccaaattgtaataattataggataattgaat

SEQ ID NO: 72 - DNA, 40 bp, Artificial Sequence
  Synthetic Polynucleotide
acgacggccagtgaattcccagttactaactgctctaatg

SEQ ID NO: 73 - DNA, 40 bp, Artificial Sequence
  Synthetic Polynucleotide
acgacggccagtgaattcccagttactaactgttctaatg

SEQ ID NO: 74 - DNA, 26 bp, Artificial Sequence
  Synthetic Polynucleotide
cctctagctcaaattaacgcatctgt

SEQ ID NO: 75 - DNA, 23 bp, Artificial Sequence
  Synthetic Polynucleotide
tggctctacatgcttagggttcc

SEQ ID NO: 76 - DNA, 44 bp, Artificial Sequence
  Synthetic Polynucleotide
tcttcgagtgtgaagaccatatatatttacctcctcttagttgc

SEQ ID NO: 77 - DNA, 43 bp, Artificial Sequence
  Synthetic Polynucleotide
ctaagaggaggtaaatatatatggtcttcacactcgaagattt

SEQ ID NO: 78 - DNA, 40 bp, Artificial Sequence
  Synthetic Polynucleotide
attcaattatcctataattattacgccagaatgcgttcgc

SEQ ID NO: 79 - DNA, 43 bp, Artificial Sequence
  Synthetic Polynucleotide
gcgaacgcattctggcgtaataattataggataattgaataaa

SEQ ID NO: 80 - DNA, 65 bp, Artificial Sequence
  Synthetic Polynucleotide
aaaacgttcatagcaactaataataagaggaggtaaatatatatggtcttcacactcgaagattt

SEQ ID NO: 81 - DNA, 47 bp, Artificial Sequence; identical to SEQ ID NO: 56
  Synthetic Polynucleotide

SEQ ID NO: 82 - DNA, 24 bp, Artificial Sequence
  Synthetic Polynucleotide
tgctatattataggaacatgggaa

SEQ ID NO: 83 - DNA, 21 bp, Artificial Sequence
  Synthetic Polynucleotide
tgcttacatgccagtaggggt

SEQ ID NO: 84 - DNA, 20 bp, Artificial Sequence
  Synthetic Polynucleotide
gtatgaaggtctgagcggcg

SEQ ID NO: 85 - DNA, 20 bp, Artificial Sequence
  Synthetic Polynucleotide
gatctggcccatttggtcgc

SEQ ID NO: 86 - DNA, 20 bp, Artificial Sequence
  Synthetic Polynucleotide
cgcatagaactgcctgcgtc

SEQ ID NO: 87 - DNA, 20 bp, Artificial Sequence
  Synthetic Polynucleotide
caccccaacatcttcgacgc

SEQ ID NO: 88 - DNA, 20 bp, Artificial Sequence
  Synthetic Polynucleotide
gcgcaactgcaactccgata

SEQ ID NO: 89 - protein, 6 aa, Artificial Sequence
  Synthetic Polypeptide
HHHHHH

SEQ ID NO: 90 - DNA, 76 bp, Artificial Sequence
  Synthetic Polynucleotide
attcaattatcctataattattaatggtgatggtgatgatgacctccacctgctgccgccagaatgcgttcgcaca

SEQ ID NO: 91 - DNA, 46 bp, Artificial Sequence
  Synthetic Polynucleotide
atcatcaccatcaccattaataattataggataattgaataaaaac

SEQ ID NO: 92 - DNA, 67 bp, Artificial Sequence
  Synthetic Polynucleotide
attcaattatcctataattattaatggtgatggtgatgatgtgctgccgccagaatgcgttcgcaca

SEQ ID NO: 93 - DNA, 80 bp, Artificial Sequence
  Synthetic Polynucleotide
taataagaggaggtaaatatatatgcatcatcaccatcaccatggtggaggtgcagcagtcttcacactcgaagatttcg

SEQ ID NO: 94 - DNA, 80 bp, Artificial Sequence
  Synthetic Polynucleotide
agcaactaataataagaggaggtaaatatatatgcatcatcaccatcaccatgcagcagtcttcacactcgaagatttcg

SEQ ID NO: 95 - DNA, 18 bp, Artificial Sequence
  Synthetic Polynucleotide
catcatcaccatcaccat

SEQ ID NO: 96 - protein, 11 aa, Artificial Sequence
  Synthetic Polypeptide
AAGGGHHHHHH

SEQ ID NO: 97 - DNA, 33 bp, Artificial Sequence
  Synthetic Polynucleotide
gcagcaggtggaggtcatcatcaccatcaccat

SEQ ID NO: 98 - protein, 8 aa, Artificial Sequence
  Synthetic Polypeptide
AAHHHHHH

SEQ ID NO: 99 - DNA, 24 bp, Artificial Sequence
  Synthetic Polynucleotide
gcagcacatcatcaccatcaccat

SEQ ID NO: 100 - protein, 11 aa, Artificial Sequence
  Synthetic Polypeptide
HHHHHHGGGAA

SEQ ID NO: 101 - DNA, 33 bp, Artificial Sequence
  Synthetic Polynucleotide
catcatcaccatcaccatggtggaggtgcagca

SEQ ID NO: 102 - protein, 8 aa, Artificial Sequence
  Synthetic Polypeptide
HHHHHHAA

SEQ ID NO: 103 - DNA, 24 bp, Artificial Sequence
  Synthetic Polynucleotide
catcatcaccatcaccatgcagca

SEQ ID NO: 104 - DNA, 42 bp, Artificial Sequence
  Synthetic Polynucleotide
ttacgccaagcttggctgcaatattcgctttgcaccactagc

SEQ ID NO: 105 - DNA, 40 bp, Artificial Sequence
  Synthetic Polynucleotide
atatttacctcctcttattattaggcaggtaaagtaattg

SEQ ID NO: 106 - DNA, 35 bp, Artificial Sequence
  Synthetic Polynucleotide
cgcattttagcctaataaagactaagcccagcttc

SEQ ID NO: 107 - DNA, 38 bp, Artificial Sequence
  Synthetic Polynucleotide
acgacggccagtgaattcccttacctgctggcacgtct

SEQ ID NO: 108 - DNA, 42 bp, Artificial Sequence; identical to SEQ ID NO: 64
  Synthetic Polynucleotide

SEQ ID NO: 109 - DNA, 35 bp, Artificial Sequence
  Synthetic Polynucleotide
tgggcttagtctttattaggctaaaatgcgctcgc

SEQ ID NO: 110 - DNA, 40 bp, Artificial Sequence
  Synthetic Polynucleotide
ttacgccaagcttggctgcaccaaatctcaatgcttactt

SEQ ID NO: 111 - DNA, 47 bp, Artificial Sequence; identical to SEQ ID NO: 56
  Synthetic Polynucleotide

SEQ ID NO: 112 - DNA, 40 bp, Artificial Sequence; identical to SEQ ID NO: 66
  Synthetic Polynucleotide

SEQ ID NO: 113 - DNA, 41 bp, Artificial Sequence
  Synthetic Polynucleotide
acgacggccagtgaattcccaactactaaattctgtttggt

SEQ ID NO: 114 - DNA, 40 bp, Artificial Sequence; identical to SEQ ID NO: 65
  Synthetic Polynucleotide

SEQ ID NO: 115 - DNA, 551 bp, Artificial Sequence
  Synthetic Polynucleotide
ataattttgattaactttaaaggagataaatatatatggtattcacattggaagattttgtgggggattggagacagacagctggatataacttagaccaagtattagaacagggtggagtgtcaagcttatttcaaaacttaggtgtgtcagtgactccaattcaacgtattgtgttaagtggagaaaacggtttaaaaatagacattcatgtgattattccgtacgaaggcctcagtggtgaccaaatgggacaaatagagaaaatctttaaagtagtgtaccctgtggacgaccatcactttaaagtaatcttacactatggtacgttagtaattgatggcgtaacgccaaacatgatagactactttgggcgtccttatgaaggcattgccgtgtttgacggcaaaaagatcaccgtgacaggtactctatggaatggaaacaaaatcattgacgagcgtttaatcaacccagacggctctttactatttcgggtaacaattaacggcgtgaccggatggcgattatgcgagcgcattttagcctaa

SEQ ID NO: 116 - DNA, 42 bp, Artificial Sequence
  Synthetic Polynucleotide
tatttattattaggcaggtaaagtaattgtaacagaagaagc

SEQ ID NO: 117 - DNA, 39 bp, Artificial Sequence
  Synthetic Polynucleotide
actttacctgcctaataataaataattttgattaacttt

SEQ ID NO: 118 - DNA, 812 bp, Artificial Sequence
  Synthetic Polynucleotide
atattcgctttgcaccactagctacaacattcaatcaattacaaggttctgtaggggatacaatcactcttcctaactggaacaaaattggtaaagcagaagttgttgcagaaggtcaaacttctaatattgacactattaaccaatctcaaatctctgtaacagttaaaaaagctgttaaagctgtagctatttcagacgaactagaactagcttccgcaggtaaccctgttaatgaaattgttgaccaaatcgcaatggctttagctcaaaaagtagatgatgatttaattgctattgctcgttctgctaaaaaagctgtagacccttcaactggagaggctcttactgtagatgcaatcaacaaaattcctttagctttagctaactttggtgaaactctttacgaagatgctacatatcttttagtaagcactaacagttatgctttgttcgtttctgatgataagtttgttcctatcattaatcaaggttctatcatcatcaatggtactatcggtactctttatggatgtactgtagttctttctgataaagtacaagacggtgagttcttcttcattaaagctggtgctcttggtattgctcttaaacaagatacacgtattcttactgaatatgatttactttctcacactacattaatcagtggtgaccgccactatgctacatttatggctgatgaagataaaattgtttatgtaggtgcaggcactgctgttccagcggctccaactattgcgactcctgctacaactgcttcttctgttacaattactttacctgcctaa

SEQ ID NO: 119 - DNA, 1088 bp, Artificial Sequence
  Synthetic Polynucleotide
taaagactaagcccagcttcggctgggttataagtctattataatcgaatcggaggaataaatcaatggctgtagaaaagaaatatgatataaagaaaaatggtaacattatcgaaacgttccatgcttcaggtgactttgtggataatgatgtattaccaaaaacaaactaccaataccaagtacgtgtgcgtgaggtagactctggtgtagtaaaatctacctctgagtggactgctcctattaacgtcaaaactaagtaaggggaattagtaaatgccaaccgttaagaaatatgatattaaacgtaacggcaatgtggtagctacatatgttcccgctggaaattggagagatgataatcttcttcctaatactaactaccagtatttagttagatgtcatgaagttacaactactggtagtgacgtcagtattaaaacaagtgattggacaaacccaattaacgttaaaacactggtatctgatactagtattcatccacctgctcctattaatgttaatgtaacagatttagaatctgattctttcactattgattggggtaatggaggatttaatgctaaaacgtatgttgttcgtttaggcattaatggagatggattgccagataataatgtttttgtaactacaactaatagctttacagctactgaggatgtagtagtagcattagctccattcgctggtaagaaaattgacgtatatgtagcacaatttgctggagtttacgcggatgccacttctgctttagaaagtgcagaatacaatgaagtaactgcatggtctaacctagttactgtagatgtcccatctgcaagtccttcaatgttacgttcttctagtaataaagaaattgttagcttaactgtttctcctaaaacgtcttctaaatcttcttccggtgcattaactggtagctttacagttgcagtagaccctacagatgctacagaagcttttgaagttaaggttacagataaagatggtgctgattctagtgctataaaagctattatcgatggcttaaaagttaactatactggaacagacgtgccagcaggtaa

SEQ ID NO: 120 - DNA, 1258 bp, Artificial Sequence
  Synthetic Polynucleotide
ccaaatctcaatgcttacttggacagagaatgatttaacattctataaagacatcgctaaaaaaccagctacatctacagtagcaaaatacgatgtatacatgcaacatggtaaggtaggtcatactagatttactcgtgagattggggtagcaccagtaagtgaccctaacatccgtcaaaaaacagtaaatatgaaatttgcttccgatactaaaaacatcagtatcgcagcaggtctagtaaacaacattcaagacccaatgcaaattttgactgacgatgctatcgtaaatattgctaaaacaattgagtgggcttcattctttggagattctgacttatcagatagcccagaaccacaagcaggactagaatttgacggcttggctaaacttattaaccaagataacgttcatgatgctcgtggagctagcttgactgaaagcttgttaaaccaagcagcagtaatgattagtaaaggttatggtacacctacagatgcttacatgccagtaggggttcaagcagactttgttaaccaacaactttctaaacaaacacaacttgttcgcgataacggaaacaacgtaagcgttggtttcaacatccaaggtttccattcagctcgtggatttatcaaacttcacggttctacagtaatggaaaacgaacaaatcttagatgaacgtattcttgctttaccaacagctccacaaccagctaaggtaactgcaacacaagaagcaggtaaaaaaggacaatttagagcagaagatttagcagcacatgaatataaagttgttgtaagttctgacgatgcagagtctattgcaagtgaagtggctacagctacagttactgcaaaagatgacggcgttaaactagaaatcgaattagctccaatgtatagctctcgtccacaattcgtttcaatctatagaaaaggtgcagaaacaggtttattctacctaatcgctcgtgtacctgctagcaaagcagagaacaacgtaatcactttctacgacttaaacgactctattcctgaaacagtagacgtattcgttggtgaaatgtcggctaacgtagtacacttgtttgaattactaccaatgatgagattacctctagctcaaattaacgcatctgttacatttgcagttttatggtatggcgcattagctctaagagcacctaagaaatgggtacgtattagaaacgttaaatatattcctgtaaaaaacgttcatagcaactaa

SEQ ID NO: 121 - DNA, 763 bp, Artificial Sequence
  Synthetic Polynucleotide
taattataggataattgaataaaaacagtatagagagcagataaatactgctctctattttactaataaggaggatttaaattgctaaaaaatacaaacttagctaattataaaaaagtgaatacacggtttggaaatcttagttttgacgacaaaggtatttctaatgacttaacggaagaacagcaaaaagaattaggtaagcttagaggattcgaatatattaagacagaacagaaaacgaaagaagaacctaagaaagaagaacctaagaaagaagaacctaagaaagaagaacctaagaaagaaagtacagaaaatgaattagacagcttcttagctaaagagccttcaatcaaagaattaaaagaatttgcgagtaaaaaaggcattaaaattgaaaaaactaagaaaaatgatataattgaagaactaaagagagggtaatgtataatgtatggaggttatgaaggacaagattcttacgaatacccttactcacatgggaaccctaagcatgtagagccagaaaaagttgacgaatatgttctttctgattatggttggactgcggaaacaattaaagcatacatgtatggtgttcgtgtagtagaccctgaaacaggagaggaaatgggagacaccttctacaatcatattatagaggttgccgttgataaggcagagaaagaactagatatagctattctcccaagacgggagtcagaacaccacgattataaccaaacagaatttagtagtt

SEQ ID NO: 122 - DNA, 812 bp, Artificial Sequence; identical to SEQ ID NO: 118
  Synthetic Polynucleotide

SEQ ID NO: 123 - DNA, 1088 bp, Artificial Sequence; identical to SEQ ID NO: 119
  Synthetic Polynucleotide

SEQ ID NO: 124 - DNA, 1404 bp, Artificial Sequence; identical to SEQ ID NO: 17
  Synthetic Polynucleotide

SEQ ID NO: 125 - DNA, 1404 bp, Artificial Sequence; identical to SEQ ID NO: 17
  Synthetic Polynucleotide

SEQ ID NO: 126 - DNA, 1407 bp, Artificial Sequence; identical to SEQ ID NO: 7
  Synthetic Polynucleotide

SEQ ID NO: 127 - DNA, 1407 bp, Artificial Sequence; identical to SEQ ID NO: 7
  Synthetic Polynucleotide

SEQ ID NO: 128 - DNA, 1407 bp, Artificial Sequence; identical to SEQ ID NO: 7
  Synthetic Polynucleotide

SEQ ID NO: 129 - DNA, 1407 bp, Artificial Sequence; identical to SEQ ID NO: 7
  Synthetic Polynucleotide

SEQ ID NO: 130 - DNA, 1407 bp, Artificial Sequence; identical to SEQ ID NO: 15
  Synthetic Polynucleotide

SEQ ID NO: 131 - DNA, 1407 bp, Artificial Sequence; identical to SEQ ID NO: 21
  Synthetic Polynucleotide

SEQ ID NO: 132 - DNA, 1407 bp, Artificial Sequence; identical to SEQ ID NO: 5
  Synthetic Polynucleotide

SEQ ID NO: 133 - protein, 467 aa, Artificial Sequence; identical to SEQ ID NO: 18
  Synthetic Polypeptide

SEQ ID NO: 134 - protein, 467 aa, Artificial Sequence; identical to SEQ ID NO: 18
  Synthetic Polypeptide

SEQ ID NO: 135 - protein, 468 aa, Artificial Sequence; identical to SEQ ID NO: 6
  Synthetic Polypeptide

SEQ ID NO: 136 - protein, 468 aa, Artificial Sequence; identical to SEQ ID NO: 6
  Synthetic Polypeptide

SEQ ID NO: 137 - protein, 468 aa, Artificial Sequence; identical to SEQ ID NO: 6
  Synthetic Polypeptide

SEQ ID NO: 138 - protein, 468 aa, Artificial Sequence; identical to SEQ ID NO: 6
  Synthetic Polypeptide

SEQ ID NO: 139 - protein, 468 aa, Artificial Sequence; identical to SEQ ID NO: 6
  Synthetic Polypeptide

SEQ ID NO: 140 - protein, 468 aa, Artificial Sequence; identical to SEQ ID NO: 6
  Synthetic Polypeptide

SEQ ID NO: 141 - protein, 468 aa, Artificial Sequence; identical to SEQ ID NO: 6
  Synthetic Polypeptide

SEQ ID NO: 142 - DNA, 3786 bp, Artificial Sequence; identical to SEQ ID NO: 28
  Synthetic Polynucleotide

SEQ ID NO: 143 - DNA, 3786 bp, Artificial Sequence; identical to SEQ ID NO: 28
  Synthetic Polynucleotide

SEQ ID NO: 144 - DNA, 3789 bp, Artificial Sequence; identical to SEQ ID NO: 24
  Synthetic Polynucleotide

SEQ ID NO: 145 - DNA, 3789 bp, Artificial Sequence; identical to SEQ ID NO: 24
  Synthetic Polynucleotide

SEQ ID NO: 146 - DNA, 3789 bp, Artificial Sequence; identical to SEQ ID NO: 24
  Synthetic Polynucleotide

SEQ ID NO: 147 - DNA, 3729 bp, Artificial Sequence; identical to SEQ ID NO: 23
  Synthetic Polynucleotide

SEQ ID NO: 148 - DNA, 3729 bp, Artificial Sequence; identical to SEQ ID NO: 27
  Synthetic Polynucleotide

SEQ ID NO: 149 - DNA, 3729 bp, Artificial Sequence; identical to SEQ ID NO: 30
  Synthetic Polynucleotide

SEQ ID NO: 150 - DNA, 2649 bp, Artificial Sequence; identical to SEQ ID NO: 33
  Synthetic Polynucleotide

SEQ ID NO: 151 - DNA, 2658 bp, Artificial Sequence; identical to SEQ ID NO: 31
  Synthetic Polynucleotide

SEQ ID NO: 152 - DNA, 2598 bp, Artificial Sequence; identical to SEQ ID NO: 32
  Synthetic Polynucleotide

SEQ ID NO: 153 - DNA, 2592 bp, Artificial Sequence; identical to SEQ ID NO: 34
  Synthetic Polynucleotide

SEQ ID NO: 154 - DNA, 2613 bp, Artificial Sequence; identical to SEQ ID NO: 35
  Synthetic Polynucleotide

(19) United States
(12) Patent Application Publication
(10) Pub. No.: US 2018/0274004 A1
(43) Pub. Date: Sep. 27, 2018

(54) RECOMBINANT PHAGE AND METHODS OF DETECTING LISTERIA

(71) Applicant: Institute for Environmental Health, Inc., Lake Forest Park, WA (US)

(72) Inventors: Michael Sandor Koeris, Natick, MA (US); Michael Cappillino, Reading, MA (US); Edyta Krzymanska-Olejnik, Brookline, MA (US); Daniel Robert Brownell, Arlington, MA (US); Robert Patrick Shivers, Watertown, MA (US); Jayson L. Bowers, Cambridge, MA (US); Timothy Kuan-Ta Lu, Charlestown, MA (US)

(21) Appl. No.: 15/867,555

(22) Filed: Jan. 10, 2018

Related U.S. Application Data
(63) Continuation of application No. PCT/US2016/041529, filed on Jul. 8, 2016.
Application No. PCT/US2016/041529 is a continuation-in-part of application No. 14/707,847, filed on May 8, 2015, now Pat. No. 10,039,795.
(60) Provisional application No. 62/191,229, filed on Jul. 10, 2015.
(60) Provisional application No. 62/191,867, filed on Jul. 13, 2015.
(60) Provisional application No. 61/991,132, filed on May 9, 2014.
(60) Provisional application No. 62/044,082, filed on Aug. 29, 2014.
(60) Provisional application No. 62/053,481, filed on Sep. 22, 2014.
(60) Provisional application No. 62/086,445, filed on Dec. 2, 2014.

(51) Int. Cl. C12Q 1/04; C12N 7/00

(52) U.S. Cl. CPC C12Q 1/04; C12N 7/00

(57) ABSTRACT

Composition and methods for the detection of one or more target microbe(s) are provided. Compositions of the disclosure include at least one recombinant phage capable of infecting a target microbe, said phage comprising at least a capsid protein sequence, a ribosome binding site, and a codon-optimized marker. Compositions of the disclosure may further include an aqueous solution that enhances the ability to detect marker expression upon phage infection of the target microbe. In some embodiments the target microbe is Listeria.

### RELATED APPLICATIONS

This application is a continuation of International Patent Application No. PCT/US2016/041529, filed Jul. 8, 2016, which claims the benefit of U.S. Provisional Patent Application No. 62/191,229, filed Jul. 10, 2015, and U.S. Provisional Patent Application No. 62/191,867, filed Jul. 13, 2015, and this application is also a continuation-in-part of U.S. patent application Ser. No. 14/707,847, filed May 8, 2015, which claims the benefit of U.S. Provisional Patent Application No. 61/991,132, filed May 9, 2014, U.S. Provisional Patent Application No. 62/044,082, filed Aug. 29, 2014, U.S. Provisional Patent Application No. 62/053,481, filed Sep. 22, 2014, and U.S. Provisional Patent Application No. 62/086,445, filed Dec. 2, 2014, the disclosures of which are each herein incorporated by reference in their entirety.

### INCORPORATION OF SEQUENCE LISTING

The contents of the text file named “0066090-025US0 Sequence Listing.txt,” which was created on Jan. 10, 2018, and is 283 KB in size, are hereby incorporated by reference in their entirety.

### FIELD OF THE DISCLOSURE

This disclosure generally relates to the detection of microbes through the use of codon-optimized recombinant phage.

### BACKGROUND

Bacterial contamination and infection is a significant problem to public health and in many other areas. Bacterial food borne diseases pose a significant threat to human health, estimated to cause as many as about 76 million illnesses, 325,000 hospitalizations, and 5,000 deaths in the US annually.

For example, in 1996, juice that was contaminated with Escherichia coli was released into the public by a juice maker and resulted in one death and 66 illnesses. The company paid a $1.5 million fine, and the recall alone cost the company $6.5 million. In 2006, an E. coli O157:H7 outbreak from contaminated spinach originating from California resulted in 205 illnesses and 3 deaths. In 2011 a listeriosis outbreak from cantaloupes from Colorado in July, August and September resulted in 30 deaths. That is the second deadliest recorded U.S. outbreak in terms of the number of deaths since the Centers for Disease Control and Prevention began tracking outbreaks in the 1970s. Another recall of cantaloupes in 2012 suggests that the food supply is still not safe and highlights the general and pervasive need for additional methods and reagents for testing the food supply to identify contamination.

Another example is bovine mastitis, an infection caused by bacterial cells that results in the inflammation of the bovine breast, reduction in milk yield and a decrease in milk quality. This condition is caused by the bacteria Staphylococcus aureus and Staphylococcus agalactiae. This reduction in milk yields and quality in the western world alone have been suggested to cause annual financial losses of $3.7 billion.

Another example is bovine tuberculosis (Mycobacterium bovis), a bacteria that causes financial loses worldwide. In 2005, for example, 12 of a herd of 55 cattle in a small Michigan farm tested positive for bovine tuberculosis. The farm was forced to destroy the entire herd of cattle, along with an entire herd of hogs. Tuberculosis testing in cattle requires the animal to be held for 2 days, and tests are false positive 5 percent of the time. Often entire herds have to be quarantined or destroyed. The annual worldwide financial losses have been estimated at $3 billion.

Tuberculosis is a leading cause of death worldwide. One third of the world's population is infected with Mycobacterium tuberculosis, the bacterium that causes tuberculosis. Every day 25,000 people are infected and 5,000 people die from the disease. Furthermore, due primarily to poor diagnosis, multidrug resistant strains of M. tuberculosis are emerging and the reemergence of tuberculosis as a worldwide epidemic has become a real threat. The worldwide annual market for tuberculosis diagnostics has been estimated at $1.8 billion.

MRSA is a drug-resistant version of the common Staphylococcus aureus bacteria and is contagious, due to the nature of the MRSA bacterium. The bacteria are highly contagious and spread by touch. Approximately 86% of all infections occur within hospitals, and these infections carry a 20% mortality rate. This bacterium costs an average of $21,000 over the standard costs to treat, and kills approximately 19,000 people in the US annually.

Listeria monocytogenes is an intracellular pathogen that can cause invasive disease in humans and animals. Approximately 99% of human listeriosis infections appear to be food borne. While L. monocytogenes has been isolated from a variety of raw and ready-to-eat foods, most human listeriosis infections appear to be caused by consumption of RTE foods that permit postcontamination growth of this pathogen. Listeriosis is estimated to be responsible for about 500 deaths per year in the United States, accounting for 28% of annual deaths attributable to known food-borne patho-gens, second only to deaths due to Salmonella infections.

Methods and systems exist for detecting microbial contamination. Such methods and systems suffer from a number of drawbacks, including the need in most cases to remove a potentially contaminated sample from the environment where it is collected and transferring it to a laboratory environment, where the sample is placed in a culture environment for enrichment and growth over a long period of time, ranging from many hours to days. Additionally, because these labs are frequently offsite there is often a delay in the shipping of a sample to a laboratory. Once enriched, samples are typically analyzed using expensive equipment, traditional culturing methods, PCR and other methods. Thus, current processes often comprise a large time lag between sampling and a result, during which time the sampled conditions may have changed and the results of the assay cannot be utilized to diagnose an infection in a patient or to act on contamination in a lot of manufactured food, for example. Accordingly, new composition, methods, and kits for detecting microbial contamination are needed. Compositions and methods of the present disclosure address these needs.

### SUMMARY

Compositions and methods of the disclosure address the long-felt need in the art for compositions and methods of immediate detection of bacterial infection by a non-technical or layperson at the site of potential contamination.

The disclosure provides a composition comprising, consisting essentially of or consisting of at least one recombinant phage capable of infecting a target microbe, said phage comprising at least a capsid protein sequence, a ribosome binding site, and a codon-optimized marker. Compositions of the disclosure may comprise, consist essentially of or consist of at least two, three, four, five, or six recombinant phages capable of infecting a target microbe, each of said phage comprising at least a capsid protein sequence, a ribosome binding site, and a codon-optimized marker. Compositions of the disclosure may comprise, consist essentially of or consist of greater than six recombinant phage capable of infecting a target microbe, each of said phage comprising at least a capsid protein sequence, a ribosome binding site, and a codon-optimized marker.

The ribosome binding site of each phage may be identical in compositions of the disclosure comprising, consisting essentially of or consisting of one or more recombinant phage capable of infecting a target microbe, said phage comprising at least a capsid protein sequence, a ribosome binding site, and a codon-optimized marker. Exemplary ribosome binding sites of the phage of the disclosure may comprise, consist essentially of or consist of SEQ ID NO: 54.

Codon-optimized markers of the disclosure may comprise, consist essentially of or consist of a codon-optimized luciferase. Exemplary codon-optimized markers of the disclosure may comprise, consist essentially of or consist of SEQ ID NO: 36 (COP2). Alternatively, codon-optimized markers of the disclosure may comprise, consist essentially of or consist of SEQ ID NO: 37 (COP3) or a UTR7 variant of COP3 (SEQ ID NO: 115). In certain embodiments of the codon-optimized markers of the disclosure, codon-optimized markers of the disclosure may comprise, consist essentially of or consist of SEQ ID NO: 37 (COP3).

Compositions of the disclosure may comprise, consist essentially of or consist of at least one recombinant phage is selected from the group consisting of LP173, LP80, V18, LP22, LP143, A511, LP101, LP124, LP99, LP48, LP125, P100, and LP40. Compositions of the disclosure may comprise, consist essentially of or consist of at least one recombinant phage, wherein the phage is LP80, V18, LP22, A511, LP40 or LP124. Compositions of the disclosure may comprise, consist essentially of or consist of LP80, V18, LP22, A511, LP40 and LP124. Compositions of the disclosure may comprise, consist essentially of or consist of at least one recombinant phage, wherein the phage is A511. Compositions of the disclosure may comprise, consist essentially of or consist of at least one recombinant phage, wherein the phage is LP40. Compositions of the disclosure may comprise, consist essentially of or consist of at least one recombinant phage, wherein the phage is LP124. Compositions of the disclosure may comprise, consist essentially of or consist of at least one recombinant phage, wherein the phage is LP80. Compositions of the disclosure may comprise, consist essentially of or consist of at least one recombinant phage, wherein the phage is V18. Compositions of the disclosure may comprise, consist essentially of or consist of at least one recombinant phage, wherein the phage is LP22.

Target microbes of the disclosure may belong to the genus Listeria. Exemplary target microbes of the disclosure include, but are not limited to, Listeria innocua, Listeria monocytogenes, Listeria seeligeri, Listeria ivanovii, Listeria grayi, Listeria marthii, Listeria rocourti, Listeria welshimeri, Listeria floridensis, Listeria aquatic, Listeria cornellensis, Listeria riparia, Listeria weihenstephanensis, Listeria flieschmannii, Listeria newyorkensis and Listeria grandensis. In certain embodiments of the compositions and methods of the disclosure, the target microbe is Listeria monocytogenes.

Compositions of the disclosure may comprise, consist essentially of, or consist of a recombinant phage of the disclosure and an aqueous solution. Aqueous solutions of the disclosure may comprise, consist essentially of or consist of: a) at least one nutrient; b) at least one selective agent suitable to inhibit growth of at least one non-target microbe in an environmental sample or an agricultural sample; c) at least one vitamin; d) at least one divalent metal; and e) at least one buffering agent capable of maintaining the composition at pH 7.0-7.5.

Aqueous solutions of the disclosure may comprise, consist essentially of or consist of: a) at least one nutrient; b) at least one selective agent suitable to inhibit growth of at least one non-target microbe in an environmental sample or an agricultural sample; c) at least one vitamin; d) at least one divalent metal; e) at least one buffering agent capable of maintaining the composition at pH 7.0-7.5 and at least one agent to prevent the decomposition of a marker substrate. Exemplary agents to prevent the decomposition of a marker substrate may comprise, consist essentially of or consist of a compound to prevent the decomposition of luciferin. In certain embodiments of the aqueous solutions of the disclosure, the compound to prevent the decomposition of luciferin prevents decomposition of luciferin for between 5 and 10 hours. In certain embodiments of the aqueous solutions of the disclosure, the compound to prevent the decomposition of luciferin prevents decomposition of luciferin for less than 5 hours. In certain embodiments of the aqueous solutions of the disclosure, the compound to prevent the decomposition of luciferin prevents decomposition of luciferin for greater than 10 hours. Exemplary agents to prevent decomposition of the luciferin may comprise, consist essentially of or consist of non-ionic detergents, oxygen scavengers and/or emulsifiers. Exemplary agents to prevent decomposition of the luciferin may comprise, consist essentially of or consist of sodium metabisulfite, sodium thiosulfate, Tween-80, HEPES and/or lecithin.

Aqueous solutions of the disclosure may comprise, consist essentially of or consist of: a) at least one nutrient; b) at least one selective agent suitable to inhibit growth of at least one non-target microbe in an environmental sample or an agricultural sample; c) at least one vitamin; d) at least one divalent metal; e) at least one buffering agent capable of maintaining the composition at pH 7.0-7.5 and at least one agent suitable to neutralize a sanitizer present in an environmental sample. Aqueous solutions of the disclosure may comprise, consist essentially of or consist of: a) at least one nutrient; b) at least one selective agent suitable to inhibit growth of at least one non-target microbe in an environmental sample or an agricultural sample; c) at least one vitamin; d) at least one divalent metal; e) at least one buffering agent capable of maintaining the composition at pH 7.0-7.5, at least one agent to prevent the decomposition of a marker substrate and at least one agent suitable to neutralize a sanitizer present in an environmental sample. Exemplary agents suitable to neutralize a sanitizer may comprise, consist essentially of or consist of sodium metabisulfite, sodium pyruvate, sodium thiosulfate, Tween-80, HEPES and lecithin. In certain embodiments, an agent suitable to neutralize a sanitizer may comprise, consist essentially of or consist of sodium pyruvate. In certain embodiments, an agent suitable to neutralize a sanitizer may comprise, consist essentially of or consist of sodium pyruvate, wherein the sodium pyruvate is 1% or less of the aqueous solution.

Aqueous solutions of the disclosure may comprise, consist essentially of or consist of: a) at least one nutrient; b) at least one selective agent suitable to inhibit growth of at least one non-target microbe in an environmental sample or an agricultural sample; c) at least one vitamin; d) at least one divalent metal; and e) at least one buffering agent capable of maintaining the composition at pH 7.0-7.5. Exemplary nutrients include, but are not limited to, a culture medium, alcohol, sugar, sugar derivatives, and combinations thereof. Alternatively, or in addition, Exemplary nutrients include, but are not limited to, Brain Heart Infusion medium, Tryptic Soy Broth, glucose, glycerol, pyruvate, and combinations thereof.

Aqueous solutions of the disclosure may comprise, consist essentially of or consist of: a) at least one nutrient; b) at least one selective agent suitable to inhibit growth of at least one non-target microbe in an environmental sample or an agricultural sample; c) at least one vitamin; d) at least one divalent metal; and e) at least one buffering agent capable of maintaining the composition at pH 7.0-7.5. Exemplary selective agents suitable to inhibit growth of a non-target microbe include, but are not limited to, LiCl, acriflavine, nalidixic acid, cycloheximide, and combinations thereof.

Aqueous solutions of the disclosure may comprise, consist essentially of or consist of: a) at least one nutrient; b) at least one selective agent suitable to inhibit growth of at least one non-target microbe in an environmental sample or an agricultural sample; c) at least one vitamin; d) at least one divalent metal; and e) at least one buffering agent capable of maintaining the composition at pH 7.0-7.5. In certain embodiments, the at least one vitamin comprises, consists essentially of or consist of a yeast extract.

Aqueous solutions of the disclosure may comprise, consist essentially of or consist of: a) at least one nutrient; b) at least one selective agent suitable to inhibit growth of at least one non-target microbe in an environmental sample or an agricultural sample; c) at least one vitamin; d) at least one divalent metal; and e) at least one buffering agent capable of maintaining the composition at pH 7.0-7.5. Exemplary divalent methods include, but are not limited to, CaCl₂, MgSO4, and combinations thereof.

Aqueous solutions of the disclosure may comprise, consist essentially of or consist of: a) at least one nutrient; b) at least one selective agent suitable to inhibit growth of at least one non-target microbe in an environmental sample or an agricultural sample; c) at least one vitamin; d) at least one divalent metal; and e) at least one buffering agent capable of maintaining the composition at pH 7.0-7.5. In certain embodiments, the at least one buffering agent comprises HEPES buffer.

Aqueous solutions of the disclosure may comprise, consist essentially of or consist of Tryptic Soy Broth, LiCl, nalidixic acid, yeast extract, glucose, MgSO₄, pyruvate, and HEPES.

Aqueous solutions of the disclosure may comprise, consist essentially of or consist of Tryptic Soy Broth, LiCl, nalidixic acid, yeast extract, glucose, MgSO₄, pyruvate, HEPES, Tween-80, lecithin, and potassium phosphate.

Compositions of the disclosure may comprise, consist essentially of or consist of a recombinant phage of the disclosure, an aqueous solution, and a substrate for luciferase. The substrate for luciferase may comprise, consist essentially of or consist of luciferin.

Compositions of the disclosure may comprise, consist essentially of or consist of a recombinant phage of the disclosure, an aqueous solution, a substrate for luciferase, and a buffer to facilitate a light reaction. In certain embodiments, the buffer to facilitate a light reaction may comprise, consist essentially of or consist of at least one agent suitable to neutralize a sanitizer. Exemplary agents suitable to neutralize a sanitizer may comprise, consist essentially of or consist of sodium pyruvate. In certain embodiments, sodium pyruvate may comprise 1% or less of the buffer to facilitate a light reaction.

The disclosure provides a method of determining the presence or absence of a target microbe in an environmental sample, an agricultural sample or both, comprising: a) contacting an environmental sample, an agricultural sample, or both with a composition of the disclosure to form a test sample; and b) detecting the presence or absence of light in the test sample, thereby determining the presence or absence of a target microbe in an environmental sample or an agricultural sample.

Methods of determining the presence or absence of a target microbe in an environmental sample, an agricultural sample or both may further comprise the step of incubating the test sample at a temperature between 30° C. and 35° C., inclusive of the endpoints, prior to the detecting step. In certain embodiments, the test sample is incubated at about 35° C. In certain embodiments, the test sample is incubated at 35° C.

Methods of determining the presence or absence of a target microbe in an environmental sample, an agricultural sample or both may further comprise the step of centrifuging the test sample prior to the detecting step. The centrifugation step may be performed at a speed of between 1000 and 14000 rcf. The centrifugation step may be performed at a speed of about 1000 rcf, 3000 rcf, 5000 rcf, 9000 rcf, 14000 rcf or any rcf value in between. The centrifugation step may be performed at a speed of about 9000 rcf. The centrifugation step may be performed at a speed of 9000 rcf.

According to methods of determining the presence or absence of a target microbe in an environmental sample, an agricultural sample or both of the disclosure, the test sample may have a volume of at least 300 μl at the time the presence or absence of light is detected. The test sample may have a volume of about 600 μl at the time the presence or absence of light is detected. The test sample may have a volume of 600 μl at the time the presence or absence of light is detected.

Methods of determining the presence or absence of a target microbe in an environmental sample, an agricultural sample or both may further comprise the step of confirming a positive result of the detecting step. The confirming step may comprise contacting the detected test sample with a confirmation composition, and wherein a decrease in an abundance or intensity of light confirms that the positive result is a true result. Exemplary confirmation compositions may comprise, consist essentially of or consist of an organic solvent. The organic solvent may comprise, consist essentially of or consist of acetone or ethanol. The organic solvent may comprise, consist essentially of or consist of ethanol. The organic solvent may comprise, consist essentially of or consist of 70% ethanol.

Methods of determining the presence or absence of a target microbe in an environmental sample, an agricultural sample or both may further comprise the step of collecting the environmental sample, the agricultural sample, or both prior to the contacting step. The step of collecting may comprise contacting a sponge to a portion or a surface of the environmental sample and/or the agricultural sample to form a test sponge and subsequently contacting the test sponge to the composition. Exemplary sponges may comprise, consist essentially of or consist of polyurethane.

Environmental samples of the disclosure include, but are not limited to, an agricultural production facility, a food production facility, a container, a machine, a processing plant, a storage facility, a health care facility, an educational institution, a loading dock, a cargo hold, a sink, a vehicle, an airport, a customs facility or any portion or surface thereof.

Environmental samples of the disclosure may include a health care facility, portion or surface thereof, or sample isolated from a health care facility. Exemplary health care facility include, but are not limited to, a clinic, an emergency medical services location, a hospice, a hospital ship, a hospital train, a hospital, a military medical installation, a doctor's office, a long term care facility, respite care facility, or a quarantine station.

Environmental samples of the disclosure may include a food production facility, portion or surface thereof, or sample isolated from a food production facility. Exemplary food production facilities include, but are not limited to, a farm, a boat, a food distribution facility, a food processing plant, a food retail location, a home, or a restaurant.

Agricultural samples of the disclosure include, but are not limited to, stock feed or food supply. The food supply may be intended for human or non-human (animal) consumption. The food supply may include plant or animal matter. The food supply may be solid or liquid.

In certain embodiments, the food supply comprises, consists essentially of or consists of a dairy product, a fruit product, a grain product, a sweet, a vegetable product, a meat product, or any combination thereof. Exemplary dairy products include, but are not limited to, milk, butter, yogurt, cheese, ice cream, queso fresco, a derivative thereof or any combination thereof. Exemplary fruit products include, but are not limited to, an apple, orange, banana, berry, lemon, or any combination thereof. Exemplary grain products include, but are not limited to, wheat, rice, oats, barley, bread, pasta, or any combination thereof. Exemplary sweet products include, but are not limited to, candy, soft drinks, cake, pie, or a combination thereof. Exemplary vegetable products include, but are not limited to, spinach, carrots, onions, peppers, avocado, broccoli, or any combination thereof. The vegetable product may comprise, consist essentially of or consist of guacamole. Exemplary meat products include, but are not limited to, chicken, fish, turkey, pork, beef, or any combination thereof. In certain embodiments, the meat product comprises, consists essentially of or consists of whole muscle meat, ground meat, or a combination thereof.

The disclosure provides a kit comprising a composition of the disclosure. In certain embodiments, the kit further comprises a confirmation composition. The confirmation composition may comprise, consist essentially of or consist of an organic solvent. Exemplary organic solvents include, but are not limited to, acetone or ethanol. The organic solvent may comprise, consist essentially of or consist of ethanol. The organic solvent may comprise, consist essentially of or consist of 70% ethanol. In certain embodiments, the kit may further comprise a polyurethane sponge.

The disclosure provides a kit comprising: a first container comprising at least one recombinant phage capable of infecting a target microbe, said phage comprising at least a capsid protein sequence, a ribosome binding site, and a codon-optimized marker; a second container comprising an aqueous solution composition comprising Tryptic Soy Broth, LiCl, nalidixic acid, yeast extract, glucose, MgSO₄, pyruvate, HEPES, Tween-80, lecithin, and potassium phosphate; a third container containing a substrate; and a fourth container containing a buffer to optimize light detection.

The disclosure provides a kit comprising: a first container comprising at least one recombinant phage capable of infecting a target microbe, said phage comprising at least a capsid protein sequence, a ribosome binding site, and a codon-optimized marker and an aqueous solution composition; a second container comprising an aqueous solution composition comprising Tryptic Soy Broth, LiCl, nalidixic acid, yeast extract, glucose, MgSO4, pyruvate, HEPES, Tween-80, lecithin, and potassium phosphate; a third container containing a substrate; a fourth container containing a buffer to optimize light detection; and a fifth container comprising a confirmation composition. The confirmation composition may comprise, consist essentially of or consist of an organic solvent. Exemplary organic solvents include, but are not limited to, acetone or ethanol. The organic solvent may comprise, consist essentially of or consist of ethanol. The organic solvent may comprise, consist essentially of or consist of 70% ethanol. In certain embodiments, the kit may further comprise a polyurethane sponge.

Kits of the disclosure may contain an aqueous solution that may comprise, consist essentially of or consist of Tryptic Soy Broth, LiCl, nalidixic acid, yeast extract, glucose, MgSO₄, pyruvate, HEPES, Tween-80, lecithin, and potassium phosphate.

Kits of the disclosure may contain buffer to optimize light detection that may comprise, consist essentially of or consist of Tween 80, lecithin, and HK₂PO₄ at pH 7.4. Kits of the disclosure may contain buffer to optimize light detection that may comprise, consist essentially of or consist of 28% Tween 80, 4% lecithin, and HK₂PO₄ at pH 7.4.

The disclosure provides a method of making a recombinant phage capable of infecting a target microbe, said phage comprising at least a capsid protein sequence, a ribosome binding site, and a codon-optimized marker, comprising (a) inserting into a phage targeting vector (PTV), a nucleic acid sequence encoding the capsid protein sequence, a nucleic acid sequence encoding a ribosome binding site, and a nucleic acid sequence encoding a codon-optimized marker, (b) transforming the PTV of (a) into a phage host cell, and (c) incubating the phage host cell of (b) with a starting phage, thereby generating a recombinant phage capable of infecting a target microbe. In certain embodiments of this method, the at least one of the nucleic acid sequence encoding the capsid protein sequence, the nucleic acid sequence encoding a ribosome binding site, and the nucleic acid sequence encoding a codon-optimized marker are a heterologous nucleic acid sequence. In certain embodiments of this method, the nucleic acid sequence encoding the capsid protein sequence, the nucleic acid sequence encoding a ribosome binding site, and the nucleic acid sequence encoding a codon-optimized marker are each a heterologous nucleic acid sequence. In certain embodiments of this method, a contiguous nucleic acid molecule comprises the nucleic acid sequence encoding the capsid protein sequence, the nucleic acid sequence encoding a ribosome binding site, and the nucleic acid sequence encoding a codon-optimized marker.

Host cells of the methods of the disclosure may be isolated and/or derived from a strain selected from the group consisting of 1816, 1817, 1823, 1825, 1826, 1828, 1832, 1836, 1883, 1886, 1890, 1892, 1893, 1894, 1899, 1900, 1907, 1909, 1912, 1916, 1951, 1962, 1978, 1979, 1981, 1990, 1991, 1992, 1993, 1994, 1995, 2006, 2010, 2011, 2012, 2013, 2067, 2071, 2080, 2081, 2082, 2085, 2087, 2089, 2100, 2101, 2102, 2103, 2104, 2105, 2107, 2108, 2110, 2112, 2134, 2136, 2137, 2138, B4-G7, B5-E10, B6-G7, B7-A10, B7-F6, B9-G4, BG-G10, 085-018-02 1, 085-018-02 2, 085-018-02 3, 088-013 02 S1, 088-013 02 S3, 112-009-08 1, 112-009-08 2, 112-009-08 3, 112-010-02 1, 112-010-02 1 F, 112-010-02 1 L, 112-010-02 2, 112-010-02 2 F, 112-010-02 2 L, 112-010-02 3, 112-010-02 3 F, 112-010-02 3 L, 112-019-01 1 L, 112-019-01 2 L, 112-019-01 3 L, 113-022-01 1, 113-022-01 2, 113-023-02 1 L, 113-023-02 2 L and 113-023-02 3 L.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Other systems, processes, and features will become apparent to those skilled in the art upon examination of the following drawings and detailed description. It is intended that all such additional systems, processes, and features be included within this description, be within the scope of the present disclosure, and be protected by the accompanying claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a map of the insertion site in the A511::COP2 engineered phage.

FIG. 2 is a map of the insertion site in the LP124::COP2 engineered phage.

FIG. 3 is a map of the insertion site in the LP40::COP2 engineered phage.

FIG. 4 is a graph comparing the luminescence put out (measured in Relative Light Units, RLU) by healthy and sick cells infected with a mixture of A511, LP40 and LP124 engineered phage comprising either nanoluc luciferase or codon optimized COP2 luciferase.

FIG. 5 is a graph comparing relative performance in terms of luminescence ouput of healthy and sick cells of a mixture of A511, LP40 and LP124 engineered phage comprising either the nanoluc luciferase or codon optimized COP2 luciferase.

FIG. 6 is a graph comparing the performance in terms of luminescence output of sick cells infected with A511 engineered phage comprising either nanoluc luciferase or codon optimized COP2 luciferase.

FIG. 7 is a graph comparing relative performance in terms of luminescence output of sick cells of A511 engineered phage comprising either the nanoluc luciferase or codon optimized COP2 luciferase.

FIG. 8 is a series of graphs that depict the mean light signal detected in Relative Light Units (RLU) per colony forming units (CFU) in samples of various Listeria species (total of 66 species) infected with recombinant codon-optimized phage version 2 (COP2; v.1.0.2; top panel) or with recombinant optimized phage version 3 (COP3 v.1.0.3; bottom panel). The Listeria species used in these experiments are considered “weak signal producers” that do not typically produce strong signal following infection with recombinant luciferase-encoding phages.

FIG. 9 is a graph that depicts a comparison of the amounts of mean light signal detected (shown as a percentage) in Listeria samples that were infected with either recombinant codon-optimized phage version 2 (COP2; v.1.0.2) or with recombinant optimized phage version 3 (COP3 v.1.0.3). The y-axis depicts the mean relative signal increase in percentage, and the x-axis depicts the Listeria strain that utilized.

FIG. 10 is a graph that depicts optimal phage concentration using codon-optimized, luciferase encoding, recombinant phage. The y-axis depicts RLU, and the x-axis depicts phage concentration in PFU/mL.

FIG. 11 is a graph that demonstrating the effect of the addition of various LiCl concentrations to the 1×TSB buffer in RLU output following infection of bacteria with luciferase encoding phage and normalized to RLU output obtained from 1×BHI buffer. A 3 hour and a 6 hour time point were assayed. The % values are shown on the y-axis.

FIG. 12 is a graph that summarizes the findings with respect to enzyme activity and infection rate of bacteria exposed to luciferase encoding phage while in the presence of various iterations of the infection buffer formulations and normalized to RLU output obtained from 1×BHI buffer. The % values are shown on the y-axis.

FIGS. 13A and 13B are a pair of graphs depicting the effect of the addition of HEPES buffer to the Formulation-1 infection buffer. Bacterial cells were exposed, in either Formulation-1 without HEPES or Formulation-1 with HEPES, to luciferase encoding phage, followed by assessment of RLU values normalized to RLU output obtained from 1×BHI buffer. The % values are shown on the y-axis.

FIG. 14 is a graph that demonstrates the effects of the addition of Tween-80, or Tween-80 and lecithin, or the addition of neither of these components to the Listeria growth broth in the presence of increasing concentrations of quaternary ammonium salts. Bacterial cells were exposed to luciferase encoding phage in the following buffers, Listeria growth broth, Listeria growth broth with Tween-80, or to Listeria growth broth with the addition of both Tween-80 and lecithin. The RLU values are shown on the y-axis.

FIG. 15 is a graph that summarizes the findings with respect to enzyme activity and infection rate of bacteria exposed to luciferase encoding phage while in the presence of various iterations of the infection buffer formulations and normalized to RLU output obtained from 1×BHI buffer. The % values are shown on the y-axis.

FIG. 16 is a graph that depicts the results of a lower limit of detection assay in which Formulation-2 (NIB-12) is used as the infection buffer either alone or with the addition of Tween-80. The bacteria were exposed in the presence of either of these infection buffers to luciferase encoding phage, followed by detection of RLU. RLU values are shown on the y-axis.

FIGS. 17A-17D are a series of graphs and a table that depict the effects of the NIB-14 infection buffer or a base buffer, Letheen, in the presence of various concentrations of the F29 sanitizing solution containing quaternary ammonium compounds. Bacteria were exposed to the different concentrations of the F29 solution for 30 minutes preceding the luciferase encoding phage infection step. The enzyme was also exposed to different concentrations of the F29 solution during the enzymatic activity test. RLU values were then collected. FIG. 17A depicts the effects of using either buffer in the presence of various concentrations of F29 on phage infection activity. FIG. 17B depicts the effects of using either buffer in the presence of various concentrations of F29 on enzymatic activity. FIG. 17C depicts the effects of using either buffer during exposure of the NanoGlo luciferin to various concentrations of the F29 solution. FIG. 17D depicts a table with a summary of the results obtained from this series of experiments. When graphs are depicted, the y-axis represents percentage activity compared to no addition of F29.

FIGS. 18A and 18B are a series of graphs that depict the effects of incubating Letheen or NIB-14 over time with the sanitizing solution, Clorox, prior to the downstream effects on the luciferase encoding phage infection assay. Assay time points for this study included 5, 10, 15, 30, 60 and 120 minutes. Three concentrations of Clorox were assessed over this time, including 500 ppm, 1000 ppm, and 5000 ppm.

FIG. 19 is a graph that depicts the results of a series of experiments in which stressed cells (i.e. cells that were dried for 18 hours on a stainless steel table before further processing) were incubated with various NIB infection buffers, NIB-10, NIB-12 or NIB-14. As a control for the experiments, 1×BHI was used. The stressed cells were exposed in each of these buffers to luciferase encoding phage, and subsequently followed by assessing the RLU for each condition. The enzyme activity was also assessed in the presence of each buffer iteration.

FIGS. 20A-20B are a series of graphs that depict lower limit of detection assays (also referred to herein as “LLOD”) utilizing L. monocytogenes, incubated in various foods. LLOD assays were performed with L. monocytogenes incubated in 100% (full fat) ice cream and 100% (full fat) milk (FIG. 20A) or NIB-14 (as a reference), or in Demi-Fraser broth incubated at 30 C in with raw ground beef, deli turkey, guacamole and queso fresco (FIG. 20B) for 16 hours. For L. monocytogenes in the presence of raw ground beef, deli turkey, guacamole or queso fresco, the sample was incubated in an equal volume of NIB-14 infection buffer and luciferase encoding phage used for the infection of the microbes in the sample.

FIGS. 21A-21D are a series of graphs that depict the time course of L. monocytogenes detection in various food samples. L. monocytogenes was added to the food samples for defined amounts of time, followed by infection with a recombinant, luciferase encoding phage and subsequent detection of the luciferin signal. The food samples used in the assays included turkey (FIG. 21A), queso fresco (FIG. 21B), guacamole (FIG. 21C) and beef (FIG. 21D). RLU values are on the y axis.

FIG. 22A-22M is a series of graphs that depict the time course of L. innocua and monocytogenes detection in various food samples. L. innocua or L. monocytogenes was added to the food samples for defined amounts of time, followed by infection with a recombinant, luciferase encoding phage and subsequent detection of the luciferin signal. The food samples used in the assays included potato salad (FIGS. 22A, 22B, 22H, and 22I), smoked salmon (FIGS. 22C, 22D, 22J and 22K), ground turkey (FIGS. 22E, 22F, 22L and 22M) and sour cream (FIG. 22G). RLU values are on the y axis.

FIG. 23A-B is a series of graphs that depict the time course of L. innocua and monocytogenes detection in either pepperoni or spinach.

FIG. 24A-C is a series of graphs that depict the time course of detection of L. seeligeri, L. innocua, and L. monocytogenes in turkey, queso, and in guacamole.

FIG. 25A-D is a series of graphs that depict the detection of L. monocytogenes in various food types when the assay is performed utilizing various dilution of food matrix to incubation buffer.

FIG. 26 is a sequence alignment of the CPS open reading frame of LP143 (SEQ ID NO: 124), A511 (SEQ ID NO: 125), LP101 (SEQ ID NO: 126), LP124 (SEQ ID NO: 127), LP99 (SEQ ID NO: 128), LP48 (SEQ ID NO: 129), LP125 (SEQ ID NO: 130), P100 (SEQ ID NO: 131) and LP40 (SEQ ID NO: 132) phage.

FIG. 27 is an amino acid alignment of the CPS open reading frame of LP143 (SEQ ID NO: 133), A511 (SEQ ID NO: 134), LP101 (SEQ ID NO: 135), LP124 (SEQ ID NO: 136), LP99 (SEQ ID NO: 137), LP48 (SEQ ID NO: 138), LP125 (SEQ ID NO: 139), P100 (SEQ ID NO: 140) and LP40 (SEQ ID NO: 141) phage.

FIG. 28 is a sequence alignment of recombinant LP143 (SEQ ID NO: 142), A511 (SEQ ID NO: 143), LP101 (SEQ ID NO: 144), LP124 (SEQ ID NO: 145), LP99 (SEQ ID NO: 146), LP48 (SEQ ID NO: 147), LP125 (SEQ ID NO: 148), and P100 (SEQ ID NO: 149) phage engineered with firefly luciferase.

FIG. 29 is a sequence alignment of recombinant A511 (SEQ ID NO: 150), LP124 (SEQ ID NO: 151), LP125 (SEQ ID NO: 152), P100 (SEQ ID NO: 153), and LP40 (SEQ ID NO: 154) phage with nanoluc luciferase.

FIGS. 30A and 30B are a series of bar graphs that depict the influence of the volume of Nano-Glo reagent added to the total sample volume (Listeria lysate) on the resultant relative light units (RLU) detected.

FIGS. 31A and 31B are a series of bar graphs that depict the effects of varying the volume of sample added to the detection reaction composition while maintaining a constant volume of Nano-Glo reagent in said composition in healthy cells.

FIGS. 32A-32C are a series of bar graphs that depict the effects of varying the volume of sample added to the detection reaction composition while maintaining a constant volume of Nano-Glo reagent in said composition in stressed cells.

FIGS. 33A and 33B are a series of graphs that depict the effect of increasing the incubation temperature from 30° C. to either 35° C. (FIGS. 33A, and 33B), or to 37° C. (FIG. 33B). FIG. 33A is a bar graph that indicates the ratio of signal detected at 35° C./30° C. A ratio of greater than 1 indicated an increase in signal detected (FIG. 33A).

FIG. 33B compares the average signal detected following sample incubation at 30° C., 35° C., and 37° C.

FIGS. 34A and 34B are a series of graphs that depict the number of Listeria strains detected at or above RLU cutoff based on the infection cocktail used.

FIGS. 35A and 35B are a series of graphs that depict the effect of the addition of organic solvents (i.e. acetone, isopropanol, ethanol, and propylene glycol) on the amounts of light signal emitted from a sample.

FIG. 36 is a graph that indicates the effect of sample volume and the volume of ethanol added to the sample on the light signal emitted from the sample as a means to determine a true positive signal versus a false positive signal.

FIG. 37 is a graph that depicts the signal to noise observed following incubation of specific healthy Listeria strains (x-axis) with either version 1.0.4 or with version 2.0 of the Listeria detection assay.

FIGS. 38A-38C are a series of graphs that depict the detection of stressed Listeria cells with the use of either v1.0.4 or with v. 2.0 of the Listeria detection assay.

FIGS. 39A and 39B are a series of graphs that depict the detected of Listeria strains from environmental samples with the use of either v1.0.4 or with v. 2.0.

FIG. 40 depicts the performance of Listeria detection assay v2.0 versus previous iterations of the detection assay.

FIGS. 41A-41C are a series of graphs that depict the effect of substituting individual components of the Listeria detection composition v2.0 with those used in v1.0.4 in the detection of Listeria from a control sample (FIG. 40A) or from environmental samples (FIGS. 40A and 40B).

FIG. 42 is a graph that illustrates the coefficient of variation (CV) for several perameters tested in either a polyurethane (PUR) sponge or a cellulose sponge. 3

FIG. 43 is a graph that depicts the average RLU/CFU value across 5 repeats with the standard deviation across the five repeats. Also plotted is the percent coefficient of variation (CV) of the assay.

FIG. 44 is a graph that depicts the effect of pyruvate or metabisulfite on the activity of NanoLuc enzyme.

FIG. 45 is a graph that depicts the light signal detected (RLU) in comparison to the amounts of pyruvate added to the NanoGlo buffer in the presence of 0.1% peroxide.

FIG. 46 is a graph that depicts the effect of the addition of various concentrations of pyruvate in the NanoGlo buffer on the enzymatic activity.

FIG. 47 is a graph that depicts the neutralization of solid peroxide-based sanitizer on polyurethane sponges by various concentrations of pyruvate in NanoGlo buffer.

FIG. 48 is a graph that depicts the effect of Quat Neutralizer (28% Tween 80, 4% lecithin, and 3 mM KH₂PO₄, pH 7.4) on the activity of NanoLuc enzyme.

FIG. 49 is a graph that depicts the effects of 0.25% sodium pyruvate and 2% Quat Neutralizer in the NanoGlo buffer upon incubation in the presence of varying amounts of sanitizer concentrations.

FIGS. 50A-50C are a series of graphs that depict the LP80 phage's ability to clear Listeria cultures. In FIG. 50A, each point in the graph represents a single strain tested against either LP80 or LP80 RM lysate, where LP80 RM has been produced in a different host than LP80; points in the lower right quandrant produced by the two orthogonal lines in the figure represent strains that are cleared by the alternate host lysate but not by the other host lysate. FIG. 50B is a comparison of RLU generated by LP80 produced from either an alternate host (y-axis) or typical host (x-axis); each dot represents one of the 474 Listeria strains tested against the two phage lysates. Note that RM stands for Restriction Modification system, an endogenous bacterial DNA modification system for defening against foreign DNA. Strains that show greater RLU output from the alternate host generated phage skew toward the upper left quadrant of the graph. FIG. 50C depicts a distribution of the fold signal increase across 474 Listeria strain panel. The average signal increase is roughly 10-fold across the entire panel with a maximum increase of 292-fold.

FIG. 51 shows Table 19.

FIG. 52 shows Table 20.

FIG. 53 shows Table 21.

FIG. 54 shows Table 22.

FIG. 55 shows Table 23.

FIG. 56 shows Table 24.

FIG. 57 shows Table 25.

FIG. 58 shows Table 26.

FIG. 59 shows Table 29.

FIG. 60 shows Table 31.

FIG. 61 shows Table 32.

FIG. 62 shows Table 33 (Optimization of Listeria Detection Components (V2.0) and Rationale for Use).

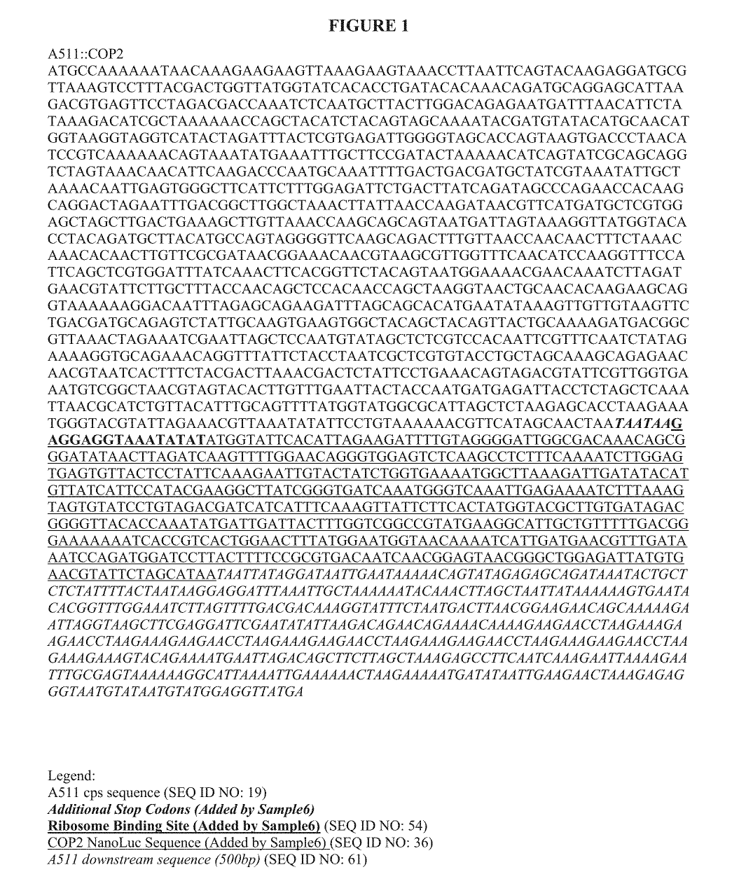

### DETAILED DESCRIPTION

Compositions, methods, and kits are presented herein for the detection of target microbes through the use of codon-optimized recombinant phage. This disclosure provides recombinant phage with sequence encoding a codon optimized marker, aqueous solutions that enable robust signal detection following contact with the target microbes in a sample. The compositions and methods of the disclosure provide broad detection coverage of a microbe genus, species or a combination of species.

The composition and buffer components necessary for robust signal detection following infection by codon optimized phage is dependent on the sampled area. For example, at least one recombinant phage is provided in combination with an aqueous solution, that, together, provide optimal for the detection of microbes in an agricultural facility. A particular challenge of detection of microbes in an agricultural facility is the potential for the presence of trace sanitation solutions that may interfere with signal detection. Another embodiment relates to the use of recombinant phage for the detection of microbes in agricultural products themselves, such as, for example, food stuffs intended for human or animal consumption. Detection of microbes in an agricultural sample presents unique challenges in that components of the agricultural sample may contain substances that interfere with signal detection. The aqueous solutions presented herein are formulated to minimize such interference.

The methods and compositions presented herein are optimized in order to allow the propagation of microbes from a test sample, infection of the microbes with a recombinant phage that encodes a detectable marker, and the quantification of the amounts of the microbes from the sample by way of detection of the recombinant phage marker.

### Methods of Making Recombinant Phase

Any method known in the art can be used to make genetically modified phage from starting phage. For example, U.S. Pat. No. 5,824,468 discloses methods of making genetically modified phage. Alternative methods are disclosed in co-pending application Ser. No. 13/627,060, filed Sep. 26, 2012, which is hereby incorporated herein by reference.

Phage infective engineering (PIE) is used herein to make recombinant phage. PIE methodology is disclosed in U.S. patent application Ser. No. 14/226,889, which is hereby incorporated herein in its entirety by reference. This method is sometimes referred to herein as phage infective engineering (PIE). This method allows insertion of a heterologous nucleic acid sequence into any desired location of a phage genome. The PIE method utilizes a phage targeting vector (PTV) that is transformed into a phage host cell. The PTV comprises a heterologous nucleic acid sequence (such as an open reading frame encoding a marker) for insertion into a phage genome. The heterologous nucleic acid sequence is flanked by upstream and downstream homology regions, which are located adjacent to the desired insertion site. In some embodiments the homology regions in the vector are directly adjacent in a starting phage genome. Such embodiments allow insertion of the heterologous nucleic acid sequence into the phage genome without a loss of endogenous phage sequence. In some embodiments the homology regions in the vector flank a region of the starting phage genome that is not included in the vector. Such embodiments allow insertion of the heterologous nucleic acid sequence into the phage genome while deleting a region of the starting phage genome at the site of insertion. Such embodiments allow, for example, the replacement of an endogenous phage sequence with a replacement sequence. In some embodiments the starting sequence that is deleted and the replacement sequence display sequence homology, such as homology of at least 50%, 60%, 70%, 80%, 90%, 95%, 96%, 97%, 98%, 99% or higher.

The upstream homology region, downstream homology region, and heterologous nucleic acid sequence are combined in a vector to make a PTV. One example of a suitable vector is pMK4; however, skilled artisans are aware of many suitable vectors that may be used for this purpose. The plasmid may be isolated in any suitable host, such as E. coli. Upon verification, the plasmid is then transformed into a phage host cell. One example of such a cell useful for many Listeria phage is the L. monocytogenes strain EGD-e.

Once the PTV is successfully transformed into the phage host, the initial recombination was performed by incubating the transformed phage host cell with starting phage.

To assess whether recombination has occurred, the infection is assayed using any suitable method to identify recombinant phage that comprise the heterologous nucleic acid sequence. PCR is one method that may be used. Alternatively, if the heterologous nucleic acid sequence comprises an open reading frame the presence of transcripts encoded by that open reading frame, the presence of the encoded gene product, or functional readouts of the encoded gene product may be screened for in cultures of cells infected with the resultant phage to identify recombinant phage.

### Codon Optimized Phase

The disclosure provides recombinant phage comprising a heterologous nucleic acid sequence encoding a codon optimized marker. The phage can be LP40, LP48, LP99, LP101, LP124, LP125, LP143, A511, or P100. The marker can be any detectable marker. In one embodiment the marker is luciferase.

The design of codon optimized phages should take into account a variety of factors, including the frequency of codon usage in a host organism, nearest neighbor frequencies, RNA stability, the potential for secondary structure formation, the route of synthesis and the intended future DNA manipulations of that gene.

The degeneracy of the genetic code permits the same amino acid sequence to be encoded and translated in many different ways. For example, leucine, serine and arginine are each encoded by six different codons, while valine, proline, threonine, alanine and glycine are each encoded by four different codons.

However, the frequency of use of such synonymous codons varies from genome to genome among kingdoms and phyla. For example, synonymous codon-choice patterns among mammals are very similar, while evolutionarily distant organisms such as yeast (S. cerevisiae), bacteria (such as E. coli) and insects (such as D. melanogaster) reveal a clearly different pattern of genomic codon use frequencies. In reference to phage codon optimization, codon selection may vary with the species, strain or ribotype of the host to be infected by a particular phage. Additionally, codon usage may vary with the environment in which the host exists, depending on factors such as temperature, pH, pressure, and other external parameters. Further, codon usage may vary with the state of growth in which the host exists, e.g. depending on rapid division vs. non-division, or within a healthy or an injured cellular state.

These differences in codon-choice patterns appear to contribute to the overall expression levels of individual genes by modulating translation initiation rates, as well as peptide elongation rates. Experimental evidence supports this argument; the rate of polypeptide synthesis depends on the character of the codons being translated, as well as the initial kinetics for transfer RNA (“tRNA”) ternary complex formation.

The preferred codon usage frequencies for a recombinant phage should reflect the codon usages of genes derived from the genome of the intended host organism.

In some embodiments, a gene can be optimized by replacing codons of the origin species with known preferred codons from a host organism encoding the same amino acid. In some embodiments, a host organism is Listeria. In some embodiments, software can be utilized which applies an algorithm to a genetic sequence which will codon optimize the sequence for a specific host organism. In some embodiments, software from DNA 2.0™ can be used to codon optimize a genetic sequence for a specific host organism. Example algorithms for codon optimization in silico have been described (see Villalobos et al. BMC Bioinformatics. Gene Designer: a synthetic biology tool for constructing artificial DNA segments. PLoS ONE. 2011 6:e19912; U.S. Pat. No. 8,635,029; U.S. Pat. No. 8,401,708; U.S. Pat. No. 8,126,653; U.S. Pat. No. 8,005,620; U.S. Pat. No. 7,805,252; U.S. Pat. No. 7,561,973; U.S. Pat. No. 7,561,72.)

In some embodiments, codon optimization allows for increased expression of phage encoded proteins in a host organism. In some embodiments, the host organism is a bacterium. In some embodiments, codon optimization allows for increased expression of reporter proteins or polypeptides encoded by a recombinant phage in a host organism. In some embodiments, codon optimization of recombinant phage allows for increased expression of a luciferase reported by Listeria.

In some embodiments, Listeria phages used for recombination may be selected from A511, LP124, and LP40. In some embodiments, recombinant phages comprise the entirety of the original phage genome. In some embodiments, recombinant phages comprise deletions to the original phage genome and addition of heterologous nucleic acid sequences. In some embodiments, recombinant phages comprise added stop codons. In some embodiments, recombinant phages comprise added ribosome binding sites. In some embodiments, recombinant phages comprise a codon optimized reporter gene. In some embodiments, a reporter gene is a sequence encoding luciferase. In some embodiments, the luciferase reporter gene is a codon optimized NanoLuc sequence optimized for expression in Listeria. In some embodiments, a recombinant phage is an A511 phage comprising added stop codons, an added ribosome binding site, and an added codon optimized NanoLuc sequence. In some embodiments, a recombinant phage is a LP124 phage comprising added stop codons, an added ribosome binding site, and an added codon optimized NanoLuc sequence. In some embodiments, a recombinant phage is a LP40 phage comprising added stop codons, an added ribosome binding site and added codon optimized NanoLuc sequence.

### Optimized Assay Aqueous Solution for Microbial Detection

The disclosure provides formulations of an aqueous solution which effectively enable bacteria isolated from a test site to be productively infected by recombinant phage. Furthermore, the aqueous solution is capable of preserving the enzymatic activity used in the phage based detection system. A major difficulty encountered in the detection of bacteria using phage based recombinant markers is the potential interactions between sanitation reagents found in the sample and the test reagent compounds that are used to quantify bacterial presence or absence. This problem is augmented given the propensity of facilities to use amounts of disinfectants in excess of the recommended guidelines presented by the Federal Drug Administration (FDA), United States Department of Agriculture (USDA), and Centers for Disease Control (CDC). Overuse of these sanitization agents may lead to obfuscation of true positive or true negative results due to (i) decreasing enzymatic activity required for the phage-based detection system, (ii) lowering the ability of phage to infect bacteria collected from the test site, or (iii) disrupting the bacterial cells to a degree that they are not detectable.

Collection and processing of a an environmental sample from a test site follows a stepwise process that includes: (a) collection of the sample by way of swabbing the surface with a sponge, followed by immediate placement of the sponge into an isolated container; (b) processing the sample begins with the addition of an aqueous solution (infection buffer), and the addition of a marker encoding phage to the sample collecting sponge; (c) incubation of the solution impregnated sponge at an appropriate temperature range; (d) isolation of the liquid from the sponge by way of centrifugation; and (e) detection of a signal in the liquid with an instrument (e.g. luciferase presence with a luminometer). The solution added to the sponge is a buffer that contains reagents that minimize the interaction with components of commonly used sanitization solutions that have been found to reduce signal detection ability (e.g. by either reducing phage infection or by reducing enzymatic activity, or by affecting the luciferin substrate). The purposes of the buffer include recovery of the isolated stressed and injured bacteria in order to optimize phage infection and to optimize downstream signal detection. The marker used for signal detection can be any detectable marker. Preferred detection signal systems include luciferase based assays.

Ideal formulations of the disclosure would allow for high amounts of signal following phage infection, high amount of signal stability, and the ability to effectively neutralize various components found in sterilization solutions without the loss of either signal or stability. The formulations presented herein make use of additives that serve as neutralizers to overcome these challenges. Commonly found sanitation chemicals that may have the ability to interfere with environmental sample test results include chlorine, quaternary ammonium salts, organic acids and peracids, iodophors, and detergents. Formulations presented herein contain remedies to overcome these agents including, for example, sodium metabisulfite, sodium thiosulfate, lecithin, Tween-80, HEPES and buffering salts.

Bacterial cells collected from the environment present many additional challenges to the downstream processing required for adequate signal detection. Many of these challenges relate to the health of the cells upon collection. The collected cells may be starved, osmotically stressed, and have underlying oxidative stress. Formulations have been developed, and described herein, to overcome these challenges encountered following the collection of the cells. For example, detailed herein, and specifically in the Examples section, are formulations to overcome osmotic stress (e.g. via addition of glycerol), cell starvation (e.g. via addition of nutrients including carbon, nitrogen source, sugars and vitamins), and oxidative stress (e.g. via the addition of vitamins including those contained in yeast extract). Interaction with non-target biologicals also poses a challenge in the downstream signal detection methods. Formulations presented herein have been optimized to overcome non-target biological interactions via the addition of either nalidixic acid and/or lithium salts.

The Examples detail various formulations that work well at preserving signal and signal stability (See Examples 6-12). A base aqueous solution of the disclosure is Formulation-1 (Table 1). A variation of the base aqueous solution Formulation-1, Formulation-1A, makes use of additives (i.e. 0.08% MgSO₄, and 0.1% pyruvate) that further aid in preservation of signal intensity and phage infection (Table 2).

TABLE 1
Base Infection Buffer Formulation (Formulation 1)

| Components | Group | Function |
|---|---|---|
| 1x Tryptic Soy Broth | Media | Support recovery and growth |
| | (nutrients) | of stressed cells (provide |
| | | hydrolyzed amino acids, |
| | | sugars, minerals) |
| 0.25% LiCl | Selective | Prevent or limit growth of |
| | agents | competing biologicals |
| 0.002% nalidixic acid | Selective | Prevent or limit growth of |
| | agents | competing biologicals |
| 0.5% yeast extract | Vitamins | Prevent oxidative stress |
| | (B complex) | |
| 0.25% glucose | Carbon | Nutrient |
| | Source | |

TABLE 2
Base Infection Formulation (Formulation 1-A)

| Components | Group | Function |
|---|---|---|
| 1x Tryptic Soy Broth | Media | Support recovery and growth |
| | (nutrients) | of stressed cells (provide |
| | | hydrolyzed amino acids, |
| | | sugars, minerals) |
| 0.25% LiCl | Selective | Prevent or limit growth of |
| | agents | competing biologicals |
| 0.002% nalidixic acid | Selective | Prevent or limit growth of |
| | agents | competing biologicals |
| 0.5% yeast extract | Vitamins | Prevent oxidative stress |
| | (B complex) | |
| 0.25% glucose | Carbon | Nutrient |
| | Source | |
| 0.08% MgSO₄ | Ions | Increases infection ability |
| 0.1% sodium pyruvate | Carbon | Nutrient |
| | Source | |

A preferred embodiment of the aqueous solutions of the disclosure is Formulation-2 (also referred to herein as “NIB-12”) (Table 3). As detailed in the Example section, the addition of 20 mM HEPES increases enzyme activity and stability, and increases the buffering capacity against pH extremes (See Example 7).

Another preferred embodiment of the aqueous solutions of the disclosure is formulation NIB-14 (Table 4). NIB-14 contains lecithin, Tween-80 and potassium phosphate added to the base components of NIB-12. NIB-14 allows for greater phage infection ability and increased enzymatic activity compared with a base medium (BHI), and also allows for greater neutralization of remnant sanitizer chemicals in comparison to other aqueous solutions tested (See Examples 8 and 11).

TABLE 3
Infection Formulation 2 (NIB-12)

| Components | Group | Function |
|---|---|---|
| 1x Tryptic Soy Broth | Media | Support recovery and growth |
| | (nutrients) | of stressed cells (provide |
| | | hydrolyzed amino acids, |
| | | sugars, minerals) |
| 0.25% LiCl | Selective | Prevent or limit growth of |
| | agents | competing biologicals |
| 0.002% nalidixic acid | Selective | Prevent or limit growth of |
| | agents | competing biologicals |
| 0.5% yeast extract | Vitamins | Prevent oxidative stress |
| | (B complex) | |
| 0.25% glucose | Carbon | Nutrient |
| | Source | |
| 0.08% MgSO₄ | Ions | Increases infection ability |
| 0.1% sodium pyruvate | Carbon | Nutrient |
| | Source | |
| 20 mM HEPES, pH 7.5 | Buffering | Neutralize environmental pH |
| | agents | extremes |

TABLE 4
Infection Formulation NIB-14

| Components | Group | Function |
|---|---|---|
| 1x Tryptic Soy Broth | Media | Support recovery and growth |
| | (nutrients) | of stressed cells (provide |
| | | hydrolyzed amino acids, |
| | | sugars, minerals) |
| 0.25% LiCl | Selective | Prevent or limit growth of |
| | agents | competing biologicals |
| 0.002% nalidixic acid | Selective | Prevent or limit growth of |
| | agents | competing biologicals |
| 0.5% yeast extract | Vitamins | Prevent oxidative stress |
| | (B complex) | |
| 0.25% glucose | Carbon | Nutrient |
| | Source | |
| 0.08% MgSO₄ | Ions | Increases phage infection |
| | | ability |
| 0.1% sodium pyruvate | Carbon | Nutrient |
| | Source | |
| 20 mM HEPES, pH 7.5 | Buffering | Neutralize environmental pH |
| | agents | extremes |
| Tween-80 1.5% | Neutralizer of | Neutralize biguanides |
| | sanitizers, | (chlorhexidine), bis-phenols |
| | Non-ionic | (hexachlorophene), phenolic |
| | detergent | compounds, cresols, formalin, |
| | | to some extent quaternary |
| | | ammonium compounds, |
| | | organic acids, parabens, |
| | | alcohols |
| Lecithin 0.22% | Nutrient and | Nutrient and Neutralizer |
| | Neutralizer | |
| 0.02% Potassium | Buffering | Neutralize environmental pH |
| Phosphate, pH 7.4 | agents | extremes |

Table 5 lists the groups and category of the reagents that are included in the aqueous solutions disclosed herein, and the affect that each of these components has on Listeria detection.

TABLE 5
Reagents in Aqueous Solution and Influence on Listeria Detection

| | | | | Effect on |
|---|---|---|---|---|
| | | | | Listeria |
| Group | Category | Components tested | Function | Detection |
| Nutrients | Carbohydrates | Glucose | | positive |
| | | Glycerol | Energy source for | neutral |
| | | | stressed cells | |
| | | Mannose | | |
| | | Fructose | | |
| | | Sucrose | | neutral |
| | Meat and plant | Beef extract | Nutrient source | |
| | extracts | Pancreatic digest of | Nitrogen source | |
| | | casein | | |
| | | Papaic digest of | Nutrient source | |
| | | soybean meal | | |
| | | Proteose peptone | Nitrogen source | |
| | | Sodium succinate | Energy source | |
| | Carbon sources | Sodium acetate | | |
| | | Acetoin | | |
| | | Sodium pyruvate | | positive |
| | | Alpha-ketoglutarate | | negative |
| | | Sodium malonate | | negative |
| | | Sodium citrate | | negative |
| | | Ferric ammonium | | neutral |
| | | citrate | | |
| | Minerals | Ferric citrate | Iron source, | neutral |
| | | | growth enhancer | |
| | | Ferric sulfate | | neutral |
| | | Sodium glutamate | | |
| | Nitrogen sources | Ammonium nitrate | | |
| | | Ammonium sulfate | | |
| | | Urea | | |
| | Amino acids | Sodium glutamate | Energy source for | neutral |
| | | | stressed cells | |
| | | Branched-chain amino | Conversion of cells | neutral |
| | | acids (leucine, | in stationary phase | |
| | | isoleucine, valine) | into growing phase | |
| | | Proline | | |
| | | Cysteine | | |
| | | Methionine | | |
| | Media | Brain Heart Infusion | Support recovery and | positive |
| | | | growth of stressed cells | |
| | | | by providing variety of | |
| | | | nutrients(provide | |
| | | | hydrolyzed amino acids, | |
| | | | sugars, minerals) | |
| | | Tryptic Soy Broth | | positive |
| | | Buffered Peptone Water | | positive |
| | | LPT-6F (BioMerieux) | | negative |
| | | Moxalactam | Prevent or limit growth | neutral |
| | | | of competing biologicals | |
| Selective | Antibiotics | Nalidixic acid | | positive |
| agents | | Polymyxin B | | |
| | | Ceftazidime | | positive |
| | | Acriflavin | | negative |
| | | Carbenicillin | | |
| | | Erythromycin | | |
| | | Mitomycin C | | |
| | | Penicillin G | | |
| | | Streptomycin | | |
| | | Tetracycline | | positive |
| | | Nitrofurantoin | | |
| | Growth inhibitors | Lithium chloride | Prevent or limit growth | optimized |
| | | | of competing biologicals | for positive |
| | Yeast growth | Cycloheximide | Antifungal agent | neutral |
| | inhibitor | | | |
| Vitamins | B complex | Yeast extract | Prevent oxidative stress | positive |
| | | Biotin | | |
| | | Riboflavin | | |
| | | Nicotinamide | | |
| | | Calcium panthotenate | | |
| | | Cyanocobalamine | | |
| | | Pyridoxine | | |
| | | Thiamine | | neutral |
| | | Lipoic acid | | |
| Divalent | | Magnesium sulphate | Support enzymatic | positive |
| metals | | | functions of cells, | |
| | | | support phage activity | |
| | | Magnesium chloride | | |
| | | Ferrous sulphate | | negative |
| | | Calcium chloride | | neutral |
| | | Zinc sulfate | | |
| Buffering | pH buffers | Sodium monobasic/ | Neutralize environmental | |
| agents | | sodium dibasic | pH extremes | |
| | | phosphates, pH 7.2 | | |
| | | HEPES, pH 7.4 | | positive |
| | | MOPS, pH 7.2 | | positive |
| Neutralizer | Oxygen scavenger | Sodium metabisulfite | Scavenge peroxides, | positive |
| of sanitizers | | | neutralize glutaraldehyde, | |
| | | | formaldehyde | |
| | | Catalase | Neutralize peroxides | |
| | | Sodium pyruvate | Scavenges peroxides | positive |
| | Halogens | Sodium thiosulfate | Neutralize halogens | negative |
| | neutralizer | | (iodine, chlorine, sodium | |
| | | | hypochlorite, chlorine | |
| | | | dioxide, also peroxides, | |
| | | | peroxyacids) | |
| | Mercury | Sodium thioglycollate | | negative |
| | neutralizer | | | |
| | Non-ionic | Polysorbate 80 | Neutralize biguanides | positive |
| | detergent/ | | (chlorhexidine), bis- | |
| | surfactant | | phenols (hexachlorophene), | |
| | | | phenolic compounds, | |
| | | | cresols, formalin, | |
| | | | to some extent | |
| | | | quaternary ammonium | |
| | | | compounds, organic acids, | |
| | | | parabens, alcohols | |
| | | Polysorbate 20 | | negative |
| | | Triton X-100 | | negative |
| | Emulsifier | Soy based lecithin | Neutralize quaternary | positive |
| | | | ammonium compounds, | |
| | | | parabens | |
| | | Soy based | | negative |
| | | hydroxylated lecithin | | |
| | | Phosphocholine chloride | | negative |
| | | O-octophosphorylcholine | | negative |

### Detection of Microbes in Agricultural Products

The detection of microbes in agricultural products is essential to maintain food safety. The disclosure provides methods and compositions to rapidly detect microbes in or on agricultural products with high sensitivity and within a timeframe that is relevant to enabling reaction within a work shirt (less than 8-10 hours). Compositions and methods of the disclosure are particularly beneficial in comparison to currently used methods of microbial detection in that the present invention, (i) has minimal sample preparation, (ii) is capable of detecting microbes in undiluted or minimally diluted matrix of certain foods resulting in less operator and cross-contamination risk, smaller volumes (less cost) and less waste, (iii) has high sensitivity and specificity, and (iv) has a total time to result of less than 8-10 hours.

Compositions and methods of the disclosure, as described in the Examples section (see Examples 13-14), incorporate the use of marker encoding phage, infection buffer/media, and a quantification of the amounts of phage marker present following phage infection of a sample in order to identify microbial presence in food samples. Preferred embodiments of the compositions and methods of the disclosure enable the detection of microbes in various food sources, including fatty foods, such as for example, whole milk, ice cream, queso fresco, and guacamole; salty foods, such as for example, deli turkey; and other foods, such as for example, beef. Preferred, although not limiting, microbial target species for the current invention include species of Listeria.

Unlike all other methods of microbial detection available today, the compositions and methods of the disclosure are capable of detecting target microbes in an undiluted food matrix. These properties contribute to the minimal sample preparation steps and associated rapid processing associated with the use of the present methods and compositions. Unlike other microbial detection methods, the use of the recombinant phage containing the codon-optimized marker sequence in the compositions and methods of the disclosure enables the rapid detection of extremely low numbers of microbes (e.g. Listeria monocytogenes in various foods, see Example 13), and the detection of microbes in lower limit of detection assays (also referred to herein as “LLOD”) of down to 1 CFU in certain foods (see Example 14). For example, in LLOD assays from whole milk samples, Listeria monocytogenes was detected in quantities as low as 50 cells in 50 mL of sample utilizing the recombinant phage based microbe detection system within a two hour period. (See Example 8). The detection of S. enterica was also assessed in various kinds of foods and was found to have a sensitivity of 1 CFU as well. Both LLOD and time course to detection assays revealed that using the compositions and methods of the disclosure enables the rapid detection of S. enterica with no enrichment (See Examples 13 and 14).

### Recombinant Phase

The phage LP40, LP48, LP99, LP101, LP124, LP125, LP143, and A511 were selected for engineering. The examples describe making recombinant versions of the phage LP40, LP48, LP99, LP101, LP124, LP125, LP143, A511, and P100, comprising a heterologous nucleic acid sequence encoding a marker. As demonstrated in the examples, those phage are useful, for example, to detect target bacteria, as further disclosed throughout this application.

Accordingly, this disclosure provides recombinant Listeria phage comprising a heterologous nucleic acid sequence encoding a marker. In some embodiments the recombinant phage comprises a genome comprising a region of at least 1 kb that comprises substantial homology to a region of at least 1 kb of the genome of at least one phage selected from LP40, LP48, LP99, LP101, LP124, LP125, LP143, A511, and P100. In some embodiments the region of homology comprises at least 2 kb, at least 3 kb, at least 4 kb, at least 5 kb, at least 6 kb, at least 7 kb, at least 8 kb, at least 9 kb, at least 10 kb, or more. In some embodiments the region of homology is the entire genome of the recombinant Listeria phage. In some embodiments the substantial homology is nucleotide sequence identity of at least 50%, 60%, 70%, 80%, 85%, 90%, 91%, 92%, 93%, 94%, 95%, 96%, 97%, 98% or 99% across the region of homology.

This disclosure provides the amino acid sequences of the cps gene of the phage LP40 (SEQ ID NO: 6), LP48 (SEQ ID NO: 8), LP99 (SEQ ID NO: 10), LP101 (SEQ ID NO: 12), LP124 (SEQ ID NO: 14), LP125 (SEQ ID NO: 16), LP143 (SEQ ID NO: 18), A511 (SEQ ID NO: 20), and P100 (SEQ ID NO: 22). Accordingly, in some embodiments this disclosure provides recombinant Listeria phage comprising a heterologous nucleic acid sequence encoding a marker, wherein the recombinant Listeria phage comprises a nucleic acid sequence that encodes a protein selected from SEQ ID NOS: 6, 8, 10, 12, 14, 16, 18, 20, and 22, and muteins thereof.

This disclosure also provides the nucleotide sequences of the open reading frames of the cps gene of the phage LP40 (SEQ ID NO: 5), LP48 (SEQ ID NO: 7), LP99 (SEQ ID NO: 9), LP101 (SEQ ID NO: 11), LP124 (SEQ ID NO: 13), LP125 (SEQ ID NO: 15), LP143 (SEQ ID NO: 17), A511 (SEQ ID NO: 19), and P100 (SEQ ID NO: 21). Accordingly, in some embodiments this disclosure provides recombinant Listeria phage comprising a heterologous nucleic acid sequence encoding a marker, wherein the recombinant Listeria phage comprises a nucleic acid sequence selected from SEQ ID NOS: 5, 7, 9, 11, 13, 15, 17, 19, and 21, and nucleic acid sequences comprising substantial homology thereto.

In some embodiments the recombinant Listeria phage comprising a heterologous nucleic acid sequence encoding a marker comprises a screenable marker. In some embodiments the marker is a luciferase. In some embodiments the luciferase is at least 70%, at least 80%, at least 90%, at least 91%, at least 92%, at least 93%, at least 94%, at least 95%, at least 96%, at least 97%, at least 98% or at least 99% identical to SEQ ID NO: 2. In some embodiments the luciferase is encoded by a nucleic acid sequence comprising SEQ ID NO: 1 or a nucleic acid sequence comprising substantial homology to SEQ ID NO: 1 capable of encoding a luciferase that is at least 70%, at least 80%, at least 90%, at least 91%, at least 92%, at least 93%, at least 94%, at least 95%, at least 96%, at least 97%, at least 98%, or at least 99 identical to SEQ ID NO: 2. In some embodiments the recombinant Listeria phage is selected from LP48::ffluc, LP99::ffluc, LP101::ffluc, LP124::ffluc, LP125::ffluc, LP143::ffluc, A511::ffluc, P100::ffluc, LP48::COP2, LP48::COP2, LP48::COP2, LP48::COP2, LP48::COP2, LP48::COP2, LP48::COP2, P100::COP2, LP48::COP3, LP48::COP3, LP48::COP3, LP48::COP3, LP48::COP3, LP48::COP3, LP48::COP3, and P100::COP3. In some embodiments the recombinant Listeria phage is selected from phage comprising genomes comprising substantial homology to at least one phage selected from LP48::ffluc, LP99::ffluc, LP101::ffluc, LP124::ffluc, LP125::ffluc, LP143::ffluc, A511::ffluc, P100::ffluc, LP48::COP2, LP48::COP2, LP48::COP2, LP48::COP2, LP48::COP2, LP48::COP2, LP48::COP2, P100::COP2, LP48::COP3, LP48::COP3, LP48::COP3, LP48::COP3, LP48::COP3, LP48::COP3, LP48::COP3, and P100::COP3.

In some embodiments the luciferase is at least 70%, at least 80%, at least 90%, at least 91%, at least 92%, at least 93%, at least 94%, at least 95%, at least 96%, at least 97%, at least 98% or at least 99% identical to SEQ ID NO: 4. In some embodiments the luciferase is encoded by a nucleic acid sequence comprising SEQ ID NO: 3 or a nucleic acid sequence comprising substantial homology to SEQ ID NO: 3 capable of encoding a luciferase that is at least 70%, at least 80%, at least 90%, at least 91%, at least 92%, at least 93%, at least 94%, at least 95%, at least 96%, at least 97%, at least 98%, or at least 99 identical to SEQ ID NO: 4. In some embodiments the recombinant Listeria phage is selected from LP040::nluc, LP124::nluc, LP125::nluc, A511::nluc, P100::nluc, LP040::COP2, LP124:: COP2, LP125:: COP2, A511:: COP2, P100:: COP2, LP040::COP3, LP124:: COP3, LP125:: COP3, A511:: COP3, P100:: COP3. In some embodiments the recombinant Listeria phage is selected from phage comprising genomes comprising substantial homology to at least one phage selected from LP040::nluc, LP124::nluc, LP125::nluc, A511::nluc, P100::nluc, LP040::COP2, LP124:: COP2, LP125:: COP2, A511:: COP2, P100:: COP2, LP040::COP3, LP124:: COP3, LP125:: COP3, A511:: COP3, P100:: COP3.

In some embodiments the heterologous nucleic acid sequence encoding a marker is operatively linked in the recombinant phage genome to at least one regulatory element that is also heterologous to the phage genome. In some embodiments expression of the heterologous nucleic acid sequence encoding a marker in target bacteria is controlled exclusively by regulatory elements that are heterologous to the phage genome.

In some embodiments the heterologous nucleic acid sequence encoding a marker is operatively linked in the recombinant phage genome to at least one regulatory element that is endogenous to the phage genome. In other words, the heterologous nucleic acid sequence encoding a marker is operatively linked to the endogenous regulatory element by virtue of the location in the starting phage genome where the heterologous nucleic acid sequence encoding a marker is placed. In some embodiments expression of the heterologous nucleic acid sequence encoding a marker in target bacteria is controlled exclusively by regulatory elements that are endogenous to the phage genome. In some embodiments expression of the heterologous nucleic acid sequence encoding a marker in target bacteria is controlled in part by regulatory elements that are endogenous to the phage genome and in part by regulatory elements that are heterologous to the phage genome.

In some embodiments the recombinant Listeria phage comprising a heterologous nucleic acid sequence encoding a marker comprises more than one heterologous nucleic acid sequence encoding a marker. In some embodiments the recombinant phage comprises multiple copies of the same nucleic acid sequence encoding a marker (i.e., copy encodes the same marker). In some embodiments the recombinant phage comprises copies of more than one type of nucleic acid sequence encoding a marker (i.e., at least two copies encode different markers). In some embodiments the more than one copy are positioned at adjacent locations in the recombinant phage genome. In other embodiments at least one (up to all) of the more than one copy are located at non-adjacent locations in the recombinant phage genome.

In some embodiments the length of the heterologous nucleic acid sequence is at least 100 bases, at least 200 based, at least 300 bases, at least 400 bases, at least 500 bases, at least 600 bases, at least 700 bases, at least 800 bases, at least 900 bases, at least 1.0 kilobase (kb), at least 1.1 kb, at least 1.2 kb, at least 1.3 kb, at least 1.4 kb, at least 1.5 kb, at least 1.6 kb, at least 1.7 kb, at least 1.8 kb, at least 1.9 kb, at least 2.0 kb, at least 2.1 kb, at least 2.2 kb, at least 2.3 kb, at least 2.4 kb, at least 2.5 kb, at least 2.6 kb, at least 2.7 kb, at least 2.8 kb, at least 2.9 kb, at least 3.0 kb, at least 3.1 kb, at least 3.2 kb, at least 3.3 kb, at least 3.4 kb, at least 3.5 kb, at least 3.6 kb, at least 3.7 kb, at least 3.8 kb, at least 3.9 kb, at least 4.0 kb, at least 4.5 kb, at least 5.0 kb, at least 5.5 kb, at least 5.5 kb, at least 6.0 kb, at least 6.5 kb, at least 7.0 kb, at least 7.5 kb, at least 8.0 kb, at least 8.5 kb, at least 9.0 kb, at least 9.5 kb, at least 10 kb, or more. In some embodiments the length of the heterologous nucleic acid sequence is 500 bases or less, 1.0 kb or less, 1.5 kb or less, 2.0 kb or less, 2.5 kb or less, 3.0 kb or less, 3.5 kb or less, 4.0 kb or less, 4.5 kb or less, 5.0 kb or less, 5.5 kb or less, 6.0 kb or less, 6.5 kb or less, 7.0 kb or less, 7.5 kb or less, 8.0 kb or less, 8.5 kb or less, 9.0 kb or less, 9.5 kb or less, or 10.0 kb or less. In some such embodiments the heterologous nucleic acid sequence comprises a length that is less than the maximum length of heterologous nucleic acid sequence that can be packaged into a phage particle encoded by the phage genome and comprising the phage genome.

In some embodiments the length of the heterologous nucleic acid sequence is from 100 to 500 bases, from 200 to 1,000 bases, from 500 to 1,000 bases, from 500 to 1,500 bases, from 1 kb to 2 kb, from 1.5 kb to 2.5 kb, from 2.0 kb to 3.0 kb, from 2.5 kb to 3.5 kb, from 3.0 kb to 4.0 kb, from 3.5 kb to 4.5 kb, from 4.0 kb to 5.0 kb, from 4.5 kb to 5.5 kb, from 5.0 kb to 6.0 kb, from 5.5 kb to 6.5 kb, from 6.0 kb to 7.0 kb, from 6.5 kb to 7.5 kb, from 7.0 kb to 8.0 kb, from 7.5 kb to 8.5 kb, from 8.0 kb to 9.0 kb, from 8.5 kb to 9.5 kb, or from 9.0 kb to 10.0 kb.

In some embodiments the ratio of the length of the heterologous nucleic acid sequence to the total length of the genome of the recombinant phage is at least 0.05, at least 0.10, at least 0.15, at least 0.20, or at least 0.25. In some embodiments the ratio of the length of the genome of the recombinant phage to the length of the genome of the corresponding starting phage is at least 1.05, at least 1.10, at least 1.15, at least 1.20, or at least 1.25.

In some embodiments the heterologous nucleic acid sequence is inserted into the starting phage genome with no loss of endogenous starting phage genome sequence. In some embodiments the inserted heterologous nucleic acid sequence replaces endogenous starting phage genome sequence. In some such embodiments the heterologous nucleic acid sequence replaces an amount of endogenous genomic sequence that is less than the length of the heterologous nucleic acid sequence. Thus, in such embodiments the length of the recombinant phage genome is longer than the length of the starting phage genome. In some such embodiments the heterologous nucleic acid sequence replaces an amount of endogenous genomic sequence that is greater than the length of the heterologous nucleic acid sequence. Thus, in such embodiments the length of the recombinant phage genome is shorter than the length of the starting phage genome. In some such embodiments the heterologous nucleic acid sequence replaces an amount of endogenous genomic sequence that is equal to the length of the heterologous nucleic acid sequence.

In some embodiments the protein or polypeptide encoded by a heterologous open reading frame is modified to reduce cleavage by proteases present in phage host cells. For example, computational algorithms can be used to identify known protease cleavage sites and the sequence of the open reading frame may be modified using conservative substitutions to remove these sites. Alternatively, directed mutagenesis is used to evolve the open reading frame sequence to encode a product that has an increased resistance to at least one protease present in a phage host cell or in the culture of a phage host cell.

This disclosure also provides isolated nucleic acids obtainable from a recombinant phage of this disclosure. In some embodiments the isolated nucleic acid is an isolated genome of a recombinant phage of this disclosure. In some embodiments the isolated nucleic acid comprises a fragment of less than the total genome of recombinant phage of this disclosure, the fragment comprising at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98% or at least 99% of the genome of the recombinant phage. In some embodiments the isolated nucleic acid comprises a fragment of less than the total genome of recombinant phage of this disclosure, the fragment comprising at least 20 bp, at least 50 bp, at least 100 bp, at least 500 bp, at least 1 kb, at least 2 kb, at least 3 kb, at least 4 kb, or at least 5 kb of the phage genome. In some embodiments the isolated nucleic acid comprises a fragment that is homologous to a fragment disclosed in this paragraph.

### Phase Target Bacteria

The recombinant phage of this disclosure may be used to detect the presence of bacteria. Detection of target bacteria is based on the ability of the recombinant phage to bind to target bacteria, transfer of the phage genome into the target bacteria, and express the heterologous nucleic acid sequence encoding a marker by the bacteria. Accordingly, the specificity of a method of detecting target bacteria using recombinant phage comprising a heterologous nucleic acid sequence encoding a marker is based on the range of bacterial types that support expression of the marker following exposure to the phage. Sometimes the range of bacterial types that support expression of the marker following exposure to the phage is referred to herein as the “host range” of the phage. The set of bacterial types that make up the host range of the phage is sometimes referred to herein as “target bacteria” for the phage.

This disclosure provides novel methods of assessing phage host range and thus of defining target bacteria for a phage. In certain embodiments the methods comprise exposing a candidate type of bacteria to a phage in a liquid culture. The ability of the phage to cause clearing of the culture, which reflects infection and lysis of bacteria in the culture by the phage, is an indication that the bacteria in the culture are target bacteria of the phage. As demonstrated in the examples this method is surprisingly more accurate in assessing the true phage host range for a phage than prior art plate-based plaque assays. In some embodiments herein, the “host range” of a phage or the “target bacteria” of a phage are defined based on a set of bacteria that a phage can clear in a liquid culture-based assay.

While the liquid culture method is an improvement over prior methods and is very useful for many purposes, it does embody all aspects of methods of using a recombinant phage to detect target bacteria. Such methods rely on the ability of the recombinant phage to bind to target bacteria, transfer of the phage genome into the target bacteria, and expression of the heterologous nucleic acid sequence encoding a marker by the bacteria. Accordingly, even if a phage is unable to lyse a liquid culture of a particular bacterial cell type the phage may nonetheless be able to bind to the bacteria type, transfer the phage genome into the target bacteria, and thus cause expression of a heterologous nucleic acid sequence encoding a marker by the bacteria. Indeed, as demonstrated by the examples, assays that detect the presence of the marker in a type of bacteria following exposure to a recombinant phage are in some embodiments more sensitive even than liquid based host range assays. Accordingly, in some embodiments herein, the “host range” of a phage or the “target bacteria” of a phage are defined by a process that comprises 1) providing a recombinant phage comprising a heterologous nucleic acid sequence encoding a marker; 2) exposing a sample to the phage; and 3) assaying for the presence of the marker in the exposed sample. This type of assay is sometimes referred to herein generally as a “marker host range assay.” In some embodiments assaying for the presence of the marker in the exposed sample is by a method comprising detection of an mRNA. In some embodiments assaying for the presence of the marker in the exposed sample is by a method comprising direct detection of marker protein, such as using an antibody. In some embodiments assaying for the presence of the marker in the exposed sample is by a method comprising functional detection of marker protein. For example, if the marker protein is a luciferase the exposed sample may be exposed to luciferin and production of light may be assayed. This method may be adapted to any type of marker disclosed herein and skilled artisans are aware that many variations on the detection method of the marker may be used.

Certain variables may modify the host range of phage under certain conditions. Conditions that sustain constant bacterial growth and therefore maximal bacteriophage infectivity are seldom found in environments where methods of detecting bacteria are useful. Oligotrophic environments and competition among microorganisms force bacteria to be able to adapt quickly to rough and changing situations. A particular lifestyle composed of continuous cycles of growth and starvation is commonly referred to as feast and famine. Bacteria have developed many different mechanisms to survive in nutrient-depleted and harsh environments, varying from producing a more resistant vegetative cell to complex developmental programs. As a consequence of prolonged starvation, certain bacterial species enter a dynamic non-proliferative state in which continuous cycles of growth and death occur until ‘better times’ come, a.k.a. restoration of favorable growth conditions and with them the favorable infective condition.

The infectivity of bacteriophages is determined in part not only by the specificity of their encoded tail fiber recognition proteins, but also by the environmental conditions that are present. That includes but is not limited to the metabolic state of the bacterium the bacteriophage is capable of recognizing. Furthermore, it includes the chemical and physical composition of the environment that the bacteriophage and the bacterium experience when the phage contacts a bacterium. Environmental factors of the solution such as but not limited to pH, osmolarity, temperature, rheological properties and others all may impact the ability of a bacteriophage to infect a bacterium.

To account for these variables, the step of exposing a sample of bacteria to a phage in the liquid clearing host-range assay and the marker host range assay may be conducted under defined conditions. The defined conditions may comprise at least one of: a defined time duration, a defined temperature, and the presence of at least one of a) at least one compound selected from carbohydrates and related compounds, b) at least compound selected from nitrogen containing compounds, c) at least compound selected from nucleic acids and related compounds, d) at least compound selected from lipid, e) at least one inorganic compound, and f) at least one organic compound.

In some embodiments the carbohydrates and related compounds are selected from sugars such as glucose, mannose, and maltose. In some embodiments the carbohydrates and related compounds are selected from carboxy sugars that are degraded by the pentose phosphate pathway, which may but need not generate more moles of NADPH per mole consumed as compared to glucose. In some embodiments the carbohydrates and related compounds are selected from compounds feeding into central metabolism, such as but not limited to a ketoglutarate, D-malic acid, or pyruvic acid. In some embodiments the carbohydrates and related compounds are selected from glycerol and other carbohydrate (or other) osmoprotectants that may but need not provide osmotic support to cells that exist in a potentially weakened or damaged state in the environment. In some embodiments glycerol functions as a volume excluder that increases the efficiency of phage infection. In some embodiments the carbohydrates and related compounds are selected from sugar alcohols, such as aminoethanol.

In some embodiments the nitrogen containing compounds are selected from ammonium, other amino acid building blocks, and free amino acids. The free amino acid may be any genome encoded standard amino acid or any non-standard amino acid. In some embodiments the amino acid is selected from glutamic acid and glutamine. In some embodiments the amino acid is selected from branched chain amino acids. In some embodiments the nitrogen containing compounds are selected from degradation products of branched amino acids such as propionic acid.

In some embodiments the nucleic acids and related compounds are selected from nucleotides, nucleosides, deoxynucleotides, and deoxynucleosides. In some embodiments the nucleic acids and related compounds are selected from metabolites of the nucleotide generation pathways such as inosine.

In some embodiments the lipid compounds are selected from fatty acids and related compounds. Tween 20, 40, and 80 are converted to fatty acids upon ester hydrolysis and can also be used. In some embodiments the lipid compounds are selected from lecithin and related compounds.

In some embodiments the inorganic compounds are selected from salts, such as for example thiosulfate.

In some embodiments the organic compounds are selected from aliphatics, aromatics, heterocyclics, and non-biogenic polymers.

In some embodiments the at least one compound is selected from:

| Compound | CAS # |
|---|---|
| 1,2-Propanediol | 57-55-6 |
| 2-Aminoethanol | 141-43-5 |
| Glucuronamide | 3789-97-7 |
| Tyramine | 60-19-5 |
| b-Phenylethylamine | 156-28-5 |
| L-Aspartic Acid | 3792-50-5 |
| L-Proline | 147-85-3 |
| D-Alanine | 338-69-2 |
| D-Serine | 312-84-5 |
| L-Glutamic Acid | 6106-04-3 |
| L-Asparagine | 70-47-3 |
| D-Aspartic Acid | 1783-96-6 |
| L-Glutamine | 56-85-9 |
| Gly-Asp | |
| D-Threonine | 632-20-2 |
| Gly-Glu | 7412-78-4 |
| L-Serine | 56-45-1 |
| L-Threonine | 72-19-5 |
| L-Alanine | 56-41-7 |
| Ala-Gly | 687-69-4 |
| Gly-Pro | 704-15-4 |
| L-Arabinose | 87-72-9 |
| N-Acetyl-D-Glucosamine | 7512-17-6 |
| D-Galactose | 59-23-4 |
| D-Trehalose | 99-20-7 |
| D-Mannose | 3458-28-4 |
| Dulcitol | 608-66-2 |
| D-Sorbitol | 50-70-4 |
| Glycerol | 56-81-5 |
| L-Fucose | 2438-80-4 |
| D,L-a-Glycerol Phosphate | 3325-00-6 |
| D-Xylose | 58-86-6 |
| D-Mannitol | 69-65-8 |
| D-Glucose-6-Phosphate | 3671-99-6 |
| D-Ribose | 50-69-1 |
| L-Rhamnose | 3615-41-6 |
| D-Fructose | 57-48-7 |
| a-D-Glucose | 50-99-7 |
| Maltose | 69-79-4 |
| D-Melibiose | 585-99-9 |
| Thymidine | 50-89-5 |
| a-Methyl-D-Galactoside | 3396-99-4 |
| a-D-Lactose | 63-42-3 |
| Lactulose | 4618-18-2 |
| Sucrose | 57-50-1 |
| Uridine | 58-96-8 |
| D-Glucose-1-Phosphate | 56401-20-8 |
| D-Fructose-6-Phosphate | 26177-86-637250-85-4 |
| b-Methyl-D-Glucoside | 709-50-2 |
| Adonitol | 488-81-3 |
| Maltotriose | 1109-28-0 |
| 2′-Deoxyadenosine | 16373-93-6 |
| Adenosine | 58-61-7 |
| m-Inositol | 87-89-8 |
| D-Cellobiose | 528-50-7 |
| Inosine | 58-63-9 |
| N-Acetyl-D-Mannosamine | 7772-94-3 |
| D-Psicose | 551-68-8 |
| L-Lyxose | 1949-78-6 |
| D-Saccharic Acid | 576-42-1 |
| Succinic Acid | 6106-21-4 |
| D-Glucuronic Acid | 14984-34-0 |
| D-Gluconic Acid | 527-07-1 |
| DL-Lactic Acid | 50-21-5 |
| Formic Acid | 141-53-7 |
| D-Galactonic Acid-g-Lactone | 2782-07-2 |
| D,L-Malic Acid | 6915-15-7 |
| Acetic Acid | 127-09-3 |
| D-Glucosaminic Acid | 3646-68-2 |
| a-Ketoglutaric Acid | 22202-68-2 |
| a-Ketobutyric Acid | 2013-26-5 |
| m-Tartaric Acid | 147-73-9 |
| a-Hydroxyglutaric Acid-g-Lactone | 21461-84-7 |
| a-Hydroxybutyric Acid | 19054-57-0 |
| Citric Acid | 6132-04-3 |
| Fumaric Acid | 17013-01-3 |
| Bromosuccinic Acid | 923-06-8 |
| Propionic Acid | 137-40-6 |
| Mucic Acid | 526-99-8 |
| Glycolic Acid | 79-14-1 |
| Glyoxylic Acid | 563-96-2 |
| Tricarballylic Acid | 99-14-9 |
| Acetoacetic Acid | 3483-11-2 |
| Mono-Methylsuccinate | 3878-55-5 |
| D-Malic Acid | 636-61-3 |
| L-Malic Acid | 138-09-0 |
| p-Hydroxyphenyl Acetic Acid | 156-38-7 |
| m-Hydroxyphenyl Acetic Acid | 621-37-4 |
| Pyruvic Acid | 113-24-6 |
| L-Galactonic Acid-g-Lactone | 1668-08-2 |
| D-Galacturonic Acid | 91510-62-2 |
| Methylpyruvate | 600-22-6 |
| Tween 20 | 9005-64-5 |
| Tween 40 | 9005-66-7 |
| Tween 80 | 9005-65-6 |

Another approach to modify the host range detected in a host range assay is to pretreat bacteria before exposing the bacterial samples to the phage. This allows for a decoupling of steps designed to modify the state of a bacterial cell (and possibly its susceptibility to phage infection) from conditions used for the infection itself. For example the metabolic rate may be increased during a pre-incubation step, which in turn may increase at least one of the replicative, transcriptive, and translative functions that influence clearing or production of a marker following infection of a bacterial cell by a phage. Furthermore, it is possible that such an incubation period also changes the surface receptor expression, or changes the composition of the cell wall of the bacterium, which may also modify whether a phage can productively infect the bacteria.

Accordingly, in some embodiments samples of bacteria are incubated in metabolic stimulation conditions before exposure to the phage for the phage host range assay. In some embodiments exposure of the cells to metabolic stimulation conditions stimulates cell division in the cells. In some embodiments exposure of the cells to metabolic stimulation conditions does not stimulate cell division in the cells. In some embodiments, exposure of the cells to metabolic stimulation conditions stimulates at least one of the replicative, transcriptive, and translative functions that influence clearing or production of a marker following infection of a bacterial cell by a phage.

As used herein, “metabolic stimulation conditions” are conditions that promote development of a microorganism metabolic state in which the microorganism is permissive to infection and maintenance of a phage life cycle and/or infection followed by expression of a marker gene produce encoded by a heterologous nucleic acid sequence in the genome of the phage. In some embodiments the microorganism prior to exposure to the metabolic stimulation conditions is not permissive to infection and maintenance of a phage life cycle. In other embodiments the microorganism prior to exposure to the metabolic stimulation conditions is in a metabolic state that reduces its susceptibility to infection and maintenance of a phage life cycle compared to a comparable microorganism grown under log phase conditions. In such embodiments exposure of the microorganism to the metabolic stimulation conditions increases the susceptibility of the microorganism to infection and maintenance of a phage life cycle. In some embodiments metabolic stimulation conditions comprise at least one of a permissive temperature, pH, Po₂, and nutrient combination. In some embodiments the target microbe undergoes at least one cell division under metabolic stimulation conditions. In some embodiments the target microbe does not undergo at least one cell division under metabolic stimulation conditions.

In some embodiments the sample is exposed to metabolic stimulation conditions before the sample is contacted with a phage. In some such embodiments the sample is then removed from metabolic stimulation conditions prior to contacting with a phage while in other embodiments the sample is maintained under metabolic stimulation conditions when contacted by a phage. In some embodiments the sample is exposed to a first set of metabolic stimulation conditions for a first period of time and then transferred to a second set of metabolic stimulation conditions. In some embodiments the recombinant phage is exposed to the sample while the sample is maintained under the second set of metabolic stimulation conditions. In some embodiments the sample is exposed to metabolic stimulation conditions for from 5 minutes to 24 hours before the sample is contacted by a phage. In some embodiments the sample is exposed to metabolic stimulation conditions for from 5 minutes to 6 hours before the sample is contacted by a phage. In some embodiments the sample is exposed to metabolic stimulation conditions for from 10 minutes to 6 hours before the sample is contacted by a phage. In some embodiments the sample is exposed to metabolic stimulation conditions for from 20 minutes to 6 hours before the sample is contacted by a phage. In some embodiments the sample is exposed to metabolic stimulation conditions for from 30 minutes to 6 hours before the sample is contacted by a phage. In some embodiments the sample is exposed to metabolic stimulation conditions for from 1 to 6 hours before the sample is contacted by a phage. In some embodiments the sample is exposed to metabolic stimulation conditions for from 2 to 6 hours before the sample is contacted by a phage. In some embodiments the sample is exposed to metabolic stimulation conditions for from 2 to 12 hours before the sample is contacted by a phage. In some embodiments the sample is exposed to metabolic stimulation conditions for from 3 to 12 hours before the sample is contacted by a phage. In some embodiments the sample is exposed to metabolic stimulation conditions for from 6 to 12 hours before the sample is contacted by a phage. In some embodiments the sample is exposed to metabolic stimulation conditions for from 12 to 24 hours before the sample is contacted by a phage. In some embodiments the sample is exposed to metabolic stimulation conditions for at least 10 minutes, at least 20 minutes, at least 30 minutes, at least 40 minutes, at least 50 minutes, at least 1 hour, at least 1.5 hours, or at least 2 hours.

By conducting a host range analysis under at least one embodiment of conditions described in this section it is possible to define conditions that provide a useful level of sensitivity and/or selectivity for a method of detecting target bacteria. In some embodiments the conditions used for the host range analysis are also used for methods of detecting target bacteria using the phage when those phage are used to detect target bacteria in other contexts (i.e., when testing environmental samples).

### Methods of Detecting Target Bacteria

The recombinant phage are useful to detect target microbes. This disclosure provides exemplary recombinant phage and methods of making further recombinant phage. This disclosure also defines the target bacteria of certain disclosed recombinant phage and provides methods of identifying the target bacteria of any phage, including any recombinant phage. Accordingly, this disclosure enables methods of detecting target microbes using recombinant phage. By, among other things, enabling a detailed characterization of the target bacteria of the recombinant phage this disclosure in certain embodiments provides useful methods not available in the prior art.

The methods are broadly applicable and in view of the teachings of this disclosure skilled artisans will understand how to apply the methods to detect any type of archaea and/or bacteria. In some embodiments the archaea is a Euryarcheota. In some embodiments the archaea is a Crenarcheota. In some embodiments the bacteria is a member of a phyla selected from Actinobacteria, Aquificae, Armatimonadetes, Bacteroidetes, Caldiserica, Chlamydiae, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Nitrospirae, Planctomycetes, Proteobacteria, Spirochaetes, Synergistets, Tenericutes, Thermodesulfobacteria, Thermotogae. In some embodiments the bacteria is at least one Firmicutes selected from Bacillus, Listeria, Staphylococcus. In some embodiments the bacteria is at least one Proteobacteria selected from Acidobacillus, Aeromonas, Burkholderia, Neisseria, Shewanella, Citrobacter, Enterobacter, Erwinia, Escherichia, Klebsiella, Kluyvera, Morganella, Shigella, Yersinia, Coxiella, Rickettsia, Legionella, Avibacterium, Haemophilus, Pasteurella, Acinetobacter, Moraxella, Pseudomonas, Vibrio, Xanthomonas. In some embodiments the bacteria is at least one Tenericutes selected from Mycoplasma, Spiroplasma, and Ureaplasma.

Common bacterial contaminates of food that are detected using the phage and methods disclosed herein include, without limitation, E. coli (including without limitation pathogenic E. coli, E. coli O157:H7, Shiga-toxin producing E. coli, E. coli 026, E. coli 111, E. coli 0103, E. coli 0121, E. coli 045 and E. coli 0145), coliform bacteria (which include without limitation, Citrobacter, Enterobacter, Hafnia, Klebsiella, Serratia), Shigella, Listeria, Clostridium (including Clostridium botulinum and Clostridium perfringens), Vibrio (including Vibrio cholera and Vibrio vulnificus), Enterobacteriacae, Staphylococcus (including Staphylococcus aureus and Staphylococcus epidermis), Bacillus (including Bacillus cereus), Campylobacter (including Campylobacter jejuni), Pseudomonas, Streptococcus, Acinetobacter, Klebsiella, Campylobacter, and Yersinia.

The methods comprise providing a sample; exposing the sample to at least a first type of recombinant phage capable of infecting at least a first set of target bacteria, comprising a heterologous nucleic acid sequence encoding at least a first marker and assay for the at least one first marker in the exposed sample. Preferably, the first type of recombinant phage comprises a heterologous nucleic acid sequence; a codon optimized at least first markers. In some embodiments, detection of the first marker in the sample indicates the presence of bacteria of the first set of target bacteria in the sample.

In certain embodiments the methods comprise providing a sample; exposing the sample to a first type of phage capable of infecting a first set of target bacteria and comprising a heterologous nucleic acid sequence encoding a first marker; exposing the sample to a second type of phage capable of infecting a second set of target bacteria and comprising a heterologous nucleic acid sequence encoding a second marker; and assaying for the presence of the first marker and the second marker in the exposed sample. In some embodiments, detection of the first marker in the sample indicates the presence of bacteria of the first set of target bacteria in the sample. In some embodiments, detection of the second marker in the sample indicates the presence of bacteria of the second set of target bacteria in the sample. In some embodiments the first marker and the second marker are the same, and detection of the marker in the sample indicates the presence of bacteria of at least one of the first set of target bacteria and the second set of target bacteria in the sample.

In some embodiments, the first set of target bacteria and the second set of target bacteria independently comprise at least two species of a single genus of bacteria. In some embodiments, the first set of target bacteria and the second set of target bacteria independently comprise at least three species of a single genus of bacteria. In some embodiments, the first set of target bacteria and the second set of target bacteria independently comprise at least four species of a single genus of bacteria. In some embodiments, the single genus of bacteria is Listeria. In some embodiments, the first set of target bacteria and the second set of target bacteria comprise at least one species of bacteria in common. In some embodiments, the first set of target bacteria and the second set of target bacteria comprise at least two species of bacteria in common. In some embodiments, the first set of target bacteria and the second set of target bacteria comprise at least three species of bacteria in common. In some embodiments, the first set of target bacteria and the second set of target bacteria comprise at least four species of bacteria in common. In some embodiments, the species of Listeria are selected from Listeria innocua, Listeria monocytogenes, Listeria seeligeri, Listeria ivanovii, Listeria marthii, Listeria rocourti and Listeria welshimeri. In some embodiments, the species of Listeria are selected from Listeria innocua, Listeria monocytogenes, Listeria seeligeri, and Listeria welshimeri.

In some embodiments, the target bacteria comprise at least one sig B allelotype of Listeria innocua selected from 11, 22, 37, and 56. In some embodiments, the target bacteria comprise at least four allelotypes of Listeria innocua. In some embodiments, the at least four allelotypes of Listeria innocua are 11, 22, 37, and 56.

In some embodiments, the target bacteria comprise at least one ribotype of Listeria monocytogenes selected from DUP-10142, DUP-1030A, DUP-1030B, DUP-1038B, DUP-1039A, DUP-1039B, DUP-1039C, DUP-1042A, DUP-1042B, DUP-1042C, DUP-1043A, DUP-1044A, DUP-1044B, DUP-1044E, DUP-1045B, DUP-1052A, DUP-1053A, DUP-1062A, and DUP-1062D. In some embodiments, the target bacteria comprise at least nineteen ribotypes of Listeria monocytogenes. In some embodiments, the at least nineteen ribotypes of Listeria monocytogenes are DUP-10142, DUP-1030A, DUP-1030B, DUP-1038B, DUP-1039A, DUP-1039B, DUP-1039C, DUP-1042A, DUP-1042B, DUP-1042C, DUP-1043A, DUP-1044A, DUP-1044B, DUP-1044E, DUP-1045B, DUP-1052A, DUP-1053A, DUP-1062A, and DUP-1062D.

In some embodiments, the target bacteria comprise at least one sig B allelotype of Listeria seeligeri selected from 3, 20, 24, and 35. In some embodiments, the target bacteria comprise at least four allelotypes of Listeria seeligeri. In some embodiments, the at least four allelotypes of Listeria seeligeri are 3, 20, 24, and 35.

In some embodiments, the target bacteria comprise at least one sig B allelotype of Listeria welshimeri selected from 15, 27, 32, and 89. In some embodiments, the target bacteria comprise at least four allelotypes of Listeria welshimeri. In some embodiments, the at least four allelotypes of Listeria welshimeri are 15, 27, 32, and 89.

In some embodiments, the first set of target bacteria are all members of the same genus. In some embodiments, the second set of target bacteria are all members of the same genus. In some embodiments, all of the target bacteria are Listeria. In some embodiments, the target bacteria do not include at least one of Bacillus cereus, Bacillus megaterium, Bacillus subtilis, Enterococcus durans, Enterococcus faceium, Enterococcus hirae, Kocuria varians, Kurthia gibsonii, Kurthia zopfii, Rhodococcus equi, Staphylococcus aureus, Staphylococcus epidermidis, Staphylococcus saprophyticus, Streptococcus equi, Streptococcus galloyticus, Lactobacillus casei, Lactobacillus buchneri, Lactobacillus lactus, Lactobacillus fermentum, Micrococcus lutues, Pseudomonas protogens, Pseudomonas florescens, Aeromonas sp, Serratia liquefaciens, Serratia proteamaculans, Serratia liquefaciens, Bacillaceae bacterium, Serratia proteamaculans, Pseudomonas florescens, Pseudomonas poae, Pseudomonas sp, Pseudomonas fragi, Providencia alcalifaciens, Serratia sp, Serratia grimesii, Hafnia sp., Serratia proteamaculans, Pseudomonas florescens, Chryseobacterium sp., Pseudomonas fragi, and Enterobacteriaceae. In some embodiments, the target bacteria do not include Bacillus cereus, Bacillus megaterium, Bacillus subtilis, Enterococcus durans, Enterococcus faceium, Enterococcus hirae, Kocuria varians, Kurthia gibsonii, Kurthia zopfii, Rhodococcus equi, Staphylococcus aureus, Staphylococcus epidermidis, Staphylococcus saprophyticus, Streptococcus equi, Streptococcus galloyticus, Lactobacillus casei, Lactobacillus buchneri, Lactobacillus lactus, Lactobacillus fermentum, Micrococcus lutues, Pseudomonas protogens, Pseudomonas florescens, Aeromonas sp, Serratia liquefaciens, Serratia proteamaculans, Serratia liquefaciens, Bacillaceae bacterium, Serratia proteamaculans, Pseudomonas florescens, Pseudomonas poae, Pseudomonas sp, Pseudomonas fragi, Providencia alcalifaciens, Serratia sp, Serratia grimesii, Hafnia sp., Serratia proteamaculans, Pseudomonas florescens, Chryseobacterium sp., Pseudomonas fragi, and Enterobacteriaceae.

In some embodiments, the methods further comprise exposing the sample to a third type of phage capable of infecting a third set of target bacteria and comprising a heterologous nucleic acid sequence encoding a third marker. In some embodiments, the methods further comprise exposing the sample to a fourth type of phage capable of infecting a fourth set of target bacteria and comprising a heterologous nucleic acid sequence encoding a fourth marker. In some embodiments, the methods further comprise exposing the sample to a fifth type of phage capable of infecting a fifth set of target bacteria and comprising a heterologous nucleic acid sequence encoding a fifth marker. In some embodiments, the methods further comprise exposing the sample to a sixth type of phage capable of infecting a sixth set of target bacteria and comprising a heterologous nucleic acid sequence encoding a sixth marker. In some embodiments, the methods further comprise exposing the sample to a seventh type of phage capable of infecting a seventh set of target bacteria and comprising a heterologous nucleic acid sequence encoding a seventh marker. In some embodiments, the methods further comprise exposing the sample to an eighth type of phage capable of infecting an eighth set of target bacteria and comprising a heterologous nucleic acid sequence encoding an eighth marker. In some embodiments, the methods further comprise exposing the sample to a ninth type of phage capable of infecting a ninth set of target bacteria and comprising a heterologous nucleic acid sequence encoding a ninth marker. In some embodiments, the methods further comprise exposing the sample to ten or more types of phage capable of infecting ten or more sets of target bacteria and comprising a heterologous nucleic acid sequences encoding ten or more markers. In some embodiments that utilize three or more types of phage, all of the three or more markers are different. In some embodiments that utilize three or more types of phage, all of the three or more markers are the same. In some embodiments that utilize three or more types of phage, two, three, four, five, six, seven, eight, or nine of the markers are the same.

In some embodiments, at least one type of phage used in the method is selected from A511, P100, LP40, LP48, LP99, LP101, LP124, LP125, and LP143, and derivatives thereof. In some embodiments, every type of phage used in the method is selected from A511, P100, LP40, LP48, LP99, LP101, LP124, LP125, and LP143, and derivatives thereof.

In some embodiments, the first marker is a screenable marker. In some embodiments, the first marker is a luciferase. In some embodiments, the luciferase is at least 70%, at least 80%, at least 90%, at least 91%, at least 92%, at least 93%, at least 94%, at least 95%, at least 96%, at least 97%, at least 98%, at least 99%, at least 99.5% or at least 99.9% identical to SEQ ID NO: 2. In some embodiments, the luciferase is at least 70%, at least 80%, at least 90%, at least 91%, at least 92%, at least 93%, at least 94%, at least 95%, at least 96%, at least 97%, at least 98%, at least 99%, at least 99.5% or at least 99.9% identical to SEQ ID NO: 4.

In some embodiments, the phage is selected from LP48::ffluc, LP99::ffluc, LP101::ffluc, LP124::ffluc, LP125::ffluc, LP143::ffluc, A511::ffluc, P100::ffluc, LP48:: COP2, LP99:: COP2, LP101:: COP2, LP124:: COP2, LP125:: COP2, LP143:: COP2, A511:: COP2, P100:: COP2, LP48:: COP3, LP99:: COP3, LP101:: COP3, LP124:: COP3, LP125:: COP3, LP143:: COP3, A511:: COP3, and P100:: COP3. In some embodiments, the phage is selected from LP40::nluc, LP124::nluc, LP125::nluc, A511::nluc, P100::nluc, LP40::COP2, LP124::COP2, LP125:: COP2, A511:: COP2, P100:: COP2, LP40::COP3, LP124::COP3, LP125:: COP3, A511:: COP3, P100:: COP3.

In some embodiments, the sample is an environmental sample.

In some embodiments, the sample is an agricultural sample. In some embodiments the agricultural sample is stock feed or food supply. In some embodiments, the food supply is for human or non-human consumption. In some embodiments, the food supply is a plant or an animal.

In some embodiments, the agricultural sample in the composition is selected from a dairy product, a fruit product, a grain product, a sweet, a vegetable product, and a meat product. In some embodiments, the dairy product includes foods derived from milk products comprising milk, butter, yogurt, cheese, ice cream and queso fresco. In some embodiments, the fruit product comprises apple, oranges, bananas, berries and lemons. In some embodiments, the grain product comprises wheat, rice, oats, barley, bread and pasta. In some embodiments, the sweet product comprises candy, soft drinks, cake, and pie. In some embodiments, the vegetable product comprises spinach, carrots, onions, peppers, avocado and broccoli. In some embodiments, the vegetable product is guacamole. In some embodiments, the meat product comprises chicken, fish, turkey, pork and beef. In some embodiments, the meat product further comprises deli meats and ground meets, as well as deli turkey and ground beef.

In some embodiments, the food sample in the composition is selected from a dairy product, a fruit product, a grain product, a sweet, a vegetable product, and a meat product. In some embodiments, the dairy product includes foods derived from milk products comprising milk, butter, yogurt, cheese, ice cream and queso fresco. In some embodiments, the fruit product comprises apple, oranges, bananas, berries and lemons. In some embodiments, the grain product comprises wheat, rice, oats, barley, bread and pasta. In some embodiments, the sweet product comprises candy, soft drinks, cake, and pie. In some embodiments, the vegetable product comprises spinach, carrots, onions, peppers, avocado and broccoli. In some embodiments, the vegetable product is guacamole. In some embodiments, the meat product comprises chicken, fish, turkey, pork and beef. In some embodiments, the meat product further comprises deli meats and ground meets, as well as deli turkey and ground beef.

In some embodiments, the marker is detected in the sample, indicating the presence of bacteria of the first set of target bacteria in the sample.

In some embodiments, the target microbe of the method is selected from the group consisting ofcoliform bacteria, Escherichia, Shigella, Listeria, Clostridium, Vibrio, Enterobacteriacae, Staphylococcus, Bacillus, Campylobacter, Pseudomonas, Streptococcus, Acinetobacter, Klebsiella, Cronobacter, Mycobacterium, Campylobacter, and Yersinia. In some embodiments, the target microbe is E. coli. In some embodiments, the target microbe is Listeria selected from the group consisting of Listeria innocua, Listeria monocytogenes, Listeria seeligeri, Listeria ivanovii, Listeria grayi, Listeria marthii, Listeria rocourti, Listeria welshimeri, Listeria floridensis, Listeria aquatic, Listeria fleischmannii, Listeria weihenstephanensis, Listeria cornellensis, Listeria riparia, Listeria newyorkensis and Listeria grandensis.

In some embodiments, the second marker is a screenable marker. In some embodiments, the second marker is a luciferase. In some embodiments, the luciferase is at least 70%, at least 80%, at least 90%, at least 91%, at least 92%, at least 93%, at least 94%, at least 95%, at least 96%, at least 97%, at least 98%, at least 99%, at least 99.5% or at least 99.9% identical to SEQ ID NO: 2. In some embodiments, the luciferase is at least 70%, at least 80%, at least 90%, at least 91%, at least 92%, at least 93%, at least 94%, at least 95%, at least 96%, at least 97%, at least 98%, at least 99%, at least 99.5% or at least 99.9% identical to SEQ ID NO: 4.

In some embodiments, the second type of phage is selected from LP48::ffluc, LP99::ffluc, LP101::ffluc, LP124::ffluc, LP125::ffluc, LP143::ffluc, A511::ffluc, P100::ffluc, LP48::COP2, LP99::COP2, LP101::COP2, LP124::COP2, LP125::COP2, LP143::COP2, A511::COP2, P100::COP2, LP48::COP3, LP99::COP3, LP101::COP3, LP124::COP3, LP125::COP3, LP143::COP3, A511::COP3, P100::COP3. In some embodiments, the second type of phage is selected from LP40::nluc, LP124::nluc, LP125::nluc, A511::nluc, P100::nluc, LP40::COP2, LP124::COP2, LP125::COP2, A511::COP2, P100::COP2, LP40::COP3, LP124::COP3, LP125::COP3, A511::COP3, P100::COP3.

In some embodiments, the method comprises exposing the sample to the first type of phage and the second type of phage at the same time.

In some embodiments, the sample is an environmental sample.

In some embodiments, the first marker is detected in or on the sample, or in situ, indicating the presence of bacteria of the first set of target bacteria in or on the sample, or in situ. In some embodiments, the second marker is detected in the sample, indicating the presence of bacteria of the second set of target bacteria in the sample. In some embodiments, the first marker and the second marker are the same, and the marker is detected in or on the sample, or in situ, indicating the presence of bacteria of at least one of the first set of target bacteria and the second set of target bacteria in or on the sample, or in the in situ location.

In some embodiments, the sample is exposed to metabolic stimulation conditions before it is exposed to the phage.

In some embodiments, the methods further comprise incubating the sample under metabolic stimulation conditions for a period of time before exposing the sample to the phage capable of infecting target bacteria.

In certain embodiments the methods comprise providing a sample; exposing the sample to at least one recombinant Listeria phage comprising a heterologous nucleic acid sequence encoding a marker, the recombinant Listeria phage selected from recombinant LP40 and derivatives thereof, recombinant LP48 and derivatives thereof, recombinant LP99 and derivatives thereof, recombinant LP101 and derivatives thereof, recombinant LP124 and derivatives thereof, recombinant LP125 and derivatives thereof, and recombinant LP143 and derivatives thereof; and assaying for the presence of the marker in the exposed sample. In some embodiments, the methods further comprise exposing the sample to at least one recombinant Listeria phage comprising a heterologous nucleic acid sequence encoding a marker, the recombinant Listeria phage selected from recombinant A511 and recombinant P100. In some embodiments, detection of the marker in the sample indicates the presence of Listeria in the sample.

In some embodiments, target bacteria of the recombinant Listeria phage comprise at least one species of Listeria selected from Listeria innocua, Listeria monocytogenes, Listeria seeligeri, Listeria ivanovii, Listeria marthii, Listeria rocourti, and Listeria welshimeri. In some embodiments, detection of the marker in the sample indicates the presence of the at least one species of Listeria selected from Listeria innocua, Listeria monocytogenes, Listeria seeligeri, Listeria ivanovii, Listeria marthii, Listeria rocourti, and Listeria welshimeri in the sample.

In some embodiments, target bacteria of the Listeria phage comprise at least one species of Listeria selected from Listeria innocua, Listeria monocytogenes, Listeria seeligeri, and Listeria welshimeri. In some embodiments, detection of the marker in the sample indicates the presence of the at least one species of Listeria selected from Listeria innocua, Listeria monocytogenes, Listeria seeligeri, and Listeria welshimeri in the sample.

In some embodiments, target bacteria of the Listeria phage comprise at least one sig B allelotype of Listeria innocua selected from 11, 22, 37, and 56, and detection of the marker in the sample indicates the presence of at least one sig B allelotype of Listeria innocua selected from 11, 22, 37, and 56. In some embodiments, the at least one Listeria phage is capable of infecting Listeria innocua sig B allelotypes 11, 22, 37, and 56.

In some embodiments, target bacteria of the Listeria phage comprise at least one ribotype of Listeria monocytogenes selected from DUP-10142, DUP-1030A, DUP-1030B, DUP-1038B, DUP-1039A, DUP-1039B, DUP-1039C, DUP-1042A, DUP-1042B, DUP-1042C, DUP-1043A, DUP-1044A, DUP-1044B, DUP-1044E, DUP-1045B, DUP-1052A, DUP-1053A, DUP-1062A, and DUP-1062D; and detection of the marker in the sample indicates the presence of at least one ribotype of Listeria monocytogenes selected from DUP-10142, DUP-1030A, DUP-1030B, DUP-1038B, DUP-1039A, DUP-1039B, DUP-1039C, DUP-1042A, DUP-1042B, DUP-1042C, DUP-1043A, DUP-1044A, DUP-1044B, DUP-1044E, DUP-1045B, DUP-1052A, DUP-1053A, DUP-1062A, and DUP-1062D. In some embodiments, target bacteria of the Listeria phage comprise Listeria monocytogenes ribotypes DUP-10142, DUP-1030A, DUP-1030B, DUP-1038B, DUP-1039A, DUP-1039B, DUP-1039C, DUP-1042A, DUP-1042B, DUP-1042C, DUP-1043A, DUP-1044A, DUP-1044B, DUP-1044E, DUP-1045B, DUP-1052A, DUP-1053A, DUP-1062A, and DUP-1062D.

In some embodiments, target bacteria of the Listeria phage comprise at least one sig B allelotype of Listeria seeligeri selected from 3, 20, 24, and 35, and detection of the marker in the sample indicates the presence of at least one sig B allelotype of Listeria seeligeri selected from 3, 20, 24, and 35. In some embodiments, target bacteria of the Listeria phage comprise Listeria seeligeri sig B allelotypes 3, 20, 24, and 35.

In some embodiments, target bacteria of the Listeria phage comprise at least one sig B allelotype of Listeria welshimeri selected from 15, 27, 32, and 89, and detection of the marker in the sample indicates the presence of at least one sig B allelotype of Listeria welshimeri selected from 15, 27, 32, and 89. In some embodiments, target bacteria of the Listeria phage comprise Listeria welshimeri sig B allelotypes 15, 27, 32, and 89.

In some embodiments, the target bacteria comprise at least two species of Listeria selected from Listeria innocua, Listeria monocytogenes, Listeria seeligeri, and Listeria welshimeri. In some embodiments, the target bacteria comprise at least three species of Listeria selected from Listeria innocua, Listeria monocytogenes, Listeria seeligeri, and Listeria welshimeri. In some embodiments, the target bacteria comprise at least four species of Listeria selected from Listeria innocua, Listeria monocytogenes, Listeria seeligeri, Listeria ivanovii, Listeria marthii, Listeria rocourti, and Listeria welshimeri. In some embodiments, the target bacteria do not include at least one of Bacillus cereus, Bacillus megaterium, Bacillus subtilis, Enterococcus durans, Enterococcus faceium, Enterococcus hirae, Kocuria varians, Kurthia gibsonii, Kurthia zopfii, Rhodococcus equi, Staphylococcus aureus, Staphylococcus epidermidis, Staphylococcus saprophyticus, Streptococcus equi, Streptococcus galloyticus, Lactobacillus casei, Lactobacillus buchneri, Lactobacillus lactus, Lactobacillus fermentum, Micrococcus lutues, Pseudomonas protogens, Pseudomonas florescens, Aeromonas sp, Serratia liquefaciens, Serratia proteamaculans, Serratia liquefaciens, Bacillaceae bacterium, Serratia proteamaculans, Pseudomonas florescens, Pseudomonas poae, Pseudomonas sp, Pseudomonas fragi, Providencia alcalifaciens, Serratia sp, Serratia grimesii, Hafnia sp., Serratia proteamaculans, Pseudomonas florescens, Chryseobacterium sp., Pseudomonas fragi, and Enterobacteriaceae. In some embodiments, the target bacteria do not include Bacillus cereus, Bacillus megaterium, Bacillus subtilis, Enterococcus durans, Enterococcus faceium, Enterococcus hirae, Kocuria varians, Kurthia gibsonii, Kurthia zopfii, Rhodococcus equi, Staphylococcus aureus, Staphylococcus epidermidis, Staphylococcus saprophyticus, Streptococcus equi, Streptococcus galloyticus, Lactobacillus casei, Lactobacillus buchneri, Lactobacillus lactus, Lactobacillus fermentum, Micrococcus lutues, Pseudomonas protogens, Pseudomonas florescens, Aeromonas sp, Serratia liquefaciens, Serratia proteamaculans, Serratia liquefaciens, Bacillaceae bacterium, Serratia proteamaculans, Pseudomonas florescens, Pseudomonas poae, Pseudomonas sp, Pseudomonas fragi, Providencia alcalifaciens, Serratia sp, Serratia grimesii, Hafnia sp., Serratia proteamaculans, Pseudomonas florescens, Chryseobacterium sp., Pseudomonas fragi, and Enterobacteriaceae.

In some embodiments the sample is exposed to the phage for a period of time before assaying for the presence of a marker in the exposed sample is conducted. In some embodiments the period of time is from 1 minute to 24 hours, from 5 minutes to 12 hours, from 5 minutes to 6 hours, from 5 minutes to 3 hours, from 5 minutes to 2 hours, from 5 minutes to 1 hour, from 5 minutes to 50 minutes, from 5 minutes to 40 minutes, from 5 minutes to 30 minutes, from 5 minutes to 20 minutes, or from 5 minutes to 10 minutes. In some embodiments the period of time is from 1 to 2 hours, from 1 to 4 hours, or from 2 to 4 hours. In some embodiments the period of time is for at least 1 minute, at least 5 minutes, at least 10 minutes, at least 20 minutes, at least 30 minutes, at least 40 minutes, at least 50 minutes, or at least 1 hour.

In some embodiments any phage and/or parts of phage in the exposed sample are substantially removed before the assaying for the presence of a marker in the exposed sample is conducted.

In some embodiments of the methods of this disclosure, the methods further comprise comparing a detected level of marker in a test sample to at least one of a positive control and a negative control. The positive and/or negative control may be used to calibrate the assay including for the purpose of defining a positive result and/or a negative result.

### Compositions

The methods of assaying phage host range provided herein allow, in certain embodiments, for the characterization of the host range of phage—and thus definition of target bacteria for phage—at a resolution not previously provided. One use of the methods and of phage characterized by the methods is to identify useful combinations of phage that may be used together in a system to detect target bacteria. In some embodiments such systems provide phage separately and the phage are then mixed before or during an assay. Alternatively, such systems comprise useful mixtures of phage, such as phage provided in a buffer for use in an assay. Compositions comprising useful combinations of phage are also, necessarily, produced during the assay in several embodiments. Accordingly, this disclosure also provides compositions that comprise phage.

In some embodiments the composition comprises: at least one recombinant Listeria phage comprising a heterologous nucleic acid sequence encoding a marker, the recombinant Listeria phage selected from recombinant A511 and derivatives thereof, recombinant P100 and derivatives thereof, recombinant LP40 and derivatives thereof, recombinant LP44 and derivatives thereof, recombinant LP48 and derivatives thereof, recombinant LP99 and derivatives thereof, recombinant LP101 and derivatives thereof, recombinant LP124 and derivatives thereof, recombinant LP125 and derivatives thereof, and recombinant LP143 and derivatives thereof and at least one non-phage component selected from Table 5 and/or from at least one of a) at least one compound selected from carbohydrates and related compounds, b) at least compound selected from nitrogen containing compounds, c) at least compound selected from nucleic acids and related compounds, d) at least compound selected from lipid, e) at least one inorganic compound, and f) at least one organic compound. In some embodiments the composition comprises at least one of 1,2-Propanediol, 2-Aminoethanol, Glucuronamide, Tyramine, b-Phenylethylamine, L-Aspartic Acid, L-Proline, D-Alanine, D-Serine, L-Glutamic Acid, L-Asparagine, D-Aspartic Acid, L-Glutamine, Gly-Asp, D-Threonine, Gly-Glu, L-Serine, L-Threonine, L-Alanine, Ala-Gly, Gly-Pro, L-Arabinose, N-Acetyl-D-Glucosamine, D-Galactose, D-Trehalose, D-Mannose, Dulcitol, D-Sorbitol, Glycerol, L-Fucose, D,L-a-Glycerol, Phosphate, D-Xylose, D-Mannitol, D-Glucose-6-Phosphate, D-Ribose, L-Rhamnose, D-Fructose, a-D-Glucose, Maltose, D-Melibiose, Thymidine, a-Methyl-D-Galactoside, a-D-Lactose, Lactulosem Sucrose, Uridine, D-Glucose-1-Phosphate, D-Fructose-6-Phosphate, b-Methyl-D-Glucoside, Adonitol, Maltotriose, 2′-Deoxyadenosine, Adenosine, m-Inositol, D-Cellobiose, Inosine, N-Acetyl-D-Mannosamine, D-Psicose, L-Lyxose, D-Saccharic Acid, Succinic Acid, D-Glucuronic Acid, D-Gluconic Acid, D,L-Lactic Acid, Formic Acid, D-Galactonic Acid-g-Lactone, D,L-Malic Acid, Acetic Acid, D-Glucosaminic Acid, a-Ketoglutaric Acid, a-Ketobutyric Acid, m-Tartaric Acid, a-Hydroxyglutaric Acid-g-Lactone, a-Hydroxybutyric Acid, Citric Acid, Fumaric Acid, Bromosuccinic Acid, Propionic Acid, Mucic Acid, Glycolic Acid, Glyoxylic Acid, Tricarballylic Acid, Acetoacetic Acid, Mono-Methylsuccinate, D-Malic Acid, L-Malic Acid, p-Hydroxyphenyl Acetic Acid, m-Hydroxyphenyl Acetic Acid, Pyruvic Acid, L-Galactonic Acid-g-Lactone, D-Galacturonic Acid, Methylpyruvate, Tween 20, Tween 40, Tween 80.

In some embodiments the systems or compositions comprise at least two recombinant Listeria phage selected from recombinant LP40 and derivatives thereof, recombinant LP48 and derivatives thereof, recombinant LP99 and derivatives thereof, recombinant LP101 and derivatives thereof, recombinant LP124 and derivatives thereof, recombinant LP125 and derivatives thereof, and recombinant LP143 and derivatives thereof. In some embodiments the systems or compositions comprise at least three, four, five, six, seven, eight, nine, or more recombinant Listeria phage, selected from recombinant LP040 and derivatives thereof, recombinant LP048 and derivatives thereof, recombinant LP99 and derivatives thereof, recombinant LP101 and derivatives thereof, recombinant LP124 and derivatives thereof, recombinant LP125 and derivatives thereof, and recombinant LP143 and derivatives thereof.

### Articles of Manufacture

In some embodiments the system and or composition comprising at least one recombinant Listeria phage comprising a heterologous nucleic acid sequence encoding a marker is provided in the form of an article of manufacture. Such an article of manufacture is useful, for example, as a means to provide the at least one recombinant Listeria phage comprising a heterologous nucleic acid sequence encoding a marker in combination with other components that can be used together to perform an assay to detect target bacteria. In some embodiments the article of manufacture comprises at least one container comprising the at least one recombinant Listeria phage comprising a heterologous nucleic acid sequence encoding a marker.

In some embodiments the article of manufacture comprises at least one container comprising at least two recombinant Listeria phage selected from recombinant LP40 and derivatives thereof, recombinant LP48 and derivatives thereof, recombinant LP99 and derivatives thereof, recombinant LP101 and derivatives thereof, recombinant LP124 and derivatives thereof, recombinant LP125 and derivatives thereof, and recombinant LP143 and derivatives thereof. In some embodiments the systems or compositions comprise at least three, four, five, six, seven, eight, nine, or more recombinant Listeria phage, selected from recombinant LP40 and derivatives thereof, recombinant LP48 and derivatives thereof, recombinant LP99 and derivatives thereof, recombinant LP101 and derivatives thereof, recombinant LP124 and derivatives thereof, recombinant LP125 and derivatives thereof, and recombinant LP143 and derivatives thereof. In some embodiments in which the article of manufacture comprises more than one phage all of the phage are provided in separate containers. In other embodiments two or more of the phage are provided in combination in a single container.

The article of manufacture comprises at least one container comprising at least one recombinant phage selected from A511, P110, LP40, LP48, LP99, LP107, LP124, LP125 and LP143, and derivatives thereof. In some embodiments, the phage comprises a heterologous nucleic acid sequence encoding a first marker. In some embodiments, the first marker is a screenable marker. In some embodiments, the first marker is a luciferase. In some embodiments, the luciferase is at least 70%, at least 80%, at least 90%, at least 91%, at least 92%, at least 93%, at least 94%, at least 95%, at least 96%, at least 97%, at least 98%, at least 99%, at least 99.5% or at least 99.9% identical to SEQ ID NO: 2. In some embodiments, the luciferase is at least 70%, at least 80%, at least 90%, at least 91%, at least 92%, at least 93%, at least 94%, at least 95%, at least 96%, at least 97%, at least 98%, at least 99%, at least 99.5% or at least 99.9% identical to SEQ ID NO: 4. In some embodiments, the luciferase is at least 70%, at least 80%, at least 90%, at least 91%, at least 92%, at least 93%, at least 94%, at least 95%, at least 96%, at least 97%, at least 98%, at least 99%, at least 99.5% or at least 99.9% identical to SEQ ID NO: 41. In some embodiments, the first type of phage is selected from LP48::ffluc, LP99::ffluc, LP101::ffluc, LP124::ffluc, LP125::ffluc, LP143::ffluc, A511::ffluc, P100::ffluc, LP48::COP2, LP99::COP2, LP101::COP2, LP124::COP2, LP125::COP2, LP143::COP2, A511::COP2, and P100::COP2, LP48::COP3, LP99::COP3, LP101::COP3, LP124::COP3, LP125::COP3, LP143::COP3, A511::COP3, and P100::COP3 and derivatives of those phage. In some embodiments, the first type of phage is selected from LP40::nluc, LP124::nluc, LP125::nluc, A511::nluc, and P100::nluc. In some embodiments, the first type of phage is selected from A511::COP2, LP124::COP2, LP40::COP2, LP125::COP2, A511::COP3, LP124::COP3, LP40::COP3, and LP125::COP3.

In some embodiments the article of manufacture further comprises an aqueous solution including one or more reagents from Table 5 and/or at least one non-phage component selected from at least one of a) at least one compound selected from carbohydrates and related compounds, b) at least compound selected from nitrogen containing compounds, c) at least compound selected from nucleic acids and related compounds, d) at least compound selected from lipid, e) at least one inorganic compound, and f) at least one organic compound. In some embodiments, the article of manufacture comprises a container comprising a solution comprising at least one of 1,2-Propanediol, 2-Aminoethanol, Glucuronamide, Tyramine, b-Phenylethylamine, L-Aspartic Acid, L-Proline, D-Alanine, D-Serine, L-Glutamic Acid, L-Asparagine, D-Aspartic Acid, L-Glutamine, Gly-Asp, D-Threonine, Gly-Glu, L-Serine, L-Threonine, L-Alanine, Ala-Gly, Gly-Pro, L-Arabinose, N-Acetyl-D-Glucosamine, D-Galactose, D-Trehalose, D-Mannose, Dulcitol, D-Sorbitol, Glycerol, L-Fucose, D,L-a-Glycerol, Phosphate, D-Xylose, D-Mannitol, D-Glucose-6-Phosphate, D-Ribose, L-Rhamnose, D-Fructose, a-D-Glucose, Maltose, D-Melibiose, Thymidine, a-Methyl-D-Galactoside, a-D-Lactose, Lactulosem Sucrose, Uridine, D-Glucose-1-Phosphate, D-Fructose-6-Phosphate, b-Methyl-D-Glucoside, Adonitol, Maltotriose, 2′-Deoxyadenosine, Adenosine, m-Inositol, D-Cellobiose, Inosine, N-Acetyl-D-Mannosamine, D-Psicose, L-Lyxose, D-Saccharic Acid, Succinic Acid, D-Glucuronic Acid, D-Gluconic Acid, D,L-Lactic Acid, Formic Acid, D-Galactonic Acid-g-Lactone, D,L-Malic Acid, Acetic Acid, D-Glucosaminic Acid, a-Ketoglutaric Acid, a-Ketobutyric Acid, m-Tartaric Acid, a-Hydroxyglutaric Acid-g-Lactone, a-Hydroxybutyric Acid, Citric Acid, Fumaric Acid, Bromosuccinic Acid, Propionic Acid, Mucic Acid, Glycolic Acid, Glyoxylic Acid, Tricarballylic Acid, Acetoacetic Acid, Mono-Methylsuccinate, D-Malic Acid, L-Malic Acid, p-Hydroxyphenyl Acetic Acid, m-Hydroxyphenyl Acetic Acid, Pyruvic Acid, L-Galactonic Acid-g-Lactone, D-Galacturonic Acid, Methylpyruvate, Tween 20, Tween 40, Tween 80. In some embodiments at least one recombinant Listeria phage present in the article of manufacture is present in the aqueous solution comprising at least one non-phage component. In other embodiments the phage and solution are provided separately and may, for example, be combined by a user.

In another embodiment, the article of manufacture includes a substrate for a light reaction, or other required component for the marker to operate. By way of non-limiting example, the substrate is luciferin.

In another embodiment, the article of manufacture includes an additional aqueous solution that is optimized for a light reaction, or that provides conditions that are optimal for detection of a marker.

In some embodiments, the article of manufacture is a kit. The kit may further comprise instructions for performing one or more of the assays described herein.

### Definitions

It is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

The methods and techniques of the present disclosure are generally performed according to conventional methods well known in the art and as described in various general and more specific references that are cited and discussed throughout the present specification unless otherwise indicated. See, e.g., Sambrook et al., Molecular Cloning: A Laboratory Manual, 3d ed., Cold Spring Harbor Laboratory Press, Cold Spring Harbor, N.Y. (2001); Ausubel et al., Current Protocols in Molecular Biology, Greene Publishing Associates (1992, and Supplements to 2002); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999). Many molecular biology and genetic techniques applicable to phage are described in Clokie et al., Bacteriophages: Methods and Protocols, Vols. 1 and 2 (Methods in Molecular Biology, Vols. 501 and 502), Humana Press, New York, N.Y. (2009), which is hereby incorporated herein by reference.

This disclosure refers to sequence database entries (e.g., UniProt/SwissProt or GENBANK records) for certain amino acid and nucleic acid sequences that are published on the internet, as well as other information on the internet. The skilled artisan understands that information on the internet, including sequence database entries, is updated from time to time and that, for example, the reference number used to refer to a particular sequence can change. Where reference is made to a public database of sequence information or other information on the internet, it is understood that such changes can occur and particular embodiments of information on the internet can come and go. Because the skilled artisan can find equivalent information by searching on the internet, a reference to an internet web page address or a sequence database entry evidences the availability and public dissemination of the information in question.

The term “comprising” as used herein is synonymous with “including” or “containing”, and is inclusive or open-ended and does not exclude additional, unrecited members, elements or method steps. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe). An assay that occurs at least in part in vivo within a microbe may nonetheless occur in vitro if parts of the assay occur outside of the microbe in culture, for example.

As used herein, the term “isolated” refers to a substance or entity that has been (1) separated from at least some of the components with which it was associated when initially produced (whether in nature or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

The term “peptide” as used herein refers to a short polypeptide, e.g., one that typically contains less than about 50 amino acids and more typically less than about 30 amino acids. The term as used herein encompasses analogs and mimetics that mimic structural and thus biological function.

The term “polypeptide” encompasses both naturally-occurring and non-naturally occurring proteins, and fragments, mutants, derivatives and analogs thereof. A polypeptide may be monomeric or polymeric. Further, a polypeptide may comprise a number of different domains each of which has one or more distinct activities. For the avoidance of doubt, a “polypeptide” may be any length greater two amino acids.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from a cell in which it was synthesized.

The term “polypeptide fragment” as used herein refers to a polypeptide that has a deletion, e.g., an amino-terminal and/or carboxy-terminal deletion compared to a full-length polypeptide, such as a naturally occurring protein. In an embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, or at least 12, 14, 16 or 18 amino acids long, or at least 20 amino acids long, or at least 25, 30, 35, 40 or 45, amino acids, or at least 50 or 60 amino acids long, or at least 70 amino acids long.

The term “fusion protein” refers to a polypeptide comprising a polypeptide or fragment coupled to heterologous amino acid sequences. Fusion proteins are useful because they can be constructed to contain two or more desired functional elements that can be from two or more different proteins. A fusion protein comprises at least 10 contiguous amino acids from a polypeptide of interest, or at least 20 or 30 amino acids, or at least 40, 50 or 60 amino acids, or at least 75, 100 or 125 amino acids. The heterologous polypeptide included within the fusion protein is usually at least 6 amino acids in length, or at least 8 amino acids in length, or at least 15, 20, or 25 amino acids in length. Fusions that include larger polypeptides, such as an IgG Fc region, and even entire proteins, such as the green fluorescent protein (“GFP”) chromophore-containing proteins, have particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

As used herein, a protein has “homology” or is “homologous” to a second protein if the nucleic acid sequence that encodes the protein has a similar sequence to the nucleic acid sequence that encodes the second protein. Alternatively, a protein has homology to a second protein if the two proteins have similar amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89.

The following six groups each contain amino acids that are conservative substitutions for one another: 1) Serine, Threonine; 2) Aspartic Acid, Glutamic Acid; 3) Asparagine, Glutamine; 4) Arginine, Lysine; 5) Isoleucine, Leucine, Methionine, Alanine, Valine, and 6) Phenylalanine, Tyrosine, Tryptophan.

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

An exemplary algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Exemplary parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62. The length of polypeptide sequences compared for homology will generally be at least about 16 amino acid residues, or at least about 20 residues, or at least about 24 residues, or at least about 28 residues, or more than about 35 residues. When searching a database containing sequences from a large number of different organisms, it may be useful to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990). For example, percent sequence identity between amino acid sequences can be determined using FASTA with its default parameters (a word size of 2 and the PAM250 scoring matrix), as provided in GCG Version 6.1, herein incorporated by reference.

In some embodiments, polymeric molecules (e.g., a polypeptide sequence or nucleic acid sequence) are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% identical. In some embodiments, polymeric molecules are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% similar. The term “homologous” necessarily refers to a comparison between at least two sequences (nucleotides sequences or amino acid sequences). In some embodiments, two nucleotide sequences are considered to be homologous if the polypeptides they encode are at least about 50% identical, at least about 60% identical, at least about 70% identical, at least about 80% identical, at least 90%, at least 91%, at least 92%, at least 93%, at least 94%, at least 95%, at least 96%, at least 97%, at least 98% or at least 99% identical for at least one stretch of at least about 20 amino acids. In some embodiments, homologous nucleotide sequences are characterized by the ability to encode a stretch of at least 4-5 uniquely specified amino acids. Both the identity and the approximate spacing of these amino acids relative to one another must be considered for nucleotide sequences to be considered homologous. In some embodiments of nucleotide sequences less than 60 nucleotides in length, homology is determined by the ability to encode a stretch of at least 4-5 uniquely specified amino acids. In some embodiments, two protein sequences are considered to be homologous if the proteins are at least about 50% identical, at least about 60% identical, at least about 70% identical, at least about 80% identical, at least 90%, at least 91%, at least 92%, at least 93%, at least 94%, at least 95%, at least 96%, at least 97%, at least 98% or at least 99% identical for at least one stretch of at least about 20 amino acids.

As used herein, a “modified derivative” refers to polypeptides or fragments thereof that are substantially homologous in primary structural sequence to a reference polypeptide sequence but which include, e.g., in vivo or in vitro chemical and biochemical modifications or which incorporate amino acids that are not found in the reference polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵S, and ³H, ligands that bind to labeled antiligands (e.g., antibodies), fluorophores, chemiluminescent agents, enzymes, and antiligands that can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002).

As used herein, “polypeptide mutant” or “mutein” refers to a polypeptide whose sequence contains an insertion, duplication, deletion, rearrangement or substitution of one or more amino acids compared to the amino acid sequence of a reference protein or polypeptide, such as a native or wild-type protein. A mutein may have one or more amino acid point substitutions, in which a single amino acid at a position has been changed to another amino acid, one or more insertions and/or deletions, in which one or more amino acids are inserted or deleted, respectively, in the sequence of the reference protein, and/or truncations of the amino acid sequence at either or both the amino or carboxy termini. A mutein may have the same or a different biological activity compared to the reference protein.

In some embodiments, a mutein has, for example, at least 70% overall sequence homology to its counterpart reference polypeptide or protein. In some embodiments, a mutein has at least 75%, at least 80%, at least 85%, or at least 90% overall sequence homology to the wild-type protein or polypeptide. In other embodiments, a mutein exhibits at least 91%, at least 92%, at least 93%, at least 94%, at least 95%, at least 96%, at least 97%, at least 98%, at least 99% at least 99.5%, at least 99.9% sequence identity, or 98%, or 99%, or 99.5% or 99.9% overall sequence identity.

As used herein, “recombinant” refers to a biomolecule, e.g., a gene or protein, that (1) is not associated with all or a portion of a polynucleotide in which the gene is found in nature, (2) is operatively linked to a polynucleotide which it is not linked to in nature, or (3) does not occur in nature. Preferably, “recombinant” refers to a biomolecule that does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. Thus, for example, a protein synthesized by a microorganism is recombinant, for example, if it is synthesized from an mRNA synthesized from a recombinant gene present in the cell. A phage is “recombinant” if it comprises a recombinant biomolecule. Preferably, a phage is “recombinant” if it comprises a recombinant biomolecule that does not occur in nature. Thus, for example and without limitation, a phage is recombinant if the genome of the phage comprises a recombinant nucleic acid sequence.

The term “polynucleotide”, “nucleic acid molecule”, “nucleic acid”, or “nucleic acid sequence” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation. The nucleic acid (also referred to as polynucleotides) may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

A “synthetic” RNA, DNA or a mixed polymer is one created outside of a cell, for example one synthesized chemically.

The term “nucleic acid fragment” as used herein refers to a nucleic acid sequence that has a deletion, e.g., a 5′-terminal or 3′-terminal deletion compared to a full-length reference nucleotide sequence. In an embodiment, the nucleic acid fragment is a contiguous sequence in which the nucleotide sequence of the fragment is identical to the corresponding positions in the naturally-occurring sequence. In some embodiments, fragments are at least 10, 15, 20, or 25 nucleotides long, or at least 20, 30, 40, 50, 60, 70, 80, 90, 100, 110, 120, 130, 140, or 150 nucleotides long. In some embodiments a fragment of a nucleic acid sequence is a fragment of an open reading frame sequence. In some embodiments such a fragment encodes a polypeptide fragment (as defined herein) of the protein encoded by the open reading frame nucleotide sequence.

As used herein, an endogenous nucleic acid sequence in the genome of an organism (including a phage) (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome. With reference to a phage, a “recombinant phage genome” is a phage genome that contains an insertion, deletion or a point mutation introduced artificially, e.g., by human intervention and does not occur in nature.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

The term “percent sequence identity” or “identical” in the context of nucleic acid sequences refers to the residues in the two sequences, which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32, and even more typically at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 50%, 60%, 70%, 80%, 85%, 90%, 91%, 92%, 93%, 94%, 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tm) for the specific DNA hybrid under a particular set of conditions. “Stringent washing” is performed at temperatures about 5° C. lower than the Tm for the specific DNA hybrid under a particular set of conditions. The Tm is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

As used herein, an “expression control sequence” refers to polynucleotide sequences that affect the expression of coding sequences to which they are operatively linked. Expression control sequences are sequences that control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to encompass, at a minimum, any component whose presence is essential for expression, and can also encompass an additional component whose presence is advantageous, for example, leader sequences and fusion partner sequences.

As used herein, “operatively linked” or “operably linked” expression control sequences refers to a linkage in which the expression control sequence is contiguous with the gene of interest to control the gene of interest, as well as expression control sequences that act in trans or at a distance to control the gene of interest.

As used herein, a “vector” is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”).

The term “recombinant host cell” (or simply “recombinant cell” or “host cell”), as used herein, is intended to refer to a cell into which a recombinant nucleic acid such as a recombinant vector has been introduced. In some instances the word “cell” is replaced by a name specifying a type of cell. For example, a “recombinant microorganism” is a recombinant host cell that is a microorganism host cell. It should be understood that such terms are intended to refer not only to the particular subject cell, but to the progeny of such a cell. Because certain modifications may occur in succeeding generations due to either mutation or environmental influences, such progeny may not, in fact, be identical to the parent cell, but are still included within the scope of the term “recombinant host cell,” “recombinant cell,” and “host cell”, as used herein. A recombinant host cell may be an isolated cell or cell line grown in culture or may be a cell which resides in a living tissue or organism.

As used herein, “bacteriophage” refers to a virus that infects bacteria. Similarly, “archaeophage” refers to a virus that infects archaea. The term “phage” is used to refer to both types of viruses but in certain instances as indicated by the context may also be used as shorthand to refer to a bacteriophage or archaeophage specifically. Bacteriophage and archaeophage are obligate intracellular parasites that multiply inside bacteria/archaea by making use of some or all of the host biosynthetic machinery (i.e., viruses that infect bacteria). Though different bacteriophages and archaeophages may contain different materials, they all contain nucleic acid and protein, and can under certain circumstances be encapsulated in a lipid membrane. Depending upon the phage, the nucleic acid may be either DNA or RNA but not both and it can exist in various forms.

As used herein, “heterologous nucleic acid sequence” is any sequence placed at a location in the genome where it does not normally occur. A heterologous nucleic acid sequence may comprise a sequence that does not naturally occur in a particular bacteria/archaea and/or phage or it may comprise only sequences naturally found in the bacteria/archaea and/or phage, but placed at a non-normally occurring location in the genome. In some embodiments the heterologous nucleic acid sequence is not a natural phage sequence; in some embodiments it is a natural phage sequence, albeit from a different phage; while in still other embodiments it is a sequence that occurs naturally in the genome of the starting phage but is then moved to another site where it does not naturally occur, rendering it a heterologous sequence at that new site.

A “starting phage” or “starting phage genome” is a phage isolated from a natural or human made environment that has not been modified by genetic engineering, or the genome of such a phage.

A “recombinant phage” or “recombinant phage genome” is a phage that comprises a genome that has been genetically modified by insertion of a heterologous nucleic acid sequence into the phage, or the genome of the phage. Preferably, a “recombinant phage” or “recombinant phage genome” is a phage that does not occur in nature, i.e., does not comprise a genome that occurs in nature. In some embodiments the genome of a starting phage is modified by recombinant DNA technology to introduce a heterologous nucleic acid sequence into the genome at a defined site. In some embodiments the heterologous sequence is introduced with no corresponding loss of endogenous phage genomic nucleotides. In other words, if bases N1 and N2 are adjacent in the starting phage genome the heterologous sequence is inserted between N1 and N2. Thus, in the resulting recombinant genome the heterologous sequence is flanked by nucleotides N1 and N2. In some cases the heterologous sequence is inserted and endogenous nucleotides are removed or replaced with the exogenous sequence. For example, in some embodiments the exogenous sequence is inserted in place of some or all of the endogenous sequence which is removed. In some embodiments endogenous sequences are removed from a position in the phage genome distant from the site(s) of insertion of exogenous sequences.

A “phage host cell” is a cell that can be infected by a phage to yield progeny phage particles.

“Operatively linked” or “operably linked” expression control sequences refers to a linkage in which the expression control sequence is contiguous with coding sequences of interest to control expression of the coding sequences of interest, as well as expression control sequences that act in trans or at a distance to control expression of the coding sequence.

A “coding sequence” or “open reading frame” is a sequence of nucleotides that encodes a polypeptide or protein. The termini of the coding sequence are a start codon and a stop codon.

The term “expression control sequence” as used herein refers to polynucleotide sequences which affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

As used herein, a “phage genome” includes naturally occurring phage genomes and derivatives thereof. Generally (though not necessarily), the derivatives possess the ability to propagate in the same hosts as the parent. In some embodiments the only difference between a naturally occurring phage genome and a derivative phage genome is at least one of a deletion and an addition of nucleotides from at least one end of the phage genome if the genome is linear or at least one point in the genome if the genome is circular.

As used herein, “target microbe” includes bacteria, however, this term may also include other unicellular pathogens that cause infection in animals and/or humans. Preferred target microbes are bacteria.

As used herein, “target bacteria” are bacteria that can be infected by a phage to yield a detectable output or signal. For example, a detectable output includes cell lysis. Thus, lysis of bacterial cells by a phage indicates that the bacterial cells are “target bacteria” of that phage. Another example of a detectable output is expression of a marker following infection of a bacterial cell by a phage. Suitable markers include RNAs and polypeptides.

As used herein, a “marker” includes selectable and/or screenable markers. As used herein, a “selectable marker” is a marker that confers upon cells that possess the marker the ability to grow in the presence or absence of an agent that inhibits or stimulates, respectively, growth of similar cells that do not express the marker. Such cells can also be said to have a “selectable phenotype” by virtue of their expression of the selectable marker. For example, the ampicillin resistance gene (AmpR) confers the ability to grow in the presence of ampicillin on cells, which possess and express the gene. (See Sutcliffe, J. G., Proc Natl Acad Sci US A. 1978 August; 75(8): 3737-3741.) Other nonlimiting examples include genes that confer resistance to chloramphenicol, kanamycin, and tetracycline. Other markers include URA3, TRP and LEU, which allow growth in the absence of said uracil, tryptophan and leucine, respectively.

As used herein, a “screenable marker” is a detectable label that that can be used as a basis to identify cells that express the marker. Such cells can also be said to have a “screenable phenotype” by virtue of their expression of the screenable marker. (In general selectable markers may also function as screenable markers in so far as the gene product of the selectable marker may be used as a basis to identify cells that express the marker independently of the function of the gene product to confer selectability on cells that express it.) Any molecule that can be differentially detected and encoded by the recombinant phage can serve as a screenable marker. A screenable marker can be a nucleic acid molecule or a portion thereof, such as an RNA or a DNA molecule that is single or double stranded. Alternatively, a screenable marker can be a protein or a portion thereof. Suitable protein markers include enzymes that catalyze formation of a detectable reaction product. An example is a chemiluminescent protein such as luciferase or variations, such as luxAB, and β-galactosidase. Another example is the horseradish peroxidase enzyme. Proteins used to generate a luminescent signal fall into two broad categories: those that generate light directly (luciferases and related proteins) and those that are used to generate light indirectly as part of a chemical cascade (horseradish peroxidase). The most common bioluminescent proteins used in biological research are aequorin and luciferase. The former protein is derived from the jellyfish Aequorea victoria and can be used to determine calcium concentrations in solution. The luciferase family of proteins has been adapted for a broad range of experimental purposes. Luciferases from firefly and Renilla are the most commonly used in biological research. These proteins have also been genetically separated into two distinct functional domains that will generate light only when the proteins are closely co-localized. A variety of emission spectrum-shifted mutant derivatives of both of these proteins have been generated over the past decade. These have been used for multi-color imaging and co-localization within a living cell. The other groups of proteins used to generate chemiluminescent signal are peroxidases and phosphatases. Peroxidases generate peroxide that oxidizes luminol in a reaction that generates light. The most widely used of these is horseradish peroxidase (HRP), which has been used extensively for detection in western blots and ELISAs. A second group of proteins that have been employed in a similar fashion are alkaline phosphatases, which remove a phosphate from a substrate molecule, destabilizing it and initiating a cascade that results in the emission of light.

Other suitable screenable markers include fluorescent proteins. Fluorescent proteins include but are not limited to blue/UV fluorescent proteins (for example, TagBFP, Azurite, EBFP2, mKalama1, Sirius, Sapphire, and T-Sapphire), cyan fluorescent proteins (for example, ECFP, Cerulean, SCFP3A, mTurquoise, monomeric Midoriishi-Cyan, TagCFP, and mTFP1), green fluorescent proteins (for example, EGFP, Emerald, Superfolder GFP, Monomeric Azami Green, TagGFP2, mUKG, and mWasabi), yellow fluorescent proteins (for example, EYFP, Citrine, Venus, SYFP2, and TagYFP), orange fluorescent proteins (for example, Monomeric Kusabira-Orange, mKOκ, mKO2, mOrange, and mOrange2), red fluorescent proteins (for example, mRaspberry, mCherry, mStrawberry, mTangerine, tdTomato, TagRFP, TagRFP-T, mApple, and mRuby), far-red fluorescent proteins (for example, mPlum, HcRed-Tandem, mKate2, mNeptune, and NirFP), near-IR fluorescent proteins (for example, TagRFP657, IFP1.4, and iRFP), long stokes-shift proteins (for example, mKeima Red, LSS-mKate1, and LSS-mKate2), photoactivatible fluorescent proteins (for example, PA-GFP, PAmCherryl, and PATagRFP), photoconvertible fluorescent proteins (for example, Kaede (green), Kaede (red), KikGR1 (green), KikGR1 (red), PS-CFP2, PS-CFP2, mEos2 (green), mEos2 (red), PSmOrange, and PSmOrange), and photoswitchable fluorescent proteins (for example, Dronpa). Several variants and alternatives to the listed examples are also well known to those of skill in the art and may be substituted in appropriate applications.

Other suitable markers include epitopes. For example, a protein comprising an epitope that can be detected with an antibody or other binding molecule is an example of a screenable marker. An antibody that recognizes the epitope can be directly linked to a signal generating moiety (such as by covalent attachment of a chemiluminescent or fluorescent protein) or it can be detected using at least one additional binding reagent such as a secondary antibody, directly linked to a signal generating moiety, for example. In some embodiments the epitope is not present in the proteins of the phage or the target microorganism so detection of the epitope in a sample indicates that the protein comprising the epitope was produced by the microorganism following infection by the recombinant phage comprising a gene encoding the protein comprising the epitope. In other embodiments the marker may be a purification tag in the context of a protein that is naturally present in the target microorganism or the phage. For example, the tag (e.g., a 6-His tag) can be used to purify the heterologous protein from other bacterial or phage proteins and the purified protein can then be detected, for example using an antibody.

As used herein, an “environmental sample” is a sample obtained from any setting other than a laboratory cell culture setting. Generally, though not necessarily, an environmental sample is obtained from a setting that comprises at least one of a) a temperature that does not support maximum growth and/or metabolism of bacterial cells, b) a nutrient profile that does not support maximum growth and/or metabolism of bacterial cells, and c) bacterial cells that are not target bacteria for a phage used in an assay. In some embodiments some or all of the bacteria present in an environmental sample are not in a metabolically active state. Without limitation, environmental samples may be obtained from industrial plants, food processing plants, veterinary sources, food, livestock, medical settings and surfaces, schools, assisted living centers, cruise ships, other confined quarters and homes. The surface may be of any material. By way of non-limiting example, the surface can be metal, glass, wood, brick, concrete, tile, rug and the like. The surface can also be on an agricultural product. The sample can also be found inside of an agricultural produce. The “environmental sample” can be in situ, in other words, the assay can be performed at the site itself, rather than removed from the site. Alternatively, the “environmental sample” can be removed for assay from a collection point, as through the use of an absorbent material, such as a cotton swab to physically collect the sample.

As used herein, “agricultural” refers to cultivated or wild plants, animals, and fungi. The term also refers to stock feed or food supply. “Food supply” encompasses food for either human or non-human animal consumption. Accordingly, an “agricultural sample” refers to a sample from of, within, or on the exterior of a plant, animal and fungi.

While the disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the disclosure. All such modifications are intended to be within the scope of the claims appended hereto.

### EXAMPLES

### Example 1: Design of Codon Optimized Phage

Recombinant phage were designed for increased expression of the luciferase reporter. Phages selected for recombination were A511, LP124, and LP40. The capsid (CPS) nucleotide sequences for A511 (SEQ ID NO: 19), LP124 (SEQ ID NO: 13) and LP40 (SEQ ID NO: 5) are provided herewith. The NanoLuc luciferase reporter was selected for recombination by phage optimization. Phage optimization was performed using DNA 2.0™ software. The software uses an algorithm described Villalobos et al. (see Villalobos et al. BMC Bioinformatics. Gene Designer: a synthetic biology tool for constructing artificial DNA segments. PLoS ONE. 2011 6:e19912.) to replace synonymous codons with those preferred by a host organism, in this case listeria.

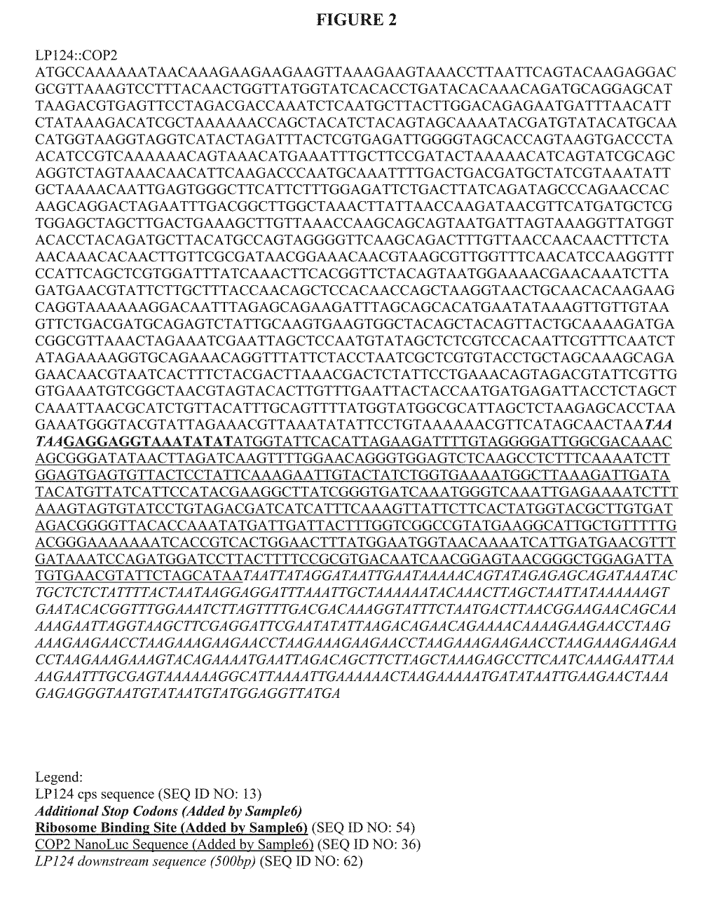

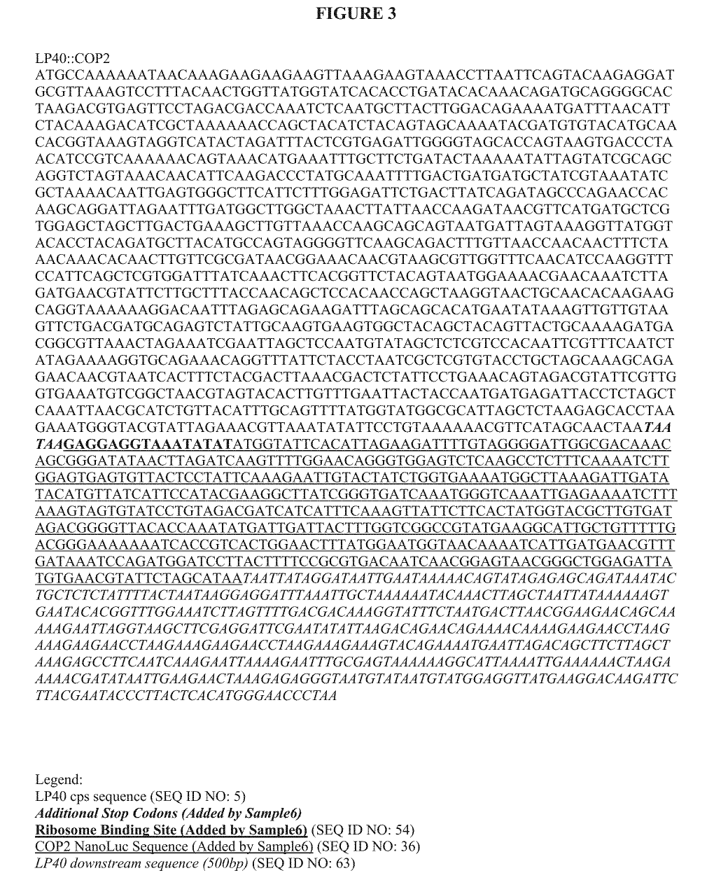

The codon optimized Nanoluc (COP2; SEQ ID NO: 36) was inserted into the phage CPS open reading frame following stop codons and a ribosome binding site (SEQ ID NO: 54) using methods as described in Example 19 herein. Primers used in engineering listeria phage include pMAK upf (SEQ ID NO: 55), dbono380 (SEQ ID NO: 56), SO472 (SEQ ID NO: 57), SO473 (SEQ ID NO: 58), SO474 (SEQ ID NO: 59) and dbono 382 (SEQ ID NO: 62) oligos. A sequence map of the insertion site for A511:: COP2 recombinant phage (SEQ ID NO: 39) recombinant phage is shown in FIG. 2 indicating the location of insertion of the COP2 reporter, ribosome binding site and the flanking sequence following CPS. A sequence map of the insertion site for LP124::COP2 recombinant phage (SEQ ID NO: 39) is shown in FIG. 2 indicating the location of insertion of the COP2 reporter, ribosome binding site and the flanking sequence following CPS. A sequence map of the insertion site for LP40::COP2 recombinant phage (SEQ ID NO: 40) is shown in FIG. 3 indicating the location of insertion of the COP2 reporter, ribosome binding site and the flanking sequence following CPS.

Recombinant phage comprising the native NanoLuc luciferase was compared to recombinant phage comprising the codon optimized COP2 luciferase. Comparisons utilized a mixture of recombinant phage. The COP2 mixture comprising the A511:COP2, LP124::COP2, and LP40::COP2 phages. These experiments were done using a cocktail of A511::COP2, LP124::COP2, and LP40::COP2. The final concentration of each phage is 1.5e7 pfu/ml, the final concentration of the mixture is 4.5e7 pfu/ml. The mixture of NanoLuc phages comprised phages selected from Example 19. Protocols for the comparison assay are as follows.

On Sponge infections with NanoLuc and COP2 Phage Mixture:

3 sponges (3M spongestick w/ Letheen broth) were used for each condition. The stick was removed from each sponge, and the sponges were squeezed to remove Letheen broth. ˜100 CFU of Listeria monocytogenes CDW 1554 were spiked onto each sponge:

Healthy cells: 5 ml overnight culture (18-24 h in 0.5×BHI) diluted 1:4 into 0.5×BHI and incubated at 30° C. shaking at 180 rpm for 2 hours. 100 μl of a 1e⁻⁶ dilution was spiked into each sponge. Healthy cells, in this case, refer to an overnight culture that has reached stationary phase being back-diluted to re-enter log phase.

Sick cells: 250 μl of a CDW 1554 overnight culture diluted to ˜1e7 CFU/ml in BHI+1% glucose was spread on a 4″×4″ square on a stainless steel table. Cells were allowed to dry overnight (18-24 h). Cells were recovered using a cotton swab moistened with Letheen Broth, and placed in a conical tube containing 2 ml of Letheen Broth. Cells were allowed to recover for 30 minutes at 30° C. Cells were diluted in 0.5×BHI to the point where 100 μl should contain ˜100 CFU. 100 μl was spiked onto each sponge. The model mimics a factory condition where cells are surviving on a steel surface that may or may not have food contact. Sick cells are less metabolically active and produce less light upon phage infection than their healthy counterparts.

Conditions:

3 sponges with NanoLuc phage mixture/no cells
3 sponges with NanoLuc phage mixture/100 CFU Healthy CDW 1554
3 sponges with NanoLuc phage mixture/100 CFU Sick CDW 1554
3 sponges with COP2 phage mixture/no cells
3 sponges with COP2 phage mixture/100 CFU Healthy CDW 1554
3 sponges with COP2 phage mixture/100 CFU Sick CDW 1554

Infection:

After cells were spiked onto sponges, phage was mixed as follows:

7 ml of phage solution (9e8 pfu/ml), (NanoLuc or COP2), was added to 77 ml of NIB-10 infection buffer.
6 ml of the appropriate phage mixture was added to each sponge, with a brief massage to mix the solution into the sponge ensuring complete coverage.
Sponges were placed at 30° C. for 6 hours.

Detection:

Sponges were squeezed to separate the liquid from the sponge. 1000 μl of liquid was removed from each sponge and placed in a microcentrifuge tube. Tubes were spun at 16,000 g for 1 minute. 300 μl was transferred to an Eppendorf microcentrifuge tube. 300 μl of Nano-Glo detection reagent was added to each tube.

Samples were read in a Berthold Sirius-L luminometer using a 20 second kinetic read. RLU values across the last 16 seconds of the read were averaged resulting in the RLU value for each sample.

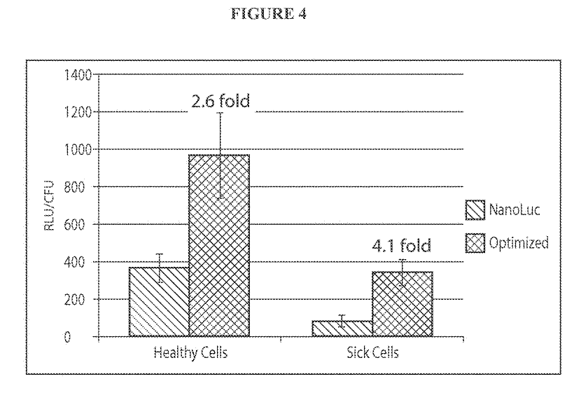

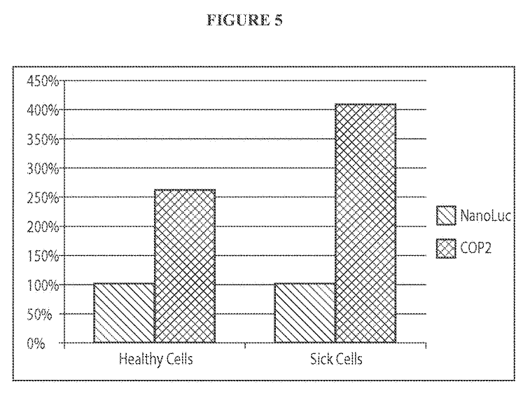

As shown in Table 6, the mixture of phages comprising codon optimized luciferase (COP2) shows a 2.6 fold increase in relative light units (RLU) per colony forming units (CFU) over recombinant phage encoding basic NanoLuc when infecting healthy Listeria cells. In a comparison of recombinant phage mixtures infected sick cells (e.g. cells that have dried on a counter surface, or been subjected to cleaning agents) the codon optimized COP2 encoding phage mixture shows a 4.1 fold increase in RLU/CFU over regular NanoLuc encoding phage mixtures (see FIG. 4). When normalized as an indication of performance, the codon optimized COP2 encoding phage perform at 264% of their NanoLuc encoding counterparts in healthy cells (see Table 7 and FIG. 5). In sick cells, the COP2 encoding phage perform at 409% of their NanoLuc encoding counterparts (see Table 12 and FIG. 5). This significant enhancement in the output of light from the recombinant phage encoding codon optimized COP2 improves the detectable limits of Listeria contamination.

TABLE 6

| | RLU/CFU Healthy | RLU/CFU Sick |
|---|---|---|
| NanoLuc | 365.50 | 83.32 |
| COP2 | 965.87 | 340.95 |

TABLE 7

| | Healthy Cells | Sick Cells |
|---|---|---|
| NanoLuc | 100% | 100% |
| COP2 | 264% | 409% |

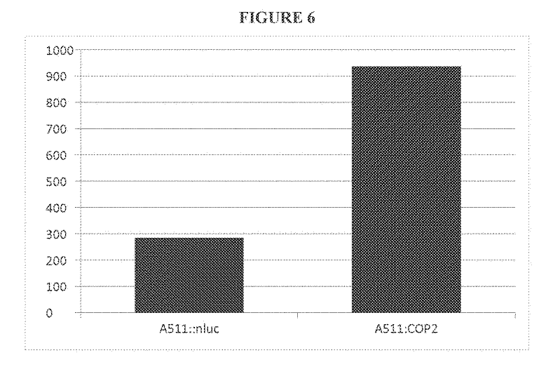

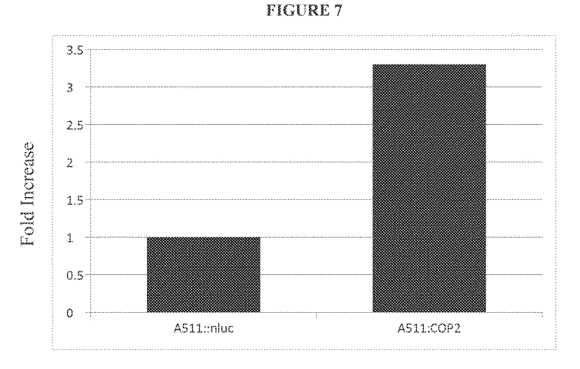

Further experiments were performed using a single A511 phage engineered with COP2 to compare reporter signal in sick cells. As shown in Table 8, the A511 phage comprising codon optimized luciferase (COP2) shows a 3.3 fold increase in relative light units (RLU) per colony forming units (CFU) over recombinant A511 phage encoding basic NanoLuc when infecting sick Listeria cells (see FIG. 6). When normalized as an indication of performance, the codon optimized COP2 encoding A511 phage perform at 330% of their NanoLuc encoding counterparts in sick cells (see Table 9 and FIG. 7). This significant enhancement in the output of light from the recombinant phage encoding codon optimized COP2 improves the detectable limits of Listeria contamination and use of a single phage type in a detection assay.

TABLE 8

| | A511::nluc | A511:COP2 |
|---|---|---|
| RLU/CFU | 283.57 | 935.82 |

TABLE 9

| | A511::nluc | A511:COP2 |
|---|---|---|
| Signal | 100.00% | 330.01% |

### Example 2: Design of Codon-Optimized Phage V3 (COP3)

Additional codon optimization was performed with recombinant phage in an effort to further increase the expression levels of the luciferase reporter. Phages selected for optimization were A511, LP124 and LP40. The NanoLuc luciferase reporter was selected for recombination by phage optimization. Coding sequence optimization was performed using DNA 2.0™ software. The software uses an algorithm described Villalobos et al. (see Villalobos et al. BMC Bioinformatics. Gene Designer: a synthetic biology tool for constructing artificial DNA segments. PLoS ONE. 2011 6:e19912.) to replace synonymous codons with those preferred by a host organism.

The purpose of this round of codon optimization was to create a custom codon optimization algorithm specific for Listeria. For these experiments, a set of 24 codon-optimized variants were designed and constructed by DNA 2.0™. These variants allowed for testing a variety of hypotheses concerning codon usage. The set of the new 24 codon-optimized variants (COP3) were cloned into the A511 vector. These plasmids were transformed into the Listeria monocytogenes strain EGD-e, and isolated using phage infective engineering (PIE) methodology described in Example 19 above.

The 24 variants of the COP3 phages were isolated and purified by ultracentrifugation. The isolated and purified COP3 phages were compared with COP2 and the non-codon optimized NanoLuc phages. The relative signal strength was generated across a subset of Listeria strains and normalized to COP2. These data were then traced back to specific changes in the codon usage profile. These data pointed to the improved COP3 phage herein referred to as W40_VIP_MLi178 (“VIP178”) (see SEQ ID NO: 37) as the most improved variant. This version of NanoLuc was used to create three new engineered phages: A511::VIP178, LP124::VIP178, and LP40::VIP178 (also referred to herein as A511::COP3, LP124::COP3 and LP40::COP3).

The phages were engineered using the primers described herein. The primers pMAK upf (see SEQ ID NO:55) and DBONO380 (see SEQ ID NO:56) were used to amplify the upstream homology fragments for each phage. The VIP178 fragment was amplified using the SO670 (see SEQ ID NO:64) and SO671 (see SEQ ID NO:65). The downstream homology fragments were amplified using the primers S0672 (see SEQ ID NO:66) and DBONO382 (see SEQ ID NO:60).

Signal intensity levels of COP2 and COP3 phages were assessed as detailed below in Example 3.

### Example 3: Signal Comparison of COP2 and COP3 Phages

In order to assess any differences in intensity or robustness of signal provided by the COP2 and COP3 phages, a screen was performed in which the signal intensity was determined by using the assay described below.

For these assays the following materials were used: Validation Plates (342 strains across Listeria species), Omni-Tray with 0.5×BHI agar, Deep-Well 96-well plate (Axygen), Clear flat-bottom 96-well plate (Evergreen), White flat-bottom 96-well plate (Greiner Bio-One), Plate-sealing film (breathable), 15 mL conical tubes, Letheen Broth, 0.5×BHI, NIB-14, Nano-Glo, Substrate, Nano-Glo Buffer, 200 μL multichannel pipette, 1000 μL multichannel pipette, 20 μL multichannel pipette, 96-pin replicator tool (frogger).

The detailed protocol used in these assays is described below.

### Protocol

### Day 0

Stamp out validation plates 1 through 3 and greatest misses plate onto 0.5×BHI agar Omni-tray plate using the 96-pin replicator tool.

Incubate plates at 35° C. overnight (18 h)

### Day 1

Fill wells of 96-well DeepWell with 1 mL of 0.5×BHI

Inoculate DeepWell with colonies from stamped-out plates

Incubate plates at 30° C. for 24 h, shaking

### Day 2

1. Dilute all phage variants in NIB-14 to 9E7 pfu/mL in a final volume of 20 mL

a. (For lower phage concentration tests, dilute variants to 3E7 pfu/mL and/or 1E7 pfu/mL)

b. Note: GM plate is not a full plate—use the empty wells to act as negative controls for the assay

2. Add 180 μL of Letheen Broth to all wells of four (4) clear, flat-bottom 96-well plates

3. Label plates from 1E−1 through 1E−4 dilution

4. Add 900 μL of Letheen Broth to all wells of a 96-well deep-well plate

a. Label deep-well plate as the 1E−5 dilution

5. Repeat previous steps 2 through 5 three additional times, one set for each plate of overnight culture

a. i.e. Validation Plates 1, 2, 3, and Greatest Misses

6. For each culture, transfer 20 μL from every well of overnight culture to corresponding well of 1E−1 dilution plate

7. Pipette mix 10-15×

8. Repeat steps 6 and 7, transferring from 1E−1 plate to 1E−2 plate, then from 1E−2 plate to 1E−3 plate, then from 1E−3 plate to 1E−4 plate.

9. Transfer 100 μL from every well of 1E−4 dilution plate to corresponding well of 1E−5 dilution plate for a total volume of 1000 μL in each well of the deep well plate

10. Repeat previous step until there is a 1E−5 cell dilution for each phage variant being tested, plus COP2, for each overnight culture

a. e.g. if testing three variants:
i. Validation plate 1—four (4) plates at 1E−5 dilution
ii. Validation plate 2—four (4) plates at 1E−5 dilution
iii. Validation plate 3—four (4) plates at 1E−5 dilution
iv. GM Plate—four (4) plates at 1E−5 dilution

11. Dilute 1E−5 dilution of 1839 from Validation Plate 2—50 μL into 450 μL of Letheen (1:10 total dilution—1E−6 dilution from overnight culture)

12. Plate 100 μL of −6 dilution onto BHI plate and incubate overnight at 35° C.

13. For each strain plate, transfer 100 μL of phage/NIB-14 mixture for each phage variant being tested

14. Start with COP2

15. Stagger each set by ≈20 minutes (or as you see fit) to allow time to read between strain plates

16. Recommended: Complete infection for all variants on Plate 1, then all variants of Plate 2, etc.

17. Incubate plates at 30° C. for 6 h

18. Mix necessary amount of Nano-glo buffer with substrate (≈5 ml/plate)

19. Transfer 40 μL of from each well of infection plate to corresponding wells of white, flat-bottom plate

Add 40 μL of mixed Nano-Glo reagent

Detect on Glomax 96

Steady Glo—0 s delay, 0.5 s integration

### Analysis:

COP3 target panel consists of strains producing between 100 RLU/CFU and 1000 RLU/CFU with COP2 assay

Compare signal of target strains with COP2 phage cocktail to COP3 phage cocktail candidate

Plate 100 μL of 1E−6 dilution for target strains onto BHI plate to calculate RLU/CFU of COP3 target strains for COP2 and COP3

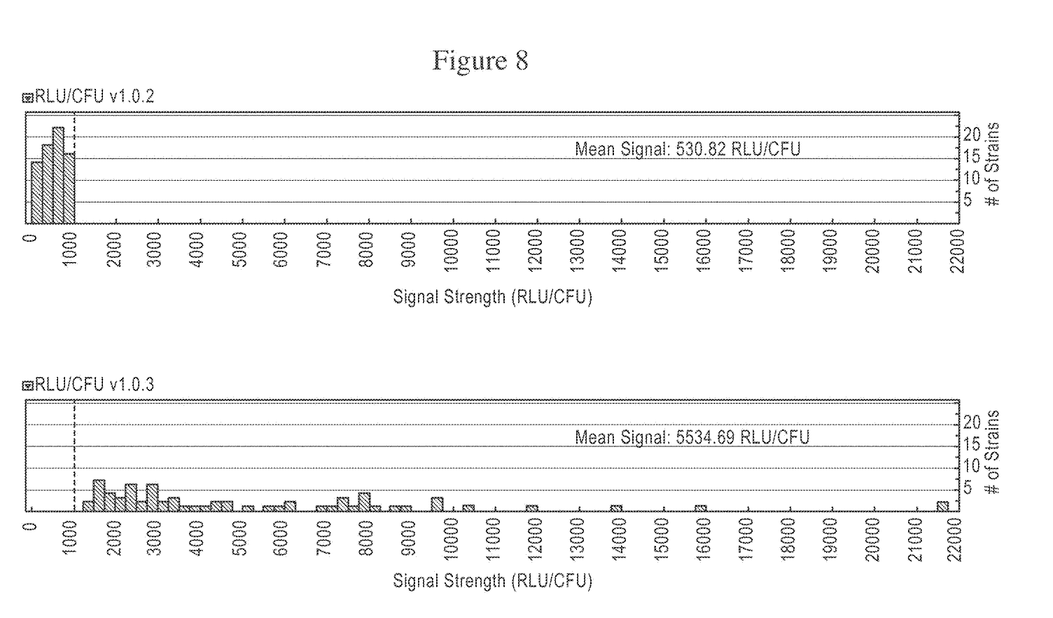

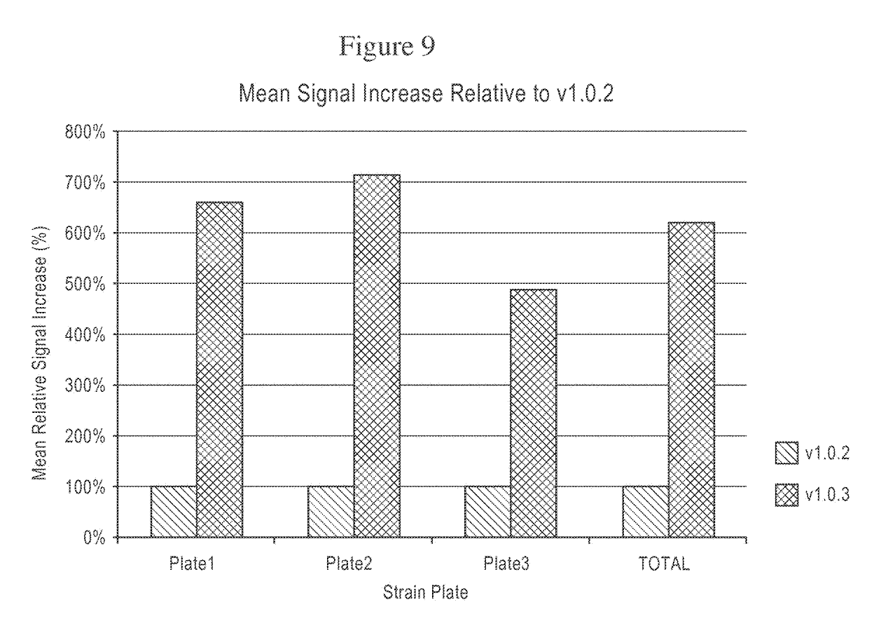

Data acquired in the comparison of the COP2 and the COP3 phages are shown in FIGS. 8 and 9. The signal comparison assays demonstrate that there was a broad increase in signal intensity across tested Listeria species with the use of the COP3 phage in comparison with the COP2 phage. The data further indicate that there was a six time (6×) mean signal increase across all the tested conditions with the use of COP3 in comparison to COP2. The full listing of Listeria strains used in these studies is shown in Table 10.

TABLE 10
Full Listing of Listeria Strains Assessed
in COP2 and COP3 Signal Intensity Assay

| | NP | | | |
|---|---|---|---|---|
| Plate | Strain # | Strain ID | Species | Well |
| 1 | 1814 | FSL C2-010 | Listeria ivanovii | a1 |
| 1 | 1815 | FSL C2-011 | Listeria ivanovii | a2 |
| 1 | 1816 | FSL H6-011 | Listeria seeligeri | a3 |
| 1 | 1817 | FSL H6-169 | Listeria seeligeri | a4 |
| 1 | 1818 | FSL H6 017 | Listeria welshimeri | a5 |
| 1 | 1819 | FSL H6-105 | Listeria welshimeri | a6 |
| 1 | 1820 | FSL C2-008 | Listeria innocua | a7 |
| 1 | 1821 | FSL X1-017 | Listeria innocua | a8 |
| 1 | 1822 | FSL J1-023 | Listeria innocua | a9 |
| | | | (hemolytic) | |
| 1 | 1823 | FSL W3-075 | Listeria innocua | a10 |
| | | | (hemolytic) | |
| 1 | 1824 | FSL S4-120 | Listeria marthii | a11 |
| 1 | 1825 | FSL S4-965 | Listeria marthii | a12 |
| 1 | 1826 | FSL F6-920 | Listeria rocourtiae | b1 |
| 1 | 1827 | FSL C1-056 | Listeria monocytogenes | b2 |
| 1 | 1828 | FSL J1-177 | Listeria monocytogenes | b3 |
| 1 | 1829 | FSL J1-094 | Listeria monocytogenes | b4 |
| 1 | 1830 | FSL C1-115 | Listeria monocytogenes | b5 |
| 1 | 1831 | FSL J1-169 | Listeria monocytogenes | b6 |
| 1 | 1832 | FSL J1-049 | Listeria monocytogenes | b7 |
| 1 | 1833 | FSL W1-112 | Listeria monocytogenes | b8 |
| 1 | 1834 | FSL J1-110 | Listeria monocytogenes | b9 |
| 1 | 1835 | FSL J1-129 | Listeria monocytogenes | b10 |
| 1 | 1836 | FSL W1-110 | Listeria monocytogenes | b11 |
| 1 | 1837 | FSL J1-107 | Listeria monocytogenes | b12 |
| 1 | 1838 | FSL J1-175 | Listeria monocytogenes | c1 |
| 1 | 1839 | FSL F6-367 | Listeria monocytogenes | c2 |
| | | (MACK) | | |
| 1 | 1840 | FSL J1-208 | Listeria monocytogenes | c3 |
| 1 | 1869 | WSLC 3009 | Listeria ivanovii | c4 |
| 1 | 1879 | FSL N4-221 | Listeria monocytogenes | c5 |
| 1 | 1878 | EGD-e | Listeria monocytogenes | c6 |
| 1 | 1880 | FSL L3-159 | Listeria monocytogenes | c7 |
| 1 | 1881 | FSL T1-323 | Listeria monocytogenes | c8 |
| 1 | 1882 | FSL H5-725 | Listeria monocytogenes | c9 |
| 1 | 1883 | FSL T1-922 | Listeria monocytogenes | c10 |
| 1 | 1884 | FSL H1-251 | Listeria monocytogenes | c11 |
| 1 | 1885 | FSL L3-501 | Listeria monocytogenes | c12 |
| 1 | 1886 | FSL R2-069 | Listeria monocytogenes | d1 |
| 1 | 1887 | FSL H1-030 | Listeria monocytogenes | d2 |
| 1 | 1888 | FSL L4-019 | Listeria monocytogenes | d3 |
| 1 | 1889 | FSL T1-027 | Listeria monocytogenes | d4 |
| 1 | 1890 | FSL T1-041 | Listeria monocytogenes | d5 |
| 1 | 1891 | FSL R8-5241 | Listeria seeligeri | d6 |
| 1 | 1892 | FSL R8-5247 | Listeria seeligeri | d7 |
| 1 | 1893 | FSL R8-5253 | Listeria seeligeri | d8 |
| 1 | 1894 | FSL R8-5513 | Listeria seeligeri | d9 |
| 1 | 1895 | FSL R8-6629 | Listeria seeligeri | d10 |
| 1 | 1896 | FSL R8-6635 | Listeria seeligeri | d11 |
| 1 | 1897 | FSL R8-6659 | Listeria seeligeri | d12 |
| 1 | 1898 | FSL R8-6665 | Listeria seeligeri | e1 |
| 1 | 1899 | FSL R8-6852 | Listeria seeligeri | e2 |
| 1 | 1900 | FSL R8-5085 | Listeria innocua | e3 |
| 1 | 1901 | FSL R8-5091 | Listeria innocua | e4 |
| 1 | 1902 | FSL R8-5098 | Listeria innocua | e5 |
| 1 | 1903 | FSL R8-5255 | Listeria innocua | e6 |
| 1 | 1904 | FSL R8-5293 | Listeria innocua | e7 |
| 1 | 1905 | FSL R8-5295 | Listeria innocua | e8 |
| 1 | 1906 | FSL R8-5306 | Listeria innocua | e9 |
| 1 | 1907 | FSL R8-5440 | Listeria innocua | e10 |
| 1 | 1908 | FSL R8-5442 | Listeria innocua | e11 |
| 1 | 1909 | FSL R8-5448 | Listeria innocua | e12 |
| 1 | 1910 | FSL R8-7026 | Listeria welshimeri | f1 |
| 1 | 1911 | FSL R8-7641 | Listeria seeligeri | f2 |
| 1 | 1912 | FSL R8-7061 | Listeria innocua | f3 |
| 1 | 1913 | FSL R8-6826 | Listeria seeligeri | f4 |
| 1 | 1914 | FSL R8-7454 | Listeria welshimeri | f5 |
| 1 | 1915 | FSL R8-7548 | Listeria innocua | f6 |
| 1 | 1916 | FSL R8-6667 | Listeria innocua | f7 |
| 1 | 1917 | FSL R8-1903 | Listeria welshimeri | f8 |
| 1 | 1945 | FSL L5-079 | Listeria welshimeri | f9 |
| 1 | 1946 | FSL S10-1450 | Listeria welshimeri | f10 |
| 1 | 1947 | FSL S10-1451 | Listeria welshimeri | f11 |
| 1 | 1948 | FSL S4-081 | Listeria welshimeri | f12 |
| 1 | 1949 | FSL S4-101 | Listeria welshimeri | g1 |
| 1 | 1950 | FSL S10-030 | Listeria seeligeri | g2 |
| 1 | 1951 | FSL S10-320 | Listeria seeligeri | g3 |
| 1 | 1952 | FSL S10-1602 | Listeria seeligeri | g4 |
| 1 | 1953 | FSL L5-075 | Listeria seeligeri | g5 |
| 1 | 1954 | FSL L5-046 | Listeria seeligeri | g6 |
| 1 | 1955 | FSL L5-104 | Listeria seeligeri | g7 |
| 1 | 1956 | FSL R8-7575 | Listeria seeligeri | g8 |
| 1 | 1957 | FSL S4-178 | Listeria seeligeri | g9 |
| 1 | 1958 | FSL S4-135 | Listeria seeligeri | g10 |
| 1 | 1959 | FSL S4-158 | Listeria innocua | g11 |
| 1 | 1960 | FSL S10-784 | Listeria innocua | g12 |
| 1 | 1961 | FSL F6-1168 | Listeria innocua | h1 |
| 1 | 1962 | FSL R8-5961 | Listeria innocua | h2 |
| 1 | 1963 | FSL R8-6922 | Listeria innocua | h3 |
| 1 | 1964 | FSL R8-7352 | Listeria innocua | h4 |
| 1 | 1965 | FSL R8-5598 | Listeria innocua | h5 |
| 1 | 1966 | FSL R8-6733 | Listeria innocua | h6 |
| 1 | 1967 | FSL R8-5942 | Listeria innocua | h7 |
| 1 | 1968 | FSL S10-034 | Listeria seeligeri | h8 |
| 1 | 1969 | FSL S10-1611 | Listeria seeligeri | h9 |
| 1 | 1970 | FSL L5-054 | Listeria seeligeri | h10 |
| 1 | 1971 | FSL L5-085 | Listeria seeligeri | h11 |
| 1 | 1972 | FSL R8-6868 | Listeria seeligeri | h12 |
| 2 | 1839 | FSL F6-367 | Listeria monocytogenes | a1 |
| | | (MACK) | | |
| 2 | 1840 | FSL J1-208 | Listeria monocytogenes | a2 |
| 2 | 1869 | WSLC 3009 | Listeria ivanovii | a3 |
| 2 | 1973 | FSL R8-6545 | Listeria seeligeri | a4 |
| 2 | 1974 | FSL R8-6949 | Listeria seeligeri | a5 |
| 2 | 1975 | FSL S4-167 | Listeria seeligeri | a6 |
| 2 | 1976 | FSL S4-180 | Listeria seeligeri | a7 |
| 2 | 1977 | FSL N1-064 | Listeria welshimeri | a8 |
| 2 | 1978 | FSL R8-8163 | Listeria welshimeri | a9 |
| 2 | 1979 | FSL R8-7524 | Listeria welshimeri | a10 |
| 2 | 1980 | FSL R8-7486 | Listeria welshimeri | a11 |
| 2 | 1981 | FSL R8-6035 | Listeria welshimeri | a12 |
| 2 | 1982 | FSL R8-5807 | Listeria welshimeri | b1 |
| 2 | 1983 | FSL S4-182 | Listeria welshimeri | b2 |
| 2 | 1984 | FSL R2-630 | Listeria welshimeri | b3 |
| 2 | 1985 | FSL F6-1131 | Listeria welshimeri | b4 |
| 2 | 1986 | FSL R8-7041 | Listeria welshimeri | b5 |
| 2 | 1987 | FSL R8-5837 | Listeria welshimeri | b6 |
| 2 | 1988 | FSL R8-6136 | Listeria welshimeri | b7 |
| 2 | 1989 | FSL S4-289 | Listeria welshimeri | b8 |
| 2 | 1990 | FSL H6-027 | Listeria seeligeri | b9 |
| 2 | 1991 | FSL H6-079 | Listeria seeligeri | b10 |
| 2 | 1992 | FSL H6-185 | Listeria seeligeri | b11 |
| 2 | 1993 | FSL R8-6874 | Listeria seeligeri | b12 |
| 2 | 1994 | FSLR8-6880 | Listeria seeligeri | c1 |
| 2 | 1995 | FSL R8-7629 | Listeria seeligeri | c2 |
| 2 | 1996 | FSL S4-544 | Listeria seeligeri | c3 |
| 2 | 1997 | FSL R8-5764 | Listeria innocua | c4 |
| 2 | 1998 | FSL R8-5802 | Listeria innocua | c5 |
| 2 | 1999 | FSL R8-6012 | Listeria innocua | c6 |
| 2 | 2000 | FSL R8-6355 | Listeria innocua | c7 |
| 2 | 2001 | FSL R8-6369 | Listeria innocua | c8 |
| 2 | 2002 | FSL R8-6476 | Listeria innocua | c9 |
| 2 | 2003 | FSL R8-7175 | Listeria innocua | c10 |
| 2 | 2004 | FSL R8-6888 | Listeria innocua | c11 |
| 2 | 2005 | FSL R8-6672 | Listeria innocua | c12 |
| 2 | 2006 | FSL S10-1311 | Listeria innocua | d1 |
| 2 | 2007 | FSL F6-1159 | Listeria innocua | d2 |
| 2 | 2008 | FSL F6-1126 | Listeria innocua | d3 |
| 2 | 2009 | FSL S6-120 | Listeria innocua | d4 |
| 2 | 2010 | FSL R8-5594 | Listeria innocua | d5 |
| 2 | 2011 | FSL R8-7181 | Listeria innocua | d6 |
| 2 | 2012 | FSL R2-632 | Listeria innocua | d7 |
| 2 | 2013 | FSL L3-851 | Listeria innocua | d8 |
| 2 | 2014 | FSL S10-1377 | Listeria innocua | d9 |
| 2 | 2015 | FSL S10-114 | Listeria welshimeri | d10 |
| 2 | 2016 | FSL S10-115 | Listeria welshimeri | d11 |
| 2 | 2017 | FSL S10-117 | Listeria welshimeri | d12 |
| 2 | 2018 | FSL S10-119 | Listeria welshimeri | e1 |
| 2 | 2019 | FSL S10-121 | Listeria welshimeri | e2 |
| 2 | 2020 | FSL R8-0056 | Listeria welshimeri | e3 |
| 2 | 2021 | FSL R8-1198 | Listeria welshimeri | e4 |
| 2 | 2022 | FSL R8-7403 | Listeria welshimeri | e5 |
| 2 | 2023 | FSL R2-631 | Listeria welshimeri | e6 |
| 2 | 2024 | FSL F6-267 | Listeria monocytogenes | e7 |
| 2 | 2025 | FSL F6-406 | Listeria monocytogenes | e8 |
| 2 | 2026 | FSL H5-592 | Listeria monocytogenes | e9 |
| 2 | 2027 | FSL H1-219 | Listeria monocytogenes | e10 |
| 2 | 2028 | FSL H1-121 | Listeria monocytogenes | e11 |
| 2 | 2029 | FSL W3-072 | Listeria monocytogenes | e12 |
| 2 | 2030 | FSL N4-239 | Listeria monocytogenes | f1 |
| 2 | 2031 | FSL N3-293 | Listeria monocytogenes | f2 |
| 2 | 2032 | FSL F3-319 | Listeria monocytogenes | f3 |
| 2 | 2033 | FSL F2-738 | Listeria monocytogenes | f4 |
| 2 | 2034 | FSL N3-881 | Listeria monocytogenes | f5 |
| 2 | 2035 | FSL N4-048 | Listeria monocytogenes | f6 |
| 2 | 2036 | FSL N4-696 | Listeria monocytogenes | f7 |
| 2 | 2037 | FSL N4-242 | Listeria monocytogenes | f8 |
| 2 | 2038 | FSL H4-364 | Listeria monocytogenes | f9 |
| 2 | 2039 | FSL H4-147 | Listeria monocytogenes | f10 |
| 2 | 2040 | FSL H4-946 | Listeria monocytogenes | f11 |
| 2 | 2041 | FSL S4-461 | Listeria monocytogenes | f12 |
| 2 | 2042 | FSL F6-206 | Listeria monocytogenes | g1 |
| 2 | 2043 | FSL F6-224 | Listeria monocytogenes | g2 |
| 2 | 2044 | FSL L3-739 | Listeria monocytogenes | g3 |
| 2 | 2045 | FSL N3-008 | Listeria monocytogenes | g4 |
| 2 | 2046 | FSL N3-022 | Listeria monocytogenes | g5 |
| 2 | 2047 | FSL J1-108 | Listeria monocytogenes | g6 |
| 2 | 2048 | FSL J1-119 | Listeria monocytogenes | g7 |
| 2 | 2049 | FSL C1-122 | Listeria monocytogenes | g8 |
| 2 | 2050 | FSL J1-126 | Listeria monocytogenes | g9 |
| 2 | 2051 | FSL F3-285 | Listeria monocytogenes | g10 |
| 2 | 2052 | FSL R6-288 | Listeria monocytogenes | g11 |
| 2 | 2053 | FSL N1-021 | Listeria monocytogenes | g12 |
| 2 | 2054 | FSL H1-208 | Listeria monocytogenes | h1 |
| 2 | 2055 | FSL N3-034 | Listeria monocytogenes | h2 |
| 2 | 2056 | FSL L5-072 | Listeria monocytogenes | h3 |
| 2 | 2057 | FSL S6-131 | Listeria monocytogenes | h4 |
| 2 | 2058 | FSL N3-278 | Listeria monocytogenes | h5 |
| 2 | 2059 | FSL R2-282 | Listeria monocytogenes | h6 |
| 2 | 2060 | FSL H5-770 | Listeria monocytogenes | h7 |
| 2 | 2061 | FSL F6-207 | Listeria monocytogenes | h8 |
| 2 | 2062 | FSL F6-236 | Listeria monocytogenes | h9 |
| 2 | 2063 | FSL H5-795 | Listeria monocytogenes | h10 |
| 2 | 2064 | FSL N3-246 | Listeria monocytogenes | h11 |
| 2 | 2065 | FSL R2-062 | Listeria monocytogenes | h12 |
| 3 | 1839 | FSL F6-367 | Listeria monocytogenes | a1 |
| | | (MACK) | | |
| 3 | 1840 | FSL J1-208 | Listeria monocytogenes | a2 |
| 3 | 1869 | WSLC 3009 | Listeria ivanovii | a3 |
| 3 | 2066 | FSL R2-437 | Listeria monocytogenes | a4 |
| 3 | 2067 | FSL M1-004 | Listeria monocytogenes | a5 |
| 3 | 2068 | FSL L4-352 | Listeria monocytogenes | a6 |
| 3 | 2069 | FSL F6-605 | Listeria monocytogenes | a7 |
| 3 | 2070 | FSL V1-001 | Listeria monocytogenes | a8 |
| 3 | 2071 | FSL F6-464 | Listeria monocytogenes | a9 |
| 3 | 2072 | FSL R8-2748 | Listeria monocytogenes | a10 |
| 3 | 2073 | FSL R6-908 | Listeria monocytogenes | a11 |
| 3 | 2074 | FSL L3-802 | Listeria monocytogenes | a12 |
| 3 | 2075 | FSL F3-056 | Listeria monocytogenes | b1 |
| 3 | 2076 | FSL J2-020 | Listeria monocytogenes | b2 |
| 3 | 2077 | FSL S4-914 | Listeria monocytogenes | b3 |
| 3 | 2078 | FSL F6-467 | Listeria monocytogenes | b4 |
| 3 | 2079 | FSL F6-655 | Listeria monocytogenes | b5 |
| 3 | 2080 | FSL F6-352 | Listeria monocytogenes | b6 |
| 3 | 2081 | FSL H5-781 | Listeria monocytogenes | b7 |
| 3 | 2082 | FSL K2-147 | Listeria monocytogenes | b8 |
| 3 | 2083 | FSL V1-026 | Listeria monocytogenes | b9 |
| 3 | 2084 | FSL H5-572 | Listeria monocytogenes | b10 |
| 3 | 2085 | FSL K2-065 | Listeria monocytogenes | b11 |
| 3 | 2086 | FSL H4-120 | Listeria monocytogenes | b12 |
| 3 | 2087 | FSL F6-184 | Listeria monocytogenes | c1 |
| 3 | 2088 | FSL F6-191 | Listeria monocytogenes | c2 |
| 3 | 2089 | FSL H1-099 | Listeria monocytogenes | c3 |
| 3 | 2090 | FSL J1-116 | Listeria monocytogenes | c4 |
| 3 | 2091 | FSL R2-192 | Listeria monocytogenes | c5 |
| 3 | 2092 | FSL J1-225 | Listeria monocytogenes | c6 |
| 3 | 2093 | FSL R2-500 | Listeria monocytogenes | c7 |
| 3 | 2094 | FSL R2-501 | Listeria monocytogenes | c8 |
| 3 | 2095 | FSL E1-159 | Listeria monocytogenes | c9 |
| 3 | 2096 | FSL F6-355 | Listeria monocytogenes | c10 |
| 3 | 2097 | FSL F6-382 | Listeria monocytogenes | c11 |
| 3 | 2098 | FSL F3-200 | Listeria monocytogenes | c12 |
| 3 | 2099 | FSL K2-143 | Listeria monocytogenes | d1 |
| 3 | 2100 | FSL N1-176 | Listeria monocytogenes | d2 |
| 3 | 2101 | FSL N1-417 | Listeria monocytogenes | d3 |
| 3 | 2102 | FSL L3-051 | Listeria monocytogenes | d4 |
| 3 | 2103 | FSL T1-107 | Listeria monocytogenes | d5 |
| 3 | 2104 | FSL T1-408 | Listeria monocytogenes | d6 |
| 3 | 2105 | FSL F6-396 | Listeria monocytogenes | d7 |
| 3 | 2106 | FSL H5-806 | Listeria monocytogenes | d8 |
| 3 | 2107 | FSL F6-551 | Listeria monocytogenes | d9 |
| 3 | 2108 | FSL F6-446 | Listeria monocytogenes | d10 |
| 3 | 2109 | FSL F6-315 | Listeria monocytogenes | d11 |
| 3 | 2110 | FSL V1-022 | Listeria monocytogenes | d12 |
| 3 | 2111 | FSL R2-132 | Listeria monocytogenes | e1 |
| 3 | 2112 | FSL R2-273 | Listeria monocytogenes | e2 |
| 3 | 2113 | FSL N3-277 | Listeria monocytogenes | e3 |
| 3 | 2114 | FSL F6-358 | Listeria monocytogenes | e4 |
| 3 | 2115 | FSL F6-194 | Listeria monocytogenes | e5 |
| 3 | 2116 | FSL R2-763 | Listeria monocytogenes | e6 |
| 3 | 2117 | FSL R2-765 | Listeria monocytogenes | e7 |
| 3 | 2118 | FSL R2-764 | Listeria monocytogenes | e8 |
| 3 | 2119 | FSL N1-225 | Listeria monocytogenes | e9 |
| 3 | 2120 | FSL N1-227 | Listeria monocytogenes | e10 |
| 3 | 2121 | FSL N1-048 | Listeria monocytogenes | e11 |
| 3 | 2122 | FSL K2-131 | Listeria monocytogenes | e12 |
| 3 | 2123 | FSL F6-222 | Listeria monocytogenes | f1 |
| 3 | 2124 | FSL F6-249 | Listeria monocytogenes | f2 |
| 3 | 2125 | FSL N3-065 | Listeria monocytogenes | f3 |
| 3 | 2126 | FSL H4-699 | Listeria monocytogenes | f4 |
| 3 | 2127 | FSL L4-241 | Listeria monocytogenes | f5 |
| 3 | 2128 | FSL S4-643 | Listeria monocytogenes | f6 |
| 3 | 2129 | FSL R2-073 | Listeria monocytogenes | f7 |
| 3 | 2130 | FSL F3-224 | Listeria monocytogenes | f8 |
| 3 | 2131 | FSL N4-334 | Listeria monocytogenes | f9 |
| 3 | 2132 | FSL R2-070 | Listeria monocytogenes | f10 |
| 3 | 2133 | FSL F6-421 | Listeria monocytogenes | f11 |
| 3 | 2134 | FSL F6-449 | Listeria monocytogenes | f12 |
| 3 | 2135 | FSL J2-054 | Listeria monocytogenes | g1 |
| 3 | 2136 | FSL S4-024 | Listeria monocytogenes | g2 |
| 3 | 2137 | FSL H1-111 | Listeria monocytogenes | g3 |
| 3 | 2138 | FSL K2-022 | Listeria monocytogenes | g4 |
| 3 | 2139 | FSL S4-066 | Listeria monocytogenes | g5 |
| 3 | 2140 | FSL R2-067 | Listeria monocytogenes | g6 |
| 3 | 2141 | FSL R2-293 | Listeria monocytogenes | g7 |
| 3 | 2142 | FSL F6-323 | Listeria monocytogenes | g8 |
| 3 | 2143 | FSL F6-216 | Listeria monocytogenes | g9 |
| 3 | 2164 | FSL F6-360 | Listeria monocytogenes | g10 |
| 3 | 2165 | FSL F6-313 | Listeria monocytogenes | g11 |
| 3 | 2166 | FSL R2-031 | Listeria monocytogenes | g12 |
| 3 | 2167 | FSL R2-050 | Listeria monocytogenes | h1 |
| 3 | 2148 | FSL T1-313 | Listeria monocytogenes | h2 |
| 3 | 2149 | FSL R8-0875 | Listeria monocytogenes | h3 |
| 3 | 2150 | FSL R2-317 | Listeria monocytogenes | h4 |
| 3 | 2151 | FSL F6-335 | Listeria monocytogenes | h5 |
| 3 | 2152 | FSL R6-653 | Listeria monocytogenes | h6 |
| 3 | 2153 | FSL L3-135 | Listeria monocytogenes | h7 |
| 3 | 2154 | FSL L3-143 | Listeria monocytogenes | h8 |
| 3 | 2155 | FSL L3-167 | Listeria monocytogenes | h9 |
| 3 | 2156 | FSL N3-031 | Listeria monocytogenes | h10 |
| 3 | 2157 | FSL J1-101 | Listeria monocytogenes | h11 |
| 3 | 2158 | FSL F6-154 | Listeria monocytogenes | h12 |

### Example 4: Effect of Phage Concentration on Signal Intensity

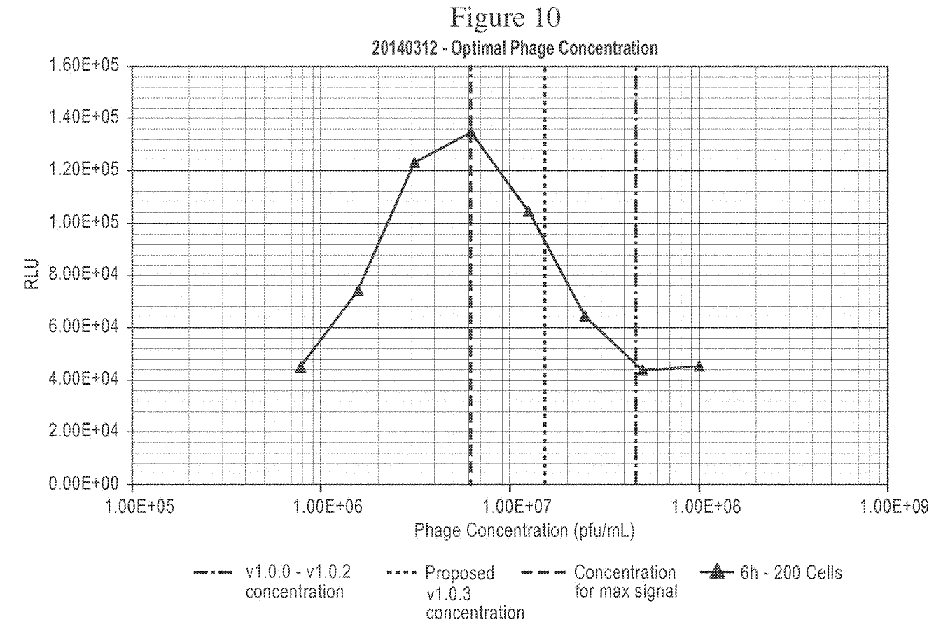

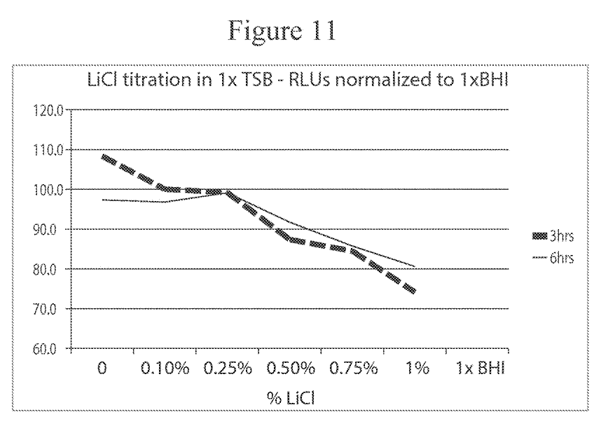

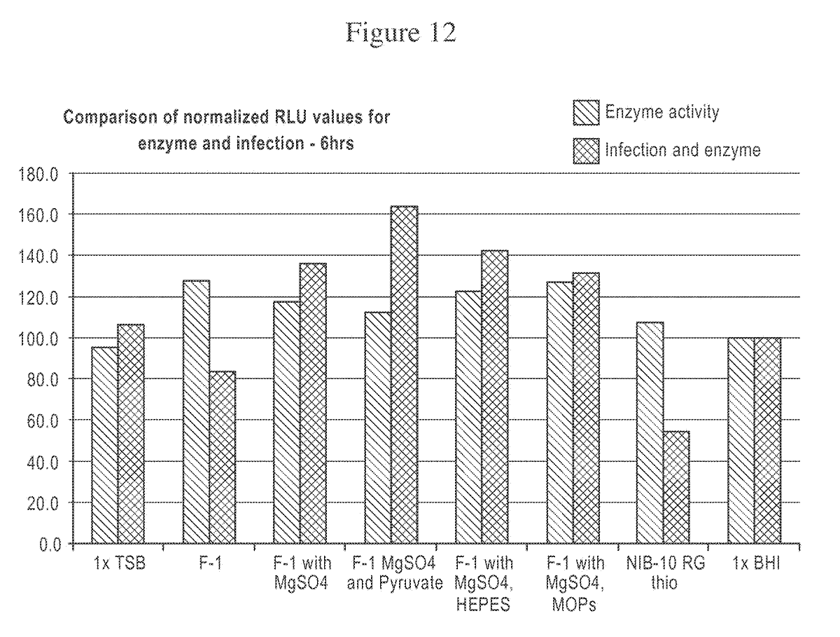

Assays were performed to determine the effect of phage concentration on the resultant signal intensity following infection with the codon-optimized phage. See FIG. 10. For these experiments, two-fold serial dilutions of the COP2 phage (v1.0.2) cocktail were added to the infection buffer. 200 CFU of Listeria monocytogenes (NP#1839) were infected at various concentrations of phage. The infected samples were incubated for 6 hours at 30° C. The luciferase signal was detected using the Glomax 96 luminometer. The resultant signal intensities for the various concentrations of phage used was plotted on a graph for comparison of signal. The data from these experiments indicate that the concentration for maximum signal is approximately between 1.5×106 to 1.8×106 pfu/mL. See FIG. 10. All assays were performed minimally in triplicate.

### Example 5: Alterations in the 5′ UTR Results in Increased Signal Intensity

Optimization of the 5′ UTR was performed by utilizing DNA 2.0 TM. The changes to the 5′ UTR included modifications of spacer DNA and/or changes in the nucleotide sequence of the ribosome binding site (RBS). These sequences, including the original UTR sequence, can be found in the informal sequence listing at SEQ ID NO: 90-97. Multiple variants were produced, several of which resulted in greater than a 200% mean signal increase over the COP2 (v1.0.2) phage signal. See Table 11. For these assays, optimized UTR variants were linked to a modified NanoLuc construct called COPD12 (see SEQ ID No. 53).

TABLE 11
Codon-optimized 5′ UTR Results in Increased Signal Intensity

| | Mean Signal |
|---|---|
| Variant | Increase over v1.0.2 |
| UTR2 | 264% |
| UTR4 | 289% |
| UTR7 | 232% |

The 5′ UTR changes that resulted in increased signal intensity were combined with the best-performing COP3 codon-optimized variant (i.e. 40_VIP178) to assess whether this combination would result in increased signal in comparison to either the 5′UTR optimization or the COP3 variant alone. The results surprisingly show that the combined variants do not have increased signal in comparison to the COP3 variant that does not contain an altered 5′ UTR sequence. See Table 12.

TABLE 12
COP3 Combined with 5′UTR Modification
Does Not Result in Greater Signal Intensity

| | Mean Signal |
|---|---|
| Variant | Increase over v1.0.2 |
| UTR2_178 | 182% |
| UTR3_178 | 185% |
| UTR7_178 | 332% |
| 40_VIP178 | 468% |
| UTR2 | 243% |
| UTR3 | 154% |
| UTR7 | 191% |

### Example 6: Selection of Base Media

Media were screened for the ability to support high infection rate and high signal intensity following infection with phage encoding a luciferase marker. Bacterial cells were purposefully stressed by way of drying for 18 hours on a stainless steel table, followed by re-suspension in brain-heart infusion medium (BHI) for 30 minutes. The recovered cells were then infected with phage containing a luciferase marker for 6 hours followed by testing of the enzymatic activity using unpurified phage lysate and NanoLuc reagent. All media tested gave similar RLU output.

BHI and TSB media were further titrated to assess whether there was an increase in RLU at different concentrations of base media. The data indicate that stressed cells recovered best in 1×TSB medium. Additional benefits of the 1×TSB medium include that the TSB does not contain animal byproducts, it contains more nutrients, and there is better consistency of the product among different lots tested.

### Example 7: Selection of Media Additives—Selective Agents, Neutralizers, and Nutrients

Select components were added to the media in order to remedy known stressors to cells, and to reduce the possibility for other chemicals interfering with the test results. To this end, lithium chloride was added at defined concentrations to the selected 1×TSB base medium, followed by infecting the cells with a luciferase containing phage, and lastly by assaying the RLU detectable as a function of the percentage of lithium chloride added to the media. Lithium chloride was selected as an additive in order to overcome, prevent or limit growth of competing biologicals. The data from these experiments indicate that, lithium chloride added at 0.25% resulted in the highest RLU at both 3 hours and 6 hours post-infection. (See FIG. 11).

Components selected to overcome cell starvation and oxidative stress, glucose and yeast extract, respectively, were titrated in 1×TSB, followed by infection of the bacteria with luciferase containing phage, and lastly by assaying the RLU detectable as a function of the percentage of either glucose or yeast extract. Based on the RLU levels at the 3 hour and 6 hour assay points, 0.25% glucose and 0.5% yeast extract were selected.

Antifungal agents were also added to the base medium and tested in order to determine whether there is a decrease in RLU activity, either as a result of loss of enzyme activity or because of reduced infection ability. The anti-fungal agents tested included cycloheximide solution in DMSO. Neither cycloheximide nor DMSO resulted in a decrease in the infection rate or in the enzymatic activity.

The effect of divalent cations added to Formulation-1 was assessed. MgSO₄ or CaCl₂ was added to Formulation-1, followed by infection of bacteria with luciferase encoding phage, and assessment of resultant RLU. Addition of MgSO₄ to Formulation-1 resulted in a marked increase in the RLU activity in comparison to the addition of CaCl₂. The beneficial activity on resultant RLU activity led to the creation of Formulation-1A, which contains 0.08% MgSO₄. (See Table 2 and FIG. 12).

The addition of alternative carbon sources, through the addition of alpha-ketoglutarate, glutamate, malonate and citrate to Formulation-1 demonstrated that glucose sustained RLU activity more effectively.

A comparison of enzyme activity and infection ability with various media formulations indicated that a preferred embodiment of the formulation includes the base Formulation-1, with the addition of 0.08% MgSO4 and 0.1% pyruvate (also referred herein as Formulation-1A). (See FIG. 27).

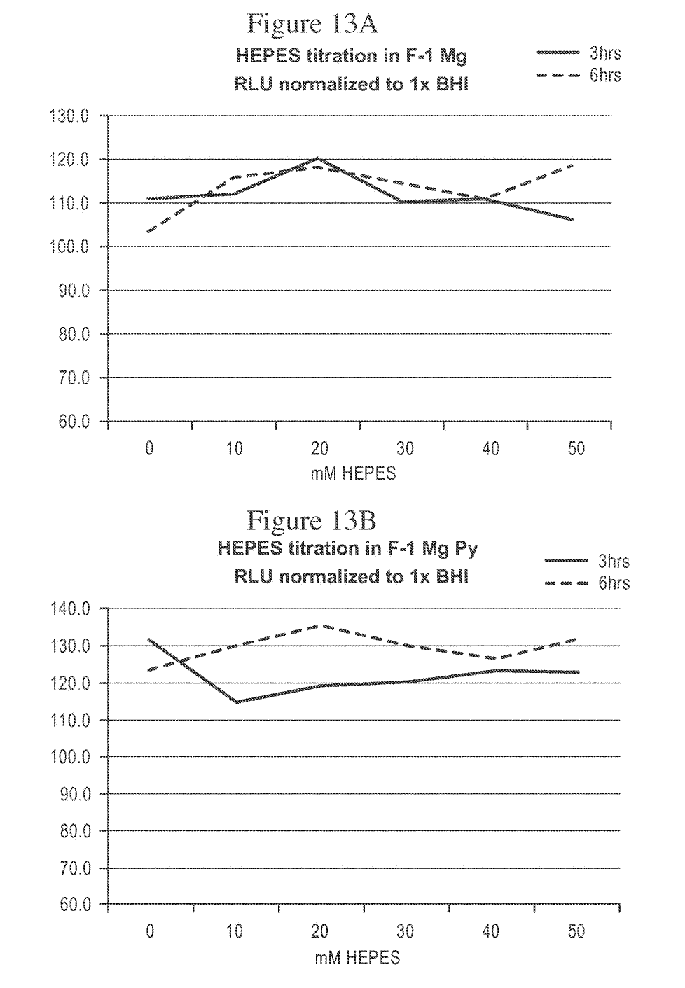

HEPES was also titrated into Formulation-1A to investigate whether addition of HEPES resulted in higher RLU activity than without the addition of the buffer. The data indicated that 20 mM HEPES was ideal in both the 3 hour and the 6 hour assay time points for high RLU activity. Furthermore, the addition of pyruvate further increased RLU activity. (See FIGS. 13A and 13B). The resultant formulation that incorporates all of the components of Formulation-1A, with the addition of HEPES, is herein referred to as Formulation-2 or as NIB-12. (See Table 3).

### Example 8: Formulation NIB-14

Based on the information gleaned from the effects acquired through the addition of additives to the base TSB formulation (see Example 7 above), other components were selectively added to generate another preferred embodiment of the formulation, which is particularly well suited for resisting the negative impact of chemicals found in sanitizing solutions on both enzymatic stability and phage infection. Of particular interest are additives that are geared towards reducing the interference from quaternary ammonium salts found in various sanitizing solutions including among others: “Sani-Step,” “Sani-Save,” “Boost-FT,” “Quorum Clear V,” “Whisper V,” “Sparkle QF-BH,” “F29”. Particularly good candidates that have the capability of reducing interference of quaternary ammonium salts are Tween-80 and lecithin.

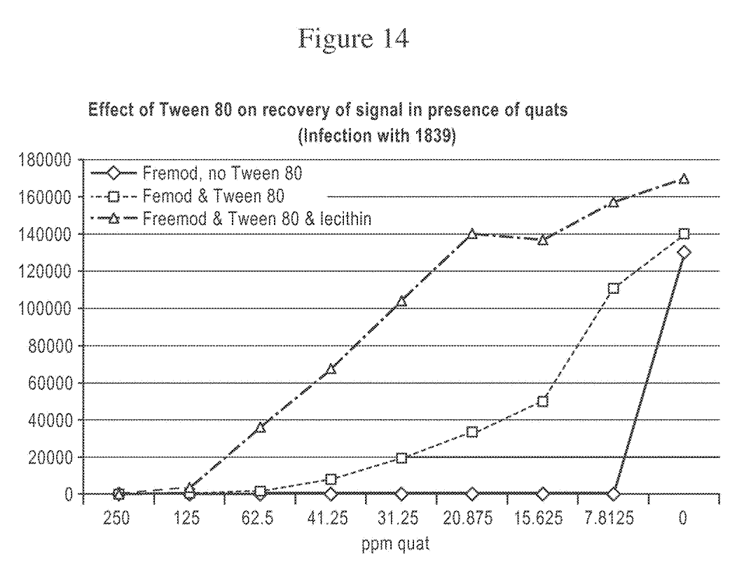

The addition of Tween-80, either alone or in combination with lecithin, to the Formulation-2 (NIB-12) medium allowed for protection of the samples from interference by quaternary ammonium salts. (See FIG. 14). Without the addition of either Tween-80 or of lecithin to Listeria Growth Broth, there was no detectable RLU when the solution contained 7.81 ppm of quaternary ammonium salts. However, the resistance to quaternary ammonium salts was also increased by the addition of Tween-80 alone. In this case, there was a sustained, detectable RLU in the presence of up to 62.5 ppm quaternary ammonium salts. The amount of resistance to quaternary ammonium salts was further increased through the addition of both lecithin and Tween-80. With the addition of both reagents to Listeria Growth Broth, there was detectable RLU up to 125 ppm quaternary ammonium salts. Another preferred embodiment of the media contains 1.5% of Tween-80 and 0.22% of lecithin. This formulation is herein referred to as NIB-14, the full formulation of which is detailed in Table 4.

The stability of the NIB-14 medium was also tested at temperatures, 4° C. and 30° C., and normalized to that of the standard base medium 0.5×BHI. The data indicate that NIB-14 remains stable at both 4° C. and 30° C.

### Example 9: Phage Infection and Enzyme Activity

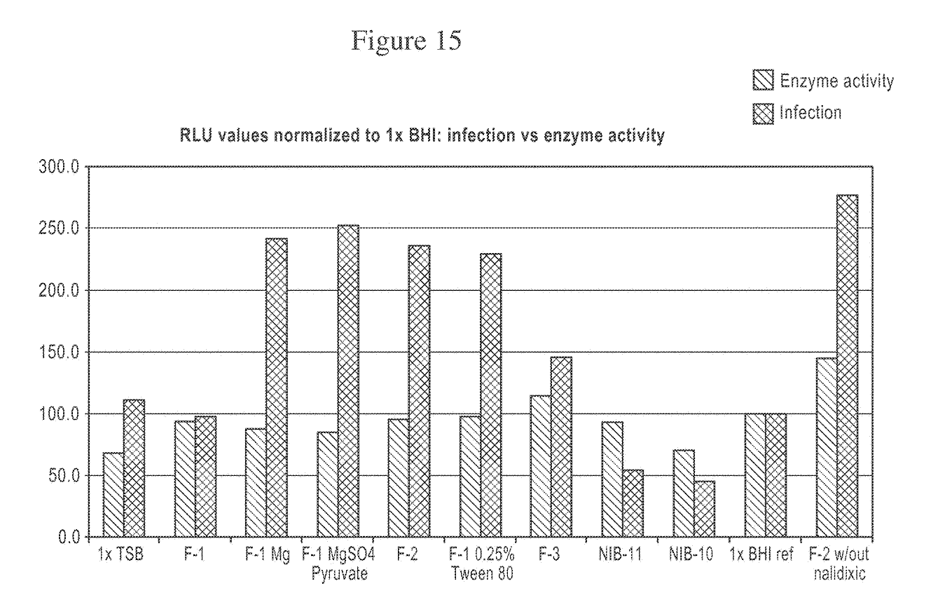

A systematic comparison of RLU values as a function of additive component was performed on various media formulations. (See FIG. 15). The effects of different additives on both enzymatic activity, as well as infection ability, was measured and compared by way of assessing RLU across the different conditions. The data indicate that the highest infection ability was found in Formulation-1 (with Mg and pyruvate) and Formulation-2 supplemented with HEPES. Specifically, addition of Mg, pyruvate and/or 0.25% Tween-80 had a large impact in increasing infection ability.

While large gains were found in the infection ability with the additions listed above, modest differences were observed with regard to promoting enzymatic activity by way of additives to the formulations.

### Example 10: Effects of Media Formulation on Lower Limit of Detection Assays

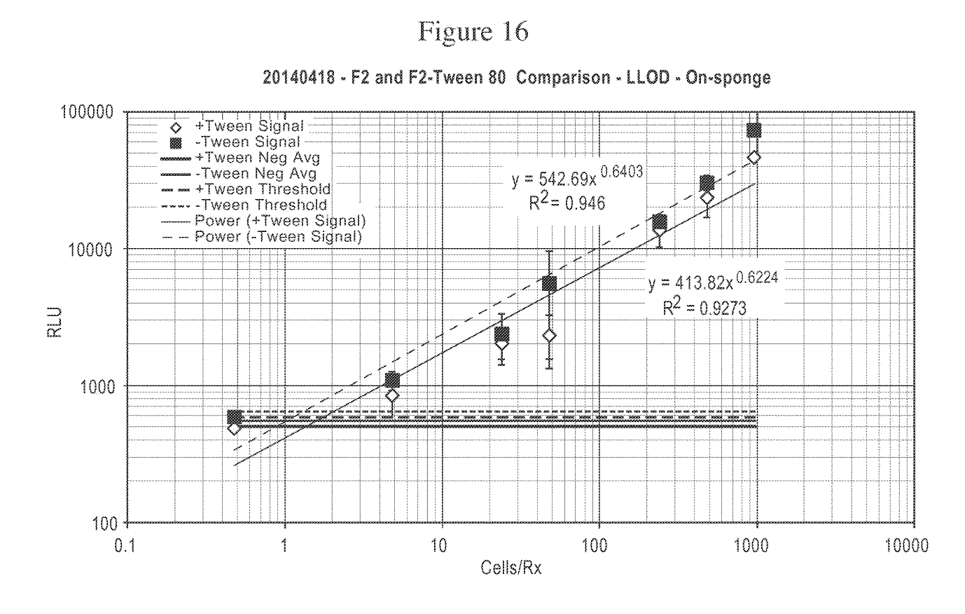

The influence of media formulation on the ability to detect small quantities of cells was assessed by performing lower limit of detection assays (also referred to herein as “LLOD”). For these experiments, two variations of Formulation-2 were assessed (see Table 3). The two conditions tested included either the standard Formulation-2 (see Table 3), or the standard Formulation-2 containing 0.25% of Tween-80. For these experiments, stressed 1554 bacterial cells were collected on sponges and incubated with the appropriate formulation medium. Cells were titrated over a range of values in order to have a graphical output in the LLOD assay ranging from 1 cell to 1000 cells. In the two conditions measured, the standard Formulation-2 (NIB-12) performed better in terms of detection sensitivity when compared with the standard formulation containing 0.25% Tween-80. (See FIG. 16).

### Example 11: Use of NIB-14 in the Presence of Varying Amounts of Sanitizers

The NIB-14 formulation included the use of neutralizers (e.g. Tween-80 and lecithin) meant to play a role in reducing the effects of remnant amounts of sanitizers in a sample. As such, NIB-14 was shown to allow for high amounts of infection and subsequent signal stability. (See Example 8). Subsequent assays, meant to ascertain the levels of protection provided by the NIB-14 formulation towards various kinds remnant sanitizer samples were performed.

For these assays, the following were taken into account: (i) evaluation of whether the products glow on their own (i.e. in the absence of cells, phage and/or enzyme), (ii) evaluation of the effect of sanitizing chemicals on NanoGlo substrate (in the absence of cells, phage and/or enzyme), (iii) evaluation of the role that NIB-14 plays in the neutralization of remnant sanitizing chemicals by comparison of the effects obtained by the addition of NIB-14 versus those of the base bacterial growth medium, Letheen formulated to neutralize quaternary ammonium compounds. These assays incorporated the evaluation of the deleterious effects that the sanitizing compounds have at either (i) the point of phage infection, (ii) enzymatic activity, or (iii) direct effect on the NanoGlo luciferin substrate. To determine the effect of the sanitizing chemicals on phage infection, the 1893 cell-type was used at a density of 900 cells per well BHI basal medium, and further incubated with serially diluted (into either NIB-14 or Letheen buffer) sanitizing chemicals for 30 minutes. Following the 30 minute incubation period, the luciferase marker containing phage was added in BHI medium, and the samples were further allowed to incubate for an additional 3 hours at 30° C. The enzyme was then detected by the addition of the NanoGlo luciferin substrate and luminescence measurement. In another variant of this assay, meant to ascertain the remnant sanitizer chemical interaction with the enzymatic activity, the sanitizer chemicals were added to the enzyme only in either NIB-14 or in Letheen for 3 hours at 30° C. Another variant of this assay involved the direct incubation of the sterilization chemical with the NanoGlo luciferin substrate in the absence of either cells, phage or enzyme. In another version of this assay, the sanitizer chemicals were added at various time intervals (e.g. 5 min to 120 min) directly to the NIB-14 or to the Letheen, followed by the addition of cells and phage and incubated for 3 hours at 30° C.

Control assays revealed that there were no false positive luminescence signals in the absence of the NanoGlo luciferase substrate.

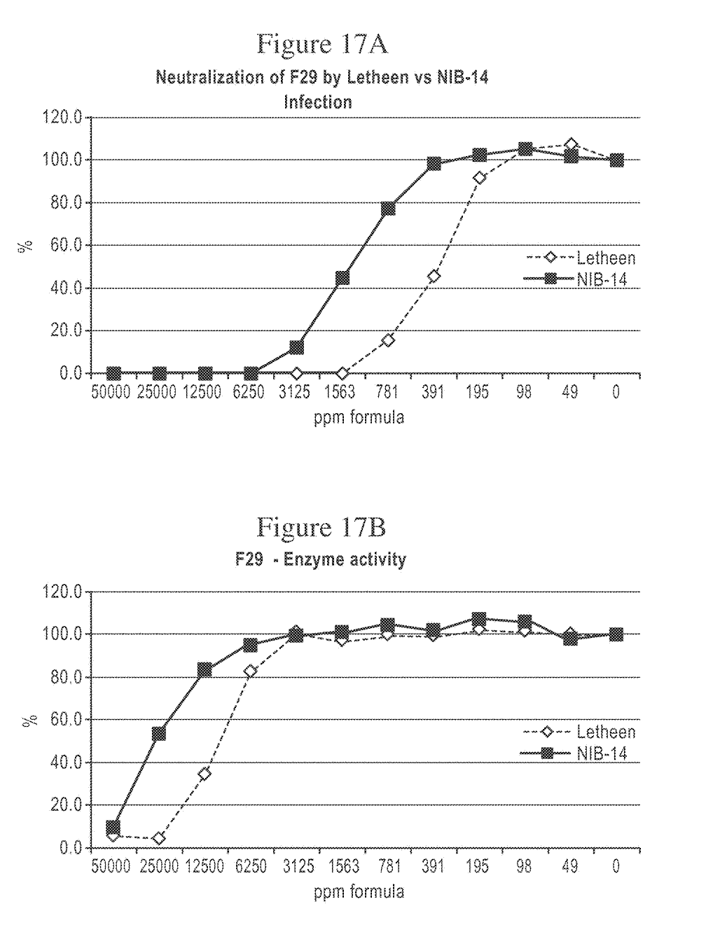

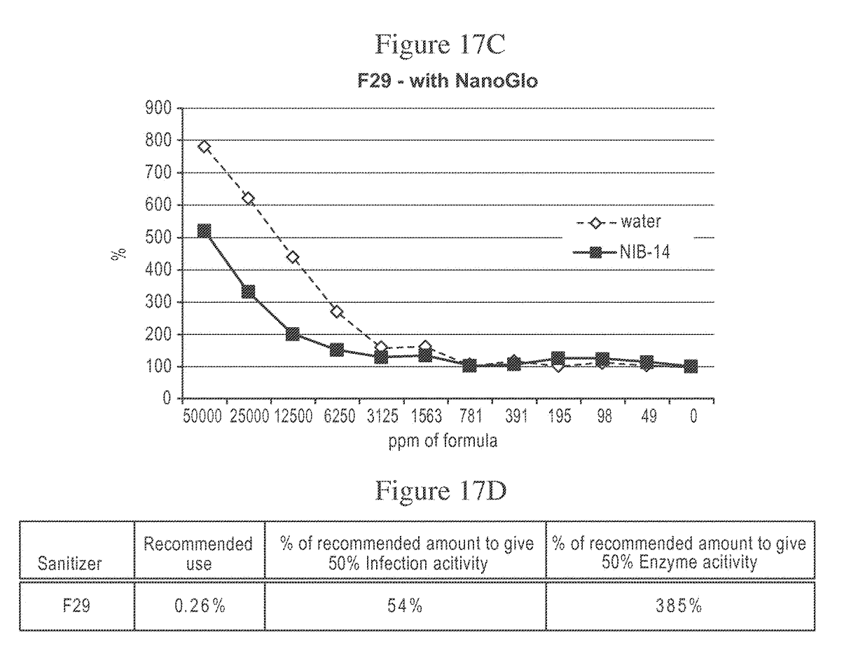

The sanitizer solution, F29, which contains quaternary ammonium salts, was diluted to a final concentration of 0.26%, for a total quaternary ammonium salt concentration of approximately 300 ppm for use in these assays. The recommended usage amounts of F29 for cleaning purposes is 150 ppm of the active ingredient for a 3 minute duration of direct contact on non-food surfaces, and 400-800 ppm in entryways. The assays indicate that both the infection ability and the enzymatic activity are protected by the use of the NIB-14 medium in comparison to the Letheen medium. (See FIGS. 17A-17D). Greater than 20% phage infection rate was retained in exposures of up to 2600 ppm of F29, and enzymatic activity of near 100% was maintained in exposures of up to 2600 ppm of F29 during incubation with the NIB-14. (See FIGS. 17A and 17B, respectively). The effect of incubating different amounts of the F29 sanitizer with the NanoGlo substrate also indicated that the addition of NIB-14 in the assays had a beneficial protective effect on preserving the NanoGlo substrate, when compared to water. (See FIG. 17C). A summary of the findings indicates that the addition of NIB-14 is beneficial at the phage infection step, the enzymatic step, and also provides protection of the NanoGlo substrate. (See FIG. 17D).

Another sanitizer used to assess the protective ability of NIB-14 was Quorum Clear. The Quorum Clear sanitizer contains quaternary ammonium salts. The recommended usage concentration for Quorum Clear is a 3% solution. Addition of the NIB-14 medium was able to preserve up to 50% phage infection ability and enzyme activity at concentrations of Quorum Clear of up to 2.0% and 3.0%, respectively. NIB-14 was also able to provide protection to the NanoGlo substrate during exposures to Quorum Clear.

Another commonly used sanitizer component that was tested in the microbial detection system assays was hydrogen peroxide (H₂O₂). As in the previously described assays, the protective ability of NIB-14 was determined in situations where various concentrations of the sanitizing component were added either during the phage infection step, the enzymatic activity step, or to the NanoGlo substrate. The data indicate that NIB-14 provides protection to peroxide presence in comparison to Letheen. (See FIGS. 17A, 17B and 17D). The data do not indicate protection by NIB-14 to the NanoGlo substrate. (See FIG. 17C). However, there are no deleterious effects to the NanoGlo substrate during exposure to peroxide at concentrations recommended for sanitizing (i.e. 500 ppm).

Boost FT was another sanitizer used in the microbial detection system assays to determine the protective effects of NIB-14 in the microbial detection assays. Boost FT contains a combination of quaternary ammonium salts, peroxide (H₂O₂), and EDTA at elevated pH. The recommended concentration of use for Boost FT is 0.7% concentration of the active ingredient. Addition of the NIB-14 medium was able to preserve up to 50% phage infection ability and enzyme activity at concentrations of Boost FT of up to 0.07% and greater than 0.7%, respectively. However, the increase noted in enzymatic activity may largely be due to oxidization of the NanoGlo substrate. The need for additional neutralization of peroxides was demonstrated.

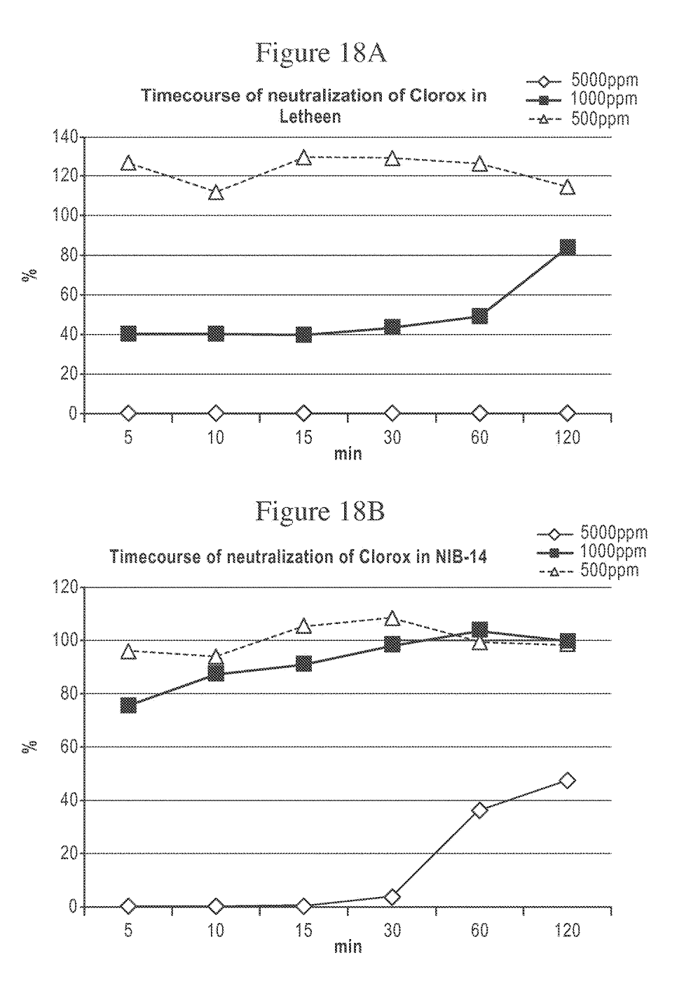

The effects of another commonly used sanitizer, Clorox, and the protective benefits of NIB-14 medium on the microbial detection system in these conditions were assayed. The data indicated that at concentrations of greater than 400 ppm of hypochlorite there was no infection signals detected. Likewise, enzymatic activity was also negatively affected in media tested, NIB-14 and Letheen. The hypochlorite concentrations that were tested did not negatively impact the NanoGlo substrate. An additional assay performed with the Clorox sanitizer was a time-course assay in which RLU was measured following incubation of Clorox for defined periods of time in either Letheen or in NIB-14, followed by addition of this solution to the microbial detection system assays. The data show that NIB-14 has a strong neutralization capacity in terms of allowing higher RLU activity with progressive time in Clorox. (See FIGS. 18A and 18B).

Further investigation into the addition of oxygen scavengers into the NIB-14 formulation was assessed. Data acquired from the microbial detection system assays demonstrated that the addition of either 2 mM sodium metabisulfite or 0.05% sodium thiosulfate reduced the oxidizing effect of peroxide in the assay. The oxidizing effect of the peroxide found in Boost FT was lowered in the assays with either neutralizer when the effect on substrate signal alone was assessed. The addition of 2 mM sodium metabisulfite was especially beneficial in lowering the signal at the highest Boost FT concentrations that were caused by peroxides when enzyme and infection activity were tested.

### Example 12: Comparison of Infection Buffers in Stressed Cell Infection Assays

A direct comparison of infection and enzymatic activity using stressed cells infected with recombinant luciferase encoding phage was performed utilizing NIB buffers. These tests measured the direct performance of NIB-10*, NIB-12, and NIB-14 with regard to enhancing either enzymatic activity or infection rate. The base buffer, 1×BHI, was used as a comparison buffer for these tests. For the stressed cell assays, bacterial cells were purposefully stressed by way of drying for 18 hours on a stainless steel table followed by downstream processing. The effect of the buffers was assessed either during the infection stage or during the enzymatic processing stage. Subsequently, the RLU activity was recorded for each of the buffer conditions. *NIB-10 is composed of: 1×BHI, 0.5% LiCl, 0.002% nalidixic acid, 0.2% yeast extract, 2 mM CaCl2, 40 mM HEPES, pH 7.4, 1 mM sodium metabisulfite, 0.1% sodium thiosulfate, 0.5% Tween-80, and 0.1% lecithin.

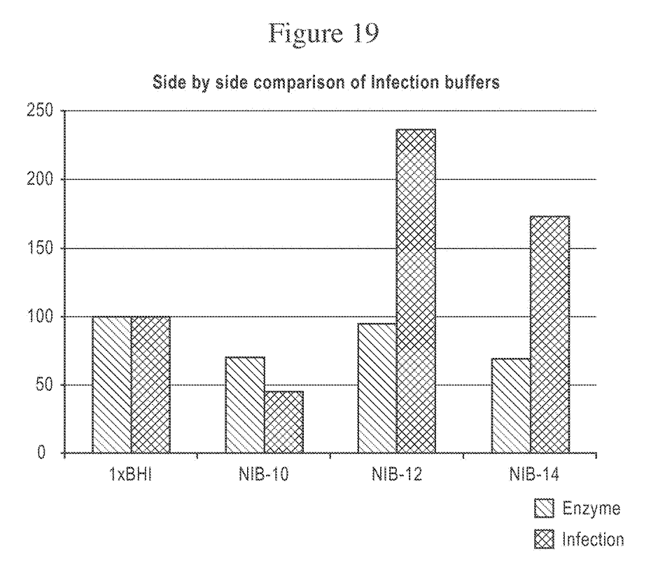

The data indicate that NIB-12 (see Table 3) had the greatest beneficial effect during the infection step, as indicated by the highest RLU values among all of the buffers tested. (See FIG. 9). The second most beneficial buffer in enhancing the infection step was NIB-14. This buffer offers instead higher neutralization power against residual sanitizers. The influence of the buffers was not as pronounced during the enzymatic activity phase; however, of the NIB buffers tested, NIB-12, supported the most enzymatic activity, followed by the similar enzymatic activity rates of both NIB-10 and NIB-14 as determined by RLU output. (See FIG. 19).

### Example 13: Lower Limit of Detection Assays in the Detection of Microbes in Food

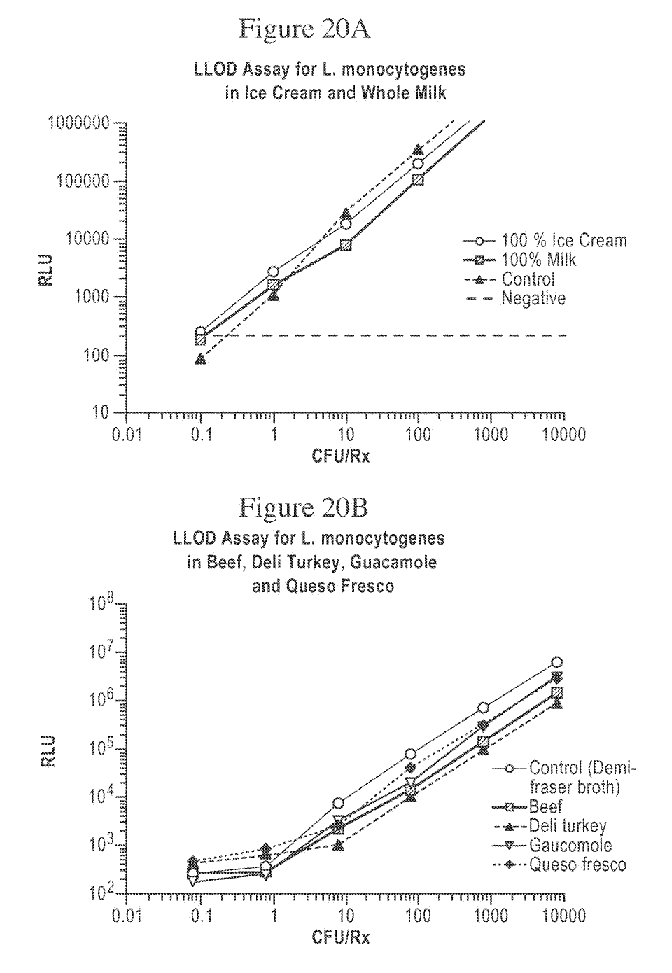

Lower limit of detection assays (“LLOD”) were performed with whole milk that had received a defined amount of Listeria monocytogenes. For these assays, 25 mL of 100% (full fat) whole milk, 25 mL of NIB-14 infection buffer (see Table 4), and 4.5×10⁷ pfu/mL recombinant marker encoding phage were used. The recombinant phage had luciferase as the recombinant marker. The results of the LLOD assays revealed that within 2 hours of the addition of L. monocytogenes to the food sample there was detectable signal in the assays, wherein up to 50 cells in a 50 mL sample was detectable. (See FIG. 20A). These data indicate that up to 50 CFU of L. monocytogenes was detectable in the assays.

LLOD assays for the detection of L. monocytogenes were also performed with other foods including raw ground beef, deli turkey, guacamole and queso fresco. (See FIG. 20B). The data from the LLOD assays with these foods also indicate that the target microbe was detectable at CFUs of between 1 and 10.

### Example 14: Time Course Detection Assays for Microbes in Food

Assays to establish the amount of time before the detection of defined amounts of L. monocytogenes is possible were performed with food samples of turkey, guacamole, queso fresco, raw ground beef, potato salad, smoked salmon, and sour cream. For these assays, L. monocytogenes was added to the food sample, followed by waiting for a defined amount of time prior to adding the recombinant luciferase encoding phage for at least 2 hours, and subsequent assessment of the luciferin signal in the microbial detection assay. Depending on the kind of food in the assay, different dilutions of food matrix to incubation buffer were performed. The dilutions for the different foods assessed were: guacamole 1:3, ground beef (80/20) 1:3, whole milk 1:1, ice cream 1:1, queso fresco 1:1 and deli turkey 1:3. For example, for the detection of L. monocytogenes in deli turkey samples, 25 g of food matrix was spiked with either 2 or 20 CFU and then incubated with 75 mL NIB-14. Following the incubation, a sample of the NIB-14 liquid was incubated with the recombinant phage for 3 hours.

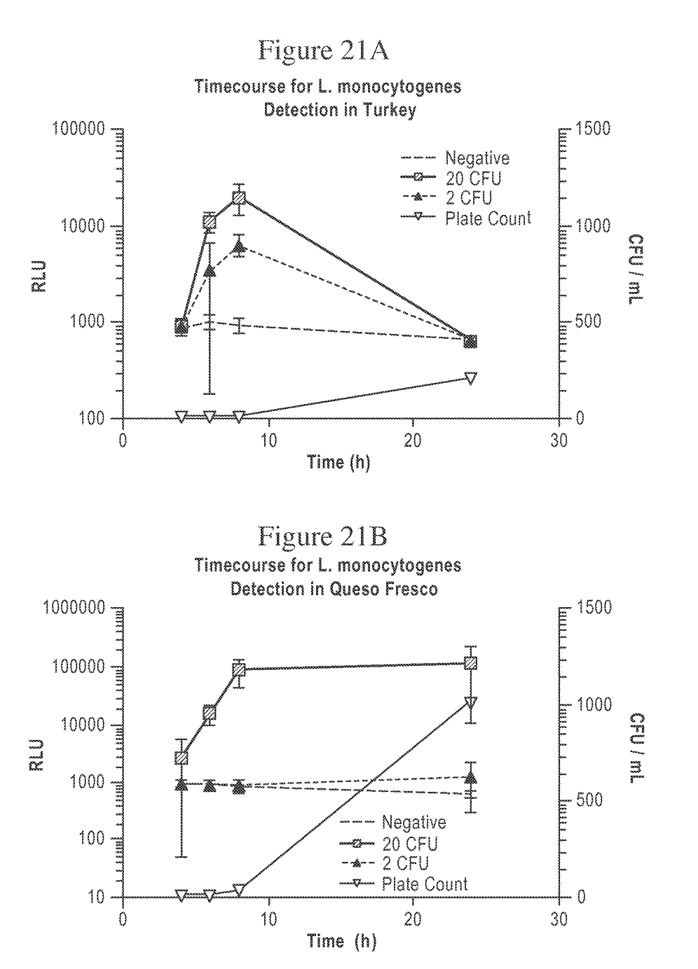

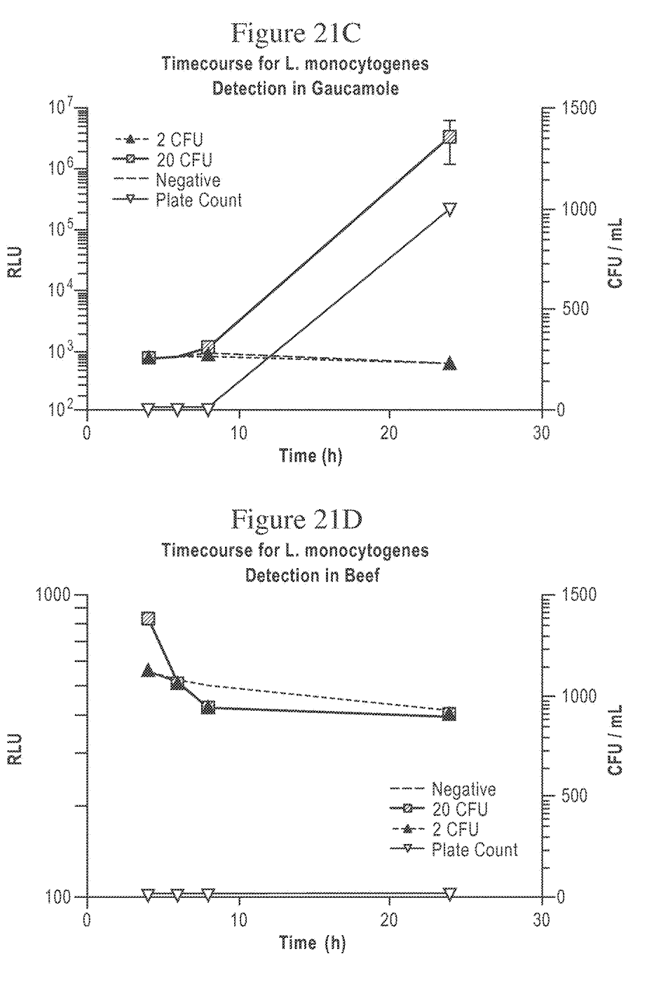

The results indicated that the detection of L. monocytogenes was found after 6 hours at both the 2 and 20 CFU conditions in the deli turkey food samples (FIG. 21A), after 4 hours for the 20 CFU condition and 24 hours for the 2 CFU condition in the queso fresco samples (FIG. 21B), after 8 hours in the 20 CFU condition for the guacamole samples (FIG. 21C), and there was no detectable L. monocytogenes found at either the 2 or the 20 CFU conditions for the beef samples (FIG. 21D). (See FIGS. 21A-D).

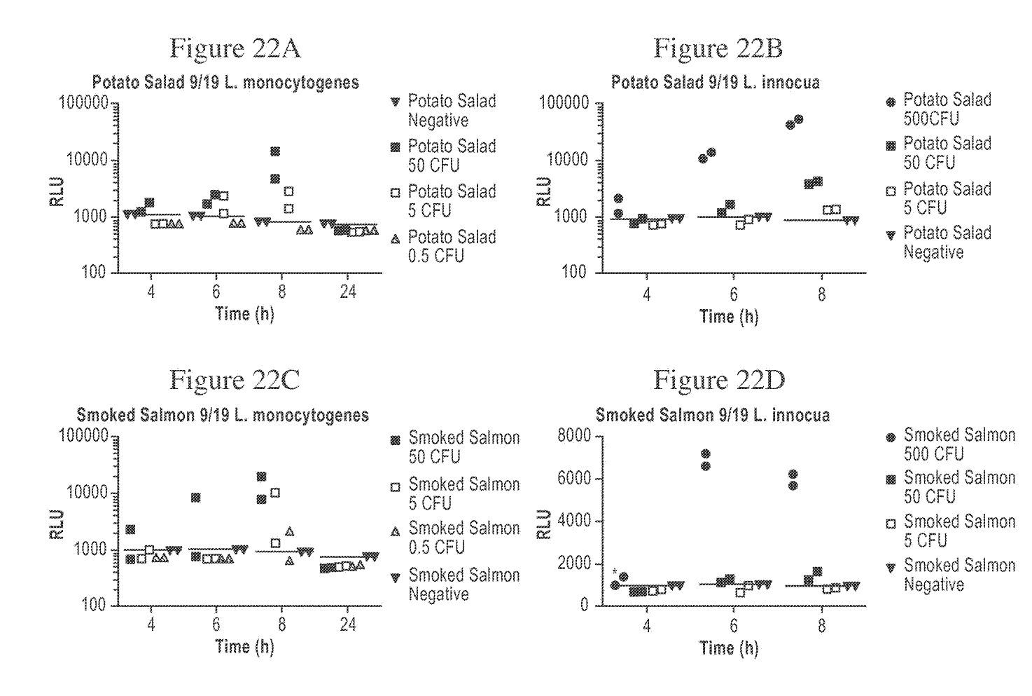

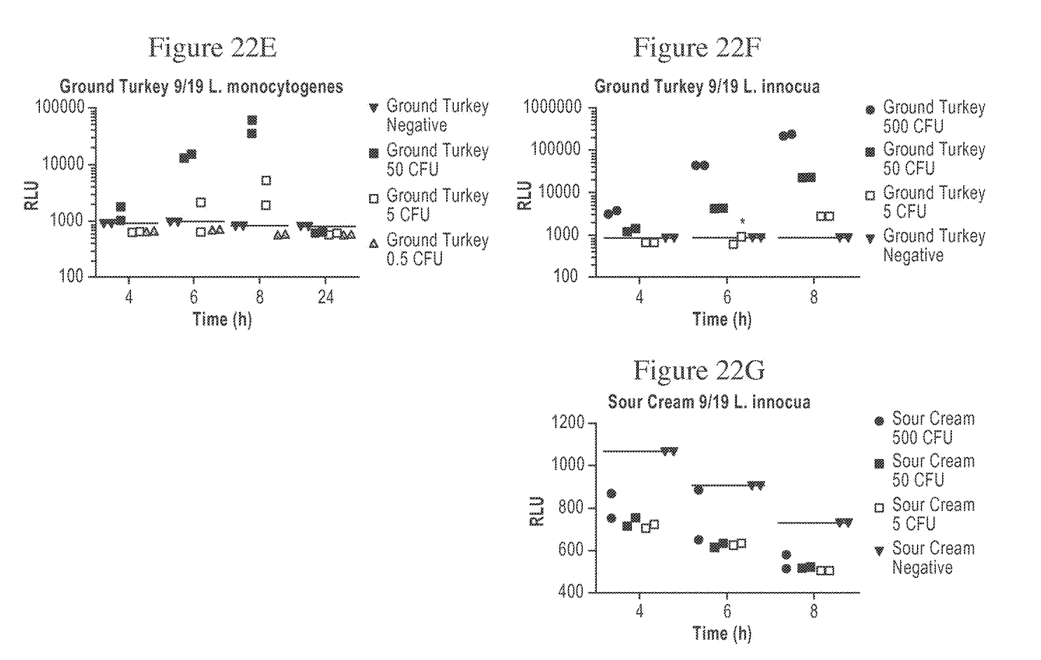

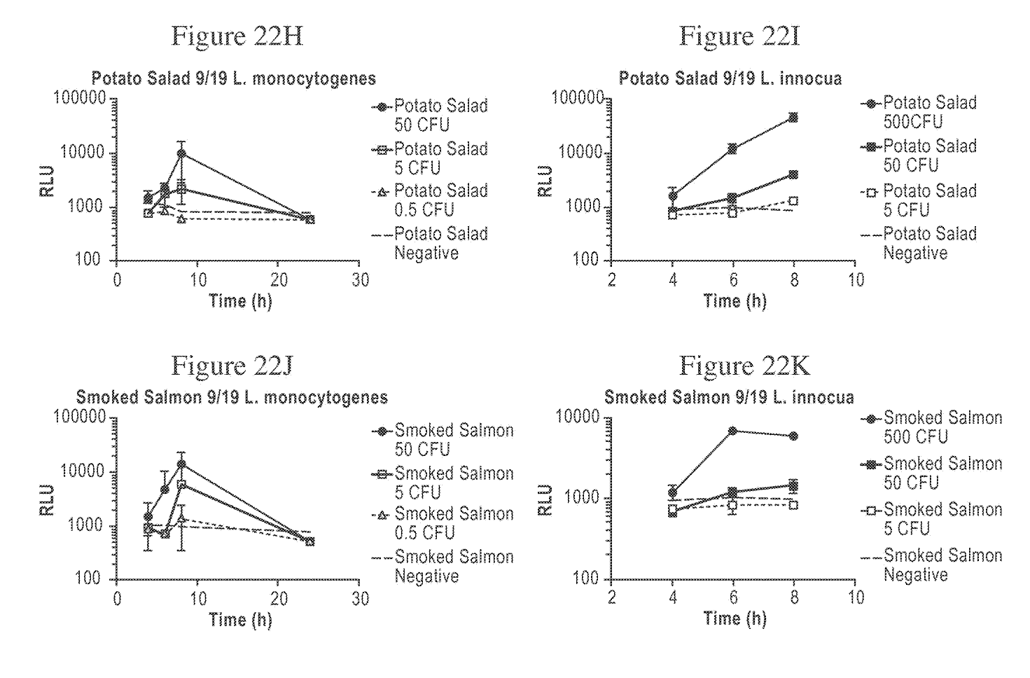

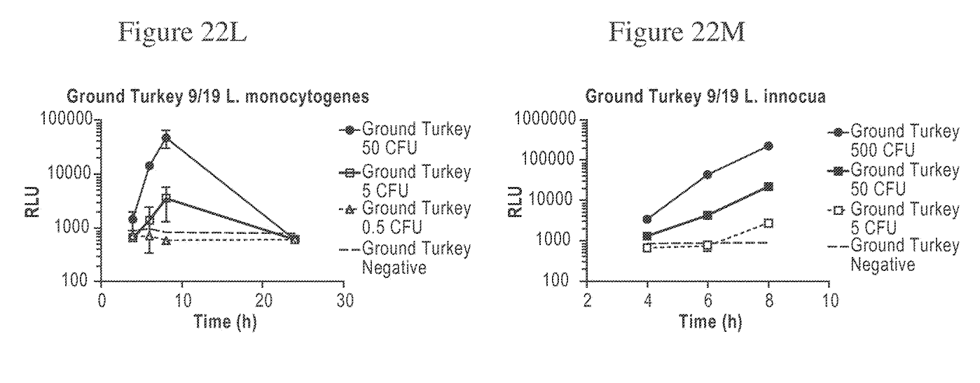

A comparison between the amounts of time before the detection of L. monocytogenes and L. innocua is presented in FIG. 22A-M. For these experiments a four hour resuscitation/enrichment of the bacteria was performed followed by a three hour incubation with the luciferase encoding phage. The data indicate that the detection of L. monocytogenes is possible at lower CFU values when compared to the detection of L. innocua. As is indicated by the graphs, both bacteria species are detectable with the assay system. (See FIG. 22 A-M).

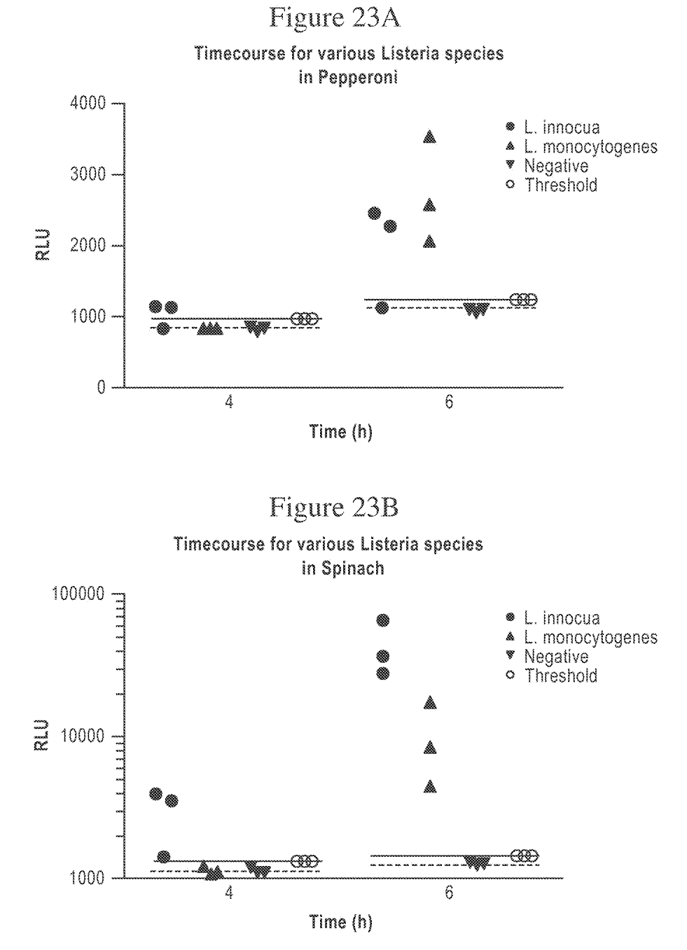

Another time course to detection was performed using L. monocytogenes and L. innocua incubated in either pepperoni or spinach. (See FIG. 23A-B). For these experiments a four hour resuscitation/enrichment of the bacteria was performed followed by a three hour incubation with the luciferase encoding phage. The data indicate there are differences with regard to the time of incubation before which the bacteria are detected in either the spinach or the pepperoni. However, both bacterial species were detectable with the assay system by 6 hours of incubation.

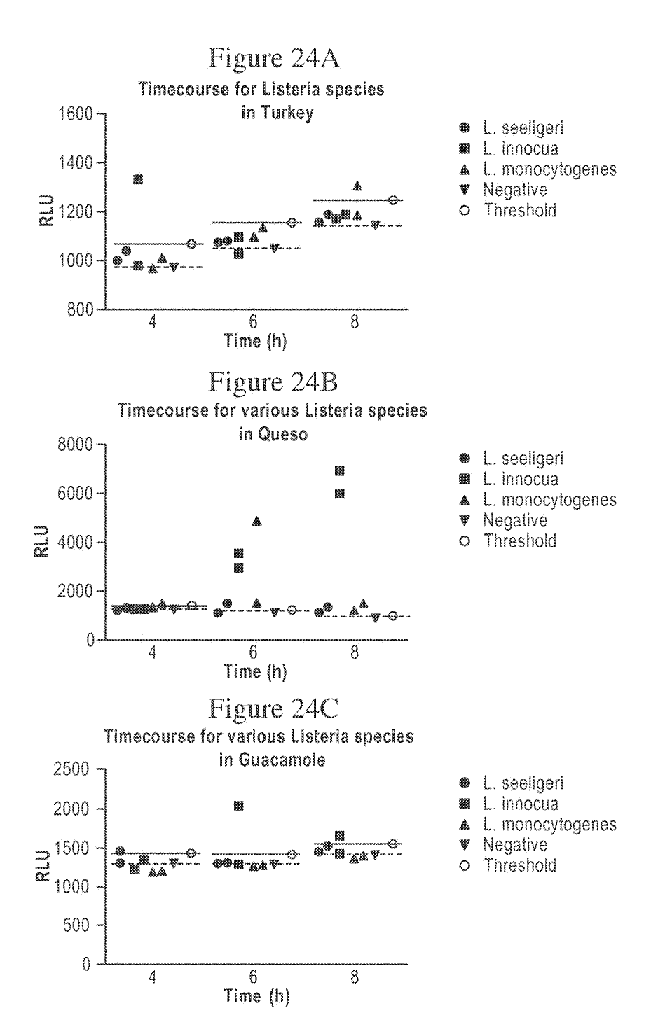

A time course to detection assay was also performed using three species of Listeria, L. seeligeri, L. innocua, and L. monocytogenes incubated in turkey, queso, and in guacamole. (See FIG. 24A-C). The data indicate that the detection of the various species of Listeria is dependent on food matrix type (e.g. shorter time to detection in Turkey) and the Listeria species.

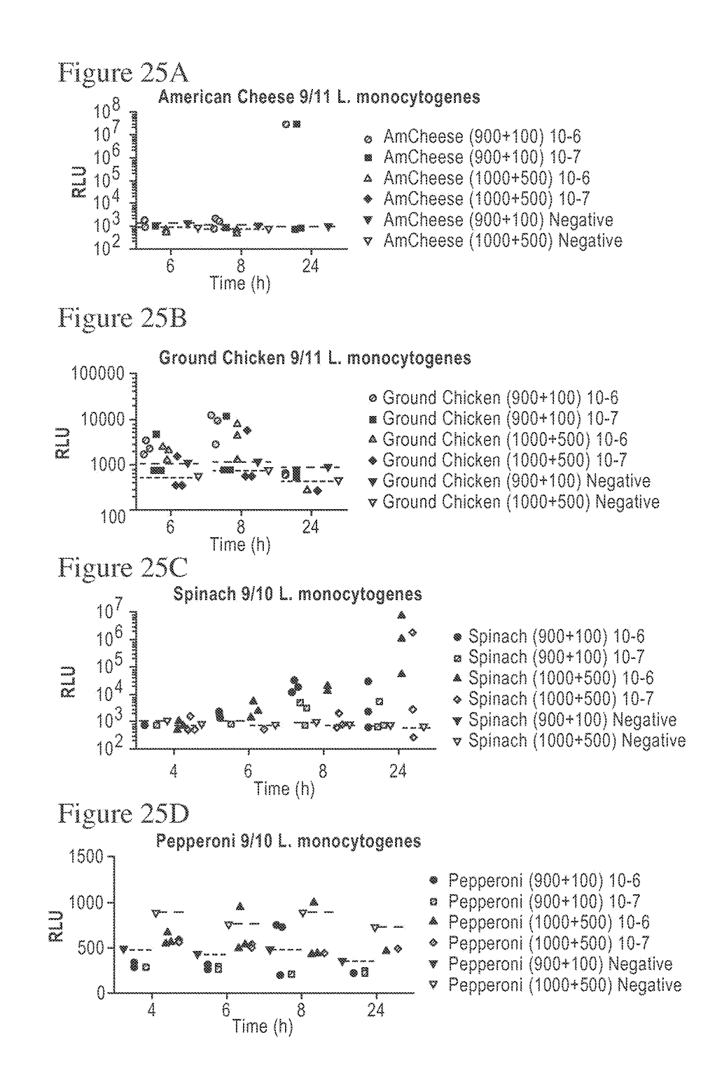

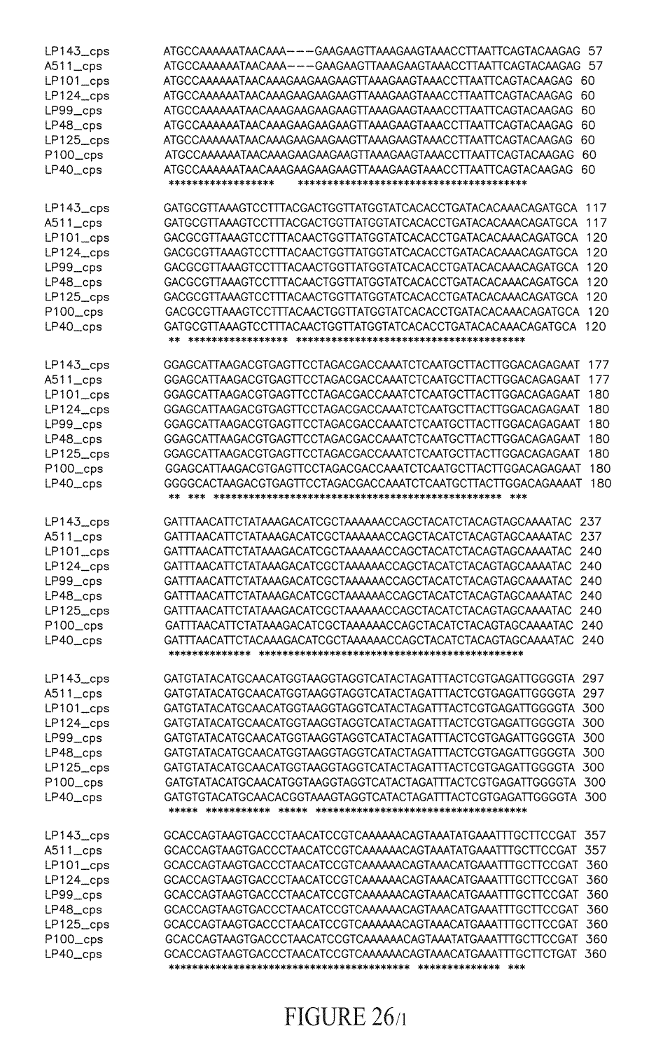

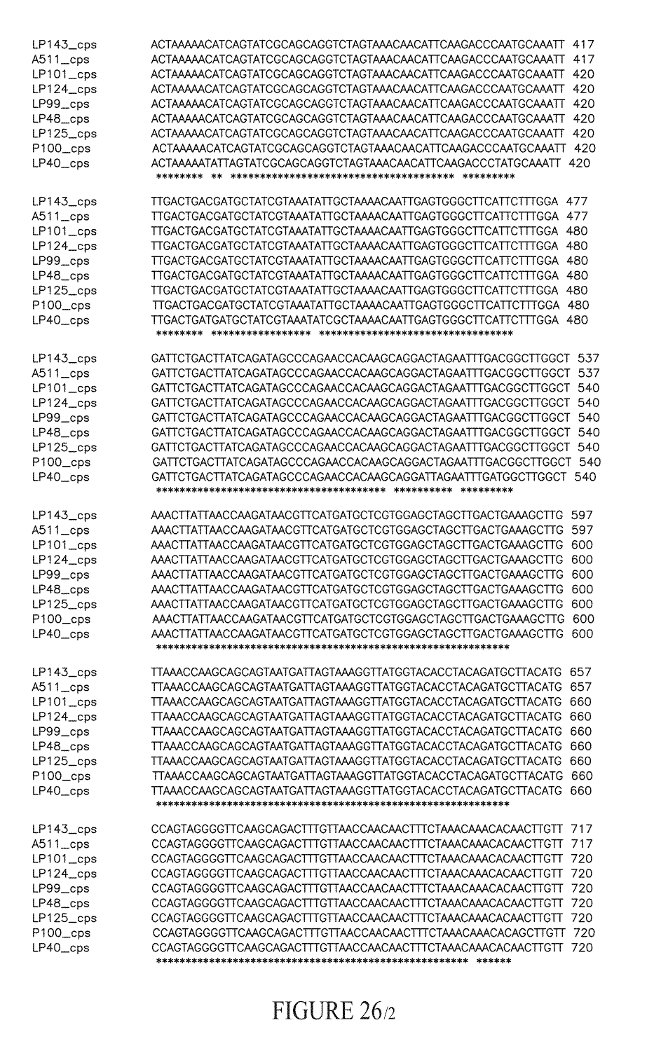

Another time course to detection assay was performed utilizing various dilutions of food matrix (American cheese, spinach, pepperoni and ground chicken) to incubation buffer. (See FIG. 25A-D). For these assays, L. monocytogenes was incubated for defined amounts of time (as illustrated in the graphs in FIG. 25A-D) with dilutions of food matrix to the incubation buffer indicated in parentheses within FIG. 25. The data indicate that food matrix, as well as the dilution of matrix to incubation buffer, has a role in the time course to detection of the Listeria species in these assays.

### Example 15: Listeria Panel

A bacterial strain panel comprising a diverse combination of Listeria species and subspecies was selected for characterization of Listeria phages. The panel comprises strains that have been isolated from various geographic and environmental niches including food processing plants and food retail locations. Special consideration was given to obtain bacterial strains from food processing environments with sufficient geographic separation to maximize natural variation within the bacterial strain panel.

The panel as assembled initially contained 272 Listeria isolates and represents the four major species of Listeria (L. monocytogenes, L. innocua, L. welshmeri and L. seelingri) (Table 13). Within each species the panel comprises representative isolates of various subspecies to ensure sufficient depth of coverage to allow for meaningful extrapolation of the data to the subspecies in general. The selection of strains for the bacterial panel were based on the prevalence of particular strains within the food environment and associated with human disease. Environmental screening of retail food stores used allelotyping to identify the most commonly identified Listeria subspecies and identified that certain allelotypes were often highly represented among the population of species identified. (Williams, S. K. et al., J Food Prot 74, 63-77 (2011); Sauders, B. D. et al., Appl Environ Microbiol 78, 4420-4433 (2012).) Ten (10) L. monocytogenes strains from each of the most common ribotypes represented from isolates from food and human disease were selected for the collection. These populations are largely overlapping and have a strong correlation in prevalence and, therefore, represent the strains most useful to identify in food processing plants. When looking at breadth of coverage of L. monocytogenes strains based on ribotypes isolated in human disease and food processing plants, the panel as constructed represents ˜86% and 91% coverage, respectively. The purpose for selecting 10 strains of each L. monocytogenes ribotype was to allow for the identification of natural variation within a group to ensure a reasonably complete coverage of the L. monocytogenes species.

To expand beyond L. monocytogenes and cover other species within the genus additional species and subspecies variation was considered to select further strains for the panel. Again, focus was placed on the species and subspecies that are commonly identified in food processing plants. Ten (10) isolates representing each of the most common allelotypes of L. welshmeri, L. innocua and L. selelingri were selected. The panel as constructed covers 96% of the L. innocua, 98% of the L. selelingri, and 100% of the L. welshmeri ribotypes identified by Saunders et al. and provides an accurate representation of the Listeria genus. The Listeria host panel as assembled thus serves as a tool for the analysis of the host range of any bacteriophage against the Listeria genus. Accordingly, this panel can be used to define target bacteria of any given phage.

The genus, species, and subspecies of the members of the panel are provided in Table 13.

TABLE 13

| Identifier | Strain Name | Genus/Species | Subspecies |
|---|---|---|---|
| NP1900 | FSL R8-5085 | Listeria innocua | sig B allelotype 11 |
| NP1901 | FSL R8-5091 | Listeria innocua | sig B allelotype 11 |
| NP1902 | FSL R8-5098 | Listeria innocua | sig B allelotype 11 |
| NP1903 | FSL R8-5255 | Listeria innocua | sig B allelotype 11 |
| NP1904 | FSL R8-5293 | Listeria innocua | sig B allelotype 11 |
| NP1905 | FSL R8-5295 | Listeria innocua | sig B allelotype 11 |
| NP1906 | FSL R8-5306 | Listeria innocua | sig B allelotype 11 |
| NP1907 | FSL R8-5440 | Listeria innocua | sig B allelotype 11 |
| NP1908 | FSL R8-5442 | Listeria innocua | sig B allelotype 11 |
| NP1909 | FSL R8-5448 | Listeria innocua | sig B allelotype 11 |
| NP1912 | FSL R8-7061 | Listeria innocua | sig B allelotype 22 |
| NP1959 | FSL S4-158 | Listeria innocua | sig B allelotype 22 |
| NP1960 | FSL S10-784 | Listeria innocua | sig B allelotype 22 |
| NP1961 | FSL F6-1168 | Listeria innocua | sig B allelotype 22 |
| NP1962 | FSL R8-5961 | Listeria innocua | sig B allelotype 22 |
| NP1963 | FSL R8-6922 | Listeria innocua | sig B allelotype 22 |
| NP1964 | FSL R8-7352 | Listeria innocua | sig B allelotype 22 |
| NP1965 | FSL R8-5598 | Listeria innocua | sig B allelotype 22 |
| NP1966 | FSL R8-6733 | Listeria innocua | sig B allelotype 22 |
| NP1967 | FSL R8-5942 | Listeria innocua | sig B allelotype 22 |
| NP1915 | FSL R8-7548 | Listeria innocua | sig B allelotype 37 |
| NP1997 | FSL R8-5764 | Listeria innocua | sig B allelotype 37 |
| NP1998 | FSL R8-5802 | Listeria innocua | sig B allelotype 37 |
| NP1999 | FSL R8-6012 | Listeria innocua | sig B allelotype 37 |
| NP2000 | FSL R8-6355 | Listeria innocua | sig B allelotype 37 |
| NP2001 | FSL R8-6369 | Listeria innocua | sig B allelotype 37 |
| NP2002 | FSL R8-6476 | Listeria innocua | sig B allelotype 37 |
| NP2003 | FSL R8-7175 | Listeria innocua | sig B allelotype 37 |
| NP2004 | FSL R8-6888 | Listeria innocua | sig B allelotype 37 |
| NP2005 | FSL R8-6672 | Listeria innocua | sig B allelotype 37 |
| NP1916 | FSL R8-6667 | Listeria innocua | sig B allelotype 56 |
| NP2006 | FSL S10-1311 | Listeria innocua | sig B allelotype 56 |
| NP2007 | FSL F6-1159 | Listeria innocua | sig B allelotype 56 |
| NP2008 | FSL F6-1126 | Listeria innocua | sig B allelotype 56 |
| NP2009 | FSL S6-120 | Listeria innocua | sig B allelotype 56 |
| NP2010 | FSL R8-5594 | Listeria innocua | sig B allelotype 56 |
| NP2011 | FSL R8-7181 | Listeria innocua | sig B allelotype 56 |
| NP2012 | FSL R2-632 | Listeria innocua | sig B allelotype 56 |
| NP2013 | FSL L3-851 | Listeria innocua | sig B allelotype 56 |
| NP2014 | FSL S10-1377 | Listeria innocua | sig B allelotype 56 |
| NP 1869 | WSLC 3009 | Listeria ivanovii | sig B allelotype 73 |
| NP 1840 | FSL J1-208 | Listeria | ribotype DUP-10142 |
| | | monocytogenes | |
| NP 1839 | FSL F6-367 | Listeria | ribotype DUP- |
| | | monocytogenes | 1030A |
| NP2024 | FSL F6-267 | Listeria | ribotype DUP- |
| | | monocytogenes | 1030A |
| NP2025 | FSL F6-406 | Listeria | ribotype DUP- |
| | | monocytogenes | 1030A |
| NP2026 | FSL H5-592 | Listeria | ribotype DUP- |
| | | monocytogenes | 1030A |
| NP2027 | FSL H1-219 | Listeria | ribotype DUP- |
| | | monocytogenes | 1030A |
| NP2028 | FSL H1-121 | Listeria | ribotype DUP- |
| | | monocytogenes | 1030A |
| NP2029 | FSL W3-072 | Listeria | ribotype DUP- |
| | | monocytogenes | 1030A |
| NP2030 | FSL N4-239 | Listeria | ribotype DUP- |
| | | monocytogenes | 1030A |
| NP2031 | FSL N3-293 | Listeria | ribotype DUP- |
| | | monocytogenes | 1030A |
| NP2032 | FSL F3-319 | Listeria | ribotype DUP- |
| | | monocytogenes | 1030A |
| NP1879 | FSL N4-221 | Listeria | ribotype DUP- |
| | | monocytogenes | 1030B |
| NP2033 | FSL F2-738 | Listeria | ribotype DUP- |
| | | monocytogenes | 1030B |
| NP2034 | FSL N3-881 | Listeria | ribotype DUP- |
| | | monocytogenes | 1030B |
| NP2035 | FSL N4-048 | Listeria | ribotype DUP- |
| | | monocytogenes | 1030B |
| NP2036 | FSL N4-696 | Listeria | ribotype DUP- |
| | | monocytogenes | 1030B |
| NP2037 | FSL N4-242 | Listeria | ribotype DUP- |
| | | monocytogenes | 1030B |
| NP2038 | FSL H4-364 | Listeria | ribotype DUP- |
| | | monocytogenes | 1030B |
| NP2039 | FSL H4-147 | Listeria | ribotype DUP- |
| | | monocytogenes | 1030B |
| NP2040 | FSL H4-946 | Listeria | ribotype DUP- |
| | | monocytogenes | 1030B |
| NP2041 | FSL S4-461 | Listeria | ribotype DUP- |
| | | monocytogenes | 1030B |
| NP2042 | FSL F6-206 | Listeria | ribotype DUP- |
| | | monocytogenes | 1038B |
| NP2043 | FSL F6-224 | Listeria | ribotype DUP- |
| | | monocytogenes | 1038B |
| NP2044 | FSL L3-739 | Listeria | ribotype DUP- |
| | | monocytogenes | 1038B |
| NP2045 | FSL N3-008 | Listeria | ribotype DUP- |
| | | monocytogenes | 1038B |
| NP2046 | FSL N3-022 | Listeria | ribotype DUP- |
| | | monocytogenes | 1038B |
| NP2047 | FSL J1-108 | Listeria | ribotype DUP- |
| | | monocytogenes | 1038B |
| NP2048 | FSL J1-119 | Listeria | ribotype DUP- |
| | | monocytogenes | 1038B |
| NP2049 | FSL C1-122 | Listeria | ribotype DUP- |
| | | monocytogenes | 1038B |
| NP2050 | FSL J1-126 | Listeria | ribotype DUP- |
| | | monocytogenes | 1038B |
| NP1880 | FSL L3-159 | Listeria | ribotype DUP- |
| | | monocytogenes | 1039A |
| NP2051 | FSL F3-285 | Listeria | ribotype DUP- |
| | | monocytogenes | 1039A |
| NP2052 | FSL R6-288 | Listeria | ribotype DUP- |
| | | monocytogenes | 1039A |
| NP2053 | FSL N1-021 | Listeria | ribotype DUP- |
| | | monocytogenes | 1039A |
| NP2054 | FSL H1-208 | Listeria | ribotype DUP- |
| | | monocytogenes | 1039A |
| NP2055 | FSL N3-034 | Listeria | ribotype DUP- |
| | | monocytogenes | 1039A |
| NP2056 | FSL L5-072 | Listeria | ribotype DUP- |
| | | monocytogenes | 1039A |
| NP2057 | FSL S6-131 | Listeria | ribotype DUP- |
| | | monocytogenes | 1039A |
| NP2058 | FSL N3-278 | Listeria | ribotype DUP- |
| | | monocytogenes | 1039A |
| NP2059 | FSL R2-282 | Listeria | ribotype DUP- |
| | | monocytogenes | 1039A |
| NP1881 | FSL T1-323 | Listeria | ribotype DUP- |
| | | monocytogenes | 1039B |
| NP2060 | FSL H5-770 | Listeria | ribotype DUP- |
| | | monocytogenes | 1039B |
| NP2061 | FSL F6-207 | Listeria | ribotype DUP- |
| | | monocytogenes | 1039B |
| NP2062 | FSL F6-236 | Listeria | ribotype DUP- |
| | | monocytogenes | 1039B |
| NP2063 | FSL H5-795 | Listeria | ribotype DUP- |
| | | monocytogenes | 1039B |
| NP2064 | FSL N3-246 | Listeria | ribotype DUP- |
| | | monocytogenes | 1039B |
| NP2065 | FSL R2-062 | Listeria | ribotype DUP- |
| | | monocytogenes | 1039B |
| NP2066 | FSL R2-437 | Listeria | ribotype DUP- |
| | | monocytogenes | 1039B |
| NP2067 | FSL M1-004 | Listeria | ribotype DUP- |
| | | monocytogenes | 1039B |
| NP2068 | FSL L4-352 | Listeria | ribotype DUP- |
| | | monocytogenes | 1039B |
| NP2069 | FSL F6-605 | Listeria | ribotype DUP- |
| | | monocytogenes | 1039C |
| NP2070 | FSL V1-001 | Listeria | ribotype DUP- |
| | | monocytogenes | 1039C |
| NP2071 | FSL F6-464 | Listeria | ribotype DUP- |
| | | monocytogenes | 1039C |
| NP2072 | FSL R8-2748 | Listeria | ribotype DUP- |
| | | monocytogenes | 1039C |
| NP2073 | FSL R6-908 | Listeria | ribotype DUP- |
| | | monocytogenes | 1039C |
| NP2074 | FSL L3-802 | Listeria | ribotype DUP- |
| | | monocytogenes | 1039C |
| NP2075 | FSL F3-056 | Listeria | ribotype DUP- |
| | | monocytogenes | 1039C |
| NP2076 | FSL J2-020 | Listeria | ribotype DUP- |
| | | monocytogenes | 1039C |
| NP2077 | FSL S4-914 | Listeria | ribotype DUP- |
| | | monocytogenes | 1039C |
| NP1882 | FSL H5-725 | Listeria | ribotype DUP- |
| | | monocytogenes | 1042A |
| NP2078 | FSL F6-467 | Listeria | ribotype DUP- |
| | | monocytogenes | 1042A |
| NP2079 | FSL F6-655 | Listeria | ribotype DUP- |
| | | monocytogenes | 1042A |
| NP2080 | FSL F6-352 | Listeria | ribotype DUP- |
| | | monocytogenes | 1042A |
| NP2081 | FSL H5-781 | Listeria | ribotype DUP- |
| | | monocytogenes | 1042A |
| NP2082 | FSL K2-147 | Listeria | ribotype DUP- |
| | | monocytogenes | 1042A |
| NP2083 | FSL V1-026 | Listeria | ribotype DUP- |
| | | monocytogenes | 1042A |
| NP2084 | FSL H5-572 | Listeria | ribotype DUP- |
| | | monocytogenes | 1042A |
| NP2085 | FSL K2-065 | Listeria | ribotype DUP- |
| | | monocytogenes | 1042A |
| NP2086 | FSL H4-120 | Listeria | ribotype DUP- |
| | | monocytogenes | 1042A |
| NP2087 | FSL F6-184 | Listeria | ribotype DUP- |
| | | monocytogenes | 1042B |
| NP2088 | FSL F6-191 | Listeria | ribotype DUP- |
| | | monocytogenes | 1042B |
| NP2089 | FSL H1-099 | Listeria | ribotype DUP- |
| | | monocytogenes | 1042B |
| NP2090 | FSL J1-116 | Listeria | ribotype DUP- |
| | | monocytogenes | 1042B |
| NP2091 | FSL R2-192 | Listeria | ribotype DUP- |
| | | monocytogenes | 1042B |
| NP2092 | FSL J1-225 | Listeria | ribotype DUP- |
| | | monocytogenes | 1042B |
| NP2093 | FSL R2-500 | Listeria | ribotype DUP- |
| | | monocytogenes | 1042B |
| NP2094 | FSL R2-501 | Listeria | ribotype DUP- |
| | | monocytogenes | 1042B |
| NP2095 | FSL E1-159 | Listeria | ribotype DUP- |
| | | monocytogenes | 1042B |
| NP2096 | FSL F6-355 | Listeria | ribotype DUP- |
| | | monocytogenes | 1042C |
| NP2097 | FSL F6-382 | Listeria | ribotype DUP- |
| | | monocytogenes | 1042C |
| NP2098 | FSL F3-200 | Listeria | ribotype DUP- |
| | | monocytogenes | 1042C |
| NP2099 | FSL K2-143 | Listeria | ribotype DUP- |
| | | monocytogenes | 1042C |
| NP2100 | FSL N1-176 | Listeria | ribotype DUP- |
| | | monocytogenes | 1042C |
| NP2101 | FSL N1-417 | Listeria | ribotype DUP- |
| | | monocytogenes | 1042C |
| NP2102 | FSL L3-051 | Listeria | ribotype DUP- |
| | | monocytogenes | 1042C |
| NP2103 | FSL T1-107 | Listeria | ribotype DUP- |
| | | monocytogenes | 1042C |
| NP2104 | FSL T1-408 | Listeria | ribotype DUP- |
| | | monocytogenes | 1042C |
| NP1883 | FSL T1-922 | Listeria | ribotype DUP- |
| | | monocytogenes | 1043A |
| NP2105 | FSL F6-396 | Listeria | ribotype DUP- |
| | | monocytogenes | 1043A |
| NP2106 | FSL H5-806 | Listeria | ribotype DUP- |
| | | monocytogenes | 1043A |
| NP2107 | FSL F6-551 | Listeria | ribotype DUP- |
| | | monocytogenes | 1043A |
| NP2108 | FSL F6-446 | Listeria | ribotype DUP- |
| | | monocytogenes | 1043A |
| NP2109 | FSL F6-315 | Listeria | ribotype DUP- |
| | | monocytogenes | 1043A |
| NP2110 | FSL V1-022 | Listeria | ribotype DUP- |
| | | monocytogenes | 1043A |
| NP2111 | FSL R2-132 | Listeria | ribotype DUP- |
| | | monocytogenes | 1043A |
| NP2112 | FSL R2-273 | Listeria | ribotype DUP- |
| | | monocytogenes | 1043A |
| NP2113 | FSL N3-277 | Listeria | ribotype DUP- |
| | | monocytogenes | 1043A |
| NP1884 | FSL H1-251 | Listeria | ribotype DUP- |
| | | monocytogenes | 1044A |
| NP2114 | FSL F6-358 | Listeria | ribotype DUP- |
| | | monocytogenes | 1044A |
| NP2115 | FSL F6-194 | Listeria | ribotype DUP- |
| | | monocytogenes | 1044A |
| NP2116 | FSL R2-763 | Listeria | ribotype DUP- |
| | | monocytogenes | 1044A |
| NP2117 | FSL R2-765 | Listeria | ribotype DUP- |
| | | monocytogenes | 1044A |
| NP2118 | FSL R2-764 | Listeria | ribotype DUP- |
| | | monocytogenes | 1044A |
| NP2119 | FSL N1-225 | Listeria | ribotype DUP- |
| | | monocytogenes | 1044A |
| NP2120 | FSL N1-227 | Listeria | ribotype DUP- |
| | | monocytogenes | 1044A |
| NP2121 | FSL N1-048 | Listeria | ribotype DUP- |
| | | monocytogenes | 1044A |
| NP2122 | FSL K2-131 | Listeria | ribotype DUP- |
| | | monocytogenes | 1044A |
| NP1885 | FSL L3-501 | Listeria | ribotype DUP- |
| | | monocytogenes | 1044B |
| NP2123 | FSL F6-222 | Listeria | ribotype DUP- |
| | | monocytogenes | 1044B |
| NP2124 | FSL F6-249 | Listeria | ribotype DUP- |
| | | monocytogenes | 1044B |
| NP2125 | FSL N3-065 | Listeria | ribotype DUP- |
| | | monocytogenes | 1044B |
| NP2126 | FSL H4-699 | Listeria | ribotype DUP- |
| | | monocytogenes | 1044B |
| NP2127 | FSL L4-241 | Listeria | ribotype DUP- |
| | | monocytogenes | 1044B |
| NP2128 | FSL S4-643 | Listeria | ribotype DUP- |
| | | monocytogenes | 1044B |
| NP2129 | FSL R2-073 | Listeria | ribotype DUP- |
| | | monocytogenes | 1044B |
| NP2130 | FSL F3-224 | Listeria | ribotype DUP- |
| | | monocytogenes | 1044B |
| NP2131 | FSL N4-334 | Listeria | ribotype DUP- |
| | | monocytogenes | 1044B |
| NP1886 | FSL R2-069 | Listeria | ribotype DUP- |
| | | monocytogenes | 1044E |
| NP2132 | FSL R2-070 | Listeria | ribotype DUP- |
| | | monocytogenes | 1044E |
| NP1887 | FSL H1-030 | Listeria | ribotype DUP- |
| | | monocytogenes | 1045B |
| NP2133 | FSL F6-421 | Listeria | ribotype DUP- |
| | | monocytogenes | 1045B |
| NP2134 | FSL F6-449 | Listeria | ribotype DUP- |
| | | monocytogenes | 1045B |
| NP2135 | FSL J2-054 | Listeria | ribotype DUP- |
| | | monocytogenes | 1045B |
| NP2136 | FSL S4-024 | Listeria | ribotype DUP- |
| | | monocytogenes | 1045B |
| NP2137 | FSL H1-111 | Listeria | ribotype DUP- |
| | | monocytogenes | 1045B |
| NP2138 | FSL K2-022 | Listeria | ribotype DUP- |
| | | monocytogenes | 1045B |
| NP2139 | FSL S4-066 | Listeria | ribotype DUP- |
| | | monocytogenes | 1045B |
| NP2140 | FSL R2-067 | Listeria | ribotype DUP- |
| | | monocytogenes | 1045B |
| NP2141 | FSL R2-293 | Listeria | ribotype DUP- |
| | | monocytogenes | 1045B |
| NP2142 | FSL F6-323 | Listeria | ribotype DUP- |
| | | monocytogenes | 1052A |
| NP2143 | FSL F6-216 | Listeria | ribotype DUP- |
| | | monocytogenes | 1052A |
| NP2144 | FSL F6-321 | Listeria | ribotype DUP- |
| | | monocytogenes | 1052A |
| NP2145 | FSL V1-117 | Listeria | ribotype DUP- |
| | | monocytogenes | 1052A |
| NP2146 | FSL H5-846 | Listeria | ribotype DUP- |
| | | monocytogenes | 1052A |
| NP2147 | FSL L3-055 | Listeria | ribotype DUP- |
| | | monocytogenes | 1052A |
| NP2148 | FSL T1-313 | Listeria | ribotype DUP- |
| | | monocytogenes | 1052A |
| NP2149 | FSL R8-0875 | Listeria | ribotype DUP- |
| | | monocytogenes | 1052A |
| NP2150 | FSL R2-317 | Listeria | ribotype DUP- |
| | | monocytogenes | 1052A |
| NP1888 | FSL L4-019 | Listeria | ribotype DUP- |
| | | monocytogenes | 1053A |
| NP2151 | FSL F6-335 | Listeria | ribotype DUP- |
| | | monocytogenes | 1053A |
| NP2152 | FSL R6-653 | Listeria | ribotype DUP- |
| | | monocytogenes | 1053A |
| NP2153 | FSL L3-135 | Listeria | ribotype DUP- |
| | | monocytogenes | 1053A |
| NP2154 | FSL L3-143 | Listeria | ribotype DUP- |
| | | monocytogenes | 1053A |
| NP2155 | FSL L3-167 | Listeria | ribotype DUP- |
| | | monocytogenes | 1053A |
| NP2156 | FSL N3-031 | Listeria | ribotype DUP- |
| | | monocytogenes | 1053A |
| NP2157 | FSL J1-101 | Listeria | ribotype DUP- |
| | | monocytogenes | 1053A |
| NP2158 | FSL F6-154 | Listeria | ribotype DUP- |
| | | monocytogenes | 1053A |
| NP2159 | FSL R2-499 | Listeria | ribotype DUP- |
| | | monocytogenes | 1053A |
| NP1889 | FSL T1-027 | Listeria | ribotype DUP- |
| | | monocytogenes | 1062A |
| NP2160 | FSL F6-325 | Listeria | ribotype DUP- |
| | | monocytogenes | 1062A |
| NP2161 | FSL F6-220 | Listeria | ribotype DUP- |
| | | monocytogenes | 1062A |
| NP2162 | FSL F6-319 | Listeria | ribotype DUP- |
| | | monocytogenes | 1062A |
| NP2163 | FSL F6-365 | Listeria | ribotype DUP- |
| | | monocytogenes | 1062A |
| NP2164 | FSL F6-360 | Listeria | ribotype DUP- |
| | | monocytogenes | 1062A |
| NP2165 | FSL F6-313 | Listeria | ribotype DUP- |
| | | monocytogenes | 1062A |
| NP2166 | FSL R2-031 | Listeria | ribotype DUP- |
| | | monocytogenes | 1062A |
| NP2167 | FSL R2-050 | Listeria | ribotype DUP- |
| | | monocytogenes | 1062A |
| NP2168 | FSL R2-078 | Listeria | ribotype DUP- |
| | | monocytogenes | 1062A |
| NP1890 | FSL T1-041 | Listeria | ribotype DUP- |
| | | monocytogenes | 1062D |
| NP2169 | FSL F6-264 | Listeria | ribotype DUP- |
| | | monocytogenes | 1062D |
| NP2170 | FSL F3-146 | Listeria | ribotype DUP- |
| | | monocytogenes | 1062D |
| NP2171 | FSL F3-194 | Listeria | ribotype DUP- |
| | | monocytogenes | 1062D |
| NP2172 | FSL H4-122 | Listeria | ribotype DUP- |
| | | monocytogenes | 1062D |
| NP2173 | FSL H4-286 | Listeria | ribotype DUP- |
| | | monocytogenes | 1062D |
| NP2174 | FSL R6-646 | Listeria | ribotype DUP- |
| | | monocytogenes | 1062D |
| NP2175 | FSL T1-041 | Listeria | ribotype DUP- |
| | | monocytogenes | 1062D |
| NP2176 | FSL F7-002 | Listeria | ribotype DUP- |
| | | monocytogenes | 1062D |
| NP2177 | FSL X1-005 | Listeria | ribotype DUP- |
| | | monocytogenes | 1062D |
| NP 1878 | EGD-e | Listeria | |
| | | monocytogenes | |
| NP1911 | FSL R8-7641 | Listeria seeligeri | sig B allelotype 20 |
| NP1950 | FSL S10-030 | Listeria seeligeri | sig B allelotype 20 |
| NP1951 | FSL S10-320 | Listeria seeligeri | sig B allelotype 20 |
| NP1952 | FSL S10-1602 | Listeria seeligeri | sig B allelotype 20 |
| NP1953 | FSL L5-075 | Listeria seeligeri | sig B allelotype 20 |
| NP1954 | FSL L5-046 | Listeria seeligeri | sig B allelotype 20 |
| NP1955 | FSL L5-104 | Listeria seeligeri | sig B allelotype 20 |
| NP1956 | FSL R8-7575 | Listeria seeligeri | sig B allelotype 20 |
| NP1957 | FSL S4-178 | Listeria seeligeri | sig B allelotype 20 |
| NP1958 | FSL S4-135 | Listeria seeligeri | sig B allelotype 20 |
| NP1913 | FSL R8-6826 | Listeria seeligeri | sig B allelotype 24 |
| NP1968 | FSL S10-034 | Listeria seeligeri | sig B allelotype 24 |
| NP1969 | FSL S10-1611 | Listeria seeligeri | sig B allelotype 24 |
| NP1970 | FSL L5-054 | Listeria seeligeri | sig B allelotype 24 |
| NP1971 | FSL L5-085 | Listeria seeligeri | sig B allelotype 24 |
| NP1972 | FSL R8-6868 | Listeria seeligeri | sig B allelotype 24 |
| NP1973 | FSL R8-6545 | Listeria seeligeri | sig B allelotype 24 |
| NP1974 | FSL R8-6949 | Listeria seeligeri | sig B allelotype 24 |
| NP1975 | FSL S4-167 | Listeria seeligeri | sig B allelotype 24 |
| NP1976 | FSL S4-180 | Listeria seeligeri | sig B allelotype 24 |
| NP1891 | FSL R8-5241 | Listeria seeligeri | sig B allelotype 3 |
| NP1892 | FSL R8-5247 | Listeria seeligeri | sig B allelotype 3 |
| NP1893 | FSL R8-5253 | Listeria seeligeri | sig B allelotype 3 |
| NP1894 | FSL R8-5513 | Listeria seeligeri | sig B allelotype 3 |
| NP1895 | FSL R8-6629 | Listeria seeligeri | sig B allelotype 3 |
| NP1896 | FSL R8-6635 | Listeria seeligeri | sig B allelotype 3 |
| NP1897 | FSL R8-6659 | Listeria seeligeri | sig B allelotype 3 |
| NP1898 | FSL R8-6665 | Listeria seeligeri | sig B allelotype 3 |
| NP1899 | FSL R8-6852 | Listeria seeligeri | sig B allelotype 3 |
| NP1990 | FSL H6-027 | Listeria seeligeri | sig B allelotype 35 |
| NP1991 | FSL H6-079 | Listeria seeligeri | sig B allelotype 35 |
| NP1992 | FSL H6-185 | Listeria seeligeri | sig B allelotype 35 |
| NP1993 | FSL R8-6874 | Listeria seeligeri | sig B allelotype 35 |
| NP1994 | FSL R8-6880 | Listeria seeligeri | sig B allelotype 35 |
| NP1995 | FSL R8-7629 | Listeria seeligeri | sig B allelotype 35 |
| NP1996 | FSL S4-544 | Listeria seeligeri | sig B allelotype 35 |
| NP1910 | FSL R8-7026 | Listeria welshimeri | sig B allelotype 15 |
| NP1945 | FSL L5-079 | Listeria welshimeri | sig B allelotype 15 |
| NP1946 | FSL S10-1450 | Listeria welshimeri | sig B allelotype 15 |
| NP1947 | FSL S10-1451 | Listeria welshimeri | sig B allelotype 15 |
| NP1948 | FSL S4-081 | Listeria welshimeri | sig B allelotype 15 |
| NP1949 | FSL S4-101 | Listeria welshimeri | sig B allelotype 15 |
| NP1977 | FSL N1-064 | Listeria welshimeri | sig B allelotype 27 |
| NP1978 | FSL R8-8163 | Listeria welshimeri | sig B allelotype 27 |
| NP1979 | FSL R8-7524 | Listeria welshimeri | sig B allelotype 27 |
| NP1980 | FSL R8-7486 | Listeria welshimeri | sig B allelotype 27 |
| NP1981 | FSL R8-6035 | Listeria welshimeri | sig B allelotype 27 |
| NP1982 | FSL R8-5807 | Listeria welshimeri | sig B allelotype 27 |
| NP1983 | FSL S4-182 | Listeria welshimeri | sig B allelotype 27 |
| NP1984 | FSL R2-630 | Listeria welshimeri | sig B allelotype 27 |
| NP1985 | FSL F6-1131 | Listeria welshimeri | sig B allelotype 27 |
| NP1914 | FSL R8-7454 | Listeria welshimeri | sig B allelotype 32 |
| NP1986 | FSL R8-7041 | Listeria welshimeri | sig B allelotype 32 |
| NP1987 | FSL R8-5837 | Listeria welshimeri | sig B allelotype 32 |
| NP1988 | FSL R8-6136 | Listeria welshimeri | sig B allelotype 32 |
| NP1989 | FSL S4-289 | Listeria welshimeri | sig B allelotype 32 |
| NP1917 | FSL R8-1903 | Listeria welshimeri | sig B allelotype 89 |
| NP2015 | FSL S10-114 | Listeria welshimeri | sig B allelotype 89 |
| NP2016 | FSL S10-115 | Listeria welshimeri | sig B allelotype 89 |
| NP2017 | FSL S10-117 | Listeria welshimeri | sig B allelotype 89 |
| NP2018 | FSL S10-119 | Listeria welshimeri | sig B allelotype 89 |
| NP2019 | FSL S10-121 | Listeria welshimeri | sig B allelotype 89 |
| NP2020 | FSL R8-0056 | Listeria welshimeri | sig B allelotype 89 |
| NP2021 | FSL R8-1198 | Listeria welshimeri | sig B allelotype 89 |
| NP2022 | FSL R8-7403 | Listeria welshimeri | sig B allelotype 89 |
| NP2023 | FSL R2-631 | Listeria welshimeri | sig B allelotype 89 |

### Example 16: Plate-Based Phage Host Range Assay

In order to quantify the host range a given bacteriophage the plaque forming efficiency of the bacteriophage on a given isolate was standardized to a reference strain for the bacteriophage, normally the strain used for bacteriophage production. To determine the plaque forming efficiency a dilution series for the phage is generated and titered on each host. Before the work reported herein, this was the standard method of phage host range analysis. See, e.g., Sambrook et al., Molecular Cloning: A Laboratory Manual, 3d ed., Cold Spring Harbor Laboratory Press, Cold Spring Harbor, N.Y. (2001).

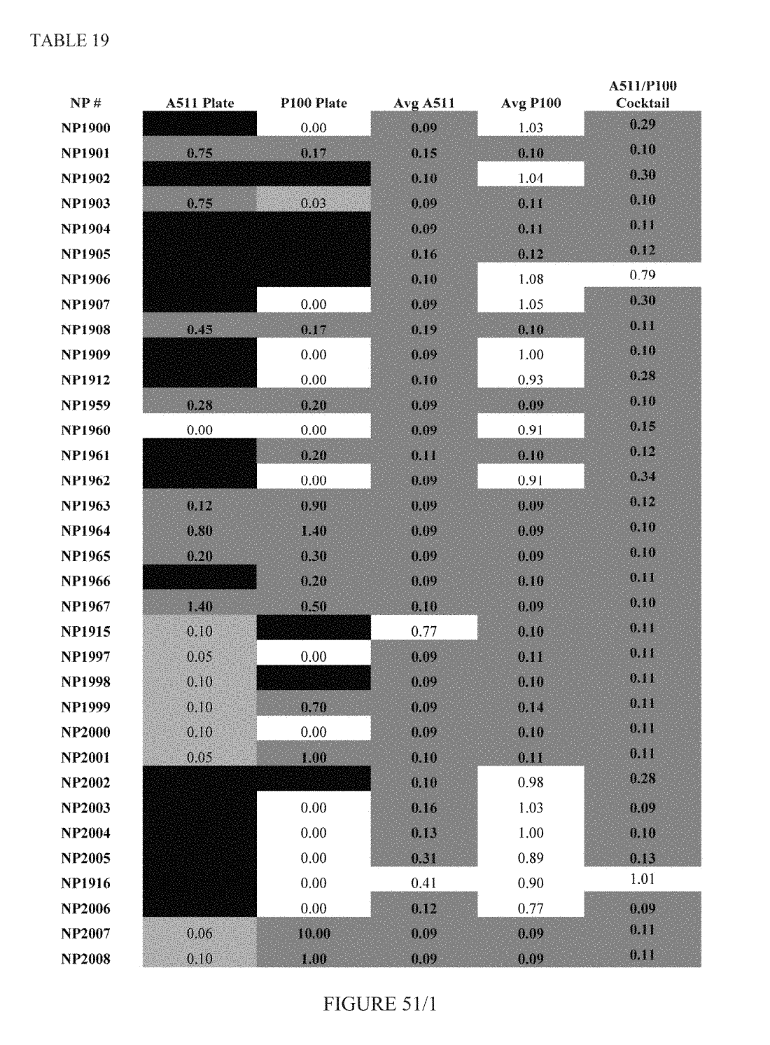

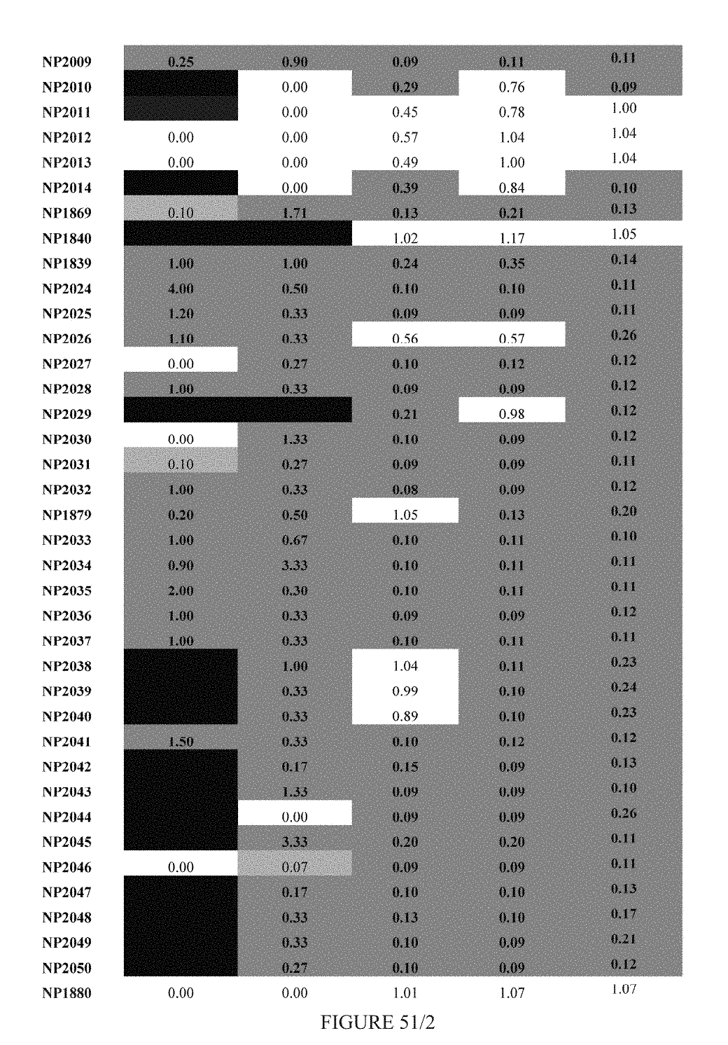

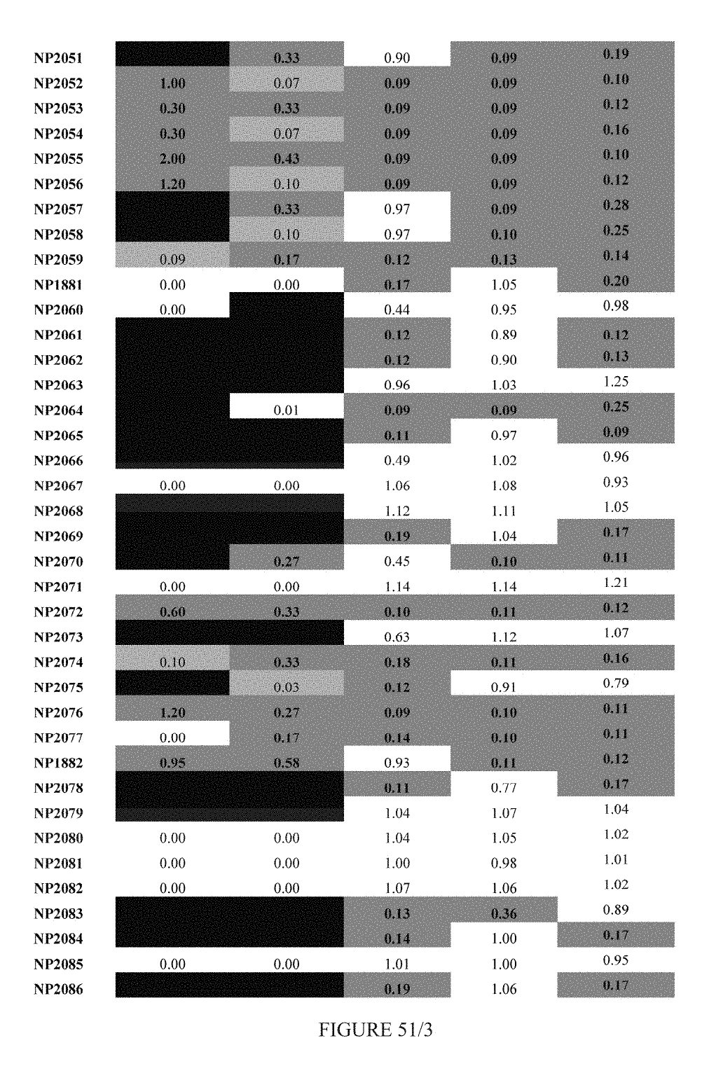

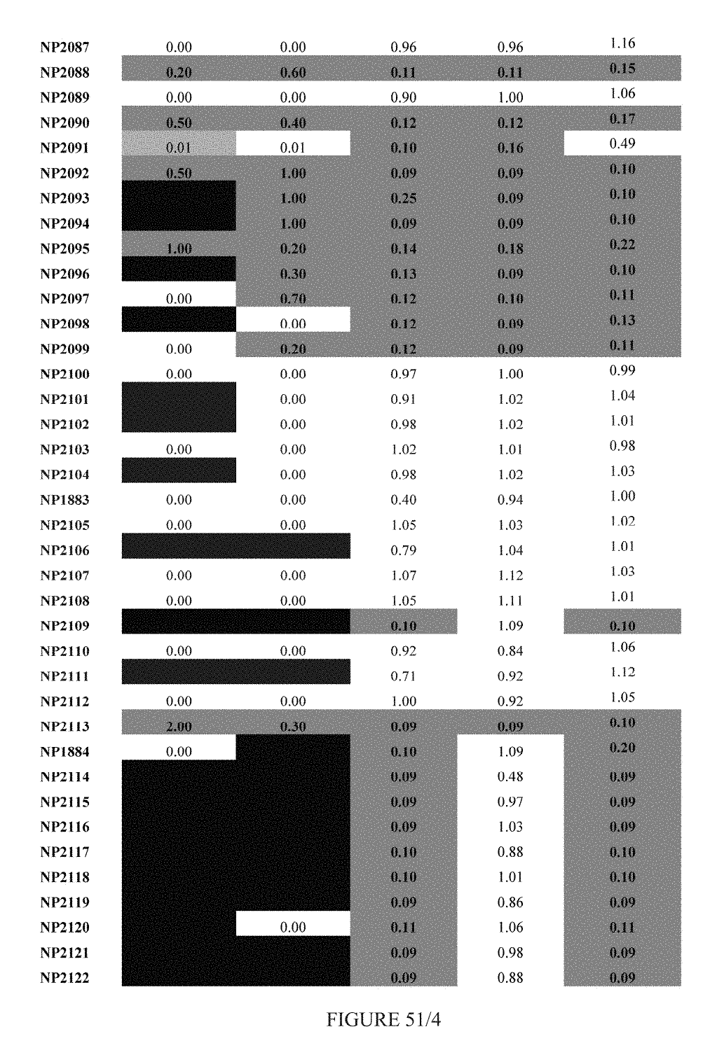

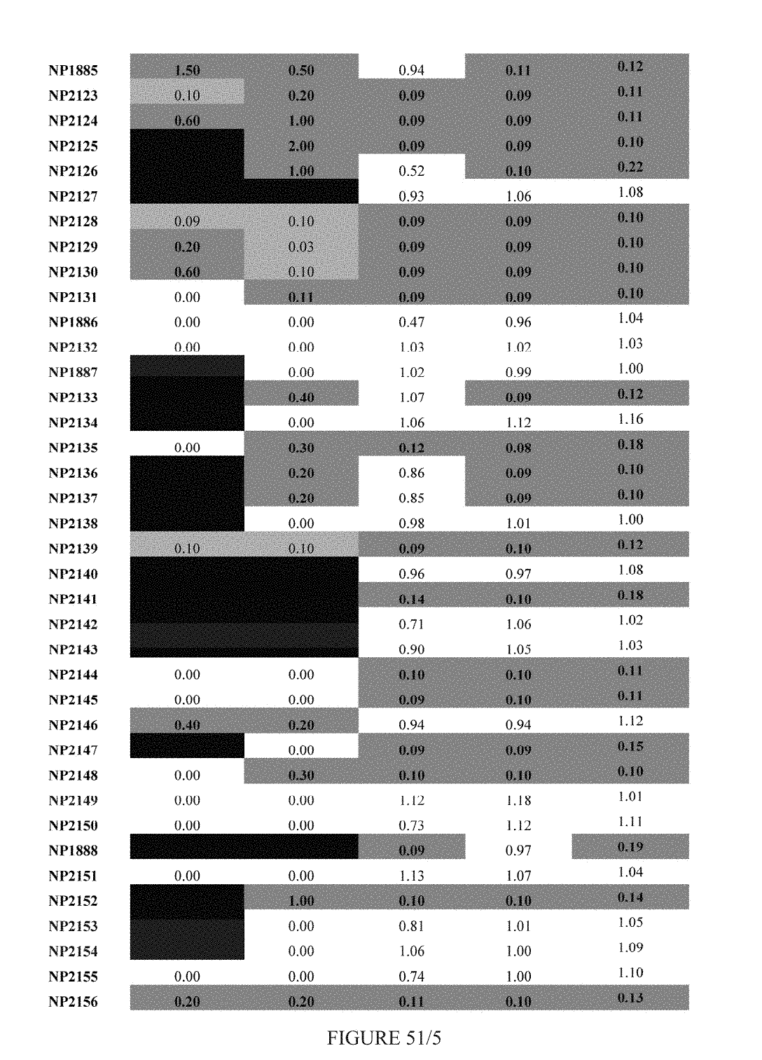

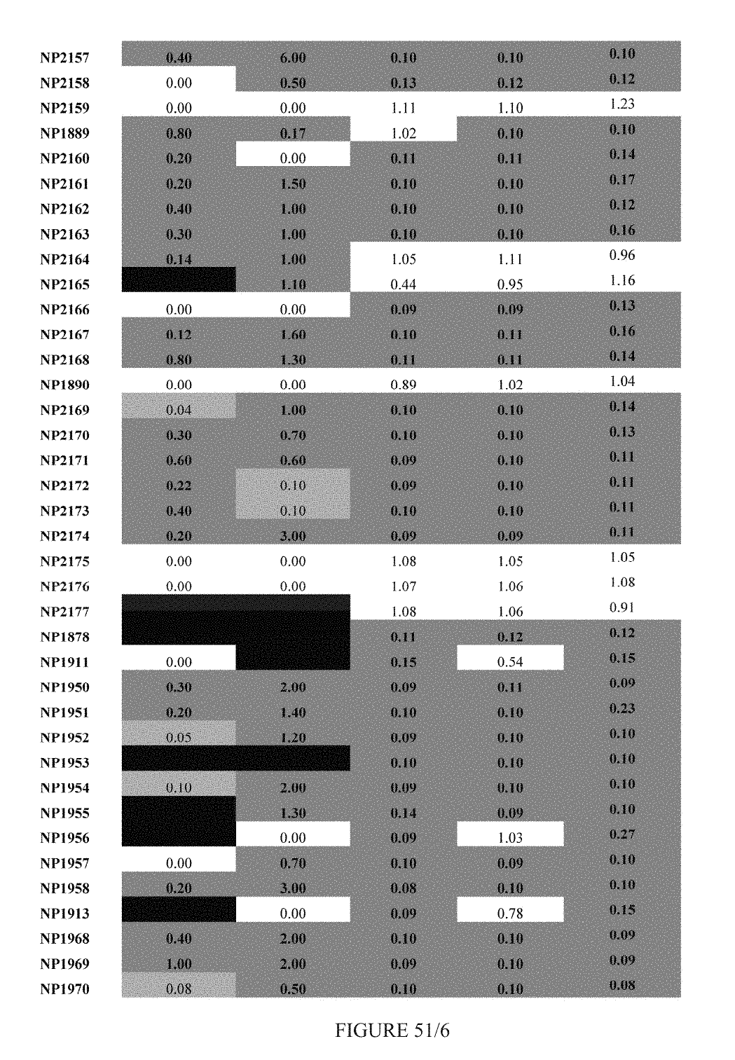

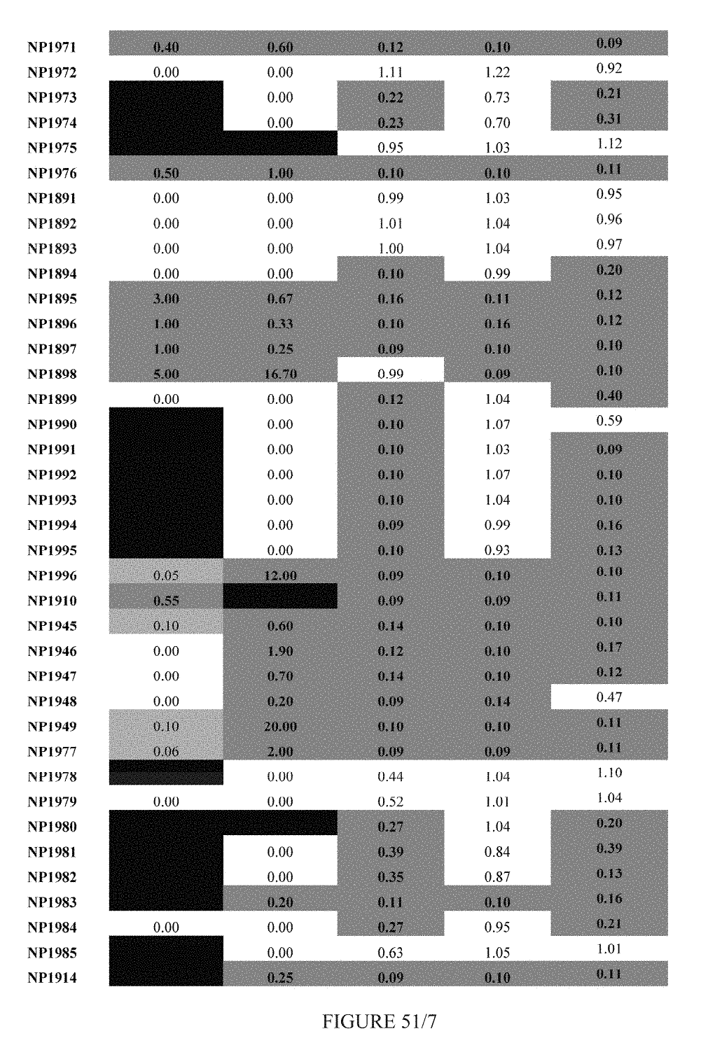

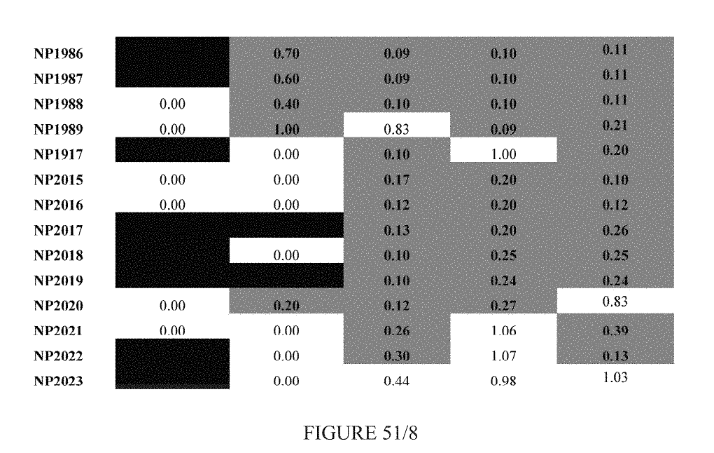

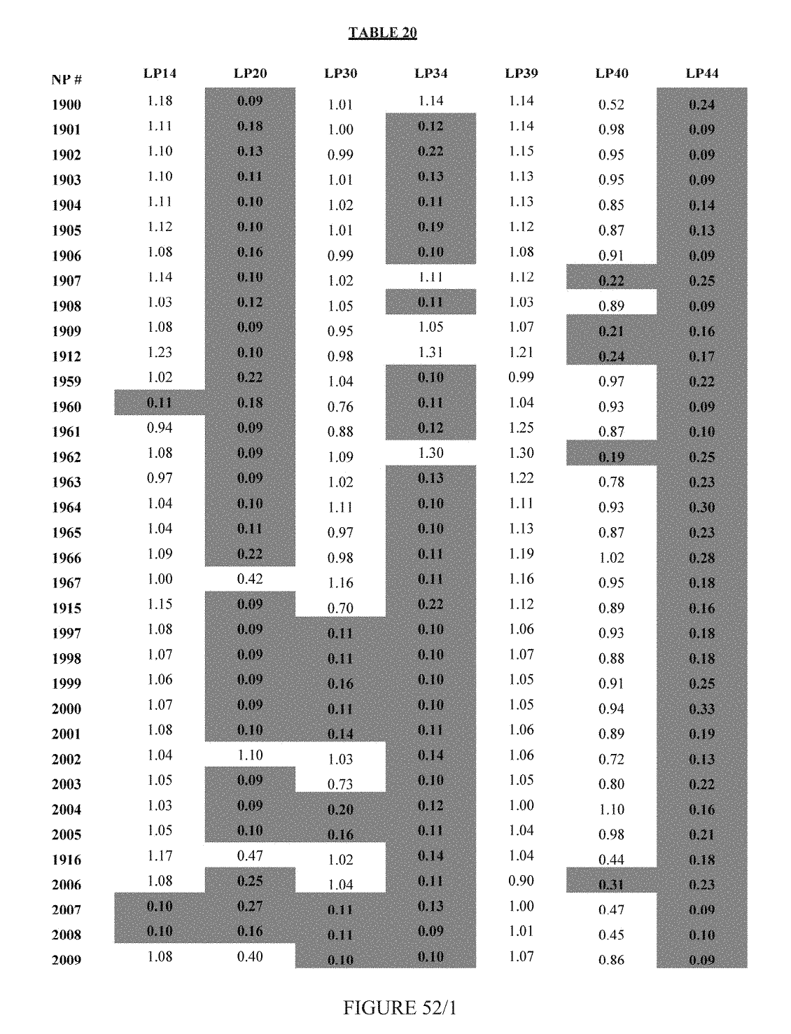

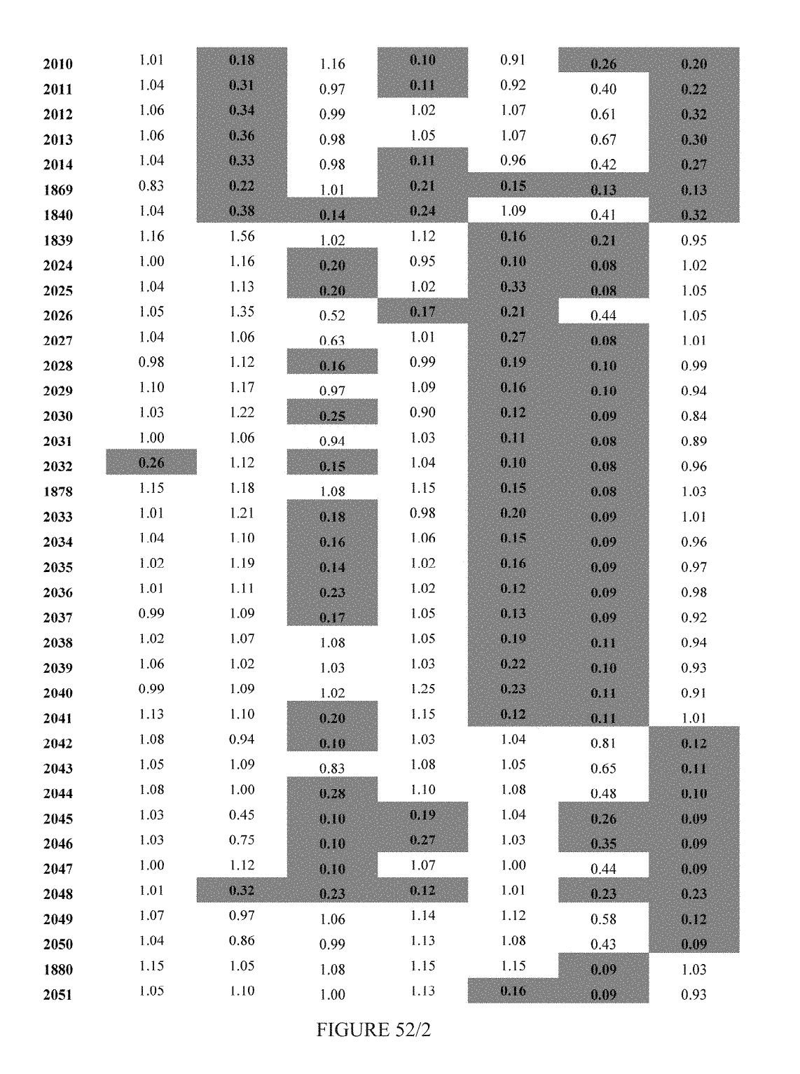

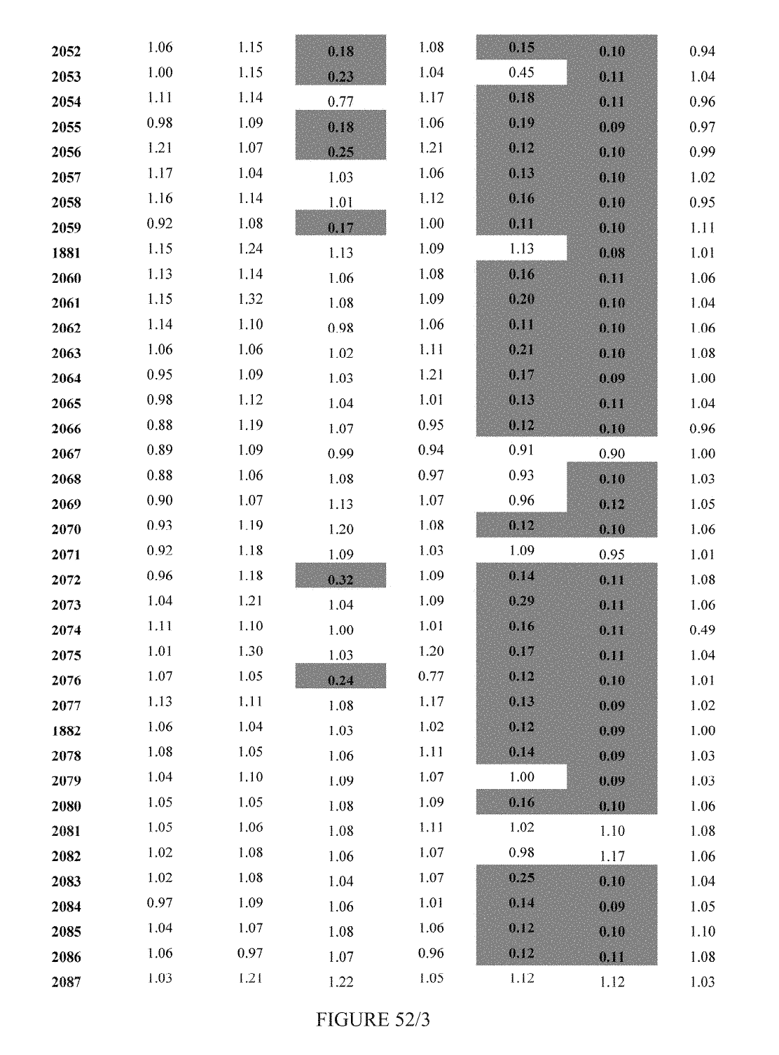

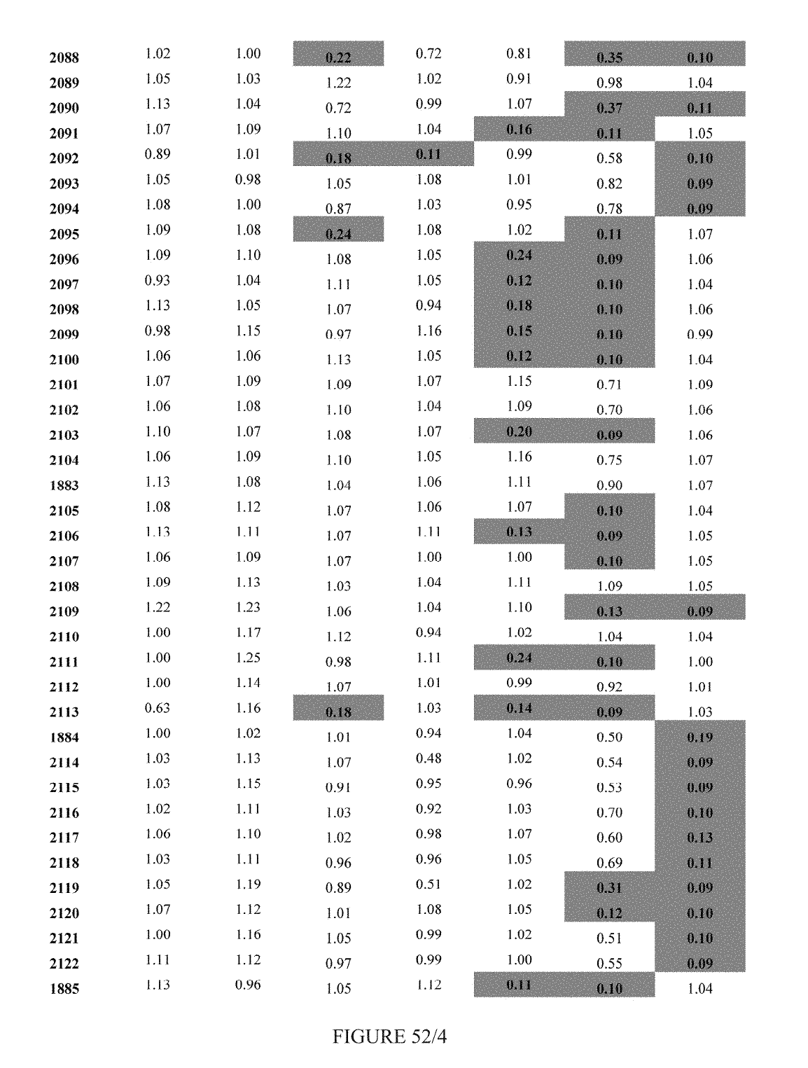

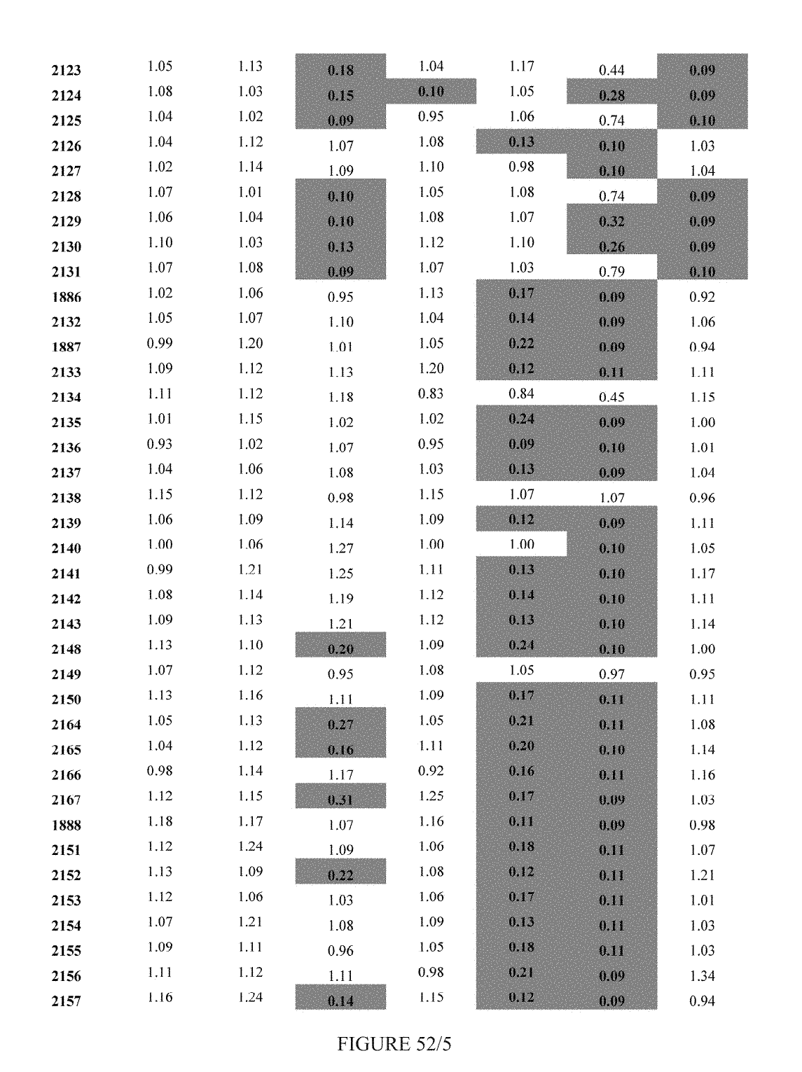

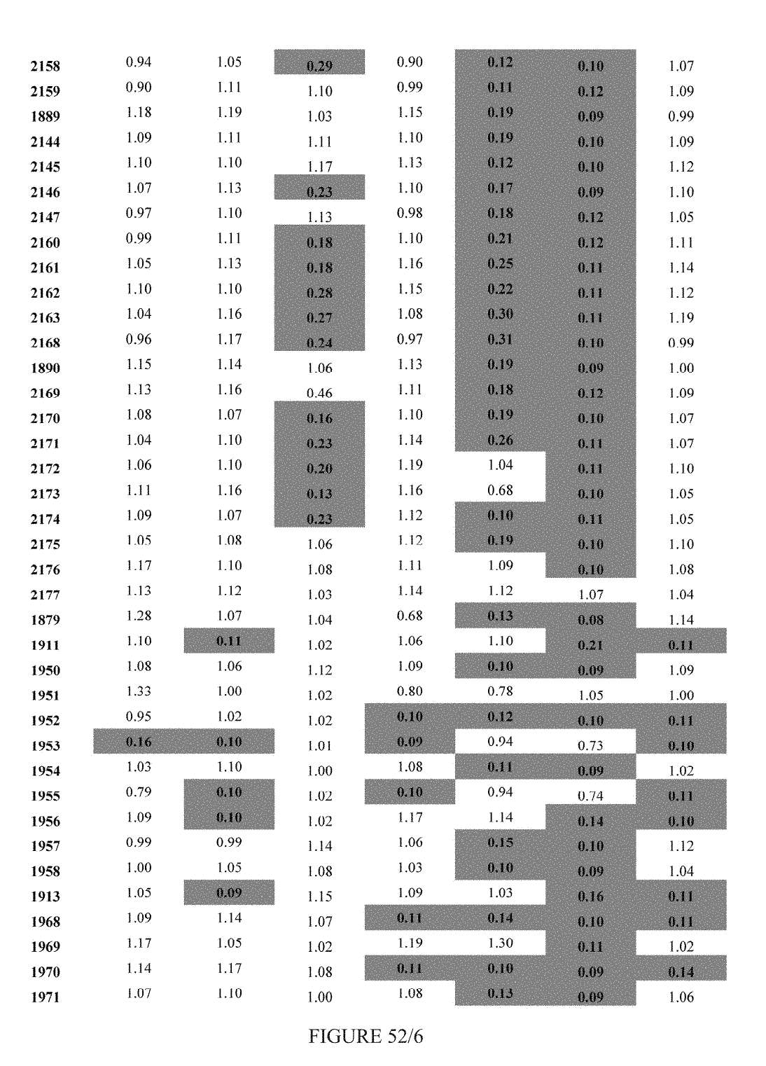

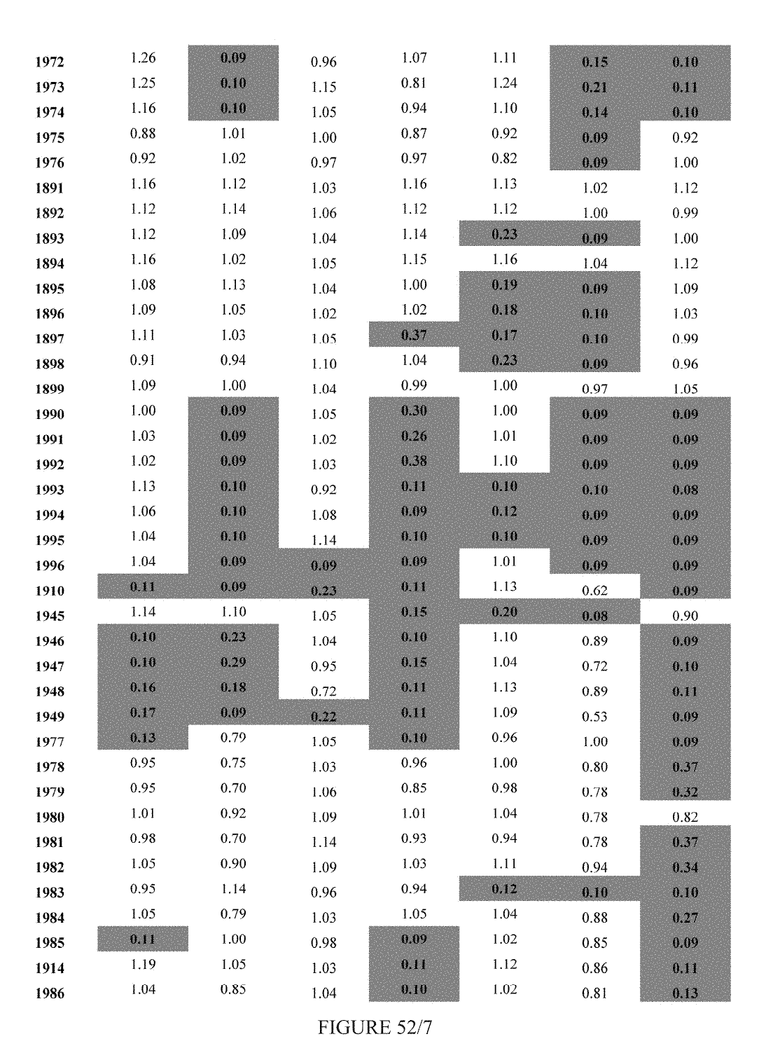

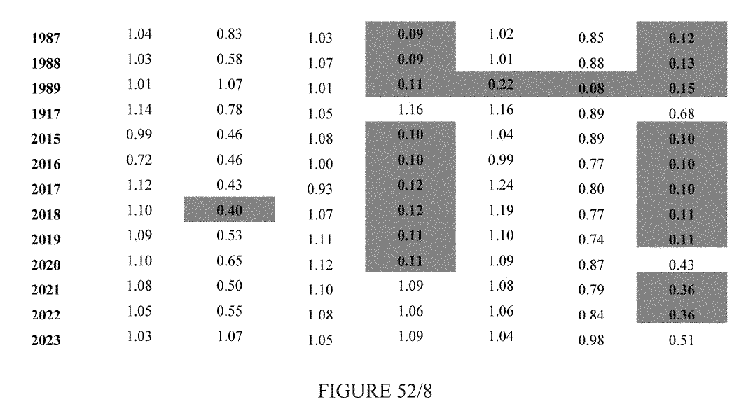

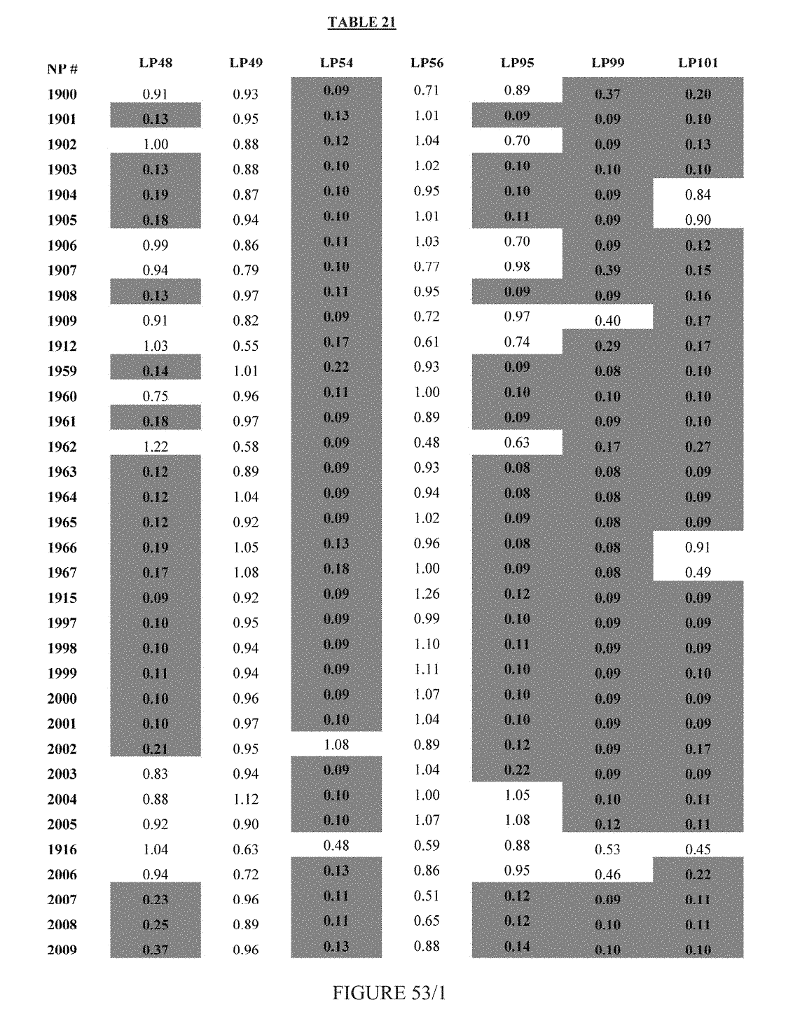

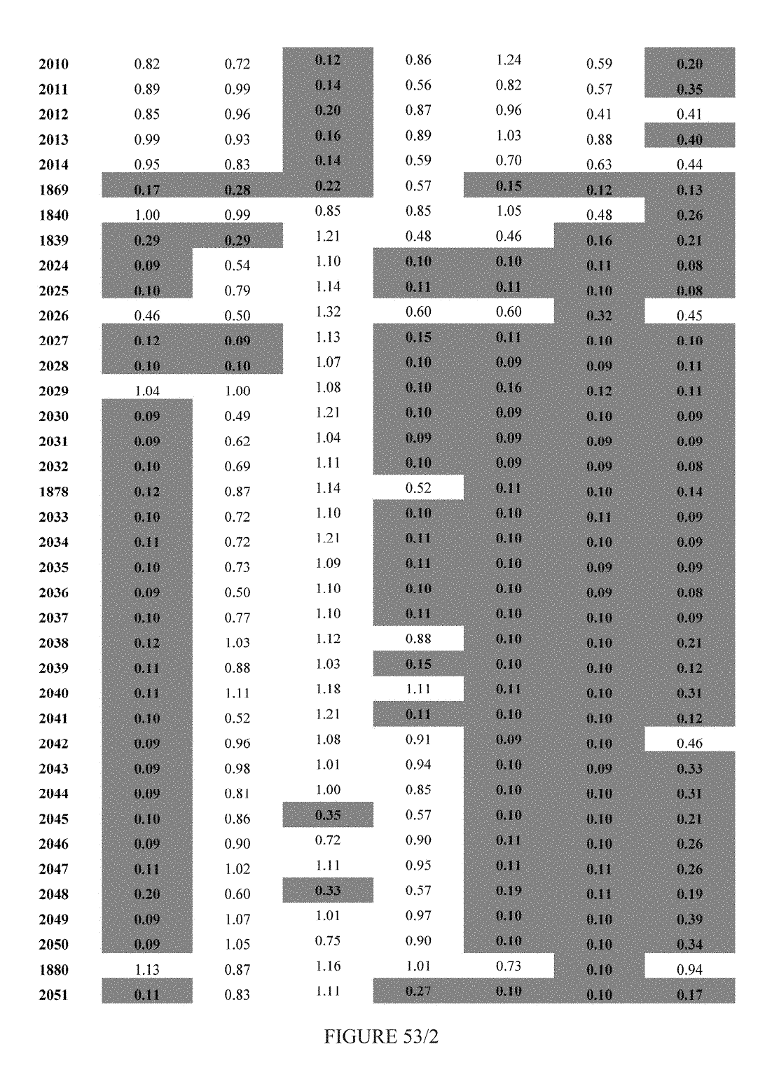

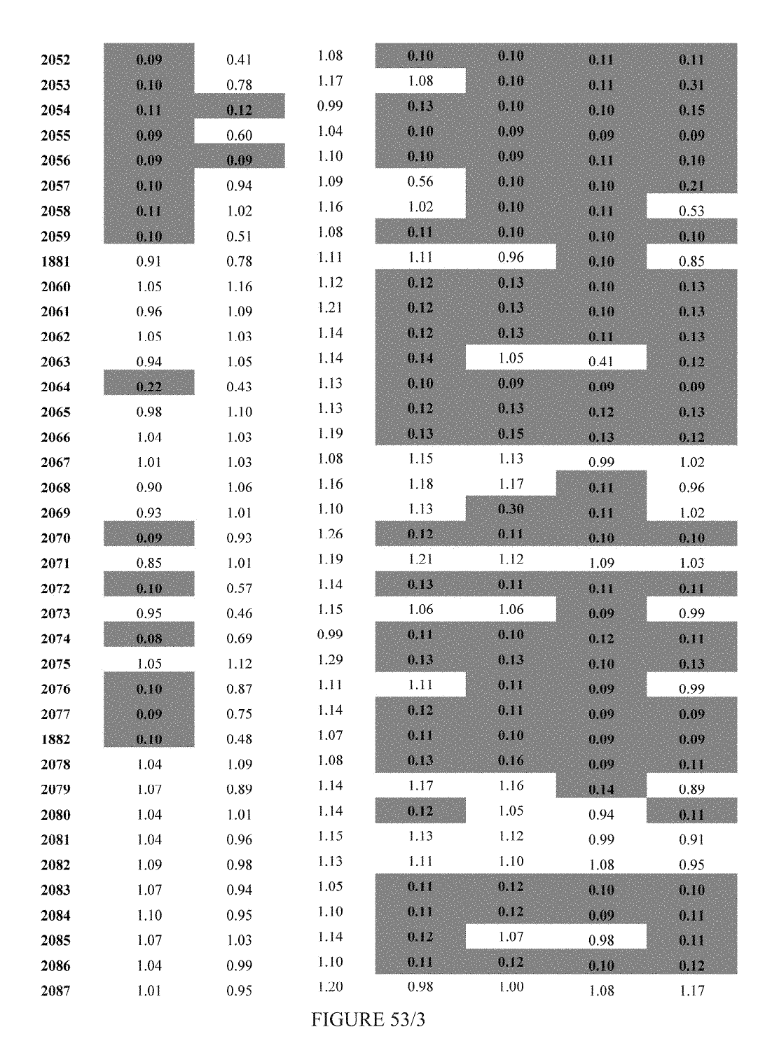

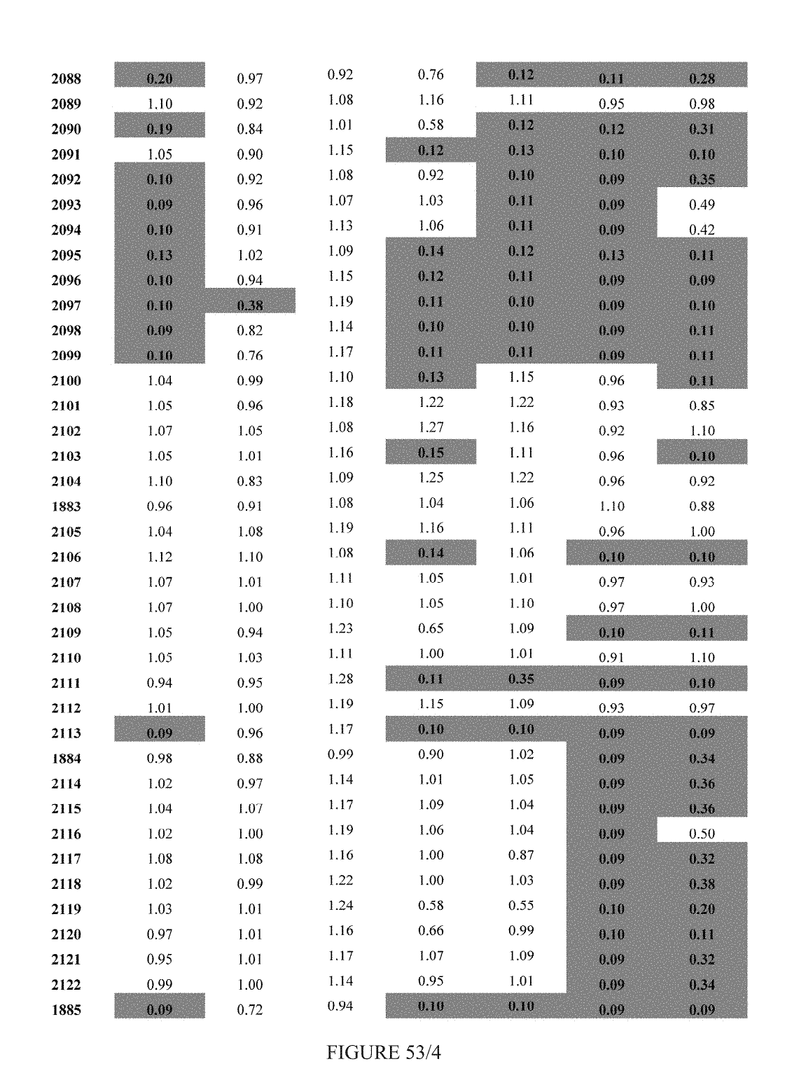

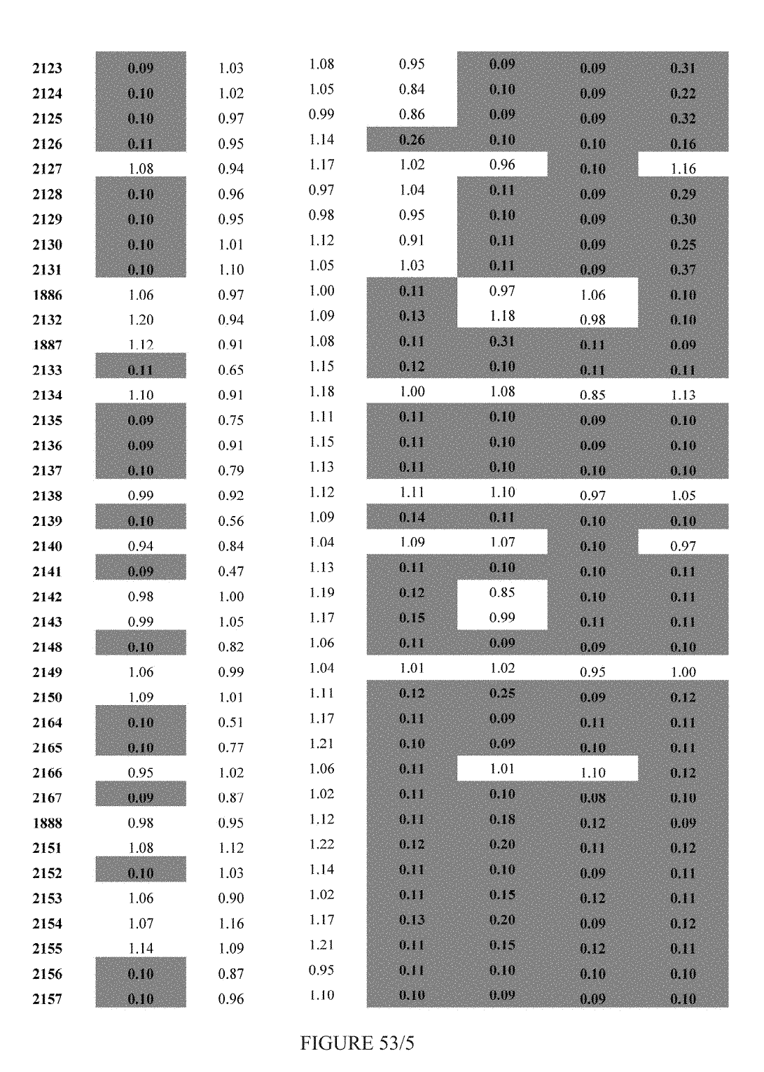

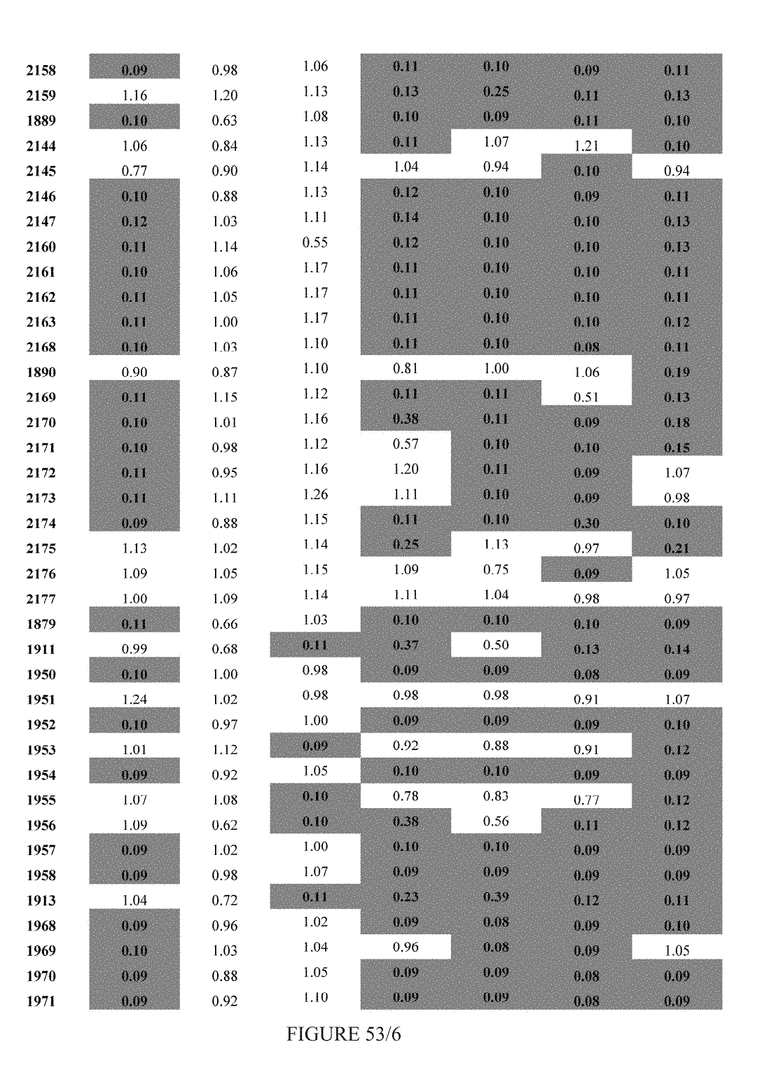

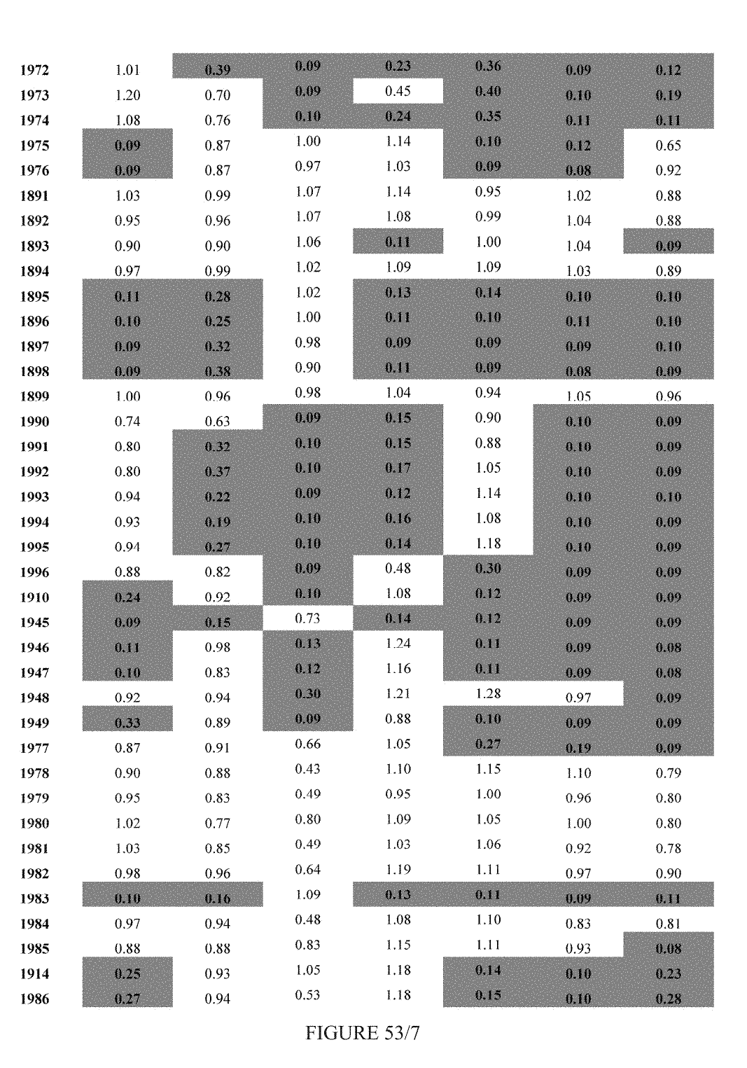

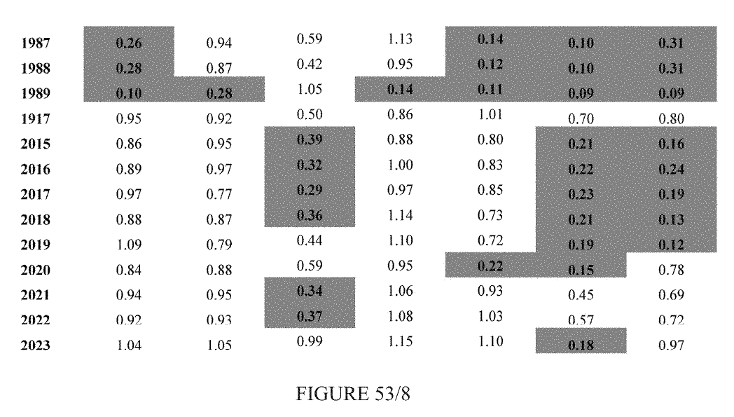

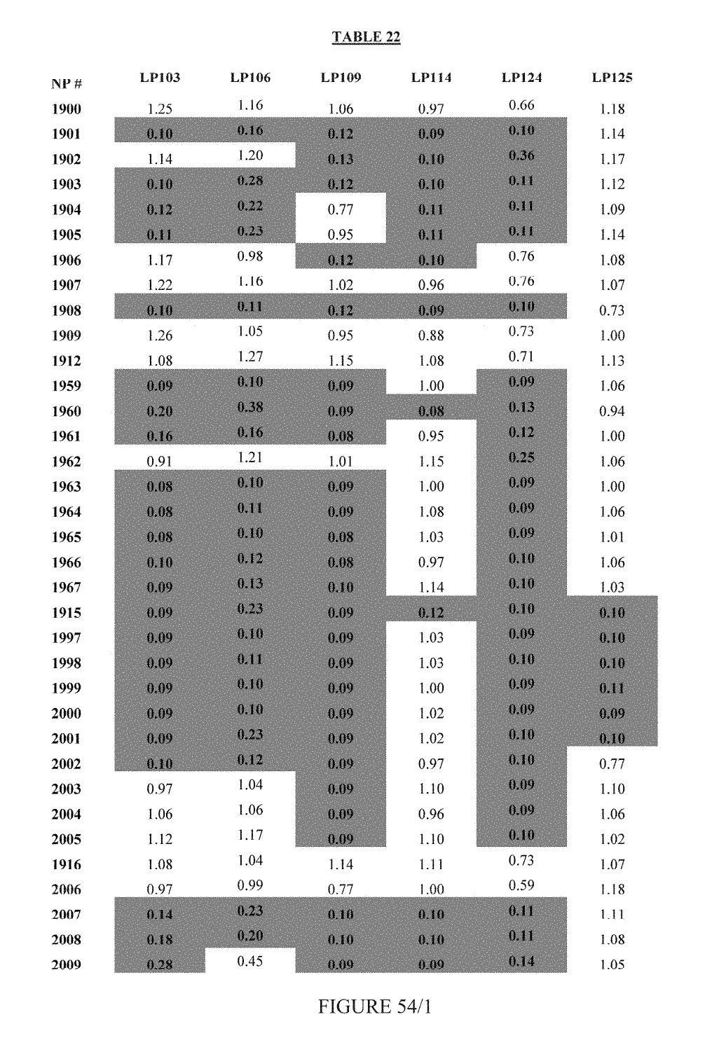

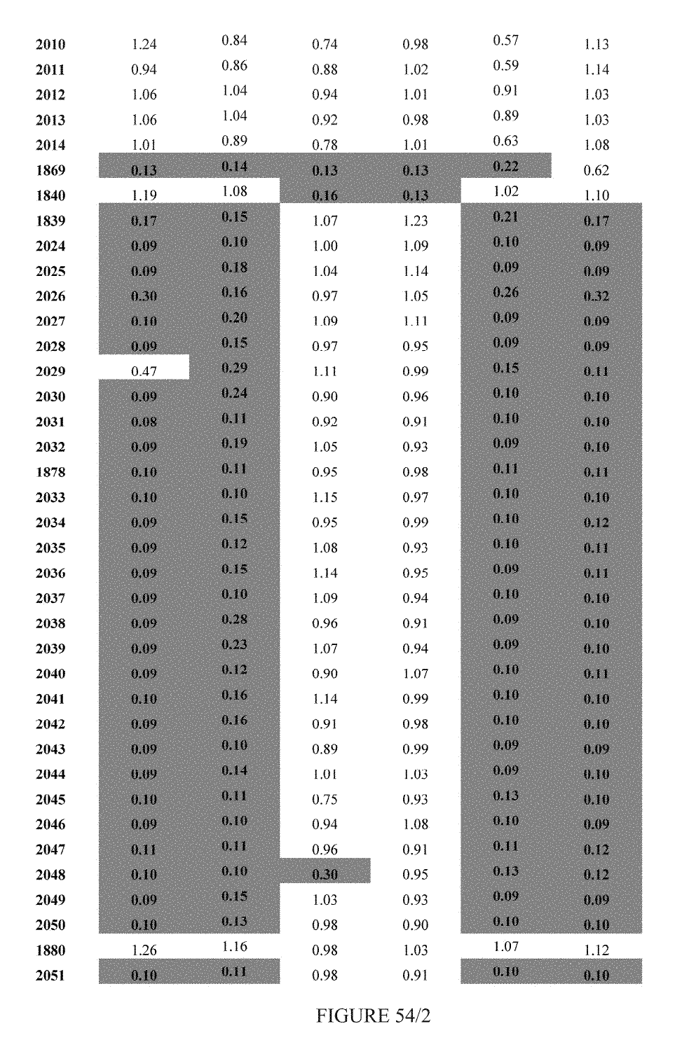

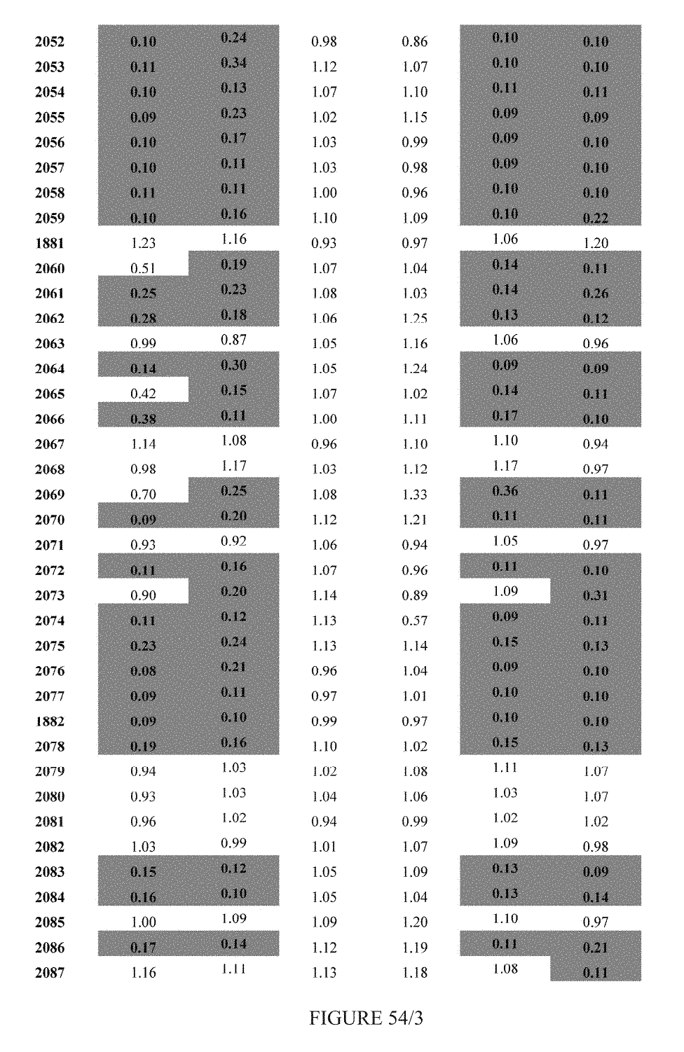

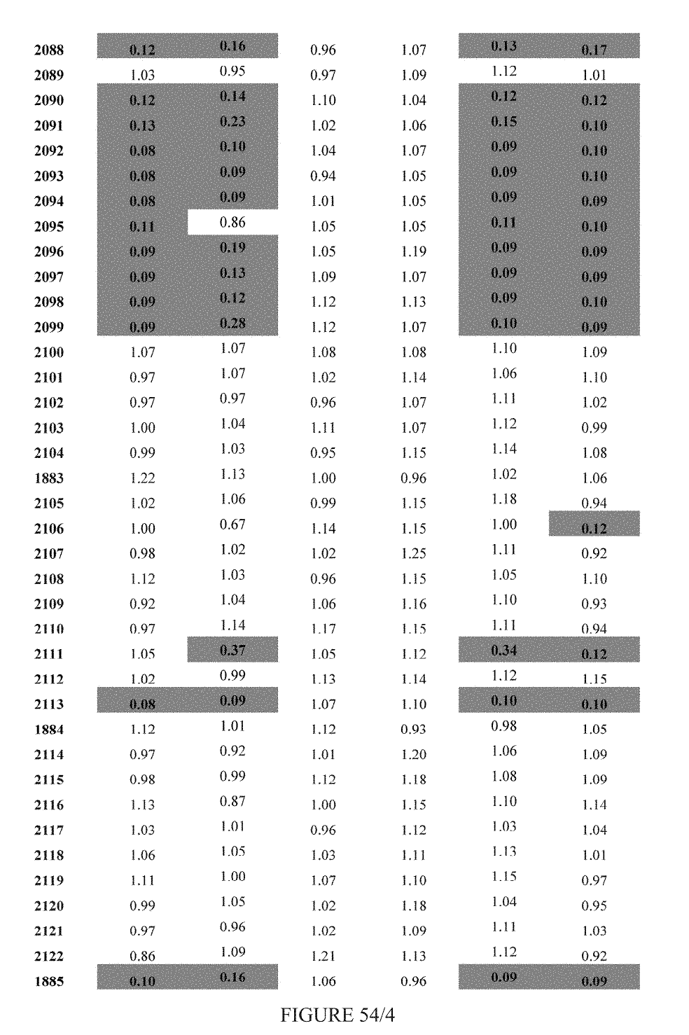

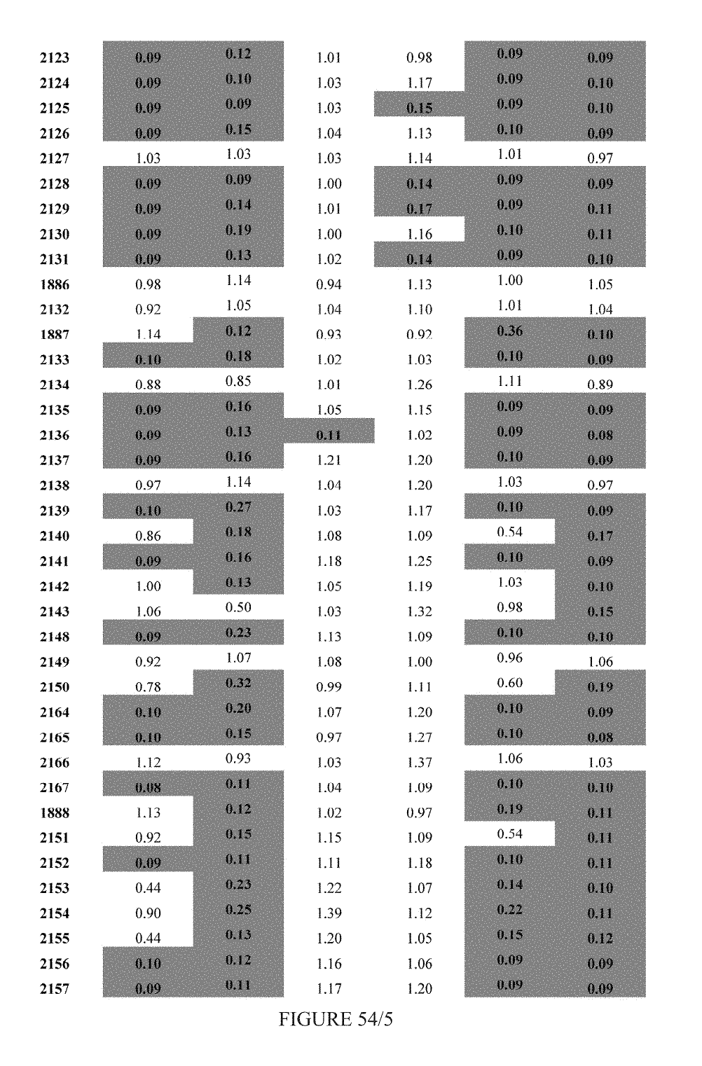

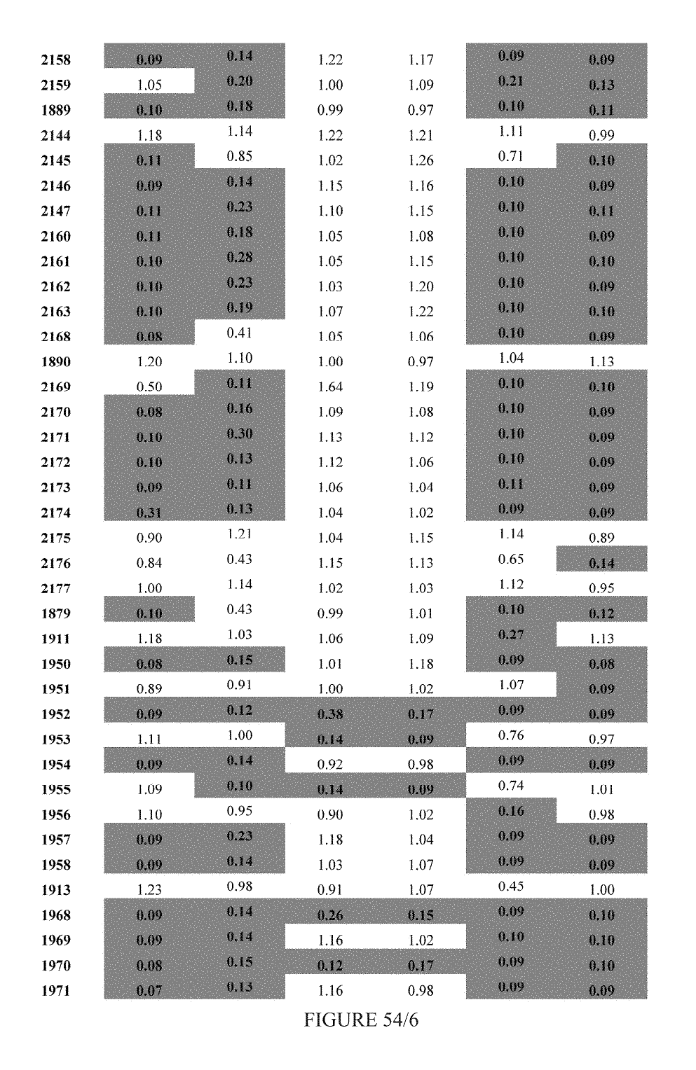

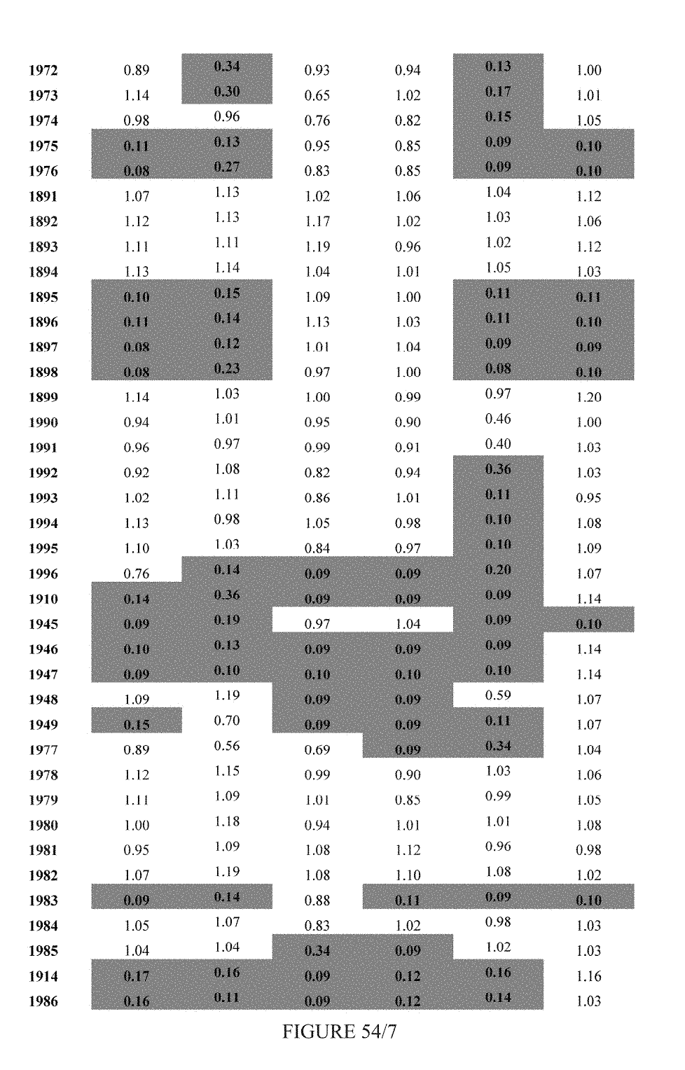

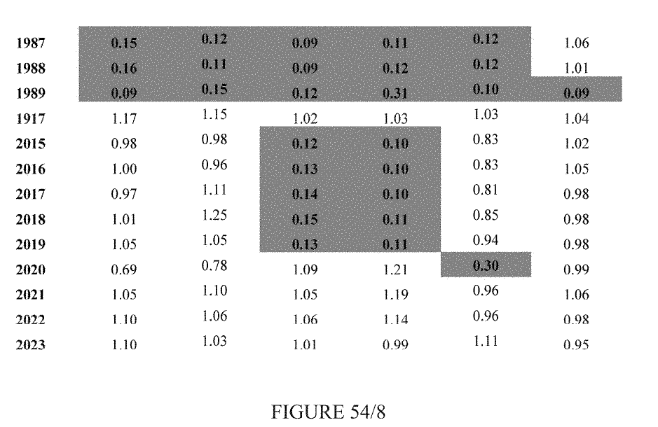

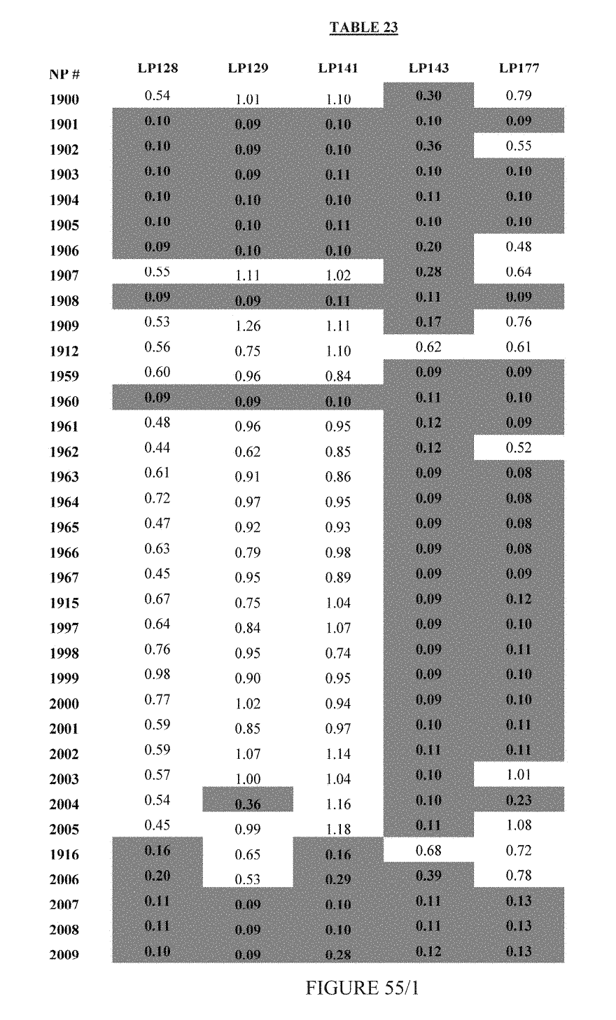

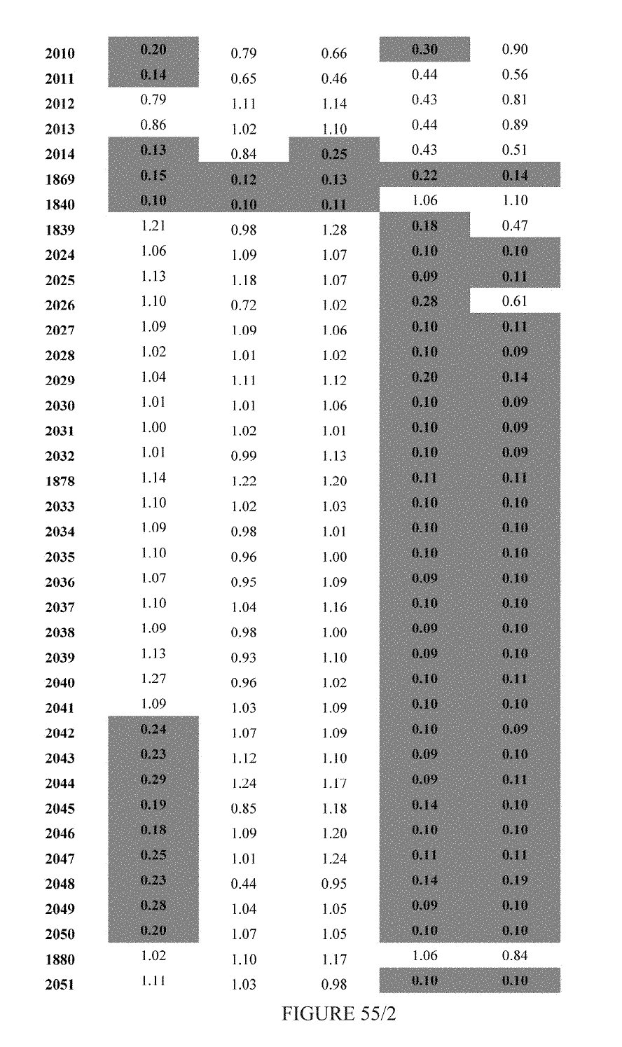

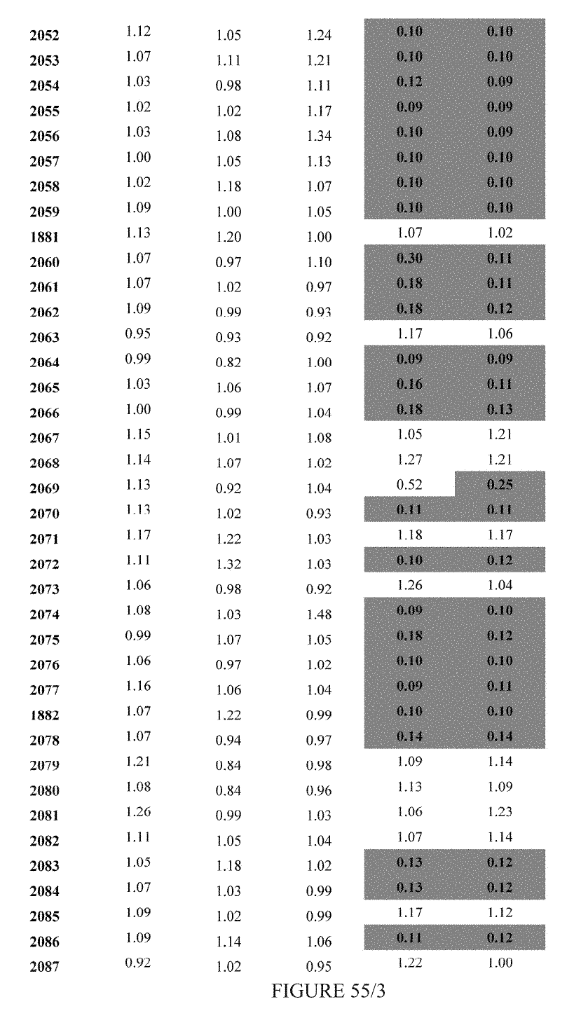

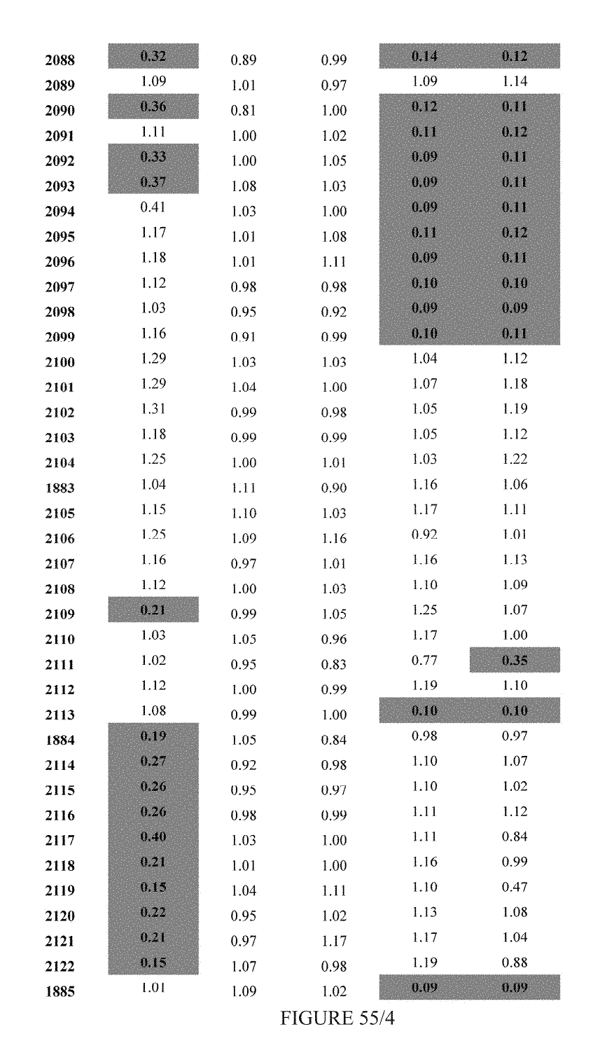

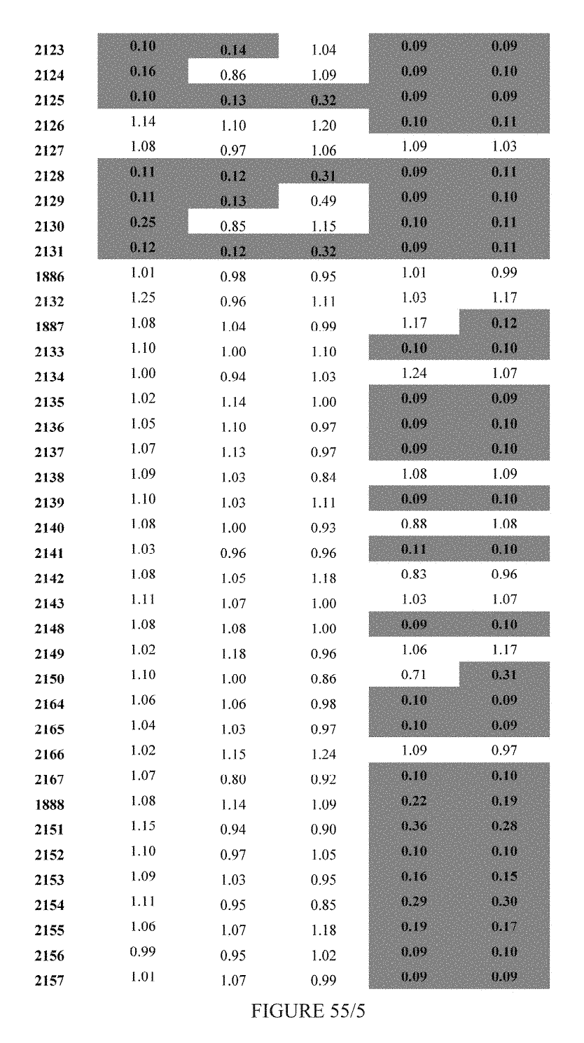

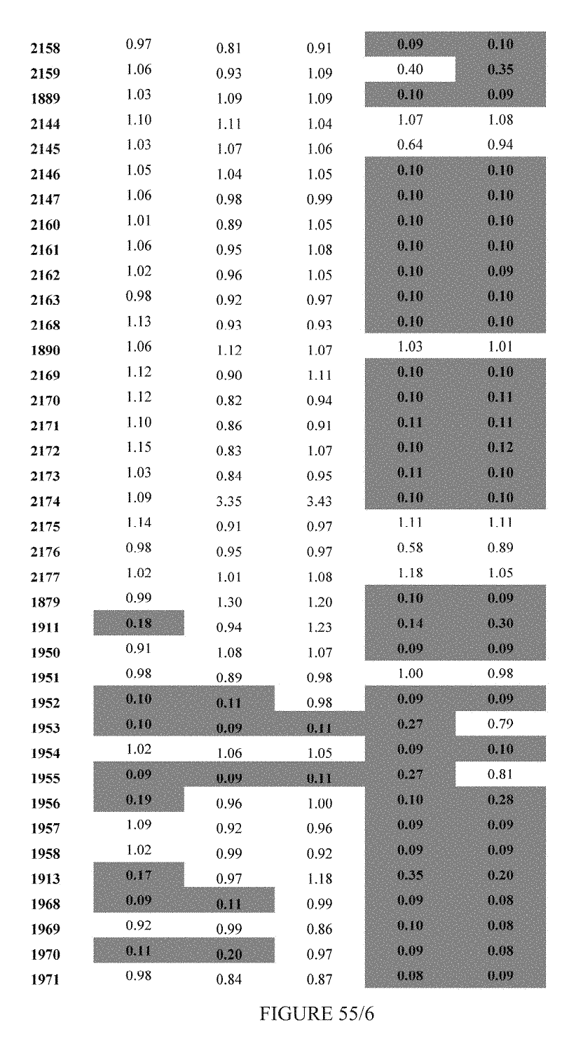

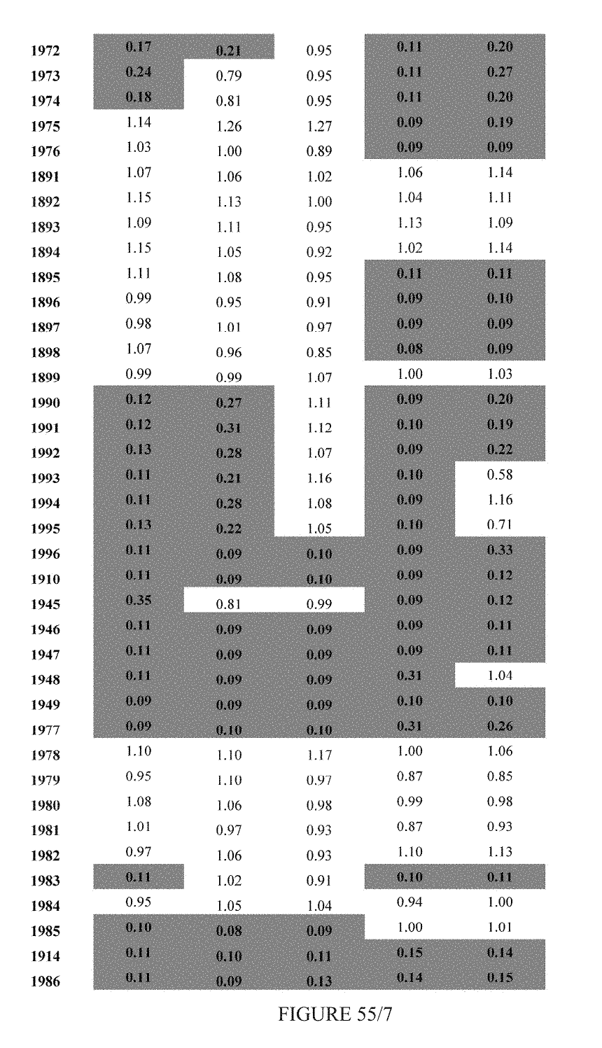

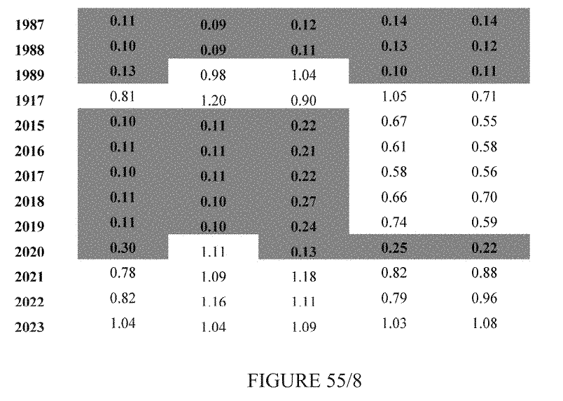

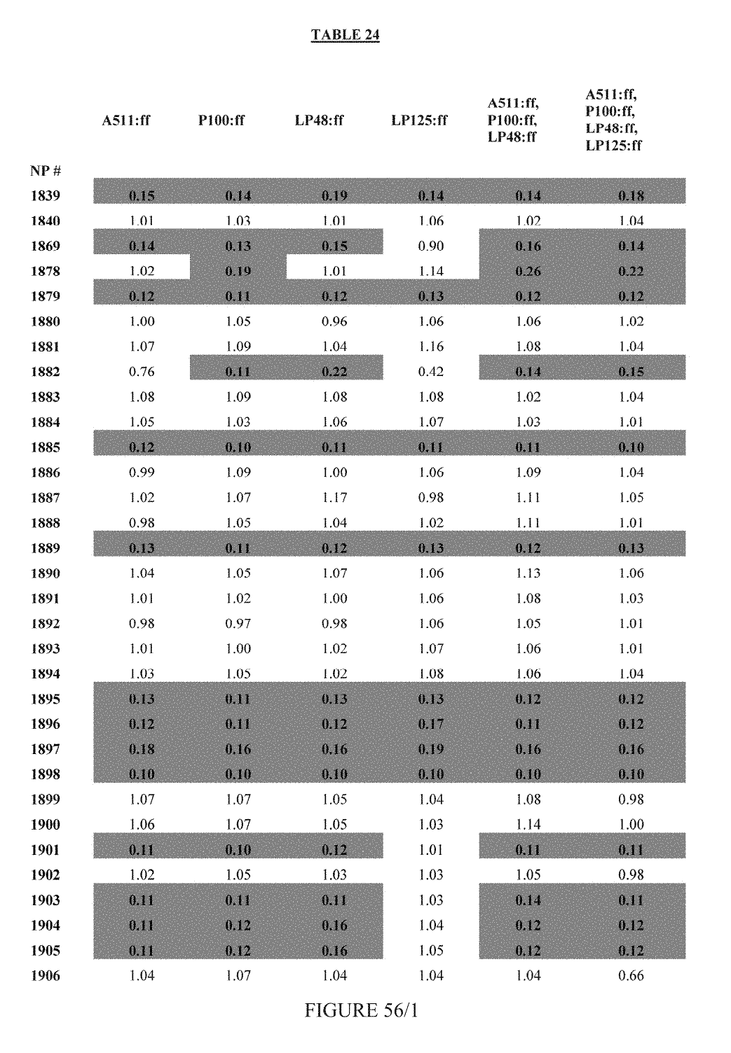

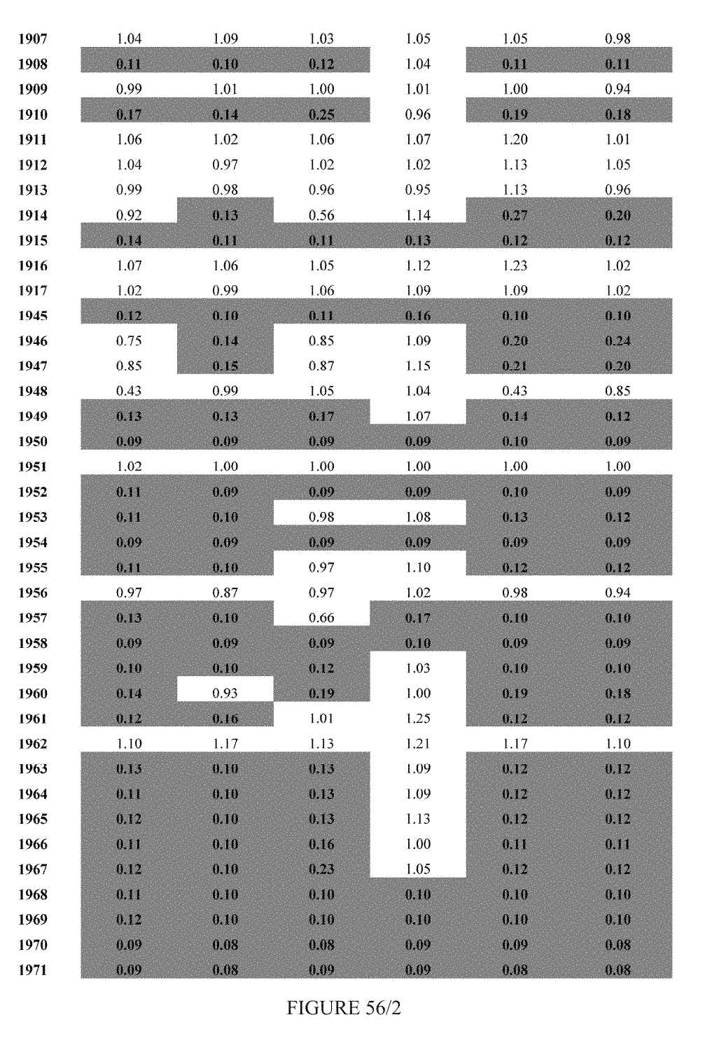

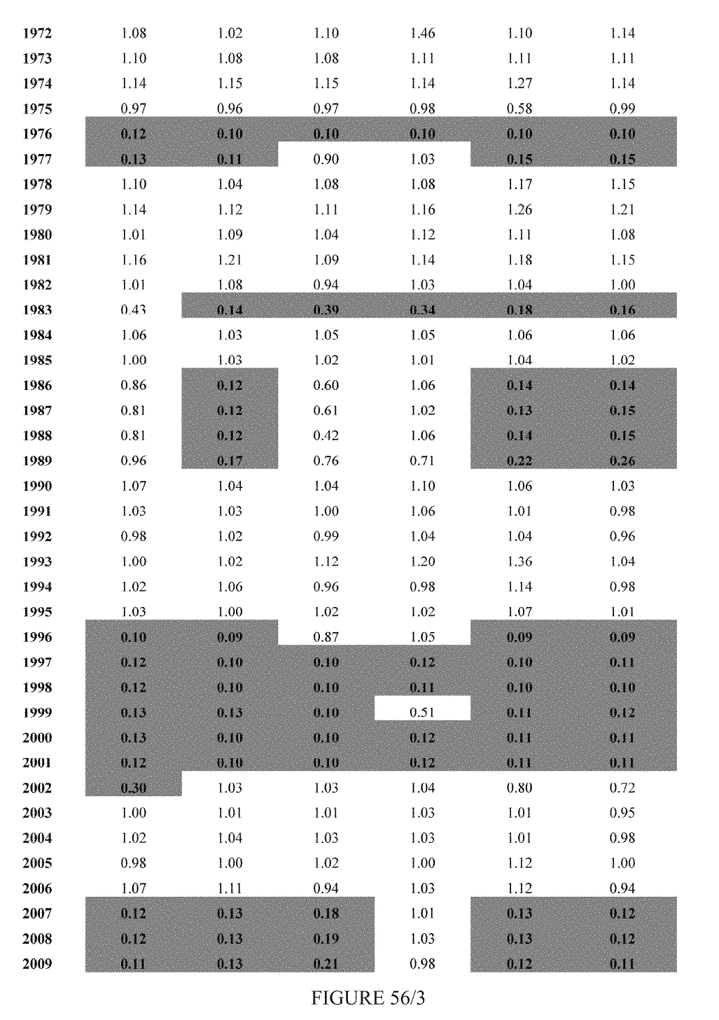

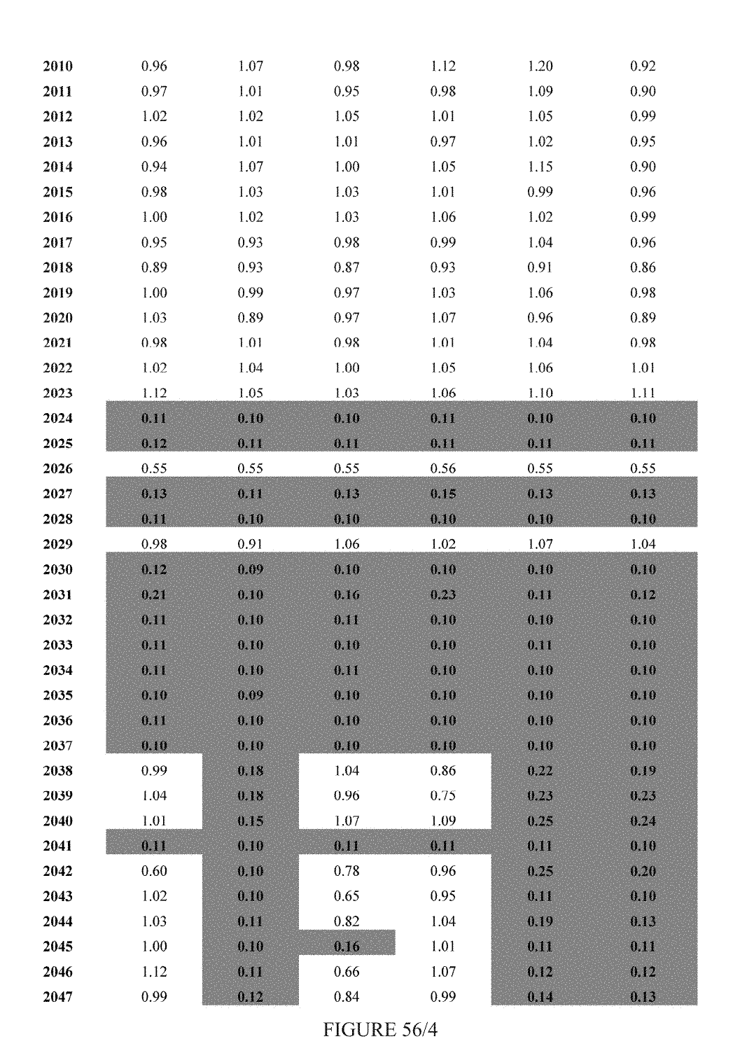

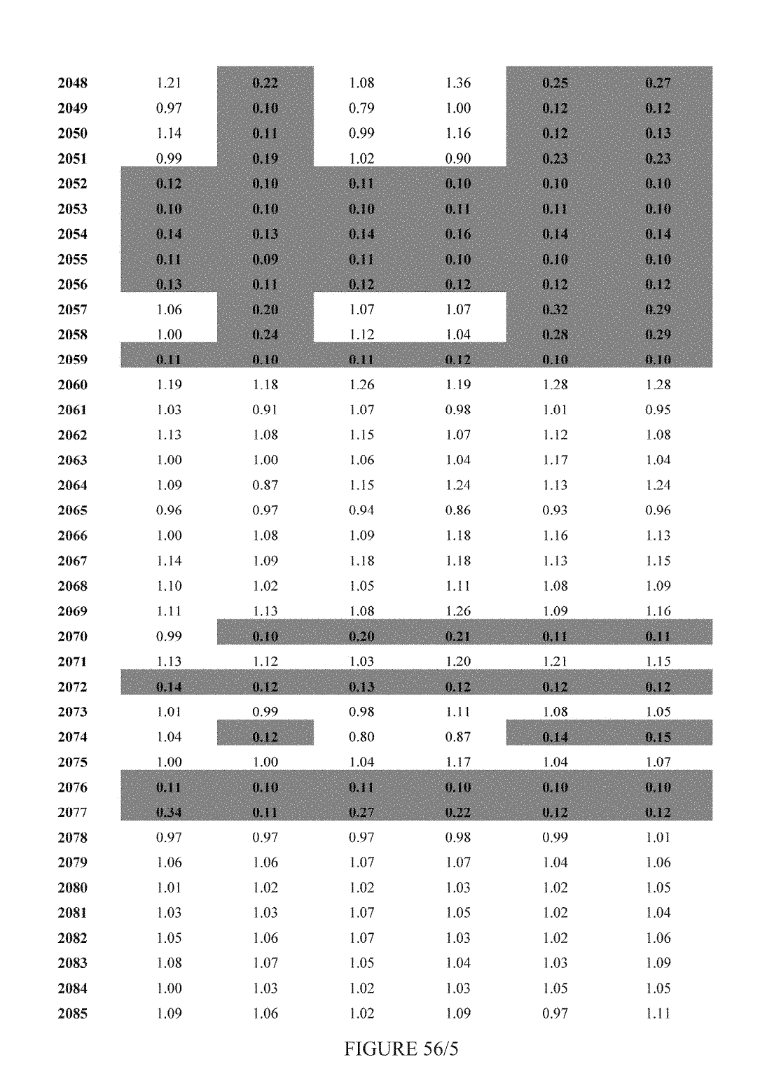

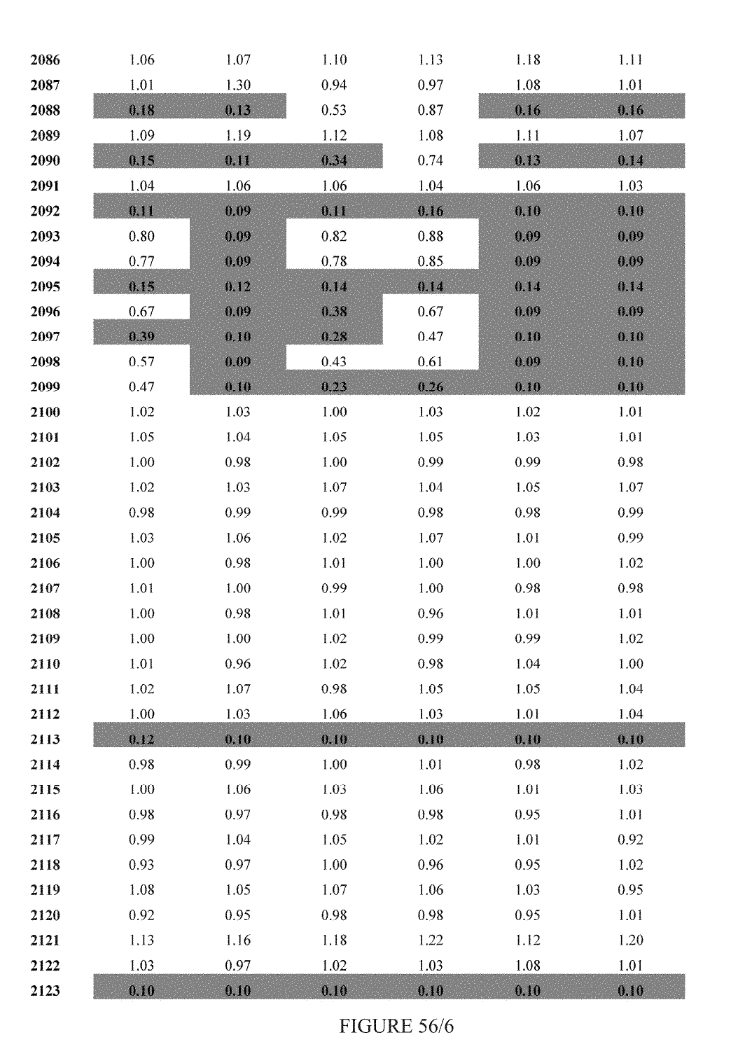

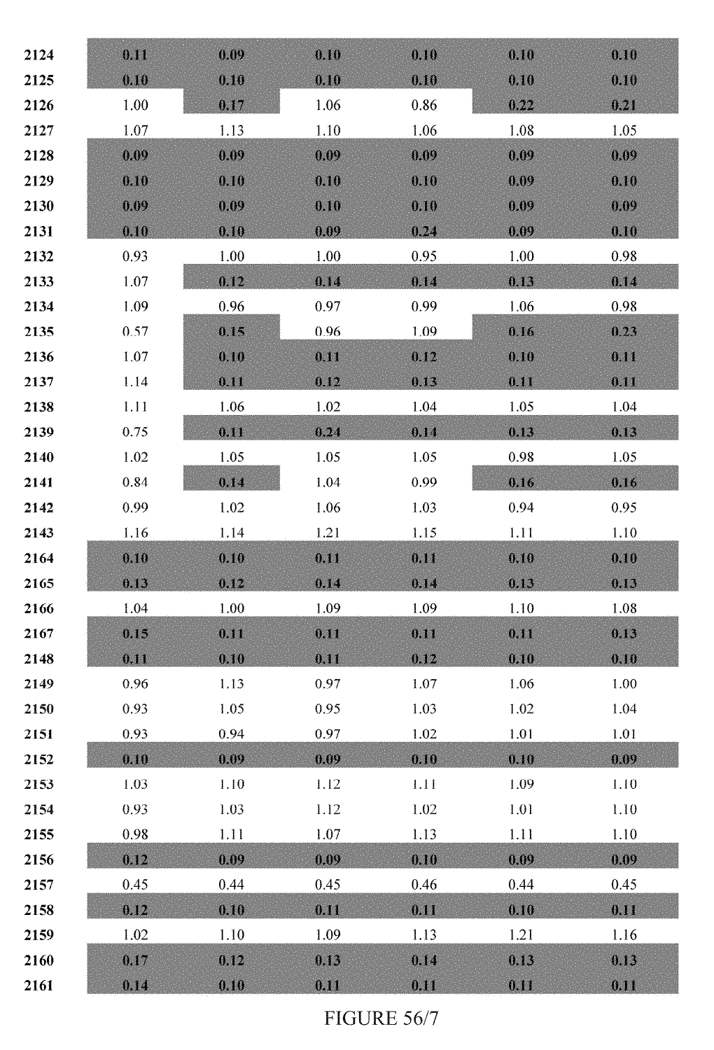

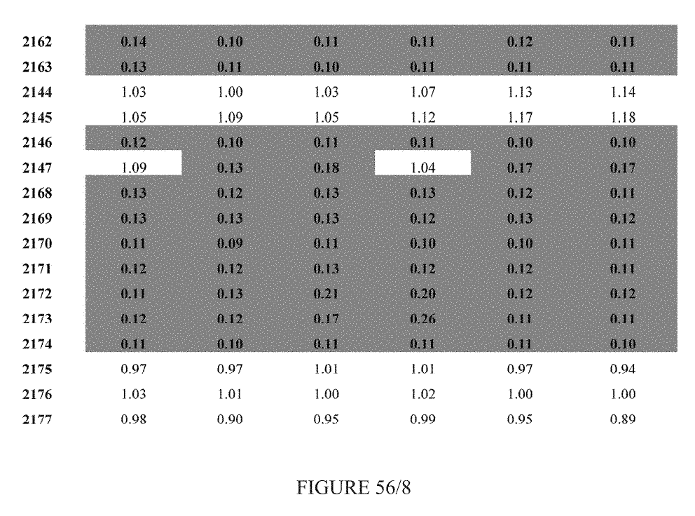

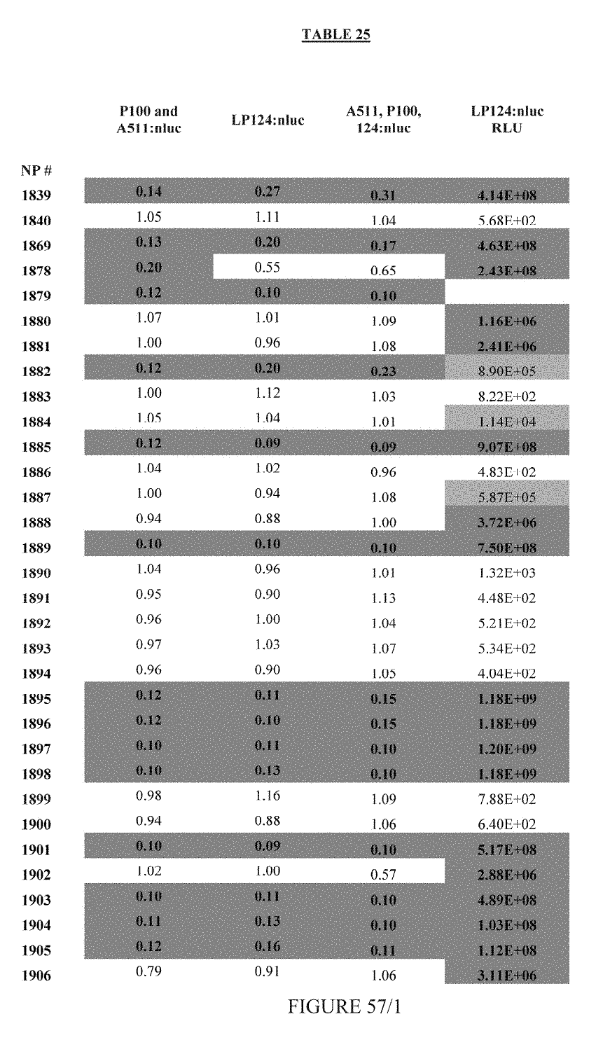

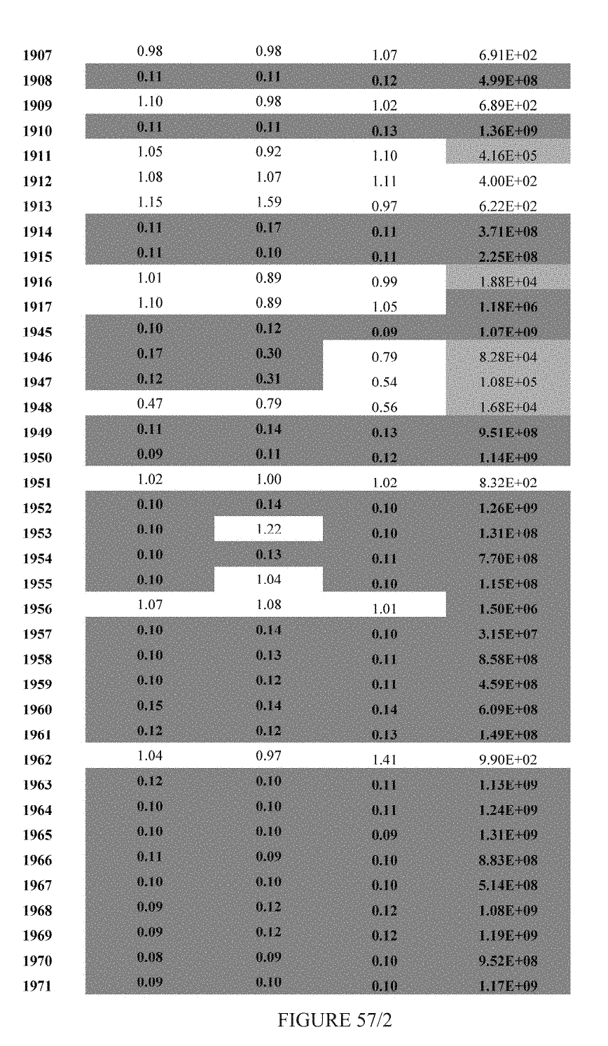

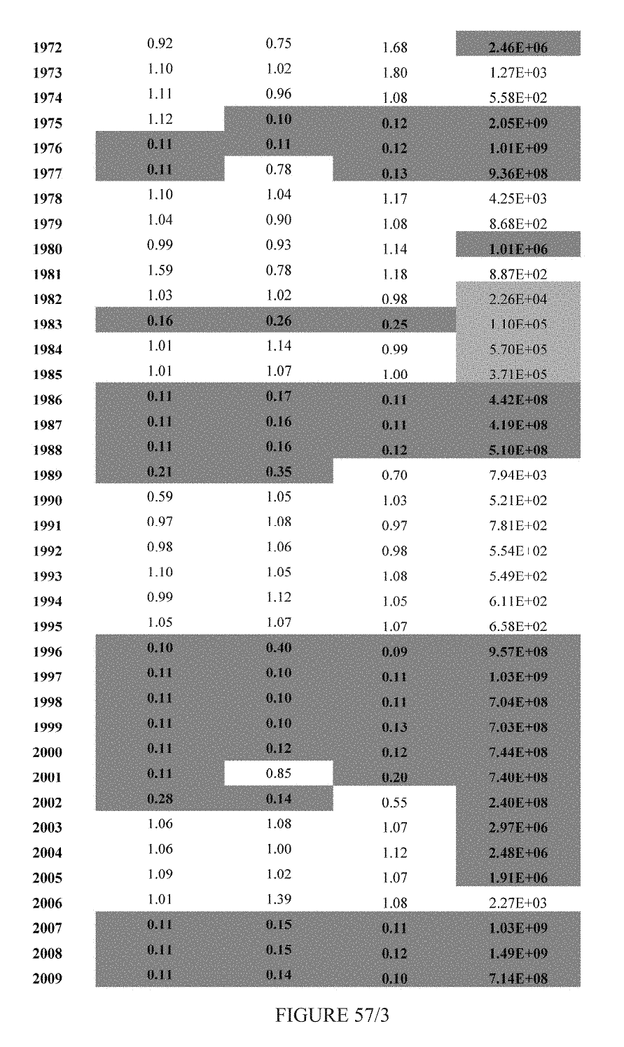

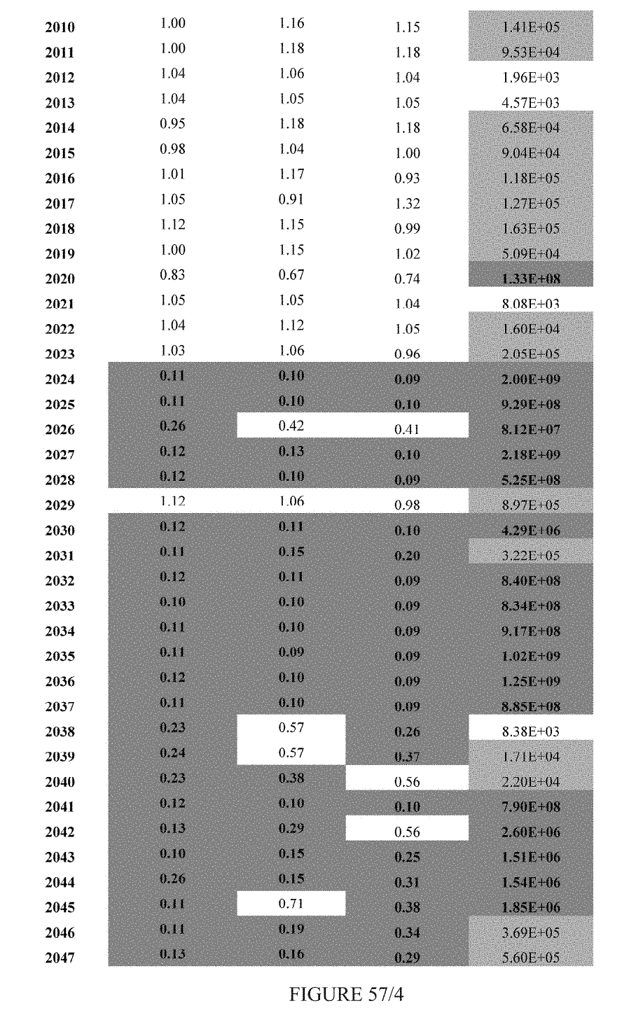

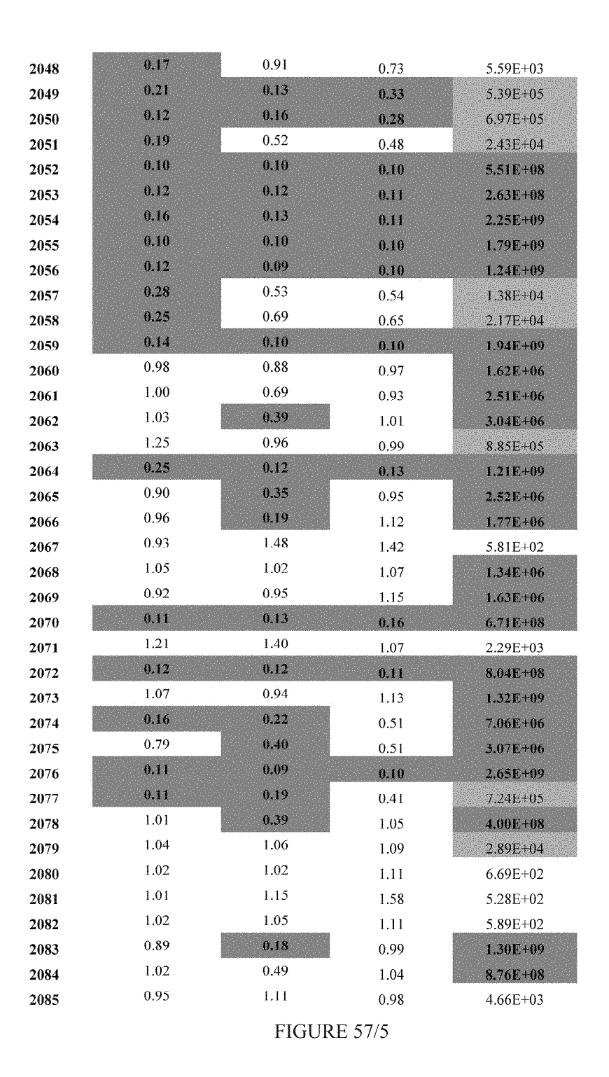

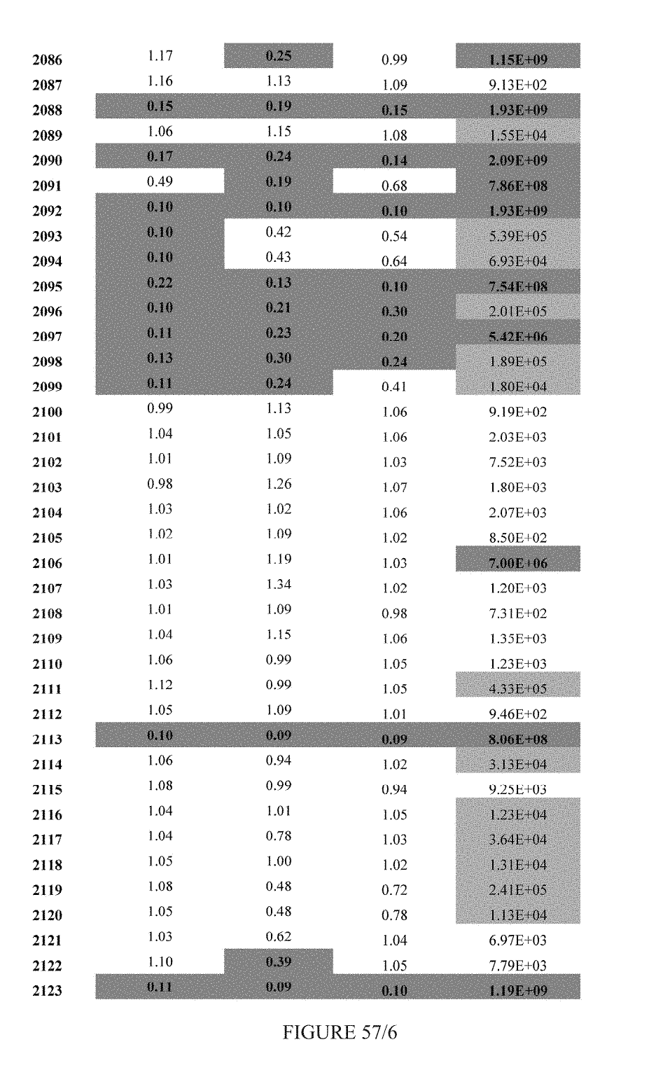

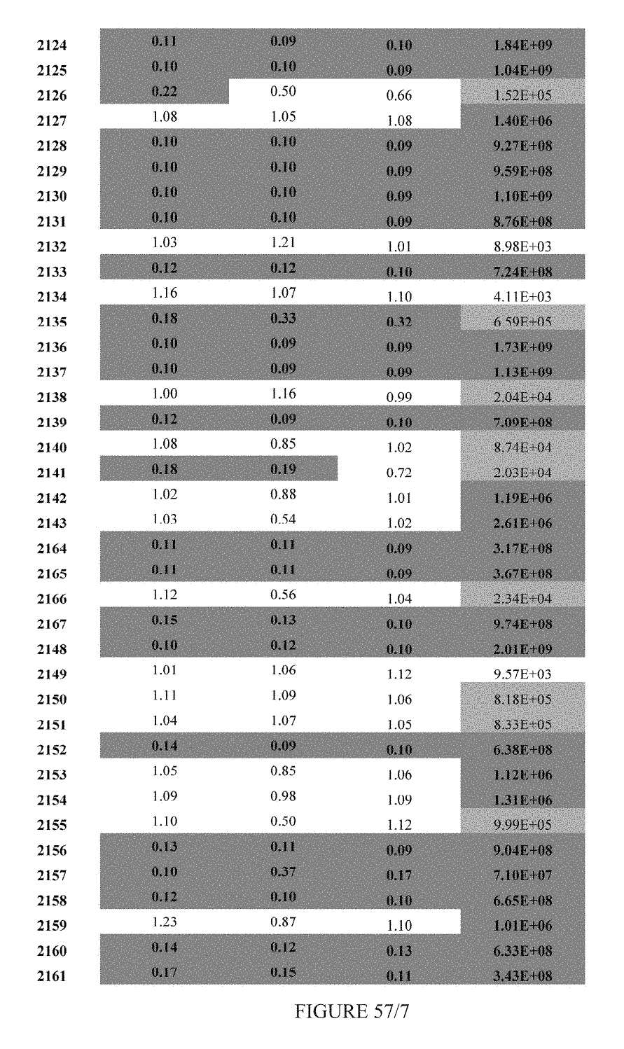

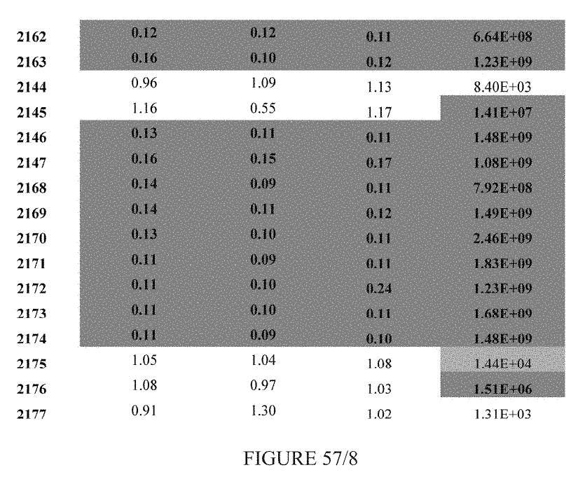

The Listeria bacterial strain panel was used to determine the host range for a particular bacteriophage. To do this a culture of each Listeria strain to be tested was started in 5 ml of LBL1 and grown overnight at 30 C in an orbital shaker and allowed to grow for 16 hours. For each bacterial host strain 30 μl of the 16-hour culture was mixed with 270 μl of fresh LBL1 medium. To each cell dilution, 4 ml of LBL1 soft agar was added and overlayed onto LBL1 agar in 100 mm petri dish. The soft agar overlay was allowed to cool and solidify at room temperature. Additionally, a reference strain (FSL F6-367 for A511 and P100) was treated in a similar manner to the host range isolates. A 10-fold dilution series of the bacteriophage in LBL1 medium was prepared from 10⁻¹ to 10⁻⁸. 5 μl of each dilution of the bacteriophage was spotted onto the soft agar overlay and the liquid was allowed to adsorb and then the plate was incubated at 30 C for 16 hours. After incubation the plaques present at each dilution series were counted and compared to the reference strain to provide an efficiency of plaquing for each host range isolate. The host range was represented as a percentage of the titer observed on the experimental host compared to the reference strain. Bacterial strains that showed a plaquing efficiency greater than 10% (FIG. 51 (Table 19), dark gray shading) of the reference strain were considered to be within the host range. Bacterial strains that showed a plaquing efficiency less than 10% but greater than 0.01% (FIG. 51 (Table 19), light gray shade) of the reference strain were considered to be weakly susceptible to the phage. Bacterial strains that showed a plaquing efficiency less than 0.01% (FIG. 51 (Table 19), unshaded) of the reference strain were considered to be outside of the host range for a phage. A phenomenon that was seen for many of the bacterial strains tested was what has been described in the literature and art as “extracellular killing” (ECK) (FIG. 51 (Table 19), black), see e.g. Shaw et al. (J Immunol Methods. 1983; 56(1):75-83). A strain was defined as demonstrating ECK for a particular phage when at high phage concentration completely cleared the lawn, however, subsequent dilutions did not produce clearing.

The plate-based host range determination allowed for a rough approximation of the host range of A511 and P100 against the Listeria isolate library. Of the 272 strains tested in the bacterial strain library 67 and 120 strains supported plaque formation by A511 and P100, respectively (FIG. 51(Table 19)). The greatest limitations of this method were the length of time needed to process the entire library for a give bacteriophage and the inability to determine the entire host range due to the ECK phenomenon. For the bacteriophage A511 and P100, of the 272 bacterial strains in the host range panel tested, 117 and 42, respectively, showed ECK and hence provided no information about the host range for these strains. Additionally, in view of the ECK phenomenon and because of the general differences between bacteria growing on a plate and bacteria growing in a liquid culture, it was hypothesized that the plate-based method for determining host range may not represent the host range for a liquid-based application.

### Example 17: A Liquid Culture Phage Host Range Assay

The prevalence of the extra-cellular killing (ECK) phenomenon exhibited by both A511 and P100 in the plate-based host range method demonstrates that the plate based is not as useful as it could be for determining the host range for either phage. To overcome those deficiencies a novel liquid-based host range assay was developed. The liquid-based host range assay is an end point assay where the ability of a phage to infect a particular bacterial isolate is determined by comparing the optical density of a culture with or without bacteriophage.

The Listeria host panel strain collection (Table 13) was struck out on Brain Heart Infusion (BHI) agar plates and single colonies were inoculated in 1 ml BHI liquid in a 2-ml 96-deep well dish, covered with a sterile breathable sterile membrane and grown at 30 C for 16 hours. Each of the 16-hour cultures from the 96-well plates were diluted 1:10,000 in 198 μl of LBL1 in a 300 μl flat-bottom optical 96-well plate and then either 1×10⁵ pfu of the bacteriophage or an equivalent volume of LBL1 was added to each well of the 96-well plate. This concentration of bacteriophage and bacterial cell dilutions was to approximate a multiplicity of infection (MOI) of 1 in each well. After addition of the phage or control, the plates were incubated at 26 C with shaking at 50 rpm for 16-hours. Plates were placed in a 96-well plate reader (Biotek Eon Microplate Reader) and agitated for 3 seconds with orbital shaking to resuspend cells that had settled out of culture. After the agitation, the optical density of each well was measure at 600 nm (OD600) wavelength light. The ratio of OD600 of the bacterial isolate in the presence of bacteriophage to the uninfected bacterial isolate culture was used as a metric to determine the efficiency of infection for a bacterial strain. A bacterial strain with a ratio of less than or equal to 0.4 (Table 2, dark gray shade) was considered to be sensitive to infection by the bacteriophage.

The liquid-based host range assay identified 192 and 153 bacterial strains sensitive to A511 and P100, respectively, of the 272 strains in the bacterial strain panel (FIG. 51 (Table 19)). This data shows that A511 is capable of infecting approximately 70% and P100 is capable of infecting approximately 58% of the host range panel. In comparison to the liquid-based host range, the plate-based host range method identified 62 and 120 bacterial strains that demonstrated a plaquing-efficiency for A511 and P100, respectively. Of the strains identified in the plate-based host range methods, only 8 A511-sensitive bacterial strains and 3 P100-sensitive bacterial strains did not show clearance in the liquid-based clearance assay. Because the liquid-based assay is an endpoint assay and represents a kinetic interaction between bacteriophage infection and bacterial cell growth certain bacterial strains with increased cell growth rates may be able to saturate a culture even though the strain is susceptible to infection and this may explain the reason why a small number of strains identified in the plaque-based assay were not identified in the liquid assay.

The additional strains identified by the liquid-based host range assay were due to the ability to collect data on strains that demonstrated an ECK phenotype in the plate-based host range assay. The large number of strains that demonstrated this phenotype created a large amount of unknown information regarding the host range for A511 and P100. The liquid-based assay eliminated the ECK phenomenon, one of the large drawbacks of the plate-based host range method. Two factors contributed to the lack of ECK. First the concentration of phages used in the liquid-based assay is a set concentration that is lower than the concentrations of phage that demonstrated ECK in the plate-based host range assay. Second, the delocalized concentration of bacteriophage within the liquid infection and the low MOI decreases the number of interactions between the bacterial cells and bacteriophage. The limited interaction decreases the possibility of non-productive encounters and lowers super-infection, or infection by multiple bacteriophages of a cell. By eliminating ECK, the sensitivity for measuring susceptibility of a particular bacterial cell to a bacteriophage was increased substantially and provided a more accurate representation of the host range of a bacteriophage across the Listeria species.

The liquid-based host range assay showed substantial advances over the prior method of using a plate-based system for determining host range of a bacteriophage. Previous literature did not report the ability of growing these bacteriophages in a format other than a plate-based method. The liquid format is also useful because the speed with which the liquid-based host range assay can be performed increases the speed of determining the host range of a bacteriophage from 7-10 days for the panel as it was assembled to several hours of hands on labor. Additionally, the high-throughput nature of the scoring of host susceptibility allowed for multiple bacteriophage host ranges to be determined concurrently, a possibility that did not exist previously. The ability to process multiple bacteriophages concurrently allowed for a more direct comparison of bacteriophages by minimizing variation between bacterial culture physiology and media lots. Together, the increased speed and direct bacteriophage characterizations allowed for rapid processing of multiple phages and prioritization for bacteriophage engineering described herein. Moreover, the liquid-based host range assay allowed for a more accurate representation of the functional determination of a potential bacteriophage in a predicted product compared to a plate-based host range assay. The combination of the increased speed, ability for more direct comparison and ability to assess functionality of a bacteriophage in a more direct method to the final product makes the liquid-based host range assay significantly more useful than the plate-based host range method in most contexts.

The efficacy of a cocktail of a P100 and A511 bacteriophage can be determined by the ability of each of the bacteriophages to infect a particular strain. Infections of the host panel with a cocktail of P100 and A511 show the additive host range expected from the extrapolation of the individual host ranges. Based on observations regarding the bacteriophage concentration required for optimum luciferase production during the course of infection, the concentration of bacteriophage added was maintained at a constant total phage concentration of 1×10⁷ whether a single bacteriophage or multiple phage cocktail was used for infections. The cocktail of A511 and P100 shows coverage of 74% of the panel constructed, while the individual bacteriophages show 70% and 55% coverage, respectively. (FIG. 51 (Table 19)) This increased coverage of the panel arises from the face that while the phages have largely overlapping coverage the subset of strains susceptible to P100 infection is not full encompassed within the A511 strains. The ability to extrapolate function of a bacteriophage cocktail from the individual liquid-based host range provides as a powerful tool to identify and prioritize new bacteriophages for engineering to build a more complete cocktail.

The function of a bacteriophage cocktail of P100 and A511 on samples collected from environmental samples cannot be strictly inferred from the host panel assembled. The sites sampled in environmental testing represent diverse populations of bacteria and often have more than one species or subspecies of Listeria present at an individual location. Environmental sampling at food processing plants with geographic and source diversity identified 31 samples that have been confirmed positive for Listeria using a culture based method of detection at a third-party laboratory. Of these 31 positive samples, 10 samples contained multiple Listeria species or subspecies. The A511 and P100 cocktail was capable of detecting 24 of the 31 (77%) of the positive samples. The correlation between the liquid-based host range results and the environmental samples collected allows for further iterations on the bacteriophage cocktail to be made in order to gain more complete coverage of the Listeria genus and validated the usefulness of the liquid-based host range method.

### Example 18: Host Range Characterization of Additional Listeria Phages

Construction of a Listeria host strain panel and development of a rapid liquid-based host range assay allowed for the rapid screening of additional bacteriophages to identify those bacteriophages that would increase the breadth of coverage of the Listeria genus. Twenty five additional bacteriophages were screened against the host panel in the liquid-based host range assay and analyzed for host susceptibility based on clearance versus an uninfected control. The data are presented in Tables 21-23. Strains were considered within host range if they demonstrated a ratio of 0.4 or less (shaded dark gray). During the determination of the OD600 of the cultures there was no correction for the absorbance of the growth medium or culture plate, therefore, a ratio of 0.09 constituted a completely cleared culture by infection. Because of variations in the maximum OD600 obtained by different Listeria strains a conservative ratio of 0.4 was chosen to denote Listeria strains that were sensitive to a given bacteriophage. Strains that had an OD600 ratio of greater than 0.4 were considered to be outside of host range (Tables 21-23, unshaded). From these twenty five bacteriophages assayed, seven (7) bacteriophages were selected to proceed into engineering based on the criteria that they provided useful host panel coverage, had genome sequence availability for development of phage targeting vectors and were capable of infecting L. monocytogenes strain EGD-e, the strain of Listeria most amenable to transformation.

The seven bacteriophages selected in addition to A511 and P100 were LP44, LP40, LP48, LP99, LP101, LP124, LP125, and LP143. No individual phage assayed covers more than 78% of the Listeria host strain panel. In combination, the bacteriophages cover approximately 92% of the host strain panel as assayed by liquid-based host range assay (Tables 21-23). This combinatorial approach allows for the construction of a bacteriophage cocktail that provides the necessary coverage of the Listeria species to provide a reliable determination of the presence of Listeria in environmental sample collection.

After engineering the genome of the phages with two different genetic payloads, Firefly Luciferase and Nanoluciferase, the host range of these phages was retested to ensure that the genome modifications did not affect the fitness of the phages or compromise their ability to infect the target bacteria. To examine the result of combining bacteriophages in an infection the liquid-based host range assay was used to test the combinatorial effects of phage infection. For these infections the final concentration of phage was maintained at a constant 1×10⁵ pfu consisting of equal amounts of each of the phage within the cocktail (i.e.—a two phage cocktail would consist of 5×10⁴ pfu of each of the two component phages.

### Example 19: Engineering Listeria Phage

A novel phage engineering method was developed to create recombinant phage. This method is sometimes referred to herein as Phage Infective Engineering (PIE). This method allows insertion of a heterologous nucleic acid sequence into any desired location of a phage genome. The initial site chosen for insertion was that used in Loessner, et al. (Appl. Environ Microbiol., 62:1133-1140), downstream of the major capsid protein gene cps. The coding sequence (SEQ ID NO: 1) for the firefly luciferase (SEQ ID NO: 2) or the coding sequence (SEQ ID NO: 3) for the nanoluc luciferase (SEQ ID NO: 4) was inserted at this location.

The PIE method uses Phage Targeting Vectors PTVs which include the luciferase gene sequence flanked by ˜1 KB of phage sequence directly upstream and downstream of the desired insertion site (referred to as an upstream homology region (UHR) and downstream homology region (DHR)). Each of these inserts was created using PCR primers that would amplify the desired amplicon, while adding 20 bp of homology to facilitate assembly. Plasmids were assembled using the GeneArt Seamless Assembly Kit (Life Technologies). The 3 inserts (UHR, luc, DHR) were assembled into the gram positive/gram negative shuttle vector pMK4, which was restriction-digested with SmaI and PstI (NEB).

The A511 phage genome sequence is available in Genbank (NC_009811). A511 phage may be obtained from ATCC (PTA-4608™).

The PIE method was used to insert the firefly luciferase gene (SEQ ID NO: 1) directly after the stop codon of the cps gene of A511, between bases 46,695 and 46,696 of the genomic sequence. No sequence was deleted from the phage genome. A 16 bp sequence containing a ribosome-binding site (GAGGAGGTAAATATAT) (SEQ ID NO: 67) was placed before the start (ATG) of the firefly luciferase gene.

To engineer phage A511, 1276 bases of the cps gene were amplified using oligos “pMAK upf” and “pMAK upr”, forming the fragment “A511 UHR”. The luciferase gene was amplified using primers “pMAK lucf” and “pMAK lucr”, creating the fragment “A511 luc”. The primer “pMAK lucf” also added a ribosome binding site (Shine-Dalgarno) upstream of the luciferase gene. The 1140 bp immediately after the cps stop codon was amplified using “pMAK dnf” and “pMAK dnr”, named “A511 DHR”.

These 3 amplicons were recombineered into pMK4 which had been restriction digested with SmaI/PstI using the GeneArt Seamless Assembly Kit, according to the manufacturer's instructions. Once isolated in E. coli, the plasmid was sequenced to verify correct amplification and assembly. Upon verification, the plasmid was transformed into the L. monocytogenes strain EGD-e and selected on BHI-chloramphenicol (10 μg/ml) agar plates.

Once the PTV was successfully transformed into EGD-e, the initial recombination was performed: An overnight culture of the A511::FF PTV-containing EGD-e was diluted 1:100 and allowed to grow to an OD600 of 0.1. This culture was then diluted back to an OD600 of 0.02 and mixed with 1e5 pfu/ml of wild-type A511 phage in a 2 ml volume. This infection was cultured at 30° C., shaken at 50 rpm overnight.

To assess whether recombination had occurred, the infection was assayed on the following day. First, the lysate was mixed with chloroform to kill any remaining cells, and to destroy the background luciferase made by the PTV. The phage is chloroform-resistant, which is a common trait in bacteriophages. 4% v/v CHCl3 was added to the lysate, vortexed, spun down, and the supernatant was recovered. A test infection was done, adding a 1:10 dilution of an overnight culture of EGD-e was mixed with the recombinant lysate (90 μl cell dilution, 10 μl phage lysate). A control infection was set up without cells. The infections were incubated statically at 30° C. for 3 hr, then assayed for luminescence on the Glomax 20/20. 20 μl of the infection was mixed with 100 μl of Promega Luciferase Assay Reagent (20 μl of lysate and 20 μl of NanoGlo for the NanoLuc phages), then read using a 10 second integration (is for NanoGlo). The recombinant lysate produced light, indicating that there were recombinant phage in the lysate.

In order to enrich and isolate the recombinant phage, it needed to be separated away from the wild-type phages present in the recombinant lysate. Successive rounds of dilution and division were employed. Lysates were made with 10-fold dilutions of input phages, and screened for the presence of recombinant phage by assaying the lysates for luciferase activity.

The recombination efficiency was estimated to be 1:1e5 to 1:1e6. In order to isolate a pure recombinant lysate, the methods described in (Appl. Environ Microbiol. 62:1133-1140) were modified as follows. The initial recombinant lysate was titered. 20 l-ml lysates were set up each with 1e6, 1e5, and 1e4 pfu/ml of the recombinant lysate: 1 ml EGD-e @ OD 0.02, 1eX phages; O/N, 30 C, 50 rpm. On the following day, the CHCl3 treatment was done, as described above, for each lysate. The lysates were used to set up infections as above. Each lysate was assayed on the Glomax 20/20 (20 μl infection, 100 μl Reagent for FF, 20 μl infection, 20 μl NanoGlo for nluc). The goal was to locate the lysate that was made with the fewest number of phages that exhibits luminescence upon infection. Once this lysate was identified, it was titered and used to set up lysates with 1e3, 1e2 and 1e1 pfu/ml. Once a luminescent lysate was isolated that had been made with 1e2 phages, this lysate was plated for single plaques. Plaques were picked into SM buffer. These “soakates” were diluted 1:10 in dH2O and assayed by PCR using “DBONO360” and “DBONO361” to look for the presence of recombinant junctions between the luciferase gene and phage sequence.

The P100 phage genomic sequence is available in Genbank (DQ004855). P100 may be obtained from ATCC (PTA-4383™).

The luciferase insertion site for P100 was also downstream of the same cps gene. The location of the firefly luciferase insertion in P100 is between base 13,196 and 13,197 of the P100 genomic sequence.

P100 was engineered in the same manner as A511 with the following exceptions: the “P100 DHR” fragment was amplified using the primers “pMAK dnf” and “pMAK dnr P100”. The single recombinant plaque was identified by picking the plaque into 100 μl SM buffer. 10 μl of this soakate was mixed with 50 μl of luciferin and luminescence was seen on the luminometer. This method of identifying positives was utilized in subsequent recombinant phage isolation.

The following phages were engineered using the firefly luciferase gene and the methods described for A511::ffluc: LP48, LP124, LP125, LP99, LP101, LP143.

The following phages were engineered using the NanoLuc gene: A511, P100, LP40, LP124 and LP125.

The PTV for A511::nluc was constructed by amplifying the following PCR fragments: Using an A511 lysate as the template, the UHR fragment was generated using oligos pMAK upf and DBONO356; the DHR fragment was amplified using oligos DBONO359 and pMAK dnr. Using the Promega plasmid pNL1.1 as a template, the NanoLuc fragment was amplified using oligos DBONO357 and DBONO358. The assembly and subsequent PIE methods were similar to those described.

The PTV and engineering for P100::nluc was performed in the same way as for A511::nluc, with the exception that the DHR fragment was amplified using the oligo pMAK dnr P100 rather than pMAK dnr.

The PTVs for LP124, LP125, and LP40 were constructed in the same way as A511::nluc, with the following changes. The DHR fragment amplified was shorter to allow for more efficient assembly of the plasmid, using oligos DBONO359 and DBONO382. Also, the insertion site was modified by adding two additional stop codons (TAATAA) directly downstream of the cps gene of these phages. These 6 bases were added by creating additional primers DBONO379 and DBONO380. The UHR fragments for these phages were amplified using oligos pMAK upf and DBONO380. The NanoLuc fragments were amplified using oligos DBONO379 and DBONO358.

The following oligonucleotides were used in the PIE methods:

pMAK upf:
(SEQ ID NO: 55)
TTACGCCAAGCTTGGCTGCAACGTGAGTTCCTAGACGACC
pMAK upr:
(SEQ ID NO: 68)
ATGTTTTTGGCGTCTTCCATATATATTTACCTCCTCTTAGTTGCTATGA
ACGTTTT
pMAK lucf:
(SEQ ID NO: 69)
AAAACGTTCATAGCAACTAAGAGGAGGTAAATATATATGGAAGACGCCA
AAAACAT
pMAK lucr:
(SEQ ID NO: 70)
ATTCAATTATCCTATAATTATTACAATTTGGACTTTCCGC
pMAK dnf:
(SEQ ID NO: 71)
GCGGAAAGTCCAAATTGTAATAATTATAGGATAATTGAAT
pMAK dnr:
(SEQ ID NO: 72)
ACGACGGCCAGTGAATTCCCAGTTACTAACTGCTCTAATG
pMAK dnr P100:
(SEQ ID NO: 73)
ACGACGGCCAGTGAATTCCCAGTTACTAACTGTTCTAATG
DBONO360:
(SEQ ID NO: 74)
CCTCTAGCTCAAATTAACGCATCTGT
DBONO361:
(SEQ ID NO: 75)
TGGCTCTACATGCTTAGGGTTCC
DBONO356:
(SEQ ID NO: 76)
TCTTCGAGTGTGAAGACCATATATATTTACCTCCTCTTAGTTGC
DBONO357:
(SEQ ID NO: 77)
CTAAGAGGAGGTAAATATATATGGTCTTCACACTCGAAGATTT
DBONO358:
(SEQ ID NO: 78)
ATTCAATTATCCTATAATTATTACGCCAGAATGCGTTCGC
DBONO359:
(SEQ ID NO: 79)
GCGAACGCATTCTGGCGTAATAATTATAGGATAATTGAATAAA
DBONO379:
(SEQ ID NO: 80)
AAAACGTTCATAGCAACTAATAATAAGAGGAGGTAAATATATATGGTCT
TCACACTCGAAGATTT
DBONO380:
(SEQ ID NO: 81)
ATATTTACCTCCTCTTATTATTAGTTGCTATGAACGTTTTTTACAGG
DBONO382:
(SEQ ID NO: 60)
ACGACGGCCAGTGAATTCCCTCGTGGTGTTCTGACTCCCG.

In subsequent experiments some modifications were made to the method. During PTV construction it was discovered that the DHR fragment was often missing from the assembled plasmid. This was overcome by shortening the length of the fragment used, utilizing oligo DBONO382.

In a modified approach, following determining the titer of the recombinant lysate, the enrichment process was sometimes conducted as follows and was used to make the nanoluc phages.

96-well microtiter plates were used to grow the PIE lysates at a 200 μl volume. For the FF lysates, the initial step was making 96 lysates at 1e6 pfu/lysate (5e6 pfu/ml), 96 at 1e5, and 96 at 1e4. For the NanoLuc phages, it was found that the recombination efficiency of the recombinant lysate was significantly higher, and that dilutions down to 1e0 pfu/lysate could be used. These lysates were made by incubating at 30° C., shaking at 50 rpm overnight. The lysates were assayed using the appropriate luciferase assay system (ff or nanoglo). Instead of using the lysates to infect fresh cells, it was found that the background signal of the lysate itself was an indication of the presence of recombinant phage.

Upon identification of a lysate made from the fewest number of phages, that lysate was used to set up new 96-well lysates using fewer phages. Once an approximate recombinant frequency of 1:10-1:100 was reached, the phages were plated on agar plates to isolate single plaques as described above.

These methods were used to create recombinant phage comprising either a heterologous open reading frame encoding the ff luciferase or an open reading frame encoding the nanoluc luciferase. In order to confirm the integrity of the inserted payload and the surrounding sequence in the recombinant phages, a fragment was amplified by PCR and sequenced. This fragment spanned the inserted sequence, beginning in the cps gene, crossing through the firefly or nanoluc gene, and crossing into the downstream sequence. The full cps gene was also PCR amplified using oligos DBONO398 and pMAK upr

DBONO398:
(SEQ ID NO: 82)
TGCTATATTATAGGAACATGGGAA.

The gene was sequenced using oligos DBONO273, DBONO398, and pMAK upr.

The PCR fragment was amplified using primers:

DBONO273:
(SEQ ID NO: 83)
TGCTTACATGCCAGTAGGGGT;
and
DBONO382:
(SEQ ID NO: 60)
ACGACGGCCAGTGAATTCCCTCGTGGTGTTCTGACTCCCG

The nanoluc phages were sequenced using oligos:

DBONO273;
DBONO382;
DBONO361:
(SEQ ID NO: 75)
TGGCTCTACATGCTTAGGGTTCC;
DBONO360:
(SEQ ID NO: 74)
CCTCTAGCTCAAATTAACGCATCTGT;
DBONO362:
(SEQ ID NO: 84)
GTATGAAGGTCTGAGCGGCG
and
DBONO363:
(SEQ ID NO: 85)
GATCTGGCCCATTTGGTCGC.

The firefly phages were sequenced using oligos:

DBONO273;
DBONO382;
DBONO360;
DBONO361;
DBONO274:
(SEQ ID NO: 86)
CGCATAGAACTGCCTGCGTC;
DBONO151:
(SEQ ID NO: 87)
CACCCCAACATCTTCGACGC;
and
DBONO152:
(SEQ ID NO: 88)
GCGCAACTGCAACTCCGATA

Sequencing was performed by Genewiz, Inc. Using the Geneious software package, alignments were made and a consensus sequence was generated for each phage.

The following recombinant phages have been created and the insertion site regions sequenced as described above:

Phages containing an inserted firefly luciferase:

LP48::ffluc (SEQ ID NO: 23);

LP99::ffluc (SEQ ID NO: 24);

LP101::ffluc (SEQ ID NO: 25);

LP124::ffluc (SEQ ID NO: 26);

LP125::ffluc (SEQ ID NO: 27);

LP143::ffluc (SEQ ID NO: 28);

AS11::ffluc (SEQ ID NO: 29); and

P100::ffluc (SEQ ID NO: 30).

Phages containing an inserted nanoluc luciferase:

LP124::nluc (SEQ ID NO: 31);

LP125::nluc (SEQ ID NO: 32);

AS11::nluc (SEQ ID NO: 33);

P100::nluc (SEQ ID NO: 34); and

LP40::nluc (SEQ ID NO: 35).

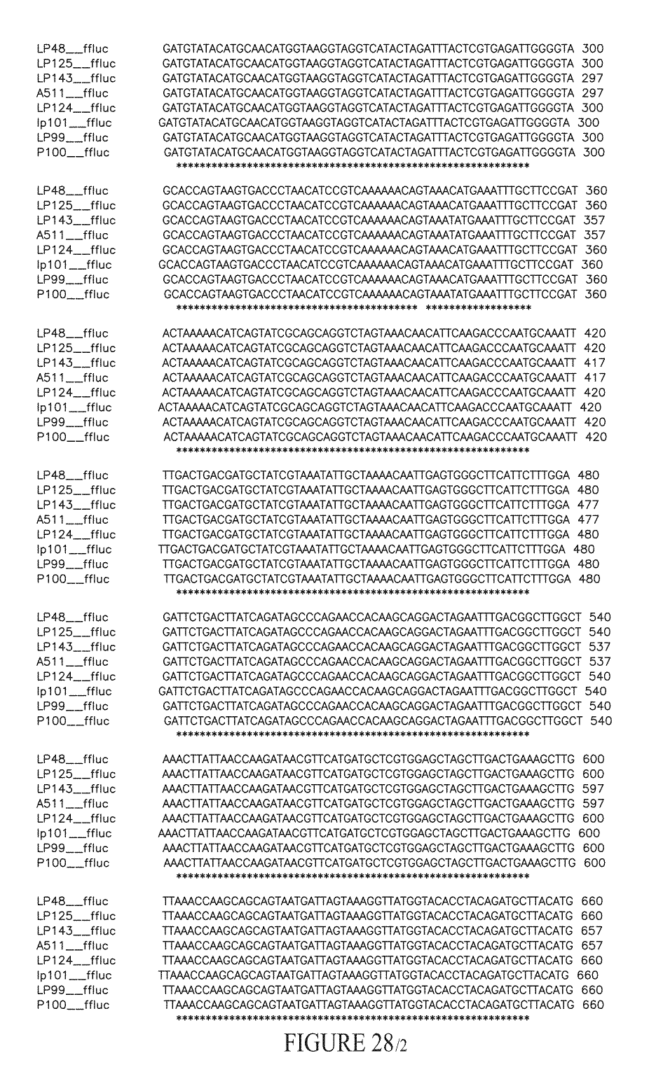

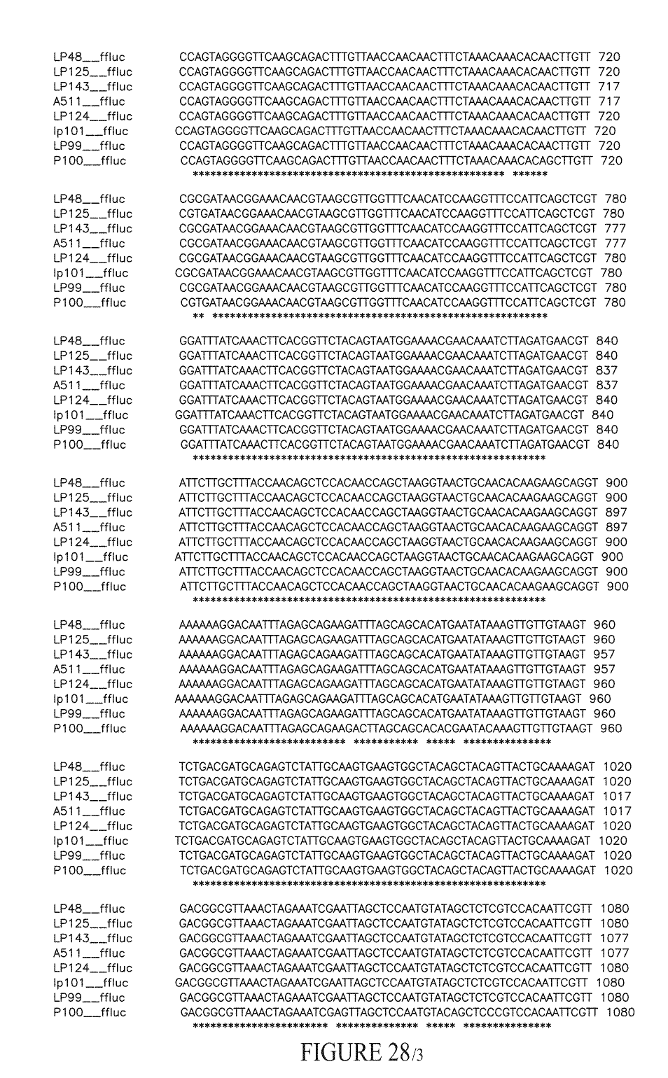

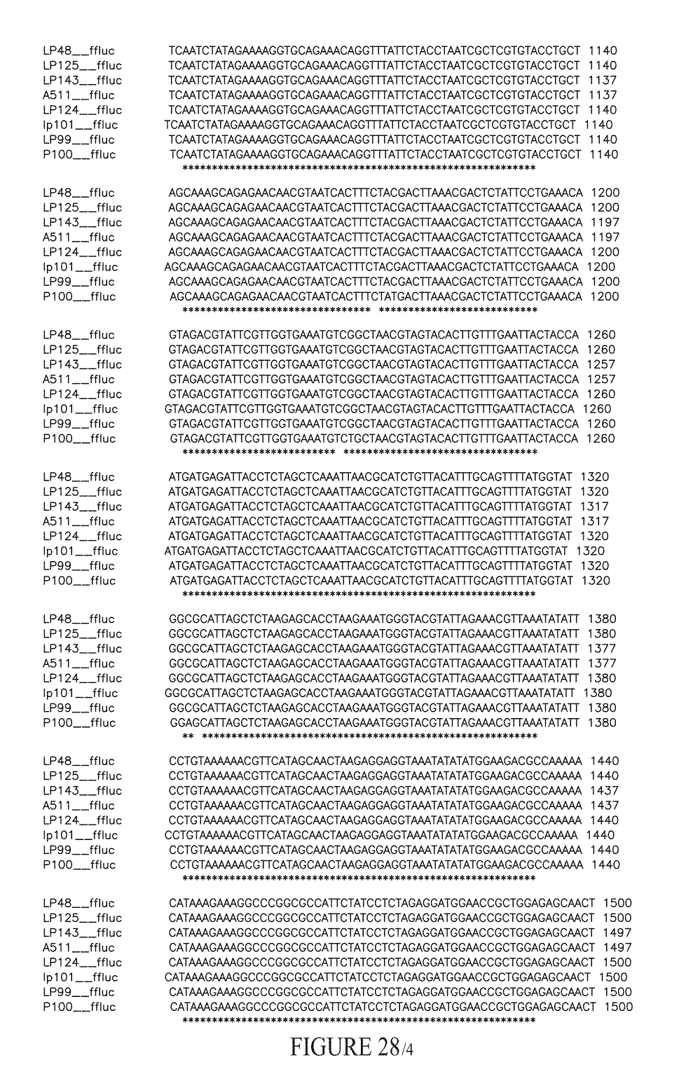

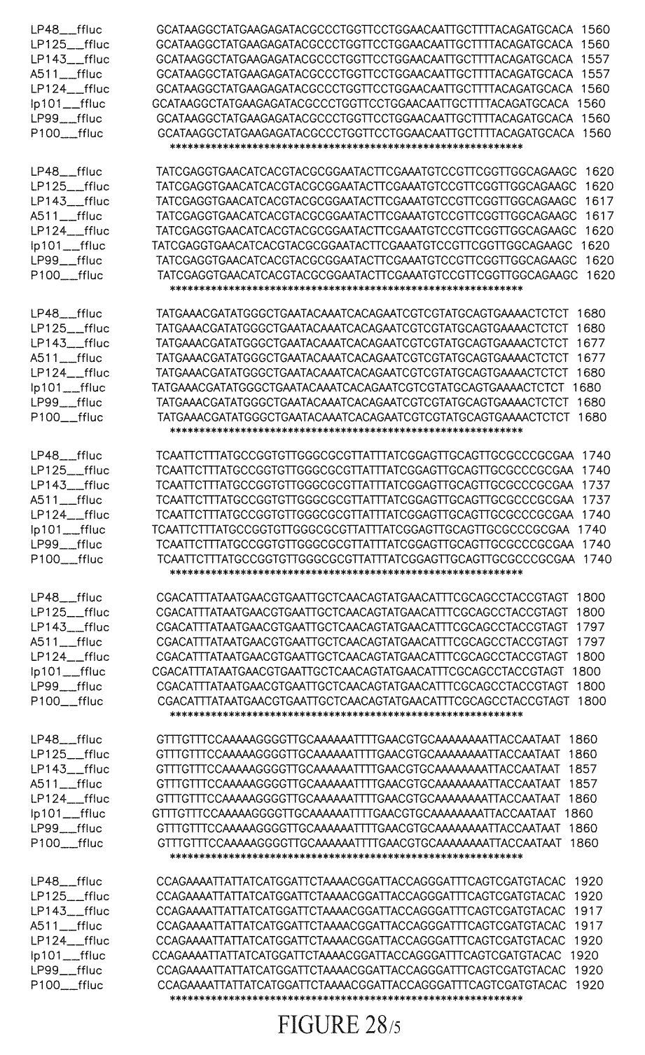

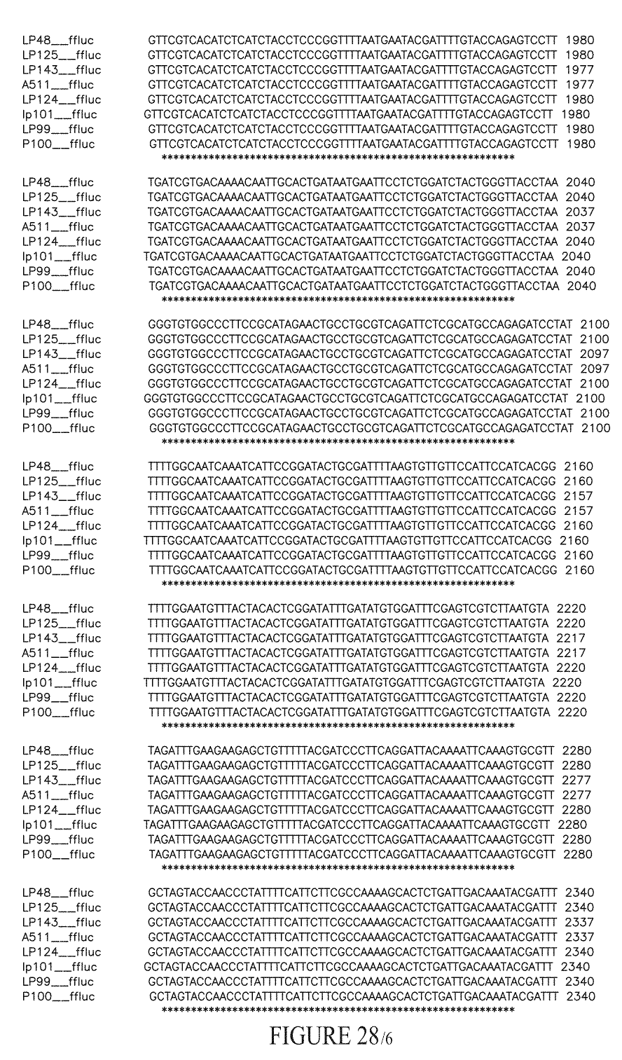

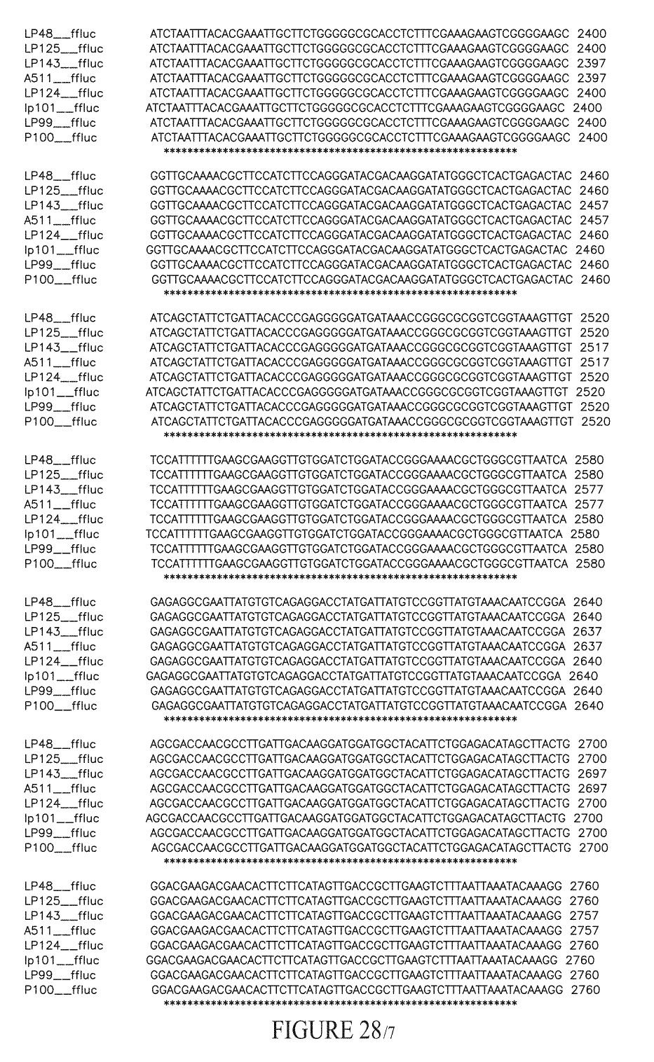

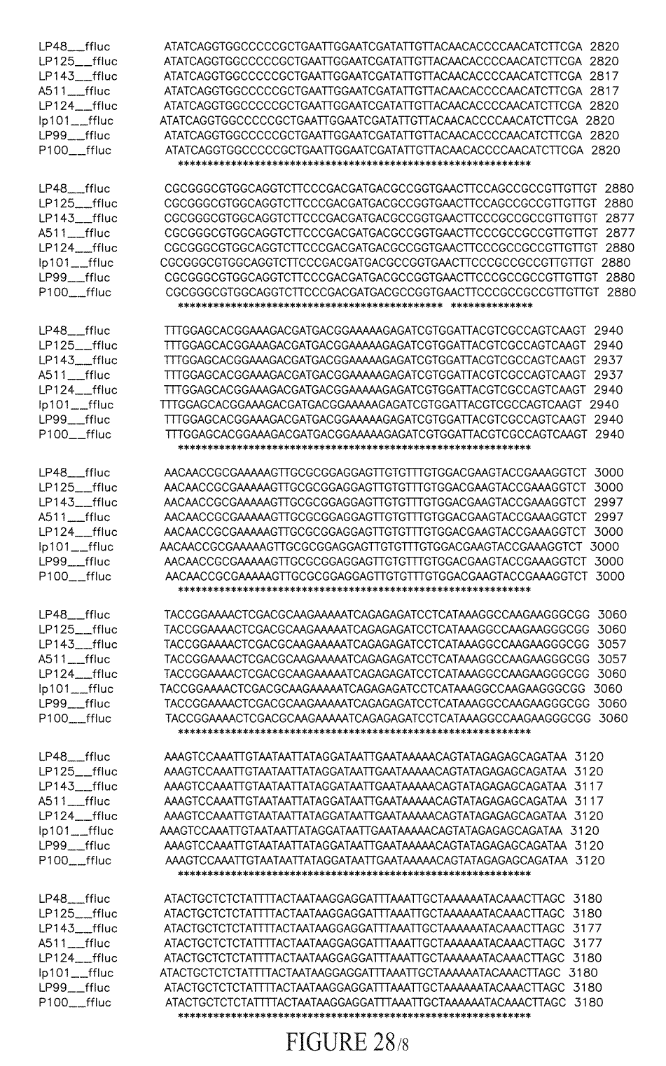

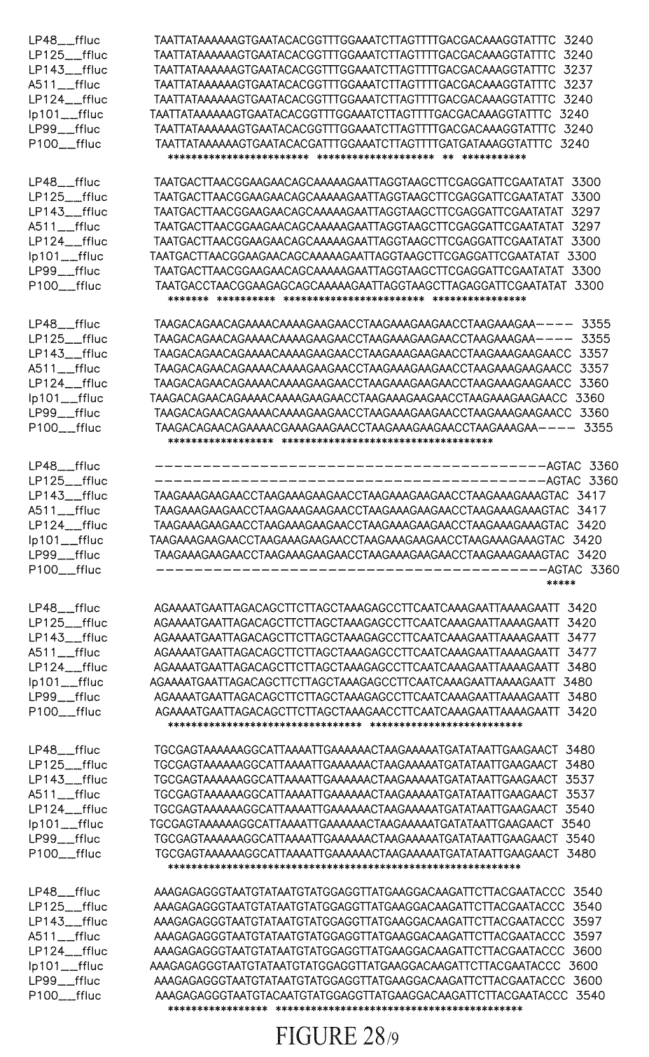

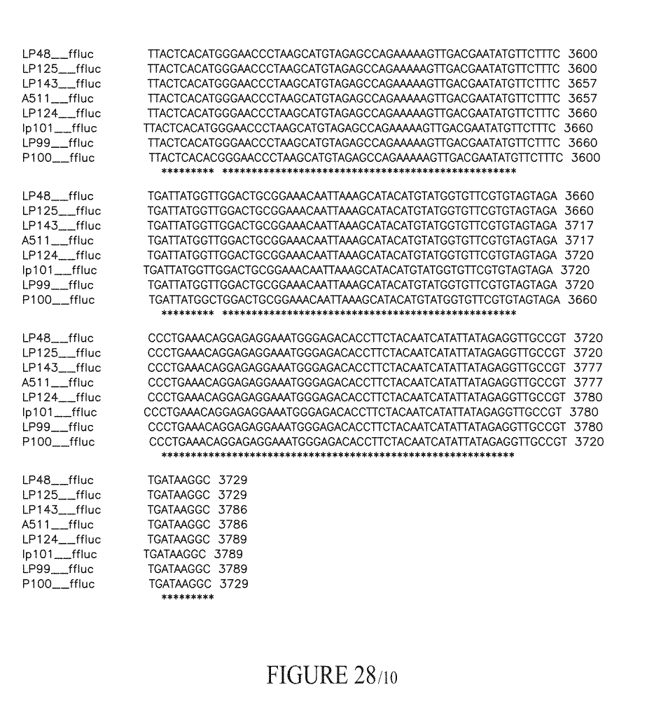

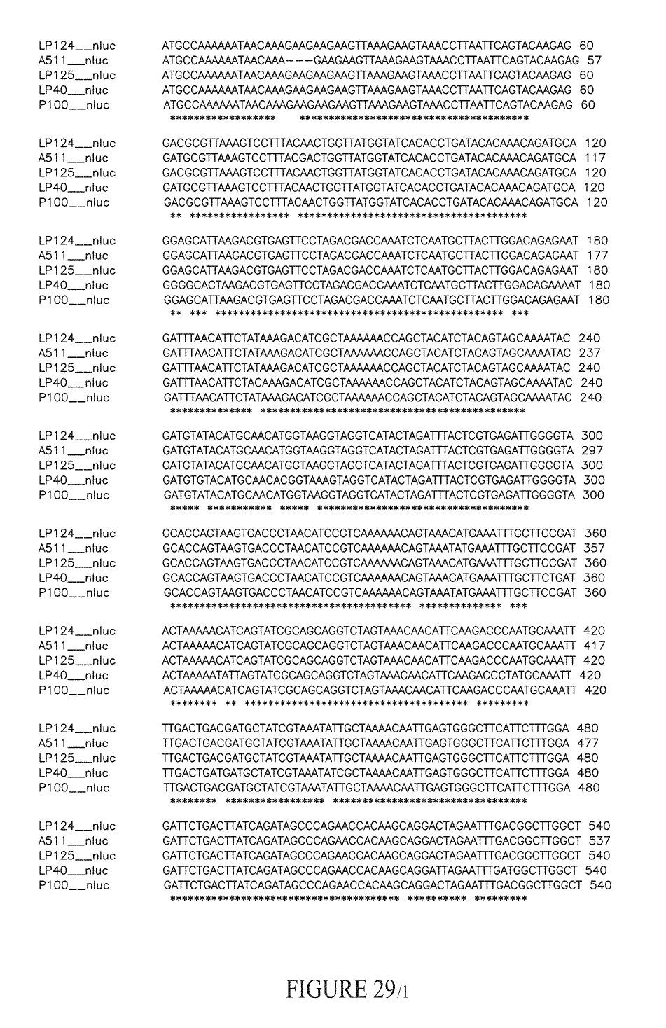

The insertion site regions of the phages comprising an inserted firefly luciferase coding sequence are aligned in FIG. 28. The insertion site regions of the phages comprising an inserted firefly luciferase coding sequence contain the following parts as indicated in Table 14.

TABLE 14

| | LP48 | LP99 | LP101 | LP124 | LP125 | LP143 | A511 | P100 |
|---|---|---|---|---|---|---|---|---|
| cps gene | 1-1407 | 1-1407 | 1-1407 | 1-1407 | 1-1407 | 1-1404 | 1-1404 | 1-1407 |
| RBS | 1408- | 1408- | 1408- | 1408- | 1408- | 1405- | 1405- | 1408- |
| (inserted) | 1423 | 1423 | 1423 | 1423 | 1423 | 1420 | 1420 | 1423 |
| Firefly | 1424- | 1424- | 1424- | 1424- | 1424- | 1421- | 1421- | 1424- |
| Luciferase | 3076 | 3076 | 3076 | 3076 | 3076 | 3073 | 3073 | 3076 |
| Downstream | 3077- | 3077- | 3077- | 3077- | 3077- | 3074- | 3074- | 3077- |
| genes | 3729 | 3789 | 3789 | 3789 | 3729 | 3786 | 3786 | 3729 |

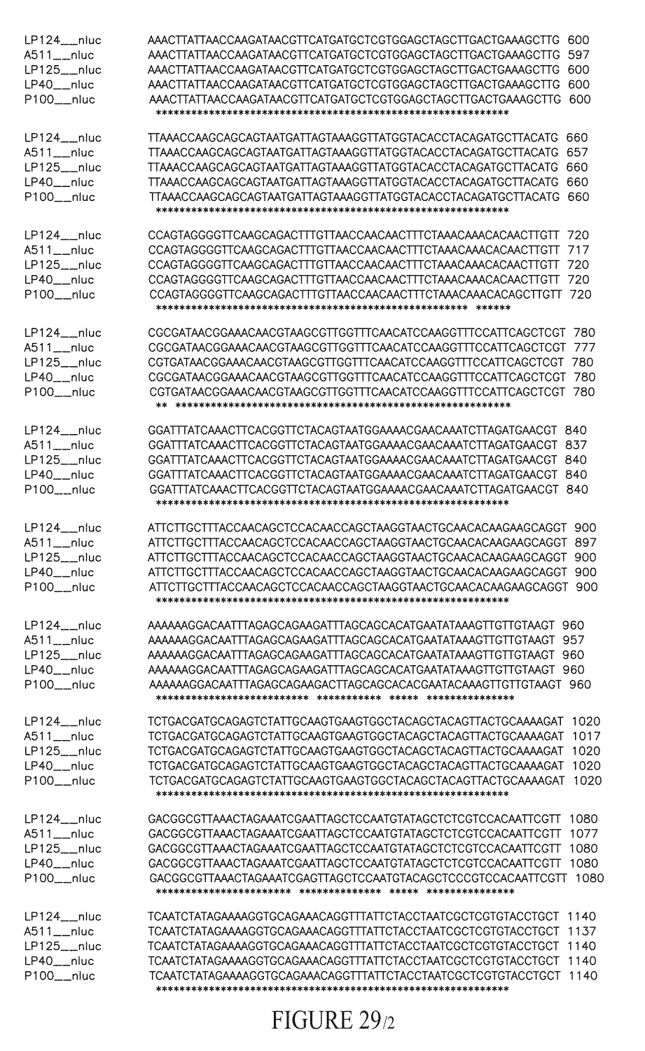

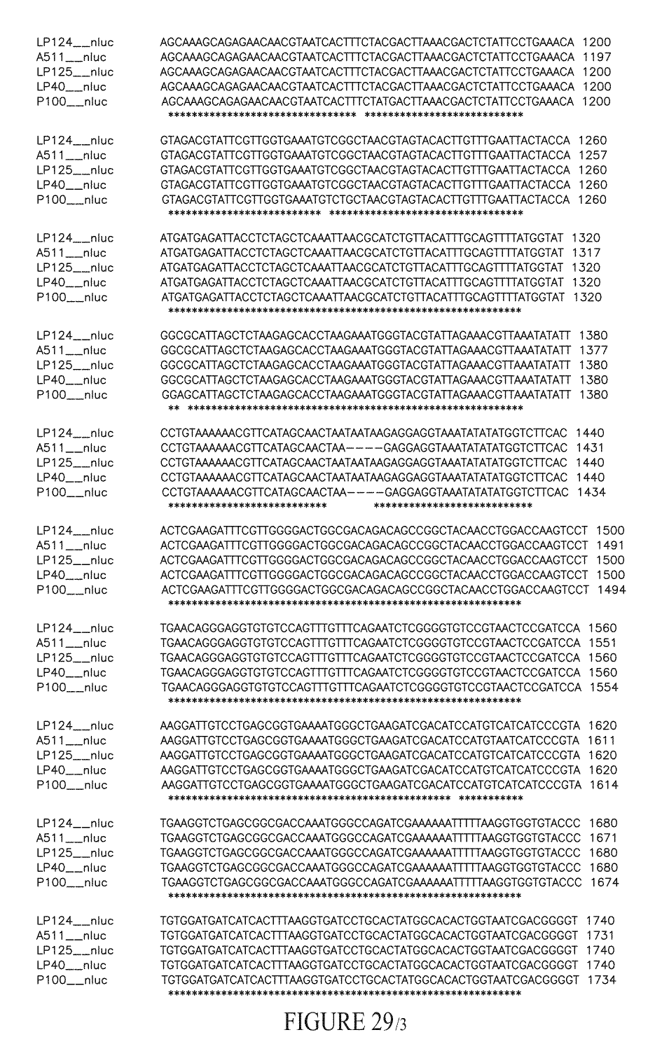

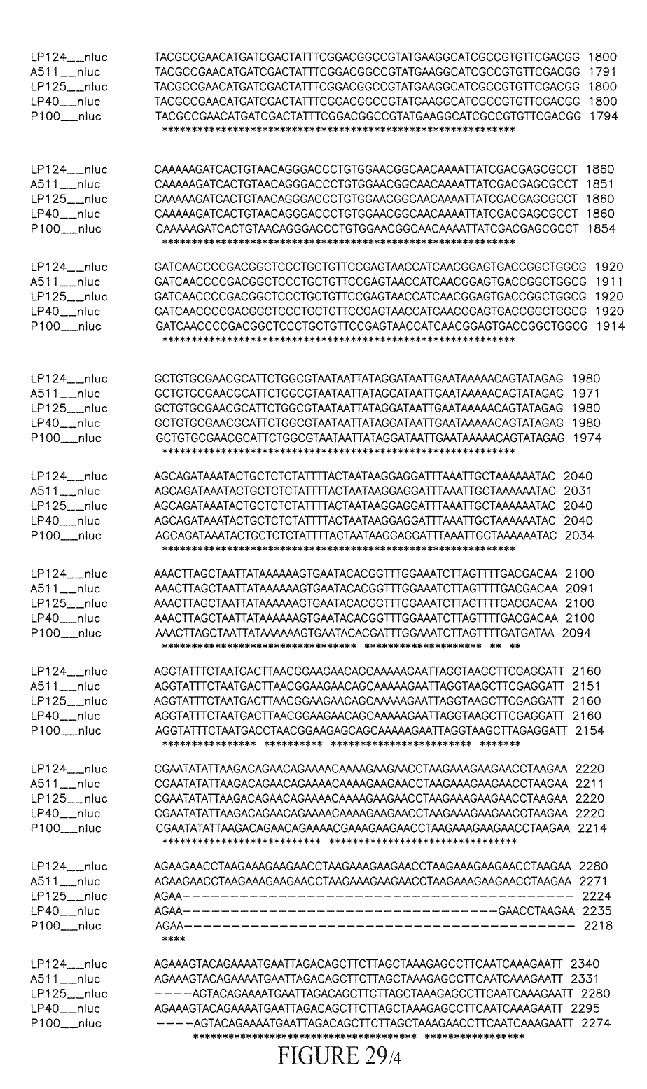

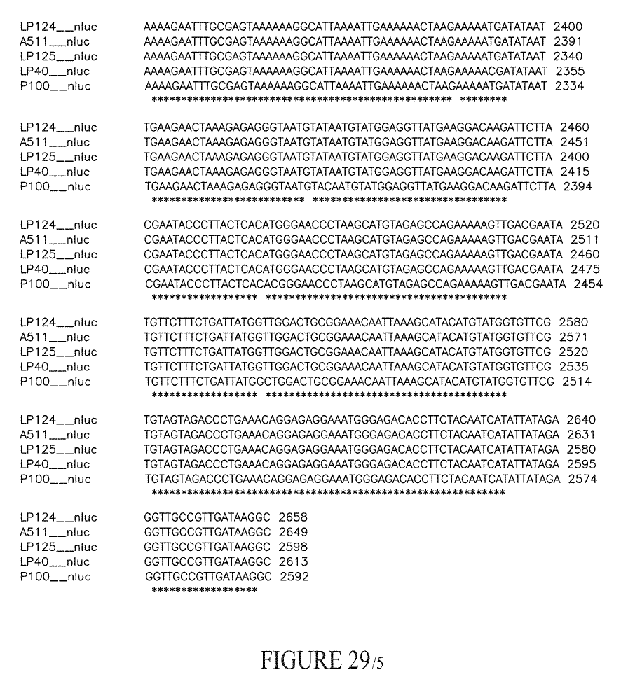

The insertion site regions of the phages comprising an inserted nanoluc luciferase coding sequence are aligned in FIG. 29. The insertion site regions of the phages comprising an inserted nanoluc luciferase coding sequence contain the following parts as indicated in Table 15.

TABLE 15

| | LP124::nluc | LP125::nluc | A511::nluc | P100::nluc | LP40::nluc |
|---|---|---|---|---|---|
| cps gene | 1-1407 | 1-1407 | 1-1404 | 1-1407 | 1-1407 |
| additional | 1408-1413 | 1408-1413 | n/a | n/a | 1408-1413 |
| stop codons | | | | | |
| (inserted) | | | | | |
| RBS | 1414-1429 | 1414-1429 | 1405-1420 | 1408-1423 | 1414-1429 |
| (inserted) | | | | | |
| NanoLuc | 1430-1945 | 1430-1945 | 1421-1936 | 1424-1939 | 1430-1945 |
| Downstream | 1946-2658 | 1946-2598 | 1937-2649 | 1940-2592 | 1946-2613 |
| genes | | | | | |

The cps open reading frames and encoded proteins for each phage are listed in Table 16.

TABLE 16

| Phage | Cps Gene Sequence | Cps Protein Sequence |
|---|---|---|
| LP40 | 5 | 6 |
| LP48 | 7 | 8 |
| LP99 | 9 | 10 |
| LP101 | 11 | 12 |
| LP124 | 13 | 14 |
| LP125 | 15 | 16 |
| LP143 | 17 | 18 |
| A511 | 19 | 20 |
| P100 | 21 | 22 |

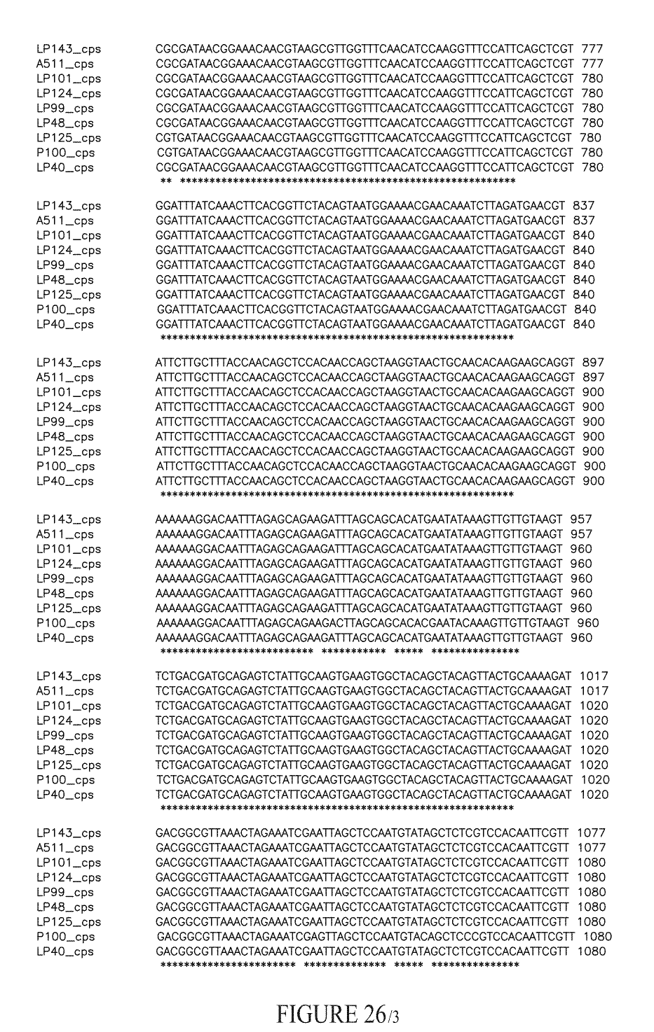

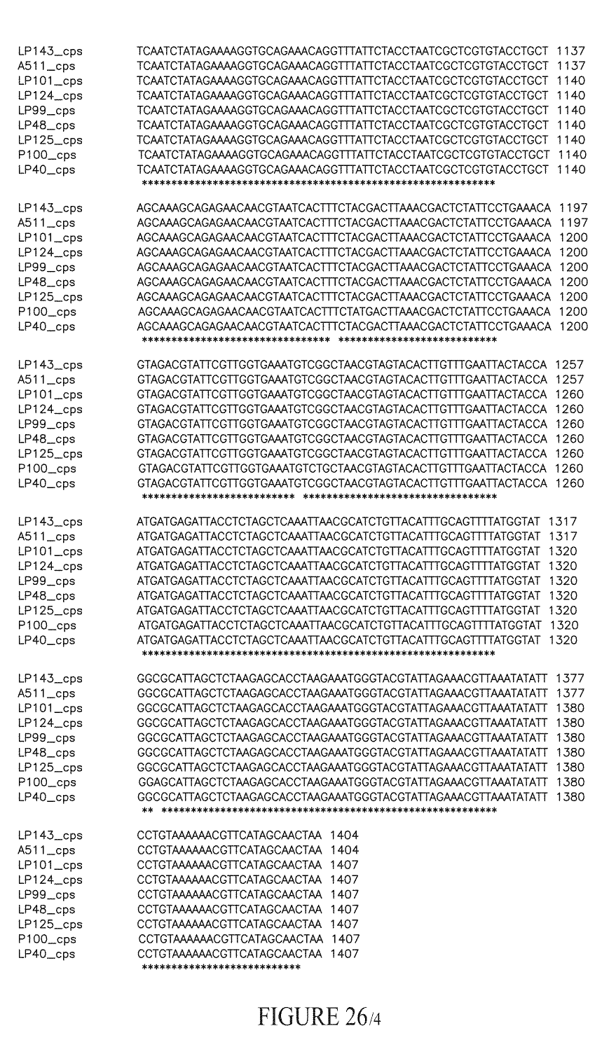

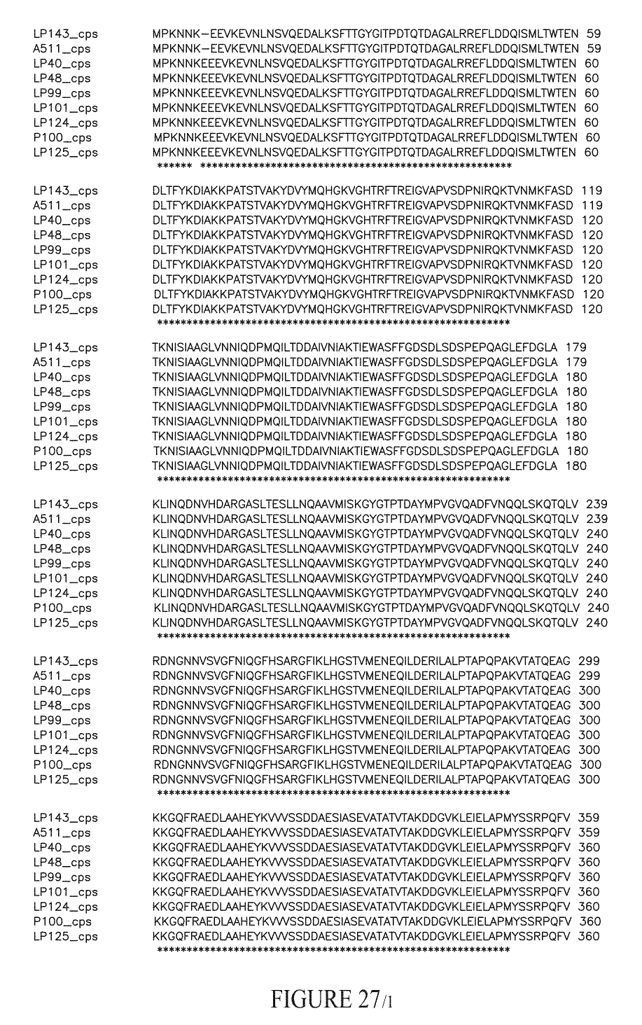

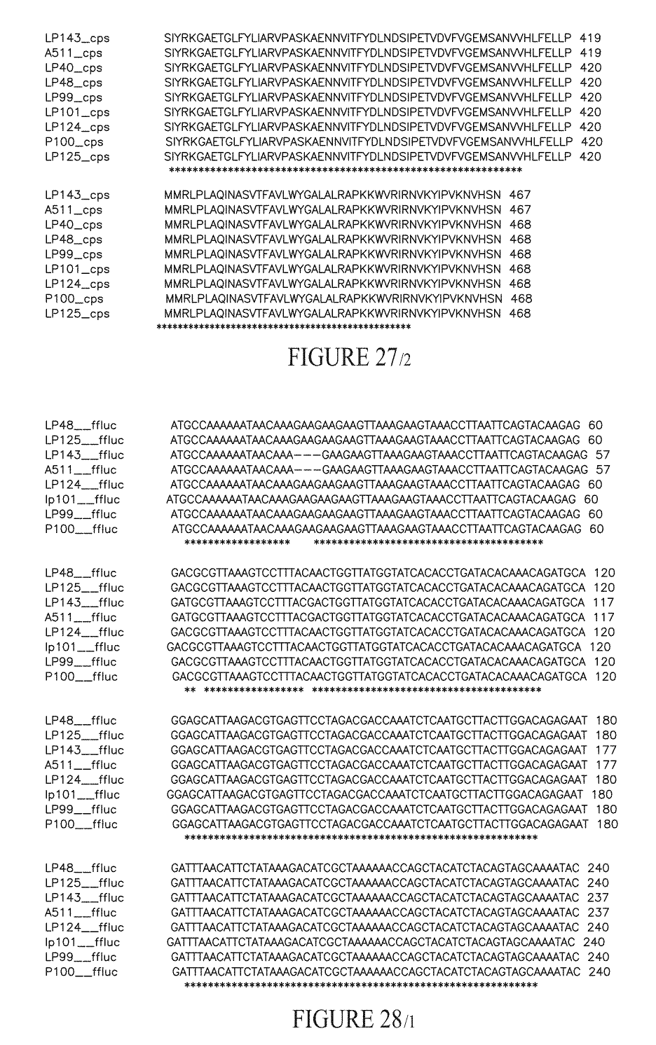

The cps gene sequences are aligned in FIG. 26 and the protein sequences in FIG. 27. The cps genes of the engineered phage display a relatively high degree of homology.

All of the above phages were engineered using the methods described above. Partial genome sequences showed that the primers used for A511 could be used to create PTVs for LP48, LP124, and LP125. No genome sequence was available at the time for LP99, LP101 or LP143. Using the A511 PTV primers, it was possible to amplify the appropriate fragments for PTV construction in the same manner as A511. This reflects homology between the cps gene regions across those phages. The luciferase gene insertion site was at the same location (after the cps gene stop codon TAA) as in A511::ffluc.

Engineering of HIS-tagged phages

To allow for the concentration of signal produced by the infection of listeria by recombinant phages, alternate versions of recombinant phage were produced that included a HIS tag. The 6×HIS tag (SEQ ID NO: 89) is a commonly used affinity tag for concentrating and purifying recombinant proteins.

HIS tags are commonly placed at the N-terminus or C-terminus of a protein, as it is often unknown a priori which location is optimal. Depending on the structure of the protein being tagged, as well as interactions with substrates, the tag sequence can interfere with, inhibit, or enhance enzyme function. For this reason phages were engineered with the HIS tag at either the N- or C-terminus.

Further, often times a spacer sequence comprising a small number of amino acid residues is place between the HIS tag and the gene being tagged. The size, charge, and other characteristics of this spacer can effect interactions with the enzyme, substrate, or HIS-binding beads/resins/antibodies. For this reason 2 different spacer were used between the HIS tag and the Nanoluc protein.

The HIS-tagged nanoluc versions of A511, LP124, and LP40 were constructed using the same methods as the untagged phages. The HIS tag and spacer were introduced during PTV construction by adding sequence to the oligos used to amplify the various DNA fragments. The oligos used in constructing the PTVs for A511, LP124 and LP40 are common to all 3 phages.

4 versions of each phage were constructed:

C-terminal long spacer

C-terminal short spacer

N-terminal long spacer

N-terminal short spacer

Oligos used to construct C-terminal long spacer PTV:

UHR fragment: pMAK upf and DBONO380

NLUC fragment: DBONO379 and DBONO400

DHR fragment: DBONO401 and DBONO382

Oligos used to construct C-terminal short spacer PTV:

UHR fragment: pMAK upf and DBONO380

NLUC fragment: DBON0379 and DBON0402

DHR fragment: DBONO401 and DBONO382

Oligos used to construct N-terminal long spacer PTV:

UHR fragment: pMAK upf and DBONO380

NLUC fragment: DBONO403 and DBONO358

DHR fragment: DBONO359 and DBONO382

Oligos used to construct N-terminal short spacer PTV:

UHR fragment: pMAK upf and DBONO380

NLUC fragment: DBONO404 and DBONO358

DHR fragment: DBONO359 and DBONO382

Once PTVs were constructed and verified, the rest of the PIE process was carried out as described above.

Oligo sequences:
DBONO400:
(SEQ ID NO: 90)
ATTCAATTATCCTATAATTATTAATGGTGATGGTGATGATGACCTCCAC
CTGCTGCCGCCAGAATGCGTTCGCACA
DBONO401:
(SEQ ID NO: 91)
ATCATCACCATCACCATTAATAATTATAGGATAATTGAATAAAAAC
DBONO402:
(SEQ ID NO: 92)
ATTCAATTATCCTATAATTATTAATGGTGATGGTGATGATGTGCTGCCG
CCAGAATGCGTTCGCACA
DBONO403:
(SEQ ID NO: 93)
TAATAAGAGGAGGTAAATATATATGCATCATCACCATCACCATGGTGGA
GGTGCAGCAGTCTTCACACTCGAAGATTTCG
DBONO404:
(SEQ ID NO: 94)
AGCAACTAATAATAAGAGGAGGTAAATATATATGCATCATCACCATCAC
CATGCAGCAGTCTTCACACTCGAAGATTTCG
HIS tag amino acid sequence:
(SEQ ID NO: 89)
HHHHHH
HIS tag DNA sequence:
(SEQ ID NO: 95)
CATCATCACCATCACCAT
C-terminal HIS with long spacer amino acid
sequence:
(SEQ ID NO: 96)
AAGGGHHHHHH
C-terminal HIS with long spacer DNA sequence:
(SEQ ID NO: 97)
GCAGCAGGTGGAGGTCATCATCACCATCACCAT
C-terminal HIS with short spacer amino acid
sequence:
(SEQ ID NO: 98)
AAHHHHHH
C-terminal HIS with short spacer DNA sequence:
(SEQ ID NO: 99)
GCAGCACATCATCACCATCACCAT
N-terminal HIS with long spacer amino acid
sequence:
(SEQ ID NO: 100)
HHHHHHGGGAA
N-terminal HIS with long spacer DNA sequence:
(SEQ ID NO: 101)
CATCATCACCATCACCATGGTGGAGGTGCAGCA
N-terminal HIS with short spacer amino acid
sequence:
(SEQ ID NO: 102)
HHHHHHAA
N-terminal HIS with short spacer DNA sequence:
(SEQ ID NO: 103)
CATCATCACCATCACCATGCAGCA

The insertion locations for each of the twelve tagged enzymes are provided in Table D. The numbering is the same as in the preceding tables in this example.

TABLE D

| Phage | Tag Location | Spacer | Inserted between bases |
|---|---|---|---|
| A511 | C-terminal | Long | 1933-1934 |
| LP124 | C-terminal | Long | 1941-1942 |
| LP40 | C-terminal | Long | 1941-1942 |
| A511 | C-terminal | Short | 1933-1934 |
| LP124 | C-terminal | Short | 1941-1942 |
| LP40 | C-terminal | Short | 1941-1942 |
| A511 | N-terminal | Long | 1423-1424 |
| LP124 | N-terminal | Long | 1432-1433 |
| LP40 | N-terminal | Long | 1432-1433 |
| A511 | N-terminal | Short | 1423-1424 |
| LP124 | N-terminal | Short | 1432-1433 |
| LP40 | N-terminal | Short | 1432-1433 |

The recombinant phage described in this example were deposited on May 16, 2013, with the American Type Culture Collection (ATCC®). The deposits were made under the terms of the Budapest Treaty on the International Recognition of the Deposit of Microorganisms for the Purposes of Patent Procedure. The ATCC® Patent Deposit Designations for the deposits are provided in Table E.

TABLE E

| | ATCC Patent Deposit |
|---|---|
| Phage | Designation |
| LP48::ffluc | PTA-120333 |
| LP125::ffluc | PTA-120334 |
| LP40::nluc | PTA-120335 |
| A511::nluc | PTA-120336 |
| P100::ffluc | PTA-120337 |
| LP124::nluc | PTA-120338 |
| LP101::ffluc | PTA-120339 |
| LP99::ffluc | PTA-120340 |
| LP143::ffluc | PTA-120341 |
| A511::ffluc | PTA-120342 |
| P100::nluc | PTA-120343 |
| LP124:ffluc | PTA-120344 |
| LP125::nluc | PTA-120345 |

### Example 20: Host Range Characterization of Combinations of Listeria Phages

After engineering the genome of the phages with two different genetic payloads, Firefly Luciferase and Nanoluciferase, the host range of these phages was retested to ensure that the genome modifications did not affect the fitness of the phages or compromise their ability to provide coverage across the Listeria strain host panel. Engineered phages were tested in the liquid-based host range assay and compared to non-modified bacteriophages. The engineered bacteriophages did not show a change in their host range compared to the non-modified wild-type versions (Tables 24-25).

The identification of bacteriophages that, when their individual host range profiles were combined, provided the necessary coverage of the Listeria genus raised the question of whether the phages when used in a combinatorial infection would provide the additive coverage expected or whether the presence of additional bacteriophages in an infection would diminish the ability of a single bacteriophage to infect a susceptible strain. To test this, combinations of bacteriophages (cocktails) were tested for the ability of a bacteriophage cocktail to provide clearance in the liquid-based host range assay. For these infections the final concentration of phage was maintained at a constant 1×10⁵ pfu consisting of equal amounts of each of the phage within the cocktail (i.e.—a two phage cocktail would consist of 5×10⁴ pfu of each of the two component phages). The combination of bacteriophages in a cocktail (either a two, three or four bacteriophage cocktail) did not cause a loss of host range and provided the expected additive effects of the host range of the individual bacteriophages (Tables 24-25). The additive effect of the bacteriophages was independent of the genomic modifications as neither the engineered Firefly luciferase and Nanoluc luciferase expressing bacteriophages had an altered liquid-based host range compared to the unengineered bacteriophages.

### Example 21: Comparison of Liquid-Based Host Range Versus Marker-Based Host Range

The ability of a bacteriophage to clear an actively growing culture is determined by a number of factors including the rate of growth of a particular strain and the rate of bacteriophage replication, in addition to the ability of the bacteriophage to infect a specific strain. Therefore, the output of culture clearance measure used in the liquid culture method disclosed herein is potentially more restrictive than the host range that could be determined by exposing bacterial strains to an recombinant phage comprising a heterologous nucleic acid sequence encoding a marker and assaying for marker production. One example of such a marker is luciferase. Therefore, the host range was determined for phage LP124:nluc by both the liquid-based host range assay and by an infection based luciferase detection assay. To carry out the infection based assay, the Listeria host panel strain collection was struck out on Brain Heart Infusion (BHI) agar plates and single colonies were inoculated in 1 ml BHI liquid in a 2-ml 96-deep well dish, covered with a sterile breathable sterile membrane and grown at 30 C for 16 hours. Each of the 16-hour cultures from the 96-well plates were diluted 1:10,000 in 198 μl of BHI. For the infection, 12.5 μl of the culture dilution were mixed added to 12.5 μl of LP124:nluc at a concentration of 1×10⁷ pfu/ml in an opaque luminescence reader plate and incubated at 30 C for 3 hours. After three hours the level of luminescence was detected using a Promega Glomax 96-well plate reader using Promega NanoGlo reaction following manufacturer's recommendations.

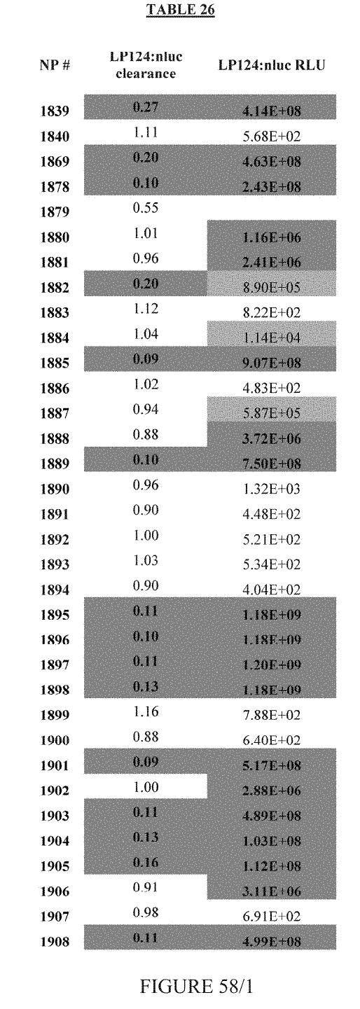

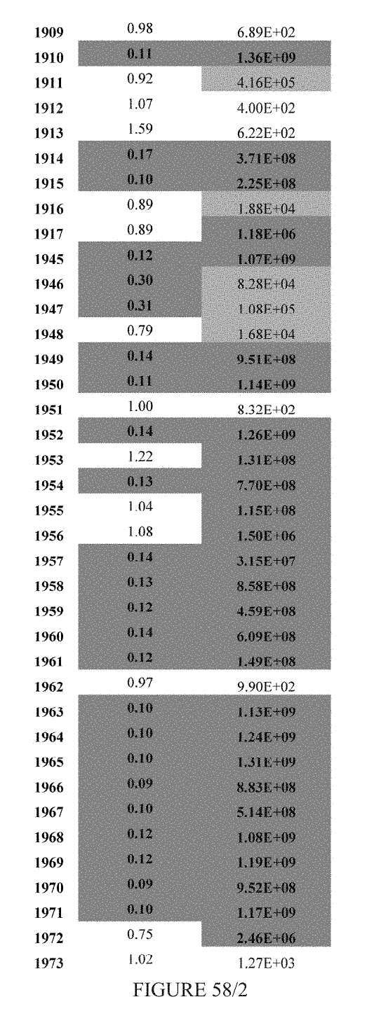

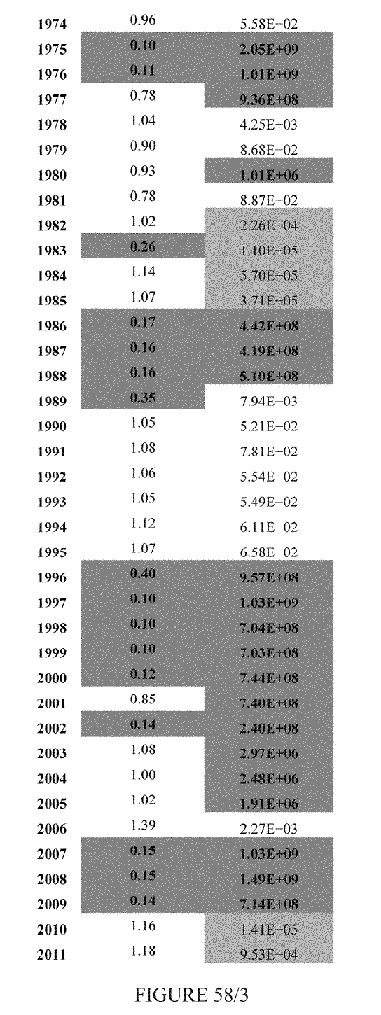

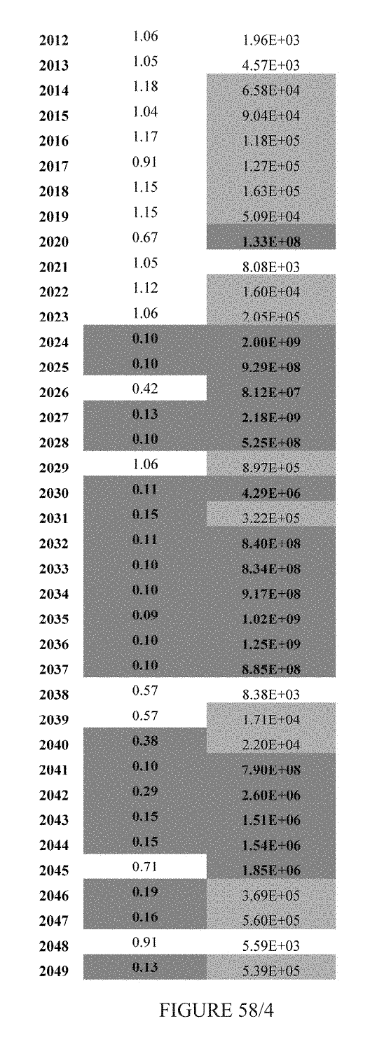

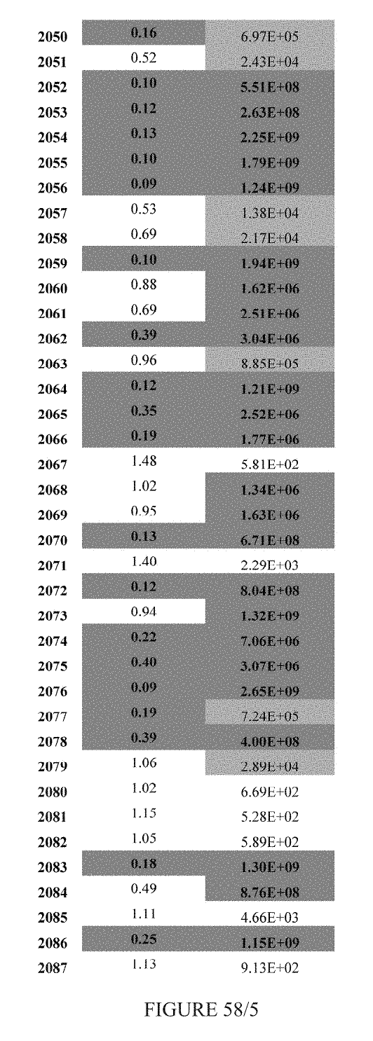

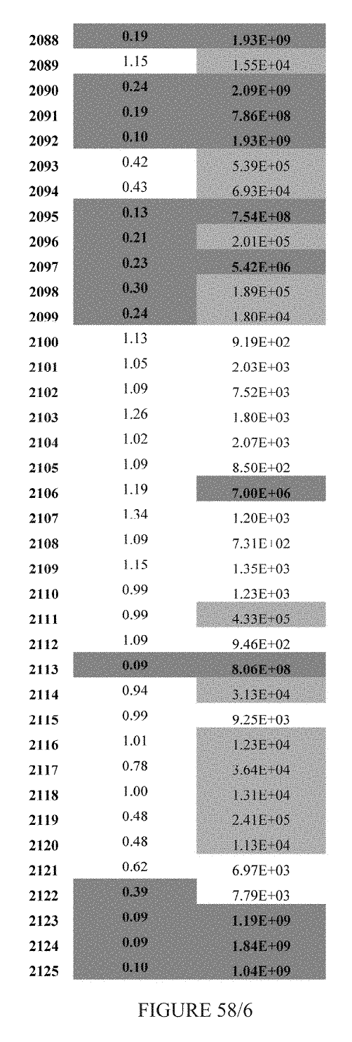

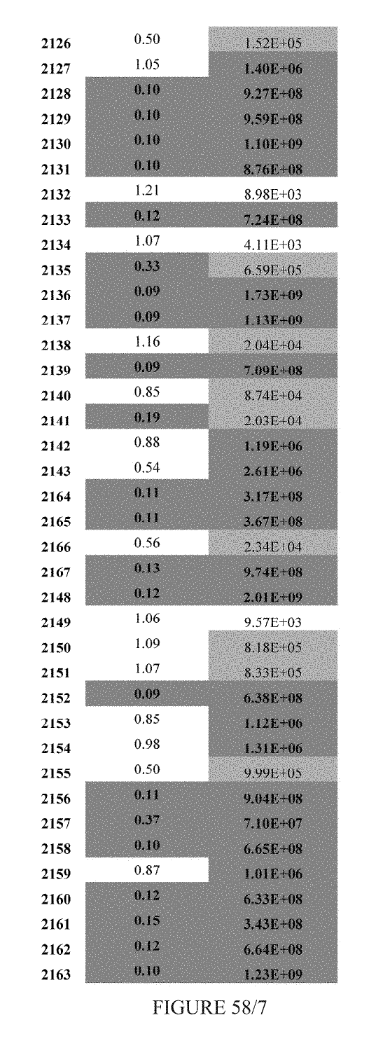

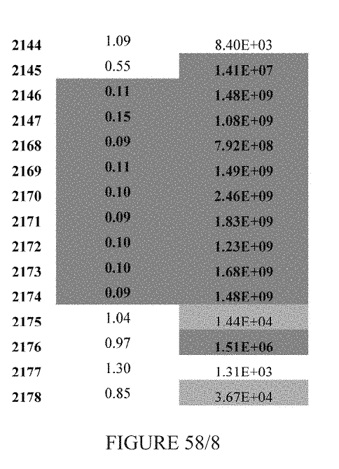

Table 5 shows the host range determined by the two methods. A strain was considered to be within host range for the clearance assay if the ratio of infected culture OD600 to the uninfected culture OD600 was less than 0.4. For the luciferase detection-based host range assay strains were stratified in three categories, high RLU strains (FIG. 58 (Table 26), dark gray shading,), medium RLU strains (FIG. 58 (Table 26), light gray shading), and low RLU strains (FIG. 58 (Table 26), unshaded). Based on the performance of the assay a strain was considered to be within the host range of the bacteriophage if the RLU measurement was greater than 10,000 Random Light Units (RLU) (FIG. 58 (Table 26), light gray shading). This luciferase activity cut-off was used because it characterizes a useful level of sensitivity in bacterial assays. Based on these criteria the liquid-based host range clearance, LP124 shows a broad host range by clearing 50.5% (140 of 276) of the Listeria strains tested. By the luciferase detection assay, 78.2% (216 of 276) of the Listeria strains tested showed high RLU levels.

The comparison between the ability of LP124::nluc to clear cultures of the Listeria host-panel to the RLU output shows that the host range measured using marker expression is greater than that defined using the liquid-based host range. This could be for several reasons. First, a bacterial strain that is not cleared by the infection but that produces light may have a growth rate that outpaces the ability of the bacteriophage to infect and replicate. In this case, the strain would never succumb completely to bacteriophage because the number of uninfected cells would outpace the bacteriophage in the culture. Second, the bacteriophage may be able to carry out the initial steps of infection (i.e. attachment, injection of DNA and translation of viral proteins) but be unable to complete the infection process (i.e. virion assembly, release from the cell). Because the bacteriophage life-cycle can be separated into discrete steps, a bacteriophage is capable to produce phage encoded proteins, in this case luciferase, without clearance of the culture or producing additional bacteriophage. While additional strains that produce luciferase without producing bacteriophage would not fall within the classical definition of host range for a bacteriophage, the strains do meet inclusion in the host range definition for the purpose of this disclosure because the host range that matters in methods of detecting target bacteria using a phage comprising a heterologous nucleic acid sequence encoding a marker is the types of bacteria that support marker production. This increased host-range observed when using the engineered bacteriophage is an advantageous byproduct of the engineering process and could not be determined a priori for the Listeria host panel.

One possible concern raised by the ability of a bacteriophage to produce light in a bacterial strain that it could not clear from a liquid-based culture is that other off-target bacterial genera may also produce luciferase in the presence of engineered phages. These bacterial species would not have been considered to be in host range of these phages because of an inability to produce bacteriophage in response to bacteriophage infections. However, the increased sensitivity for detecting early stages of infection with the engineered phages could, at least theoretically, result in production of marker (in this case luciferase-assayed by light production) in strains of bacteria not identified as hosts using the liquid culture method, for example. To address this issue, a panel of bacterial species closely related to Listeria was assembled (Table 27). This panel consisted of other Gram-positive organisms phylogenetically similar to Listeria. To determine if these strains were able to produce light in the presence of the engineered bacteriophage each of the species were grown for 16 hours under appropriate growth conditions (Table 27). The strains were diluted to a concentration of 10⁵ cfu/ml and then 90 μl of cells were mixed with 10 μl of a bacteriophage cocktail at 1×10⁷ pfu/ml and incubated for 3 hours at 30 C. The reactions were then measured for the presence of luciferase using the standard protocol. None of the bacterial species tested had detectable levels of RLU (Table 27) demonstrating that the ability of the bacteriophages to show RLUs in strains that they do not clear is not a strictly off-target effect that will decrease the accuracy of a bacteriophage reporter based assay.

A second question was whether these bacteria species that were phylogenetically similar to Listeria would decrease the sensitivity of the engineered bacteriophages to detect Listeria when the Listeria and non-Listeria bacteria species were present together in an assay. To examine this possibility the related bacterial species were grown as above and diluted to a concentration of 10⁵ cfu/ml. A Listeria strain was struck out on Brain Heart Infusion (BHI) agar plates and single colonies were inoculated in 5 ml BHI liquid and grown at 30 C for 16 hours. The overnight culture was diluter 1:5 in fresh 0.5×BHI medium and grown for 2 hours at 30 C shaking at 200 rpm in an orbital shaker. After two hours a 10-fold serial dilution of the culture was made. To perform the test 10 μl of the Listeria serial dilution that should represent ˜10 cfu total was mixed with 20 μl of the potentially inhibitory bacterial species and 10 μl of the bacteriophage cocktail (A511::nluc/LP124::nluc/P100::nluc) and the mixture was incubated for 3 hours at 30 C. After the incubation the reaction was assayed for the presence of luciferase using the Promega Glomax 20/20 luminometer and Promega NanoGlo reaction as suggested. These assays showed that there was no decrease in the ability to detect Listeria in the presence of 10⁴ greater numbers of competing bacteria (Table 27) demonstrating the sensitivity of the assay is not affected by the presence of non-target bacteria in samples.

This selection of bacteria was a limited set and did not represent all of the bacteria that could be present during environmental sampling. To generate a more exhaustive sample of bacterial species that may decrease the sensitivity and accuracy of the bacteriophage cocktail, environmental samples were collected from food processing plants and bacterial species were isolated from environmental swabs to determine the effect of these species on performance of the assay. To isolate bacterial species that were present, environmental samples were plated onto both Brain Heart Infusion Agar or R2A agar and grown overnight at 30 C. Bacteria that were present on the plates were identified based on colony morphology and struck to purity on BHI agar plates. Pure cultures of the bacterial species were grown in BHI medium at 30 C for 16 hours. The cultures were diluted to a concentration of 10⁵ cfu/ml and tested for both the production of luciferase in the presence of the bacteriophage cocktail and inhibition of Listeria infection by the bacteriophage cocktail as above. None of the bacterial species, consisting of both Gram-positive and Gram-negative bacteria, showed any luciferase production in the presence of the bacteriophage (Table 28). Additionally, incubation of Listeria in the presence of the collected samples failed to show any decrease in the production of luciferase, demonstrating that the environmentally collected bacteria do not decrease the sensitivity or accuracy of the assay.

### Example 22: Design of Phage Compositions

The increased host range observed by the RLU-based luciferase detection assay compared to the liquid-based host range assay identified a novel method for distinguishing differences between the host range of bacteriophages. Additionally, the RLU-based luciferase detection assay as a means to assess phage host range allows for a highly accurate assessment of the target bacteria identified by an engineered bacteriophage under conditions similar to those of methods of detecting target bacteria. One way this information may be used is to identify useful combinations of phage that can be combined to make a combination of phage having a useful cumulative host range.

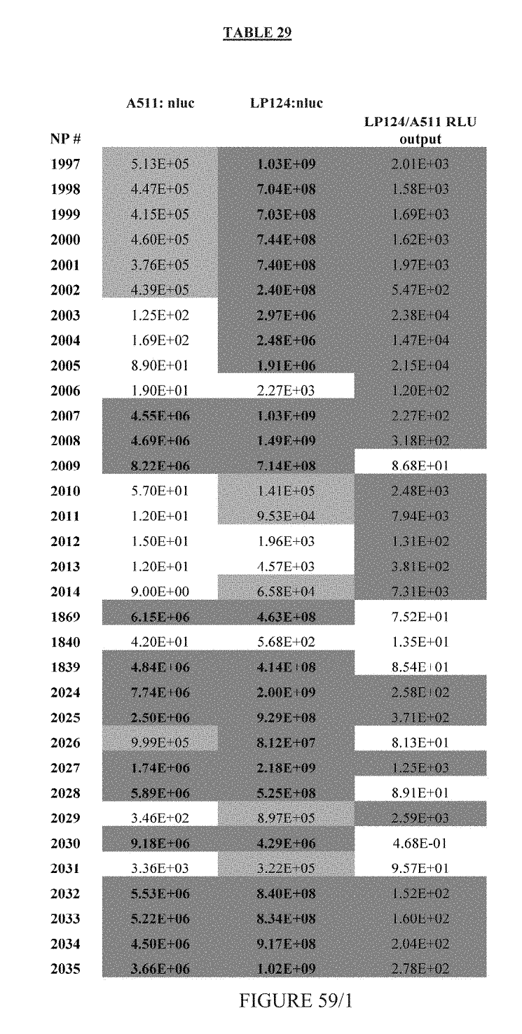

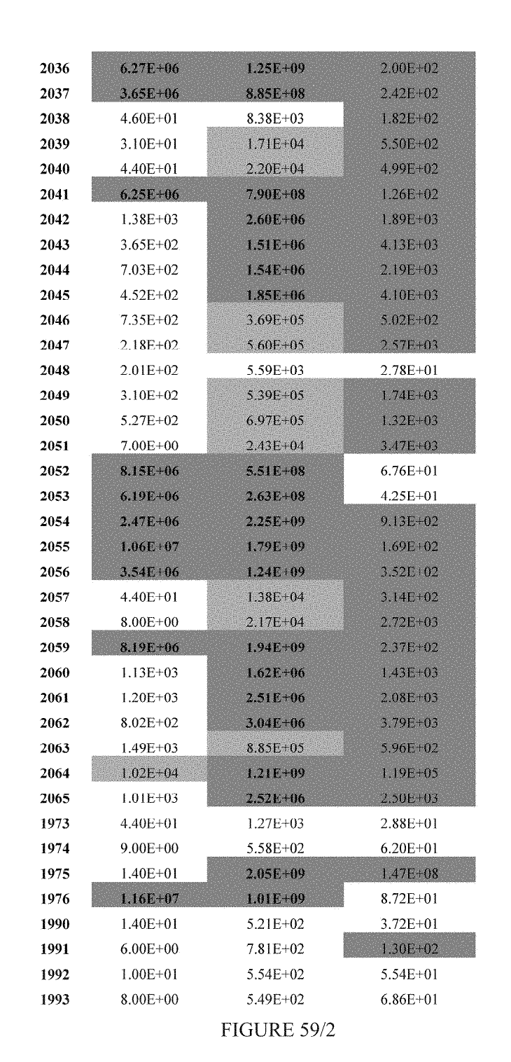

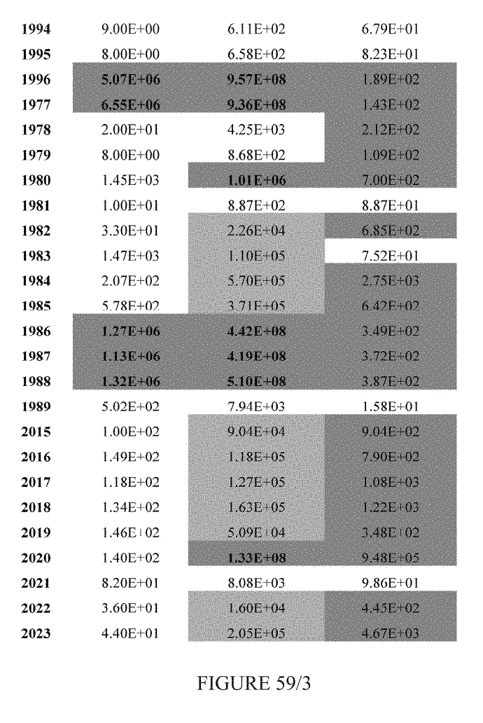

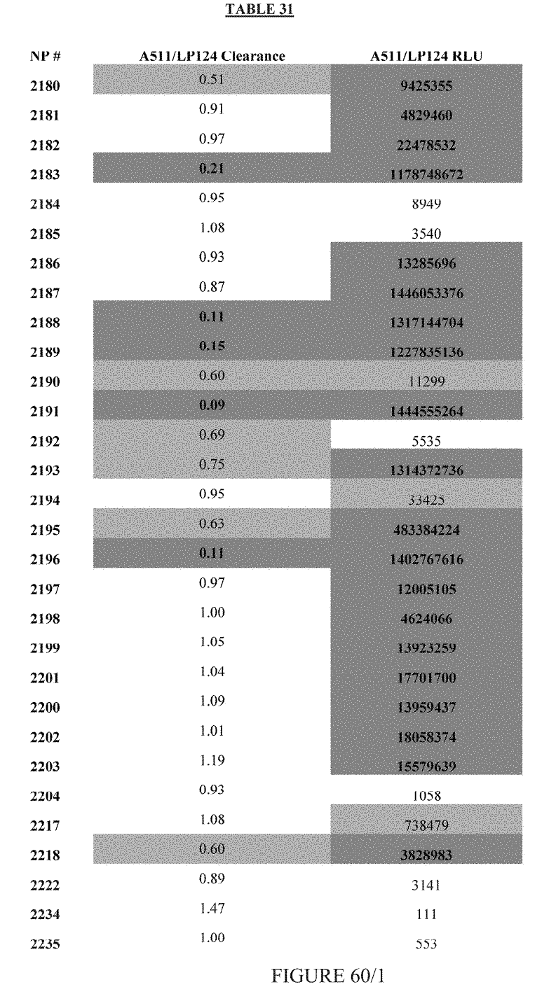

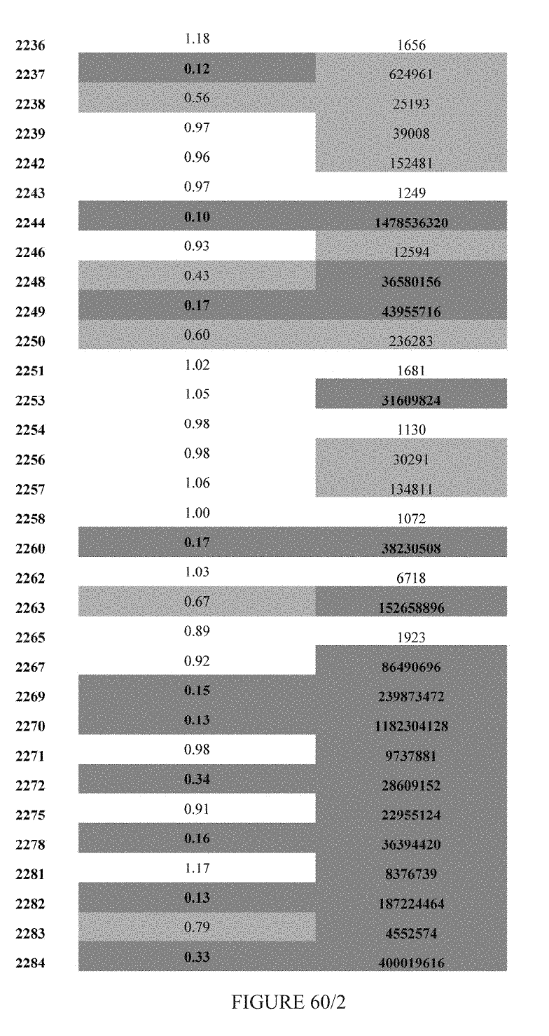

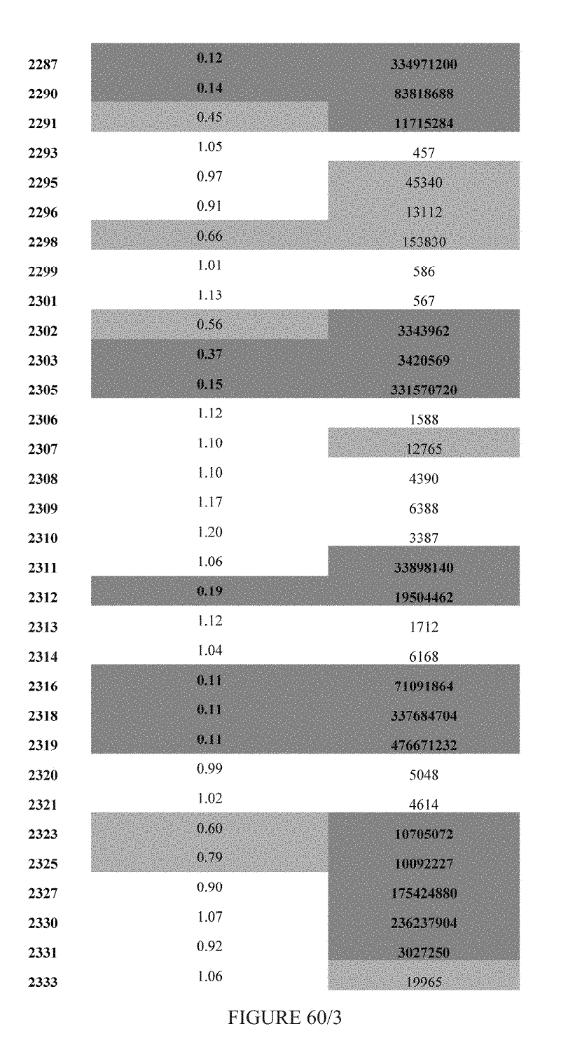

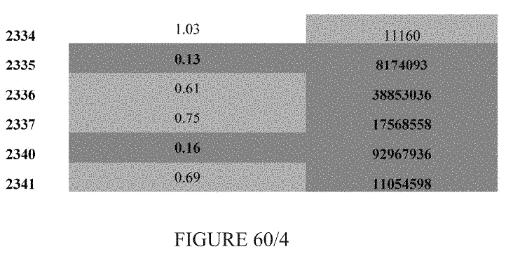

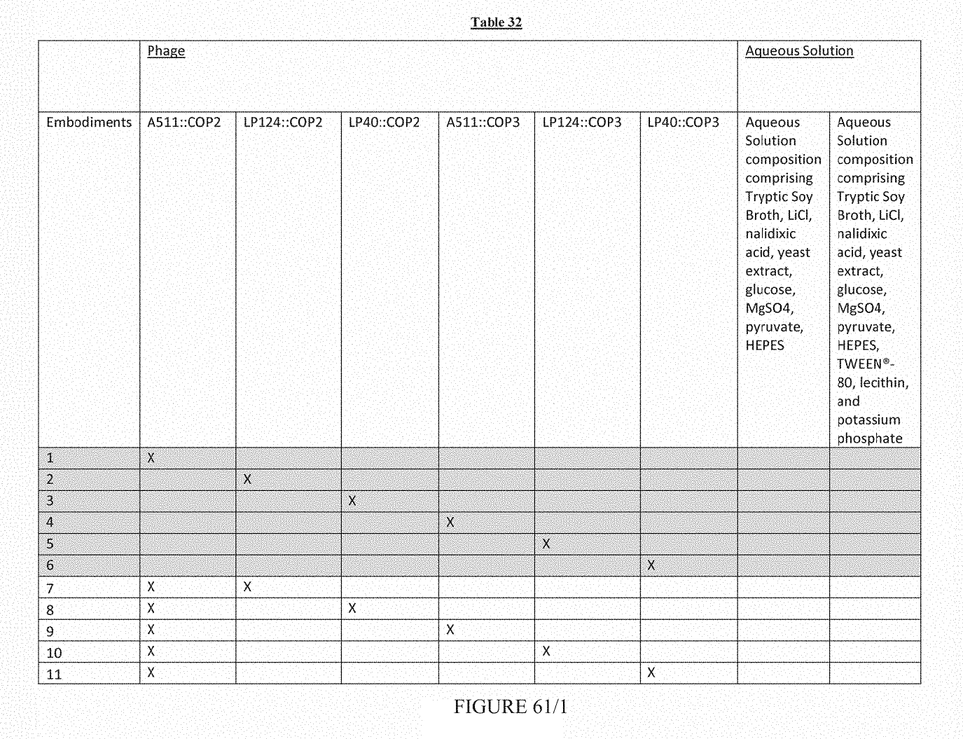

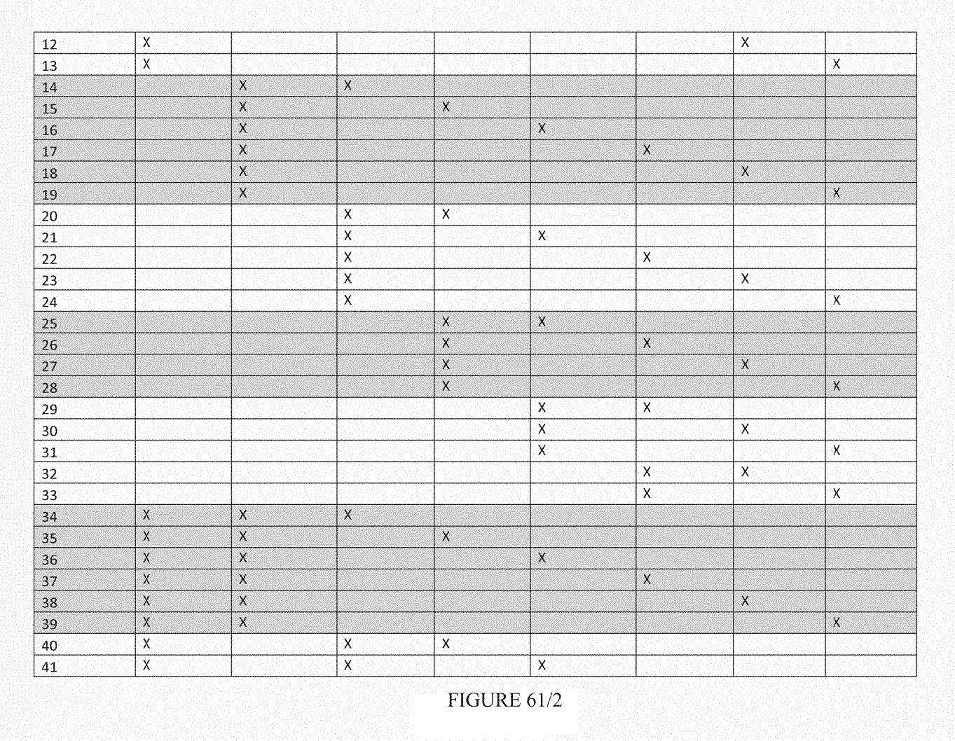

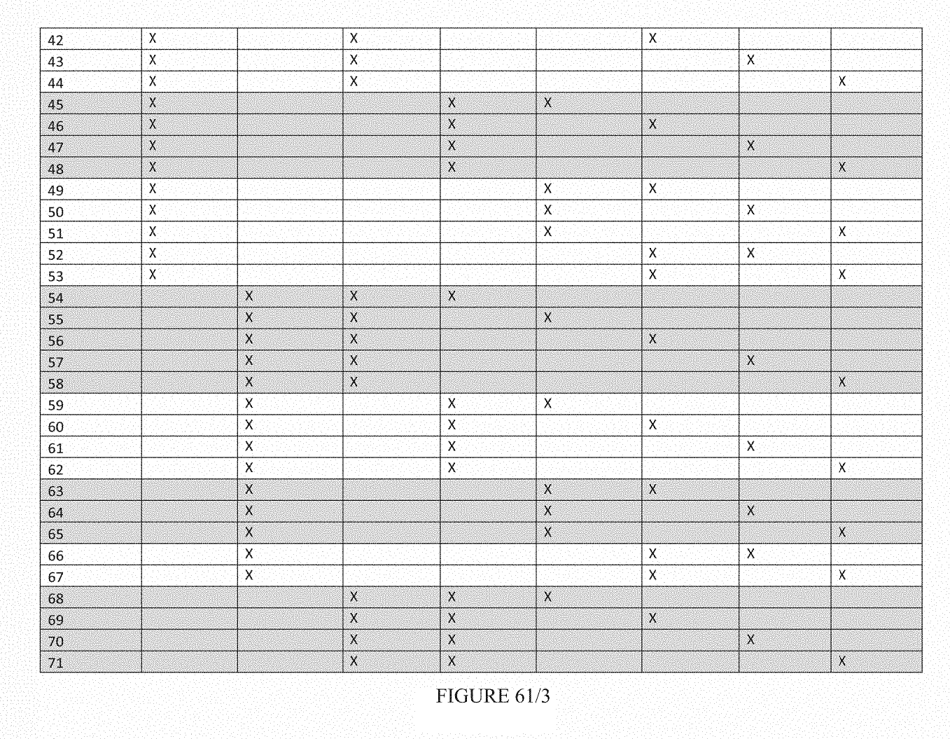

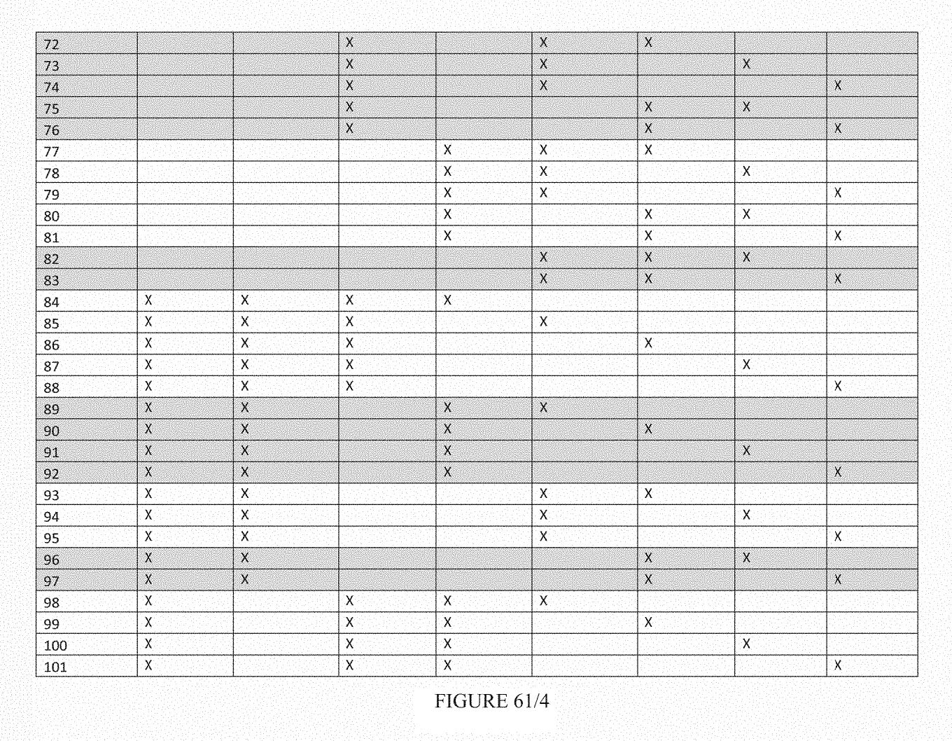

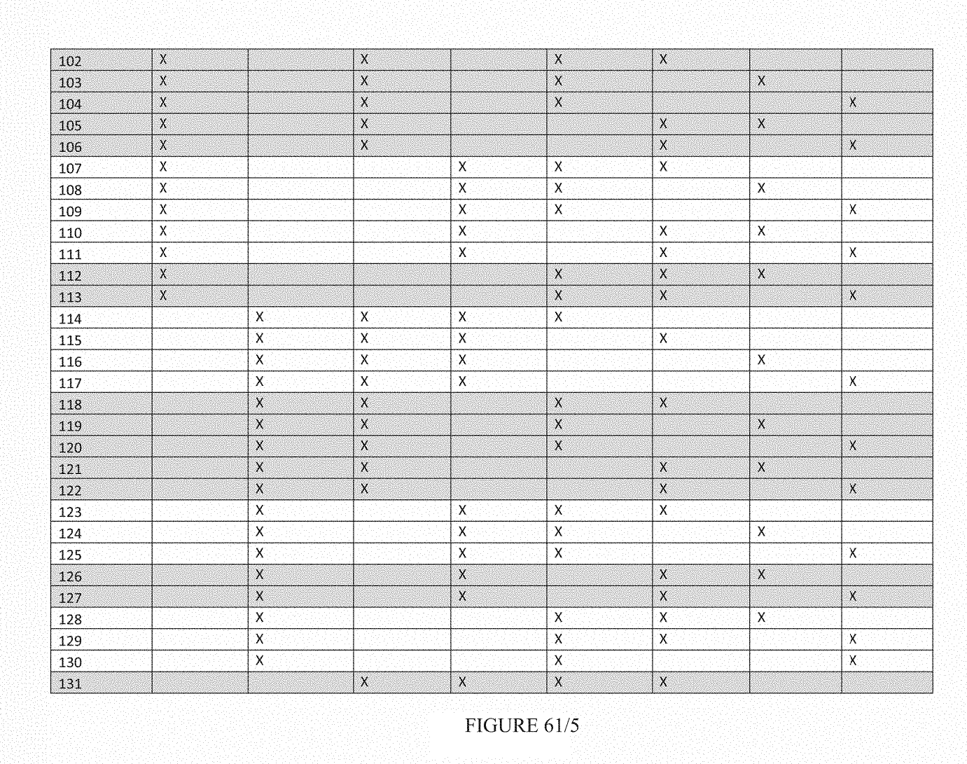

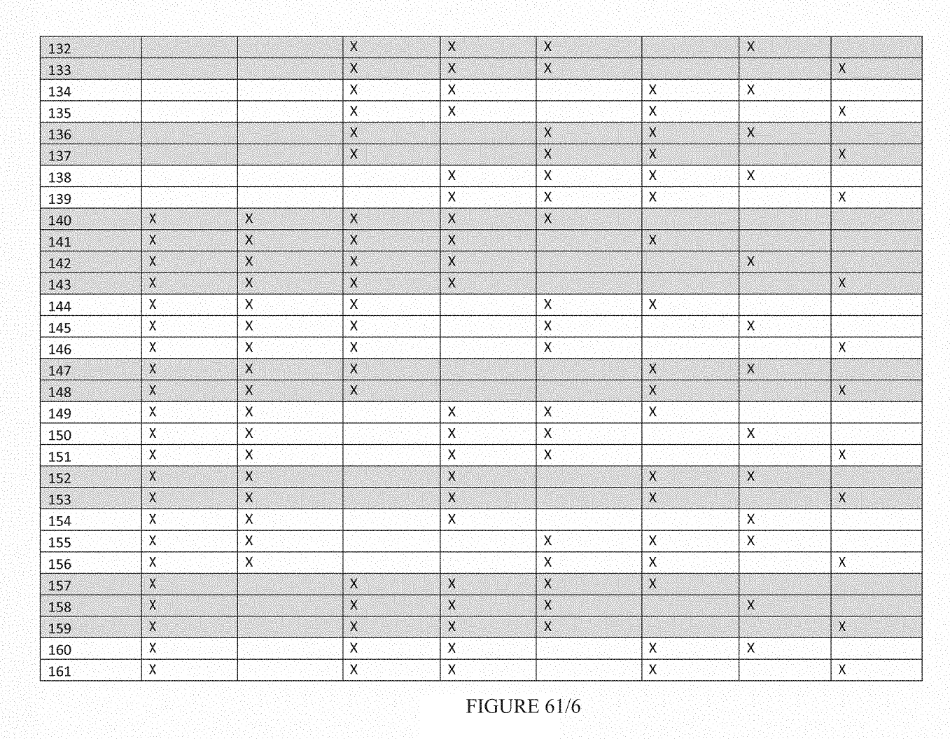

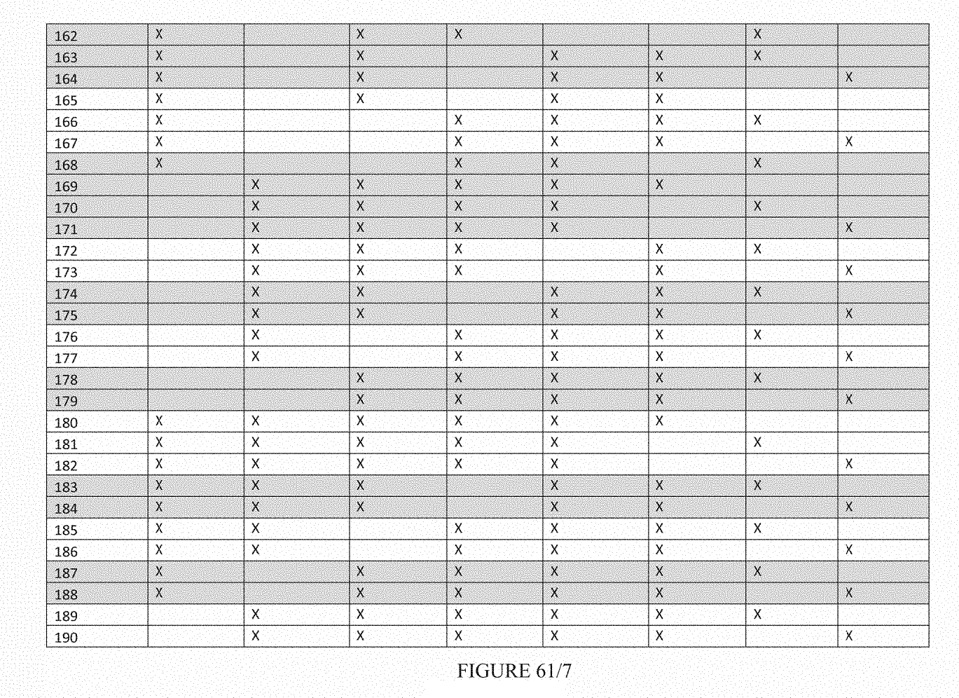

To determine the additive effect of including LP124::nluc in a bacteriophage cocktail a RLU-based luciferase detection assay was compared between AS11::nluc and LP124::nluc for a portion of the Listeria host range panel. LP124::nluc had a larger RLU-based host range (detects 77 of 96 strains, 80.2%) compared to AS11:nluc (detects 37 of 96 strains, 38.5%) (FIG. 59 (Table 29)). Moreover, LP124:nluc produces greater than 100-times higher RLU values compared to AS11:nluc in 73 of 96 strains (76%). This increased RLU output from LP124:nluc infections predicts that a bacteriophage cocktail that contains both A511 and LP124:nluc would have greater sensitivity and accuracy over a AS11:nluc alone.

To test whether LP124::nluc would increase the levels of RLU produced in the presence of A511 and P100 the RLU values were compared between samples infected with both a two-phage cocktail (AS11::nluc/P100::nluc) and a three-phage cocktail (AS11::nluc/P100::nluc/LP124::nluc). To test this, 1 ml of complex environmental samples grown in UVM medium were pelleted by centrifugation. The supernatant was removed and the cells were resuspended in 100 μl of either the two-phage or three-phage cocktail at a total bacteriophage concentration of 1×10⁷ and incubated at 30 C. RLU levels were measured by using Promega NanoGlo reagent and the Promega 20/20 luminometer. As for the Listeria host panel, the environmental samples showed higher levels of RLU in the presence of the three-phage cocktail than the two-phage cocktail (Table 30). This increase in the RLU output of the infection demonstrates a clear advantage from having LP124::nluc present over P100::nluc and A511::nluc alone.

The increased host range and RLU output of the three-phage compared to the two-phage cocktail suggested that a cocktail of AS11::nluc and LP124::nluc would provide useful coverage against environmental samples. To determine the ability of the cocktail to identify Listeria relevant to food processing plants environmental sampling was conducted in various food processing plants in the United States. These food processing plants represented seafood, dairy, meat and produce processing plants and were geographically diverse in their location. After environmental collection was performed, Listeria that were present in the environmental samples were isolated using a modified USDA isolation method. The Listeria were struck out on BHI agar plates and a single colony was used to inoculate 1 ml of 0.5×BHI medium in a 2 ml deep well dish and covered with a sterile breathable membrane and incubated for 16 hours at 30 C. Each of the 16-hour cultures from the 96-well plated were diluted 1:10,000 in 198 μl of BHI. For the infection, 12.5 μl of the culture dilution were mixed added to 12.5 μl of a bacteriophage cocktail containing A511::nluc and LP124::nluc at a total bacteriophage concentration of 1×10⁷ pfu/ml in an opaque luminescence reader plate and incubated at 30 C for 3 hours. After three hours the level of luminescence was detected using a Promega Glomax 96-well plate reader using Promega NanoGlo reaction following manufacturer's recommendations. Concurrently, a liquid-based host range assay was performed to compare the RLU output to culture clearance.

Based on the liquid-based host range assay the bacteriophage cocktail was able to clear the bacterial culture in 25 of 100 strains (25%). This decreased level of clearance is due to a greater growth rate for the environmentally isolated strains compared to common lab isolates tested in the Listeria host range panel. The RLU based host range assay identified 75 of 100 strains (75%) (Table 31). These environmental samples represented complex microbiological communities and had multiple Listeria isolates per environmental sample. The presence of multiple strains of Listeria within these microbiological communities improves the sensitivity of the assay. In this example the environmental samples were collected using sponges and the sponges were incubated for up to 24 h with media, after which an aliquot was removed and assayed for the presence or absence of the bacterial population to be detected. Based on the ability of the bacteriophage cocktail to identify individual Listeria strains identified from the same environmental samples it would have been predicted that the bacteriophage cocktail of A511 and LP124:nluc would be able to detect 48 of 57 (84.2%) Listeria positive sponges. When the environmental sponge was incubated in a growth medium and a sample of the enriched sample is tested using the assay the bacteriophage cocktail containing A511 and LP124:nluc was able to detect 49 of 57 (85.9%) Listeria-positive sponges. This increased sensitivity demonstrates that the presence of multiple Listeria strains, including those out of host range for the bacteriophage cocktail, does not diminish the sensitivity of the assay to detect Listeria strains that are sensitive to the bacteriophage cocktail.

### Example 23: Optimization of Listeria Detection-v2.0

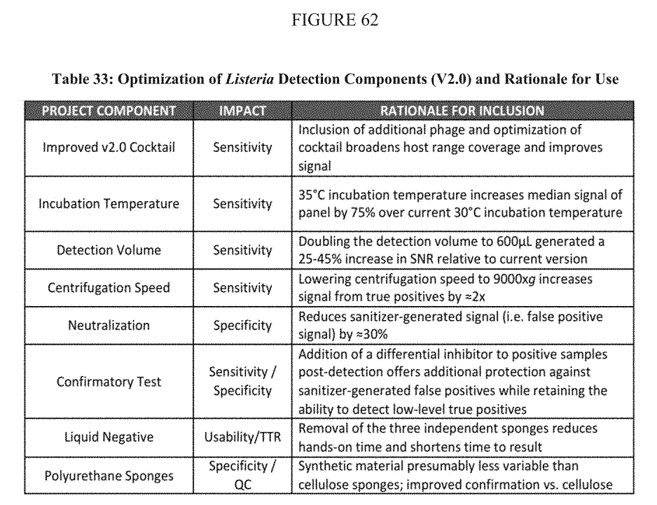

The phage detection composition was further optimized to promote sensitivity, specificity and usability. The optimized Listeria detection composition is herein referred to as “Listeria detection composition v2.0.” The methodology and the components of the detection composition were modified to achieve the 2.0 version which is superior to previous iterations of the Listeria detection composition in terms of sensitivity, specificity and ease of use. The Listeria detection composition v2.0 was generated through modification of buffer cocktails, incubation temperature, detection volume, centrifugation speed, and through the use of a different sponge material for sampling (See FIG. 62 (Table 33)). The examples that follow demonstrate the increased benefits provided by each component of the Listeria detection composition v2.0. The Listeria composition v2.0 provides approximately a 5-fold increase over a previous iteration (v1.0.4) on control strains. Moreover, the v2.0 provides increased host range coverage and light output.

### The Influence of Reagent Volume on the Detection of Listeria

The influence of the reagent volume/ratio to the sample volume (e.g. Listeria lysate) on the detection of luciferase signal was investigated. The “reagent” refers to the Nano-Glo substrate. Prior iterations of the Listeria detection composition used a 1:1 ratio of sample volume to reagent volume. For these experiments the amounts/volume/concentration of reagent was varied. For example, in these experiments the effects of the following on the amount of detected signal was investigated: less total substrate, less concentrated substrate, and a lower ratio of sample to Nano-Glo.

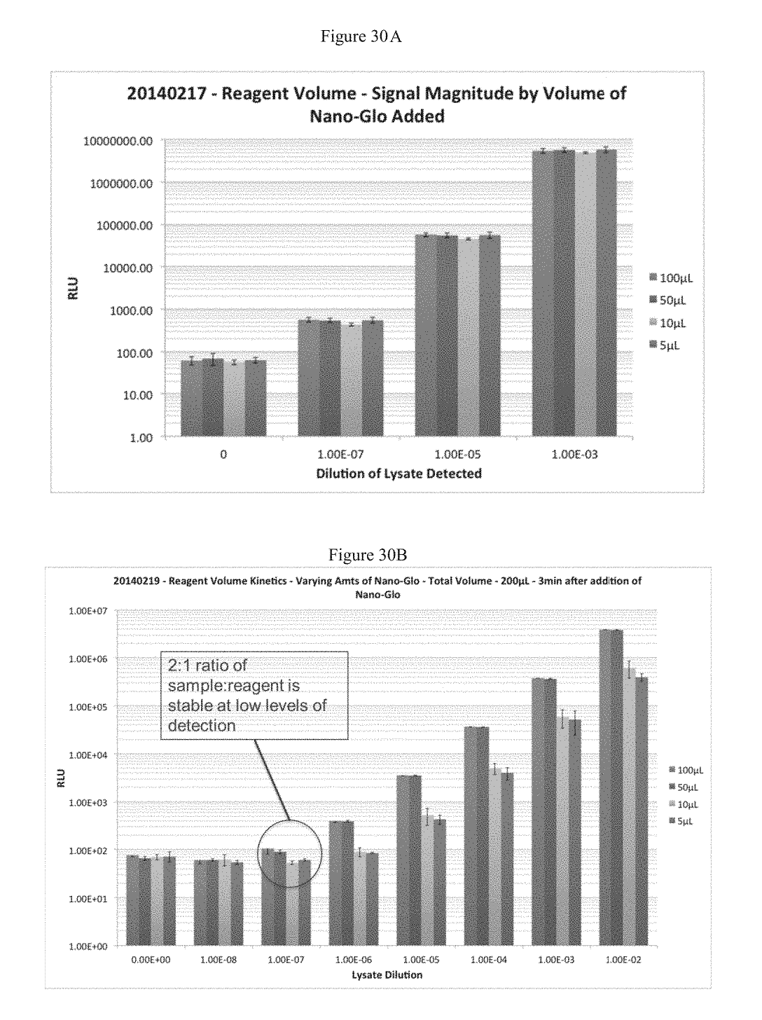

FIGS. 30A and 30B indicate that the use of a 2:1 ratio of sample volume to Nano-Glo reagent does not have a negative impact on the resultant RLU signal detected. These data indicate that it is possible to double the sample volume removed from the sampling bag without increasing the amounts of additional Nano-Glo to acquire detectable signal.

Another iteration of experiments meant to assess the impact on varying the volume of the detection reaction included varying the volume of the sample added to the detection reaction composition while maintaining a constant volume of the Nano-Glo reagent added to the reaction. For these experiments, the following protocol was followed: 1) spike 100 cells onto sponge, 2) infect with 6 mL of phage cocktail (v1.0.1 phage, v1.0.3 or 1.0.4), 3) incubate for 6 hours at 30° C., 4) remove 300 μL, 600 μL, or 900 μL of sample from bag, 5) add 300 μL of Nano-Glo reagent to each sample, and 6) detect the emitted light.

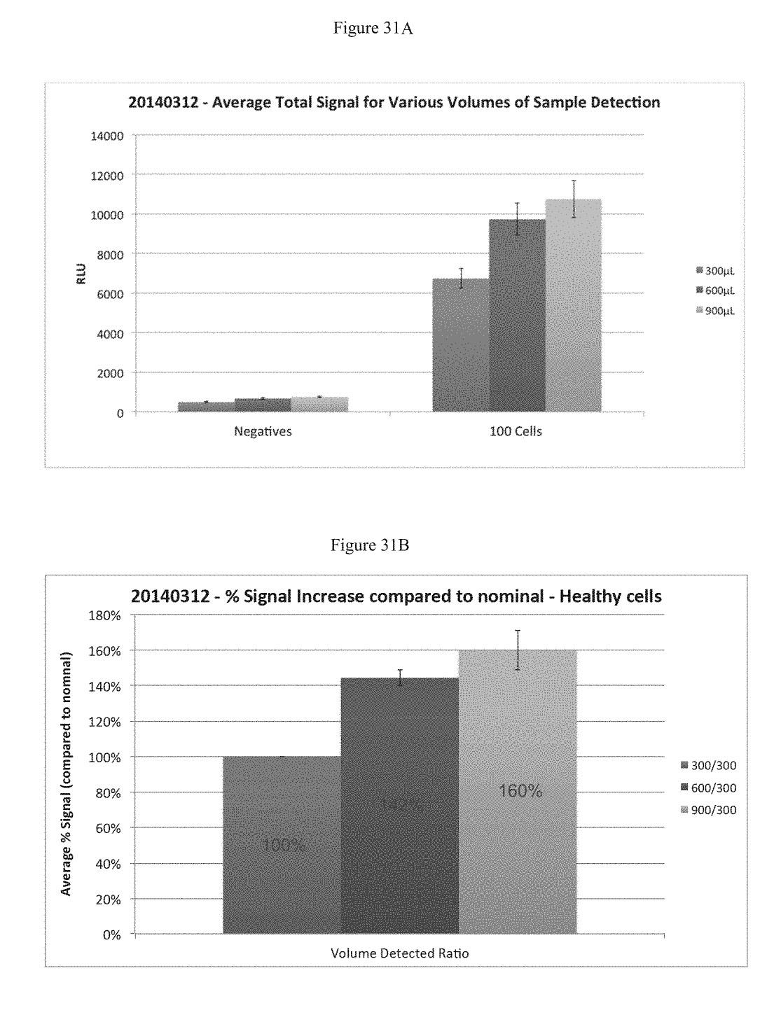

The results of these experiments indicated that there is a progressive increase in the amounts of emitted light signal that corresponds to the increased amounts of sample volume added in comparison to the standard protocol (i.e. 1:1 sample volume to Nano-Glo substrate). See FIGS. 31A and 31B.

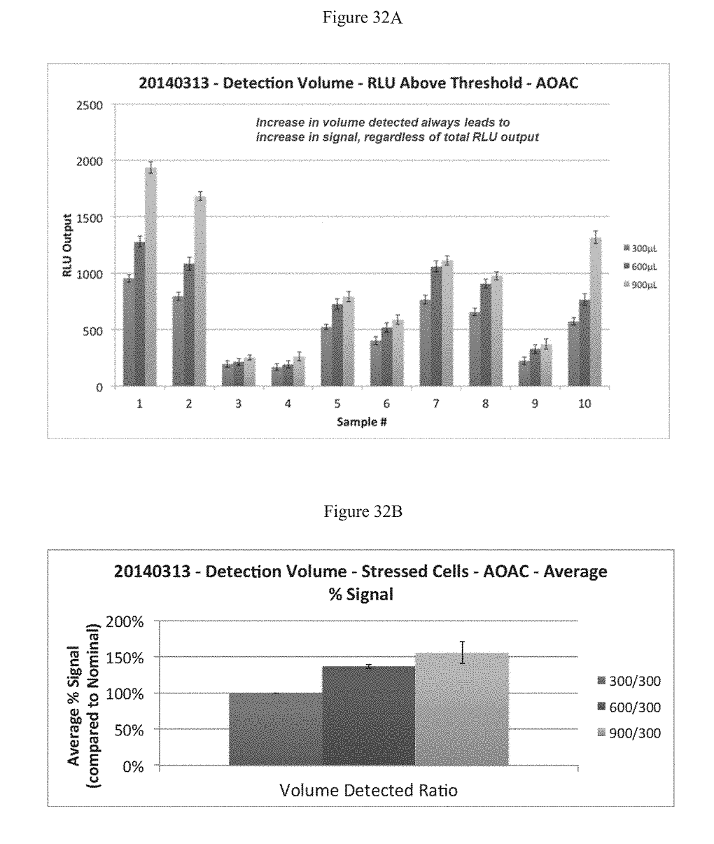

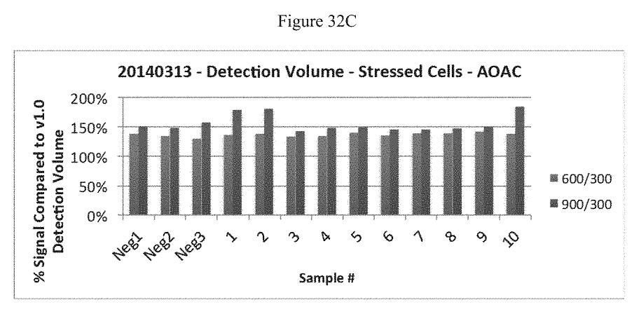

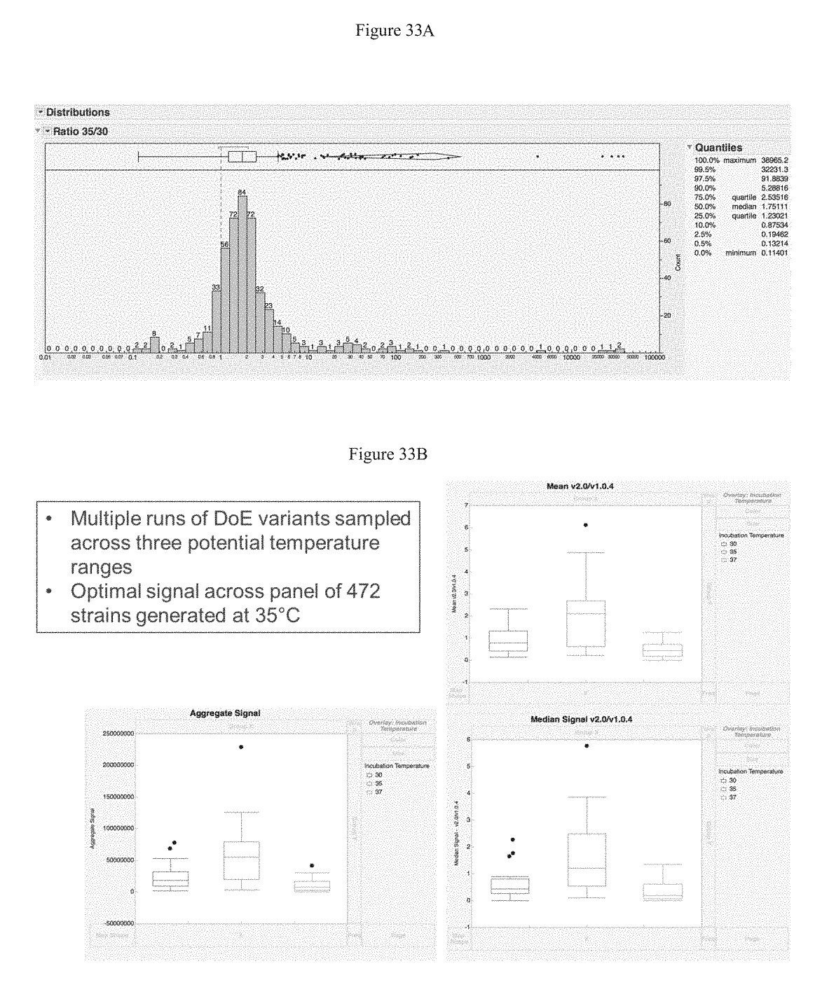

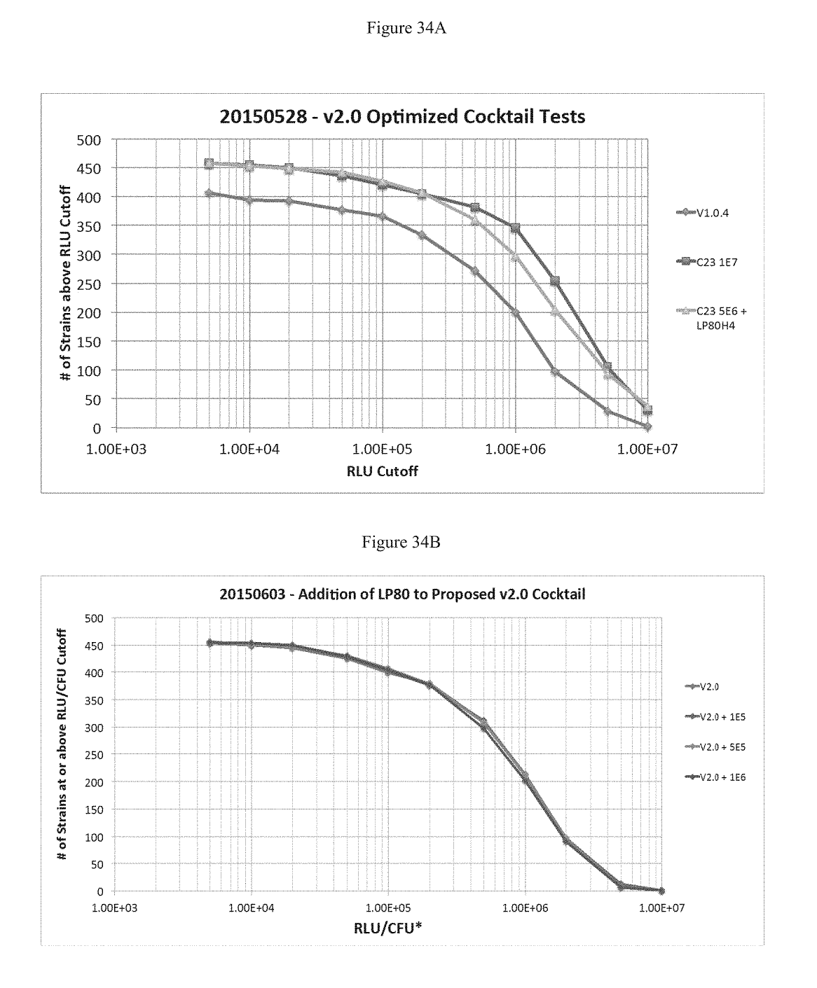

Similar experiments were conducted with stressed cells to assess the influence on varying the volume of the detection reaction while maintain a constant volume of Nano-Glo. See FIG. 32A-32B. The experimental protocol followed for these experiments were: 1) 2000 L. monocytogenes cells (CDW 1554) were spotted in the presence of 100× E. faecalis, 2) the cells were allowed to dry overnight, 3) the spots were swabbed with 3M Letheen Sponge Stick, 4) the sample was incubated for 2 hours at 25° C., 5) the sample was infected with 6 mL COP2 at the concentration of 4.5E7 PFU/mL (referred to as FCF), 6) the sample was subsequently incubated at 30° C., 7) 300 μL, 600 μL, or 900 μL of the sample was removed, 8) 300 μL of Nano-Glo reageant was added to each sample, and 9) the emitted light was detected.

The results of these experiments indicate that increasing the volume of the sample always resulted in increased amounts of signal, regardless of total RLU output. See FIGS. 32A-32C. The increase in detected signal also increased in the negative control samples (see FIG. 32 C). The data indicate that a 600 μL sample volume offers approximately 40% increase in signal over previous iterations of the detection composition, and that a 900 μL sample volume offers approximately 50-60% increase over previous iterations of the detection composition. One potential drawback of the 900 μL sample volume is the potential for increasing background (i.e. false positives). Because of this, the final volume of the sample in the detection composition is set for 600 μL.

### The Influence of Incubation Temperature on the Detection of Signal

A series of experiments were performed to assess the influence of incubation temperature on the resultant emitted light signal detected. For these experiments 472 Listeria strains were assayed using the following protocol: 1) 1000 CFU/well were placed in 100 μL of sample buffer, 2) the Listeria were infected with 100 μL of phage A511::COP3, LP124:COP3, LP40::COP3, LP22::COP3, V18::COP3, LP80H4, 3) the samples were incubated at either 30° C., 35° C., or 37° C. for 6 hours, and 4) the samples were processed per 40 μL of sample was added to 40 μL of Nano-Glo luciferin/detection buffer prepared according to the manufacterer's instructions and the luminescence was measured on a Promega GloMax96 luminometer using the built-in “SteadyGlo” protocol (1 second integration, or equivalent to detect emitted light signal.

The data indicate that the increasing the temperature at which the samples are incubated from 30° C. to 35° C. results in an increase in the median signal by 75%. See FIGS. 33A and 33B. However, the data also indicate that increasing the temperature of incubation from 30° C. to 37° C. results in a decrease in the signal detected. See FIG. 33B.

### Phage Cocktail Component Optimization

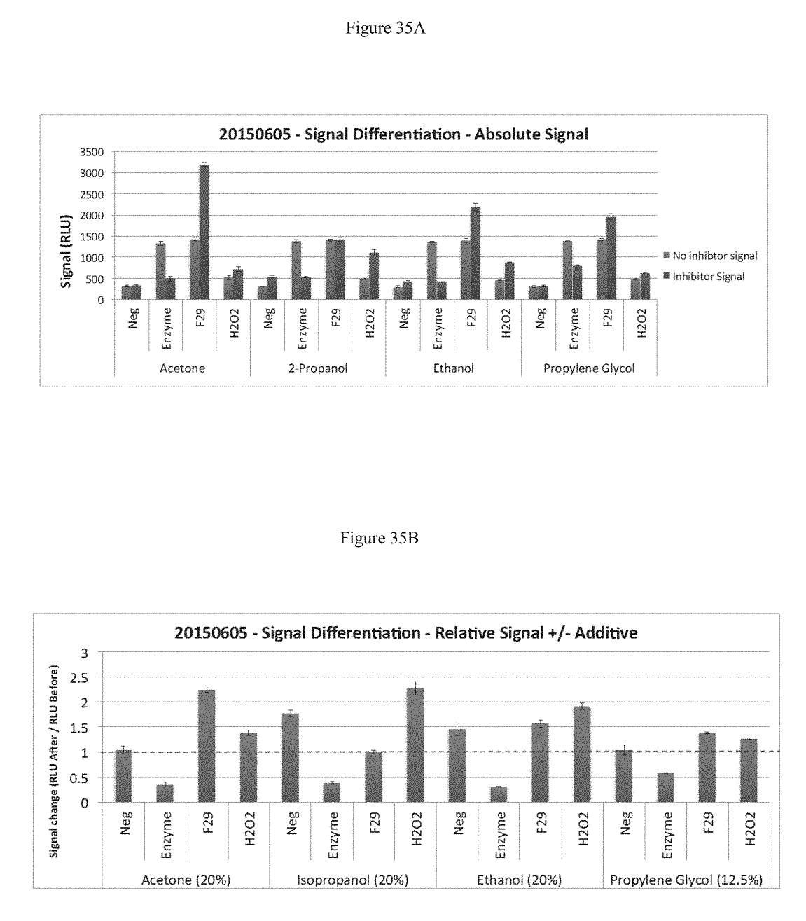

The kinds of phages and the concentrations at which these phages were added to samples were optimized. See FIGS. 34A and 34B. The performance of various iterations of optimized cocktails on the detection of Listeria strains is presented in FIG. 34A. Note that the v2.0 version of the cocktail results in superior range of detection of Listeria strains. Another part of the optimization included the addition of LP80 UTR7 (also refered to herein as LP80H4) optimized phage to the phage cocktail at various concentrations. Table 34 below is a complete list of the phage components in the v2.0 detection composition. For these experiments the detection composition v2.0 phage cocktail was maintained at a constant 1E7 pfu/mL, while the LP80 phage was added to the v2.0 cocktail at 1E5, 5E5 or 1E6 pfu/mL. LP80 is used as an additional phage for inclusion in the v2.0 cocktail because it is able to infect unique Listeria strains. The data from these experiments indicate that the addition of LP80 at 1E5 pfu/mL does not harm the aggregate signal, while adding the benefit of the detection of a greater amount of Listeria strains. See FIG. 35B and Table 35.

TABLE 34
One Embodiment of the Listeria Detection
Composition v2.0 Phage Components

| Phage | Concentration | |
|---|---|---|
| A511::COP3 | 7.15e5 | PFU/mL |
| LP40::COP3 | 3.17e6 | PFU/mL |
| LP22::COP3 | 2.86e6 | PFU/mL |
| V18::COP3 | 3.26e5 | PFU/mL |
| LP124::COP3 | 2.93e6 | PFU/mL |
| LP80::UTR7-COP3 | 1E5 | PFU/mL |

TABLE 35
Amount of Listeria strains detected at or above cutoff threshold with v2.0
cocktail, and v2.0 cocktail with the addition of LP80 at various concentrations.

| RLU | 5.00E | 1.00E | 2.00E | 5.00E | 1.00E | 2.00E | 5.00E | 1.00E | 2.00E | 5.00E | 1.00E |
|---|---|---|---|---|---|---|---|---|---|---|---|
| Cutoff | +03 | +04 | +04 | +04 | +05 | +05 | +05 | +06 | +06 | +06 | +07 |
| V2.0 | 453 | 450 | 444 | 425 | 399 | 380 | 310 | 213 | 96 | 13 | 1 |
| V2.0 + 1E5 | 454 | 450 | 447 | 428 | 403 | 376 | 311 | 212 | 97 | 11 | 1 |
| V2.0 + 5E5 | 456 | 451 | 447 | 430 | 407 | 379 | 307 | 210 | 95 | 8 | 1 |
| V2.0 + 1E6 | 455 | 453 | 449 | 429 | 404 | 377 | 297 | 202 | 90 | 6 | 1 |

### Use of Alternative Sponge Material for Sampling

The currently used sponge for the detection assays is a cellulose sponge. Some of the disadvantages of using a cellulose sponge include supply chain vulnerability, potential for residual quaternary ammonium compounds from the manufacture of the cellulose sponge, batch-to-batch inconsistency in the production of the sponges, and the possibility that the sponges can have extraneous materials from the manufacturing process such as, for example, heavy metals, chloro-organic compounds and residual sulfur from the manufacturing process.

As an alternative to the use of a cellulose sponge, polyurethane sponges were tested in the detection protocol. The benefits of polyurethane sponges include that these sponges are non-toxic, there is a greater amount of batch-to-batch consistency in the manufacturing of the polyurethane sponges, the sponges are routinely used in medical devices, they have good water absorbency and retention, and the sponges are strong, characterized by the polyurethane sponge's high tensile strength.

One of the objectives is to replace the cellulose sponges with polyurethane sponges as a sample collection device, and to assess whether there is any improvement in the consistency in liquid retention in the polyurethane sponges in comparison to the cellulose sponges. To test whether there is improved consistency in the in liquid retention in the polyurethane sponge the following protocol was followed: a set of naïve cellulose (Cell) or polyurethane (PUR) sponges was prepared; each sponge was hydrated by the manufacturer with 10 mL of buffer; the amount of liquid squeezed out from each sponge (the “squeezate”) from each sponge was collected; both the sponge (“on sponge infection”) and the squeezate (“liquid infection”) were treated with 6 mL of S6 infection buffer containing a phage cocktail followed by incubation for 6 hours at 30 C; 1 mL aliquots were collected from both infected liquids and liquid in the sponges; the aliquots were spun down for 1 minute at 14 k rpm; 300 μL of clarified liquids was transferred to optical tubes for detection; RLU data was recorded and CV (coefficient of variation) values were calculated for “liquid” vs “on sponge” infections.

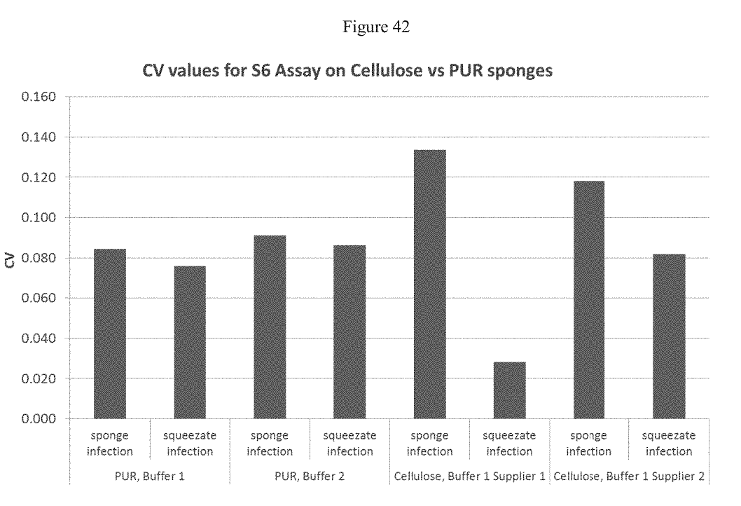

The data are presented in FIG. 42. The data demonstrate that PUR sponges perform more consistently with regard to the amount of liquid squeezed from the sponge and remaining in the sponge, while the cellulose sponges show a larger variability in sponge thickness which results in less consistent amount of liquid removed in comparison to that remaining in the sponge.

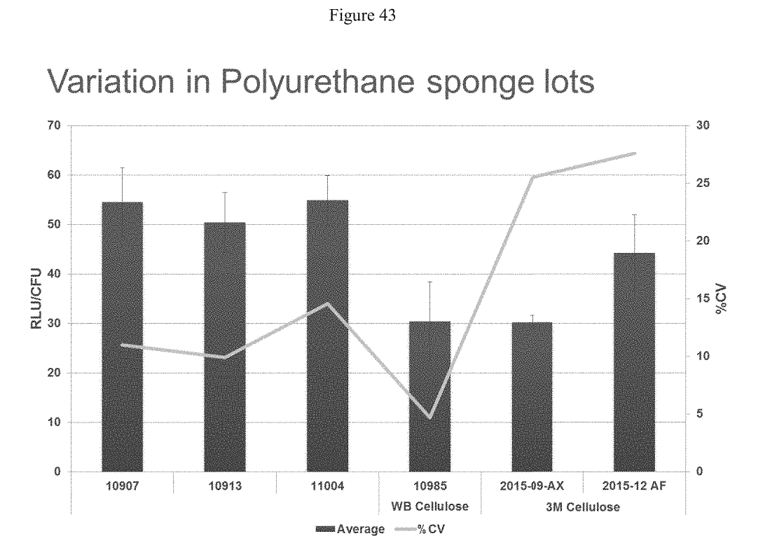

Also tested was the variation in performance of different polyurethane and cellulose sponge lots using a Listeria stressed cell model. For these experiments, cells were stressed by desiccation for 20 hours on a stainless steel surface, swabbed into Letheen and then spiked onto 5 sponges for each lot. Sponges were processed via the Listeria detection assay v2.0 protocol and a RLU/CFU was calculated for each sponge. The data are presented in FIG. 43. The data indicate less variation in the polyurethane sponge lots in comparison to the cellulose sponge lots in terms of resultant RLU/CFU detected.

### Neutralization of Residual Sanitizers

As discussed above, the presence of residual sanitizers can lead to false positive signals by interaction with luminescent substrate, for example, by oxidation. Most sanitizers used in the food industry fall into several categories: chlorine-based sanitizers (bleach, chlorine dioxide), peroxyacid compounds +/−organic acids, organic acids (ascorbic acid, citric and lactic acid, caprylic acid), quaternary ammonium compounds, iodophors, surfactants and detergens. Despite the neutralization in the previous S6 Infection buffer, in some instances sanitizers are not completely deactivated. The S6 Infection buffer is composed of: 1×Tryptic soy broth, 0.25% lithium chloride, 20 mg/L nalidixic acid, 0.5% yeast extract, 0.25% glucose, 0.08% magnesium sulfate, 0.1% sodium pyruvate, 20 mM Hepes (pH7.4), 0.22% lecithin, 1.5% Tween 80, and 0.02% potassium phosphate. Various compounds were assayed to develop a more robust neutralization of residual sanitizers in the samples. Three potential neutralizers were of interest: sodium metabisulfite, sodium pyruvate and catalase. Of these three, sodium metabisulfite and sodium pyruvate were tested in the assays.

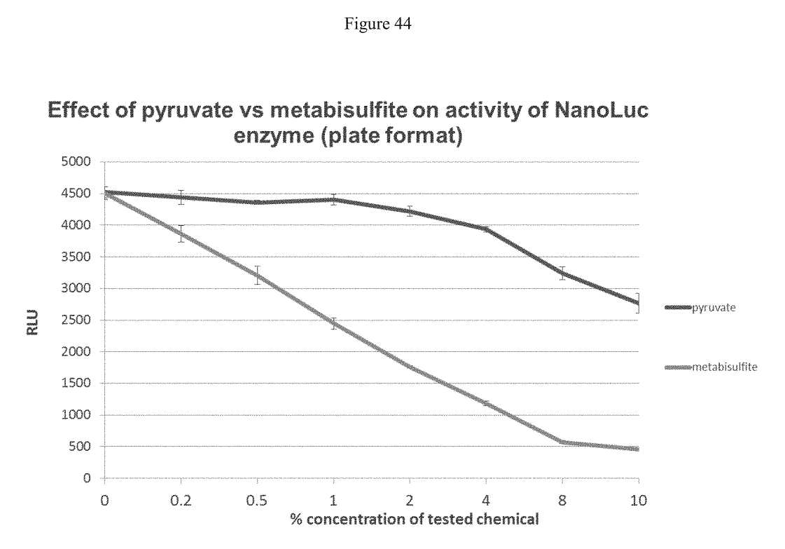

The effect of pyruvate or metabisulfite on the activity of the Nanoluc enzyme was tested. The data are presented in FIG. 44. The data indicate that the addition of pyruvate up to 1% is able to maintain high Nanoluc enzyme activity.

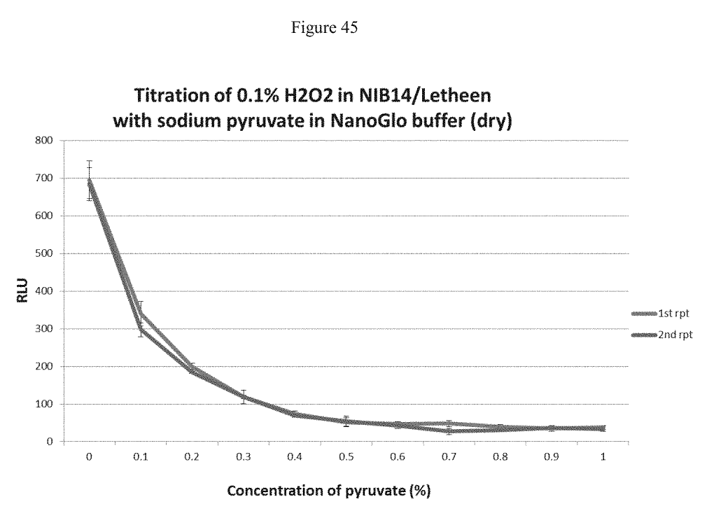

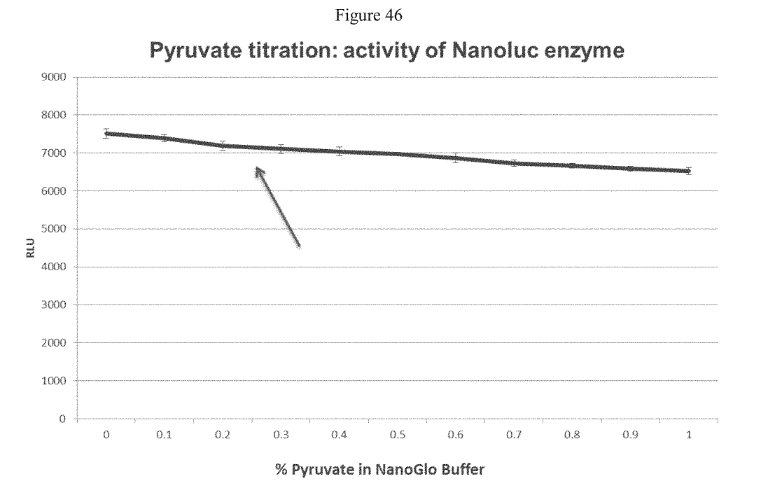

To directly test the effectiveness of pyruvate against peroxides the following protocol was followed: dry pyruvate was titrated into NanoGlo buffer; to mimic the presence of peroxides collected from the environment with sponges, a 0.1% hydrogen peroxide was prepared in a mixture of Letheen/infection buffer (4:6); and to avoid dilution of the NanoGlo buffer, dry pyruvate was dissolved directly in the NanoGlo buffer. The data are presented in FIGS. 45 and 46. In order to balance the effects of neutralization with maintenance of enzymatic activity, the pyruvate concentration selected was 0.25% pyruvate. Other concentrations of pyruvate for neutralization can include 0.05, 0.1, 0.15, 0.2, 0.25, 0.3, 0.4, 0.5, 0.6, 0.7, 0.8, and values in between.

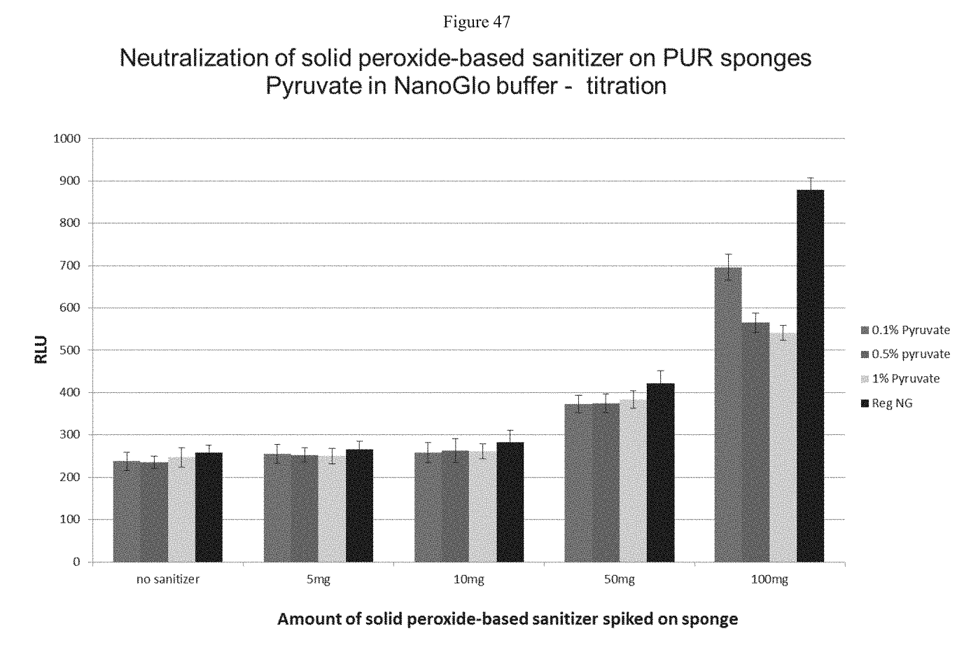

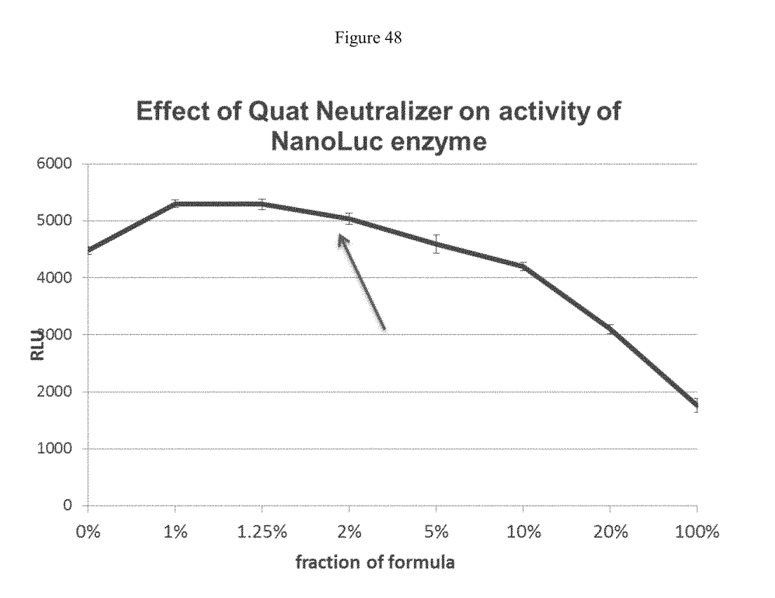

The effectiveness of pyruvate on sponges was evaluated. For these experiments the following protocol was followed: increasing amounts of solid peroxide-based sanitizer was spiked onto sponges thereby mimicking collection of sanitizer from the environment; no cells were present on the sponges; the sponges were incubated for 15 minutes at room temperature (this allows for partial dissolution of sanitizer and partial neutralization by buffer in the sponge); the squeezate was removed followed by adding infection buffer with phage to the isolated squeezate; the sample is incubated at 30 C for 6 hours (allows for decomposition of sanitizer and partial neutralization by the infection buffer); 1 mL aliquots were spun down for 1 minute at 14K rpm; 300 L of the clarified liquids were transferred to optical tubes for detection; the detection was performed using NanoGlo buffer with various amounts of sodium pyruvate; RLU values were recorded (these represent false positive signals generated by oxidation of the substrate); as a control, regular NanoGlo buffer without neutralization was also included in the assays. The data from these experiments are presented in FIGS. 47 and 48. Collectively, these data indicate that increasing concentrations of pyruvate have a decrease the amounts of background signal that is caused by residual sanitizers in the sample.

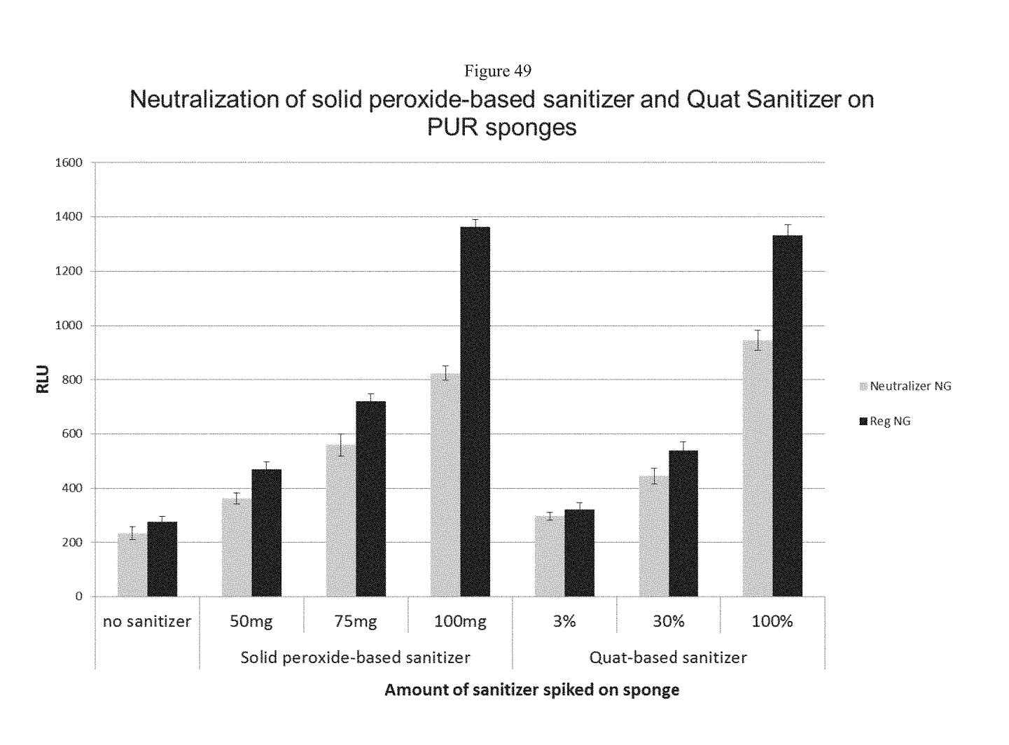

A combined formula that uses 2% of the Quat Neutralizer (28% Tween-80, 4% lecithin, and 3 mM KH₂PO₄, at pH 7.4) and 0.25% sodium pyruvate was used to supplement the NanoGlo buffer. This combined formula was used to test the neutralization power on sponges spiked with either quaternary ammonium compounds or solid peroxide-based sanitizer. To assay for the effect of the combined neutralizers the following protocol was followed: increasing amounts of sanitizers were spiked on polyurethane sponges thereby mimicking the collection of sanitizer from the environment; no cells were present on the sponges; the sponges were incubated for 15 minutes at room temperature (this allows for partial dissolution of sanitizer and partial neutralization by buffer in the sponge); the squeezate was removed followed by adding infection buffer with phage to the isolated squeezate; the sample is incubated at 30 C for 6 hours (allows for decomposition of sanitizer and partial neutralization by the infection buffer); 1 mL aliquots were spun down for 1 minute at 14K rpm; 300 μL of the clarified liquids were transferred to optical tubes for detection; the detection was performed using NanoGlo buffer with various amounts of sodium pyruvate; RLU values were recorded (these represent false positive signals generated by oxidation of the substrate); as a control, regular NanoGlo buffer without neutralization was also included in the assays. The data from these assays are presented in FIG. 49. The data indicate that the combined formula provides effective neutralization of both solid peroxide-based sanitizer and quaternary ammonium compound based background/false positive signals.

### Effect of Centrifugation Speed on the Resultant Light Emitted Signal

The effect of centrifugation speed was assessed with regards to the resultant light emitted signal obtained. Previous iterations of the detection protocol relied on centrifugation speeds of 14 k rpm. Various kinds of centrifugation speeds (expressed as relative centrifugal force-rcf) were assessed such as 1,000 rcf, 2,000 rcf, 5,000 rcf, 9,000 rcf or 14,000 rcf, including a no spin condition. The data obtained from these various centrifugation speeds indicate that centrifugation speed of 9,000 rcf resulted in the greatest amounts of light emitted signal and greatest reduction in false positives.

### Confirmatory Testing of Sample Results

An additional step is added to the Listeria detection composition v2.0 that serves as a test for determining true positive signals from false positive signals. In the sample detection process it is possible to have occasional false positive signals that are the result of remnant sanitizers found within the samples oxidizing components of the sampling and detection buffer. To greatly reduce or eliminate the false positive signals due to these interactions post-processing means were developed. The post-processing means are meant to be used when the detected signal from a sample is at the cutoff value or above.

The influence of organic solvents on the resultant post-processing signals detected was analyzed to develop a false positive signal control assay. For these experiments the following protocol was followed: sanitizers and enzyme (luciferase) were diluted in NIB-14 to generate an approximate 10³ dilution in 300 μL; 300 μL of Nano-Glo was added to 600 μL of each sample and detected; 300 μL of the organic solvent was added to the sample and immediately re-read. The analysis for these experiments was a comparison of the increase or the decrease in signal detected following incubation with various organic solvents. The solvents tested in these studies included acetone, isopropanol, ethanol, and propylene glycol.

The results from these experiments indicated that the addition of any of the organic solvents tested (e.g. acetone, isopropanol, ethanol, and propylene glycol) to samples that contained enzyme (i.e. a positive control) resulted in a decrease in the emitted light detected signal. Surprisingly, the addition of certain organic solvents (e.g. isopropanol and ethanol) to the samples that did not contain enzyme (e.g. the negative control samples) resulted in a robust increase of the emitted light signals detected. See FIGS. 35A and 35B. These data indicate that the signal derived from the enzyme (i.e. true signal) decreases after addition of a solvent, and that the sanitizer based signal (i.e. false positive signal) increases after addition of a solvent. Accordingly, this allows for the differentiation between true positives and false positives signals.

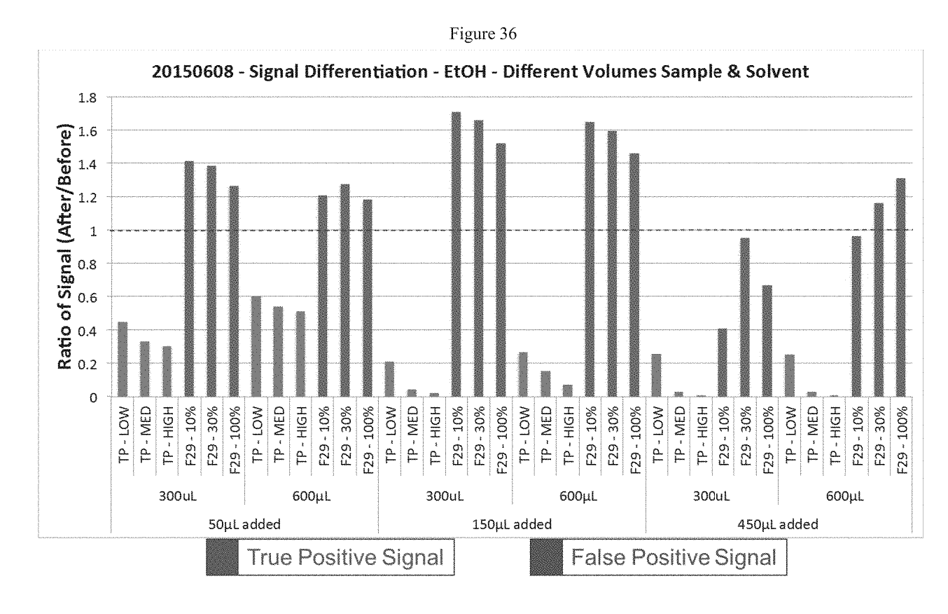

Additional experiments were run in which the volumes of the samples were adjusted followed by incubation with ethanol. For these studies the volume of the sample was either 300 μL or 600 μL, and the volume of ethanol added to the mixture was 50 μL, 150 μL, or 450 μL. See FIG. 36. These data indicate that a range of volumes (i.e. 50 μL-450 μL) of the added organic solvent can be successfully used to differentiate between true and false signals in a sample. The organic solvent can be added at various volumes per 300 μL sample, for example 2, 4, 6, 8, 10, 20, 30, 40, 50, 60, 70, 80, 90, 100, 110, 120, 150, 200, 250, 300, 350, 400, 450, 500, 550, 600, 650, 700, 800, 900 and 1000 μL can be added to the sample.

### Example 24: Comparison of Listeria Detection Composition v2.0 to Previous Iterations

The performance of the Listeria detection compositions was compared with previous iterations. For these comparisons, a previously developed version 1.0.4 (version 1.0.4 contained the following phages: A511::COP3, LP40::COP3, LP124::COP3 each at 5E6 pfu/mL; NIB-14 buffer) was compared with the optimized v2.0 to ascertain any differences in the ability to acquire signal from Listeria that are healthy, stressed or from spiked environmental samples.

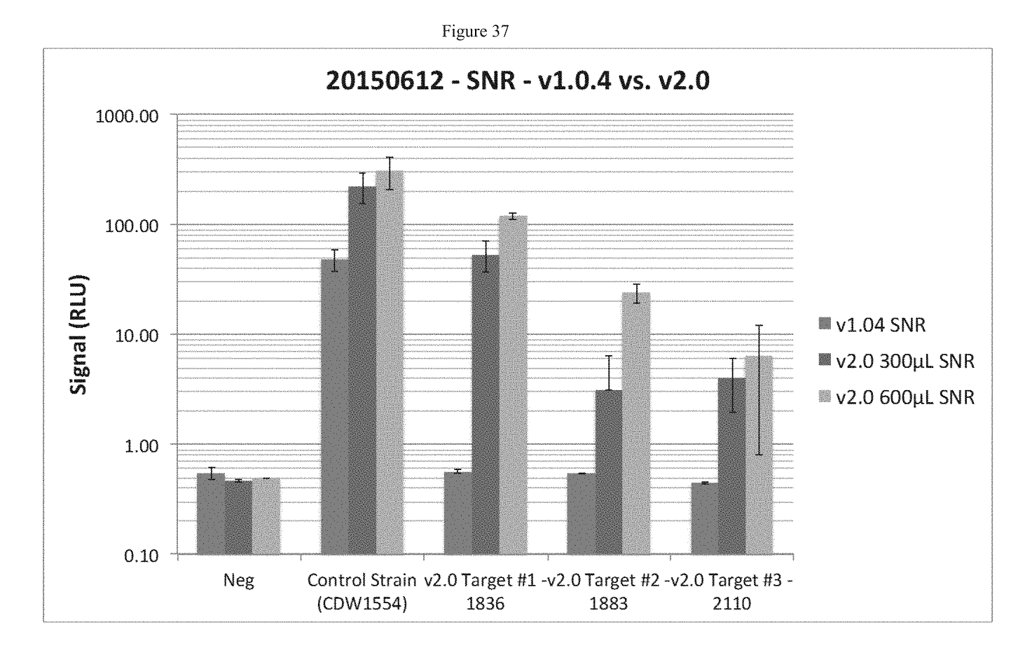

For the assays performed with healthy cells, the following strains were tested: CDW 1554 (control), NP1836 (v2.0 Target 1), NP1883 (v2.0 Target 2), and NP2110 (v2.0 target 3). Each of these strains was placed at approximately 100 cfu onto sponges and tested with either v1.0.4 or with v2.0. The data are presented in FIG. 37. The data indicate that v2.0 performed better in terms of having greater signal intensity and a more favorable signal-to-noise ratio (SNR) in comparison to the v1.0.4 for the detection of healthy cells.

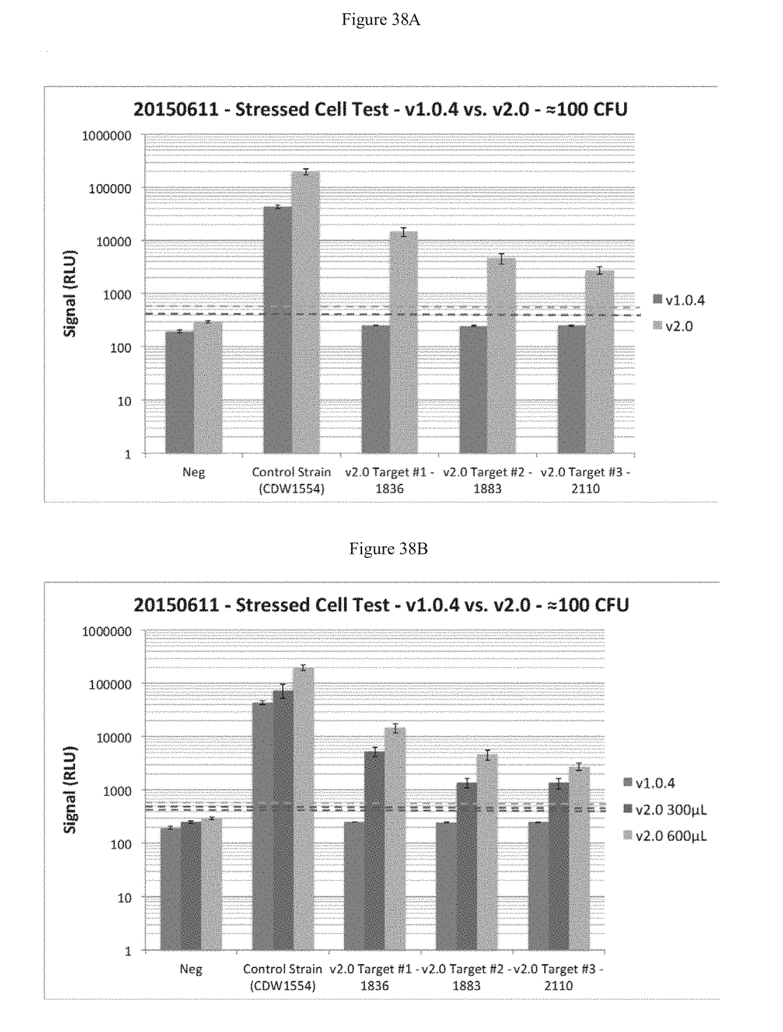

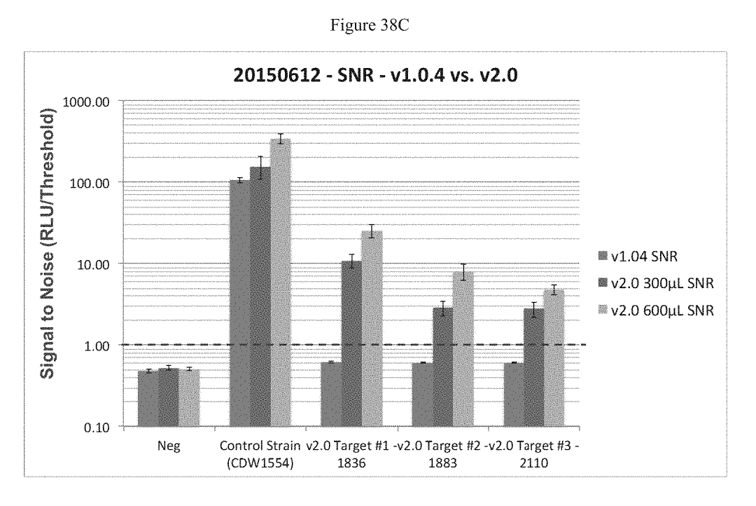

For the assays performed with stressed cells, the following strains were tested: CDW 1554 (control), NP1836 (v2.0 Target 1), NP1883 (v2.0 Target 2), and NP2110 (v2.0 target 3). Approximately, 1E7 cfu of each strain was spotted onto a stainless steel table and allowed to dry overnight. The spots were collected with a cotton-tipped swab. The presumed recovery of the sample was 1E6 cfu in 2 mL. The cells were subsequently diluted to approximately 100 cfu and spiked onto sponges and tested with either v1.0.4 or 2.0. The data are presented in FIG. 38A-38C. The data indicate that v2.0 performed better in terms of having greater signal intensity and a more favorable signal-to-noise ratio (SNR) in comparison to the v1.0.4 for the detection of stressed cells.

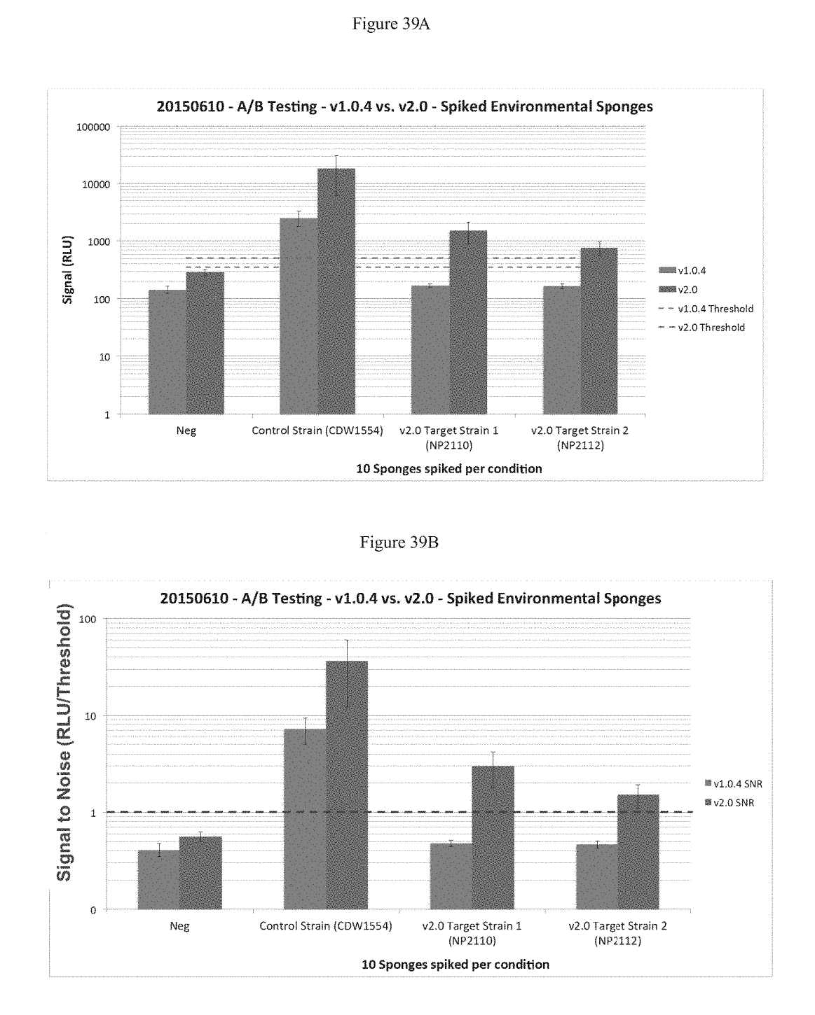

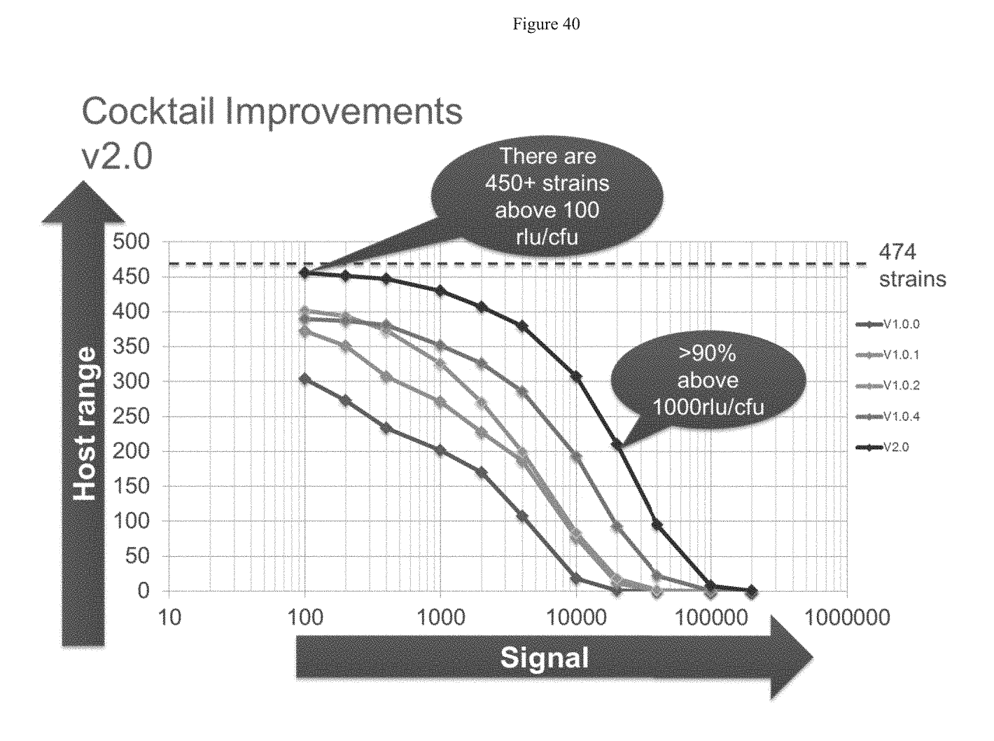

For the assays performed with environmental samples, 60 sponges were collected from a Food Production Facility and the samples were spiked with approximately 10 cfu of control strain or v2.0 target strains (CDW 1554, NP2110, NP2112). The samples were subsequently incubated overnight at 4° C. and processed with either v1.0.4 or with v2.0. The data are presented in FIGS. 39A and 39B. FIG. 40 depicts the performance of Listeria detection assay v2.0 versus previous iterations.

### Example 25: Importance of Individual Components in the Listeria Detection Composition v2.0

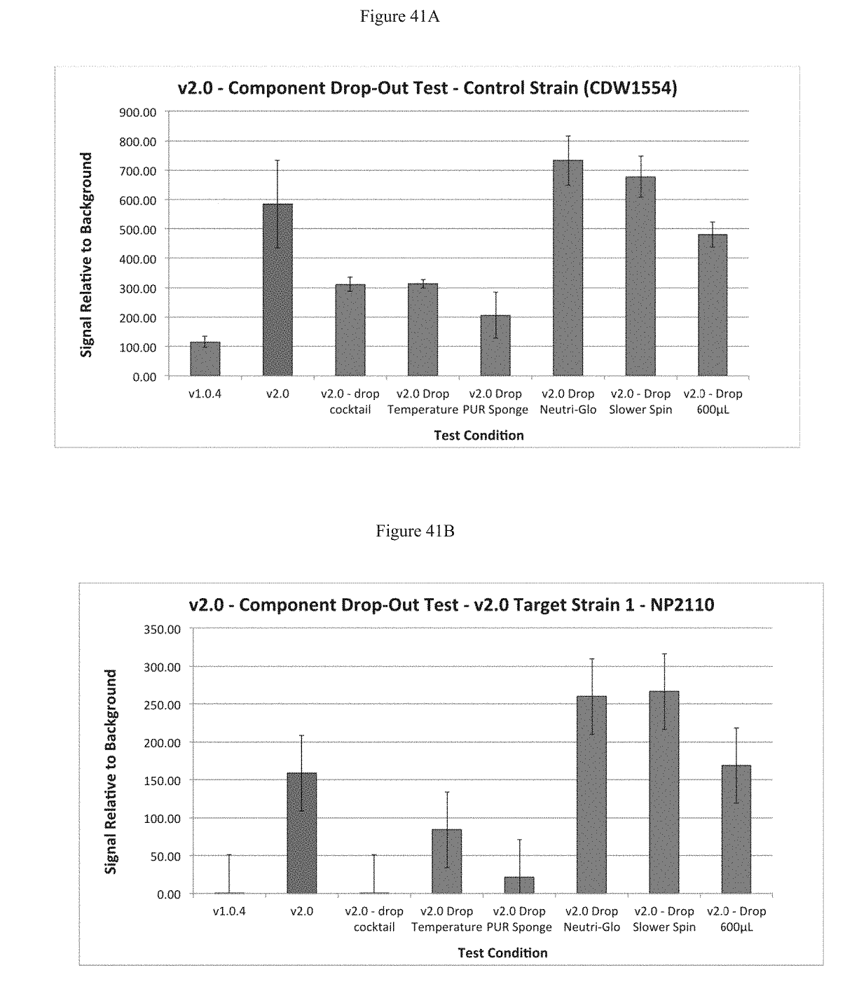

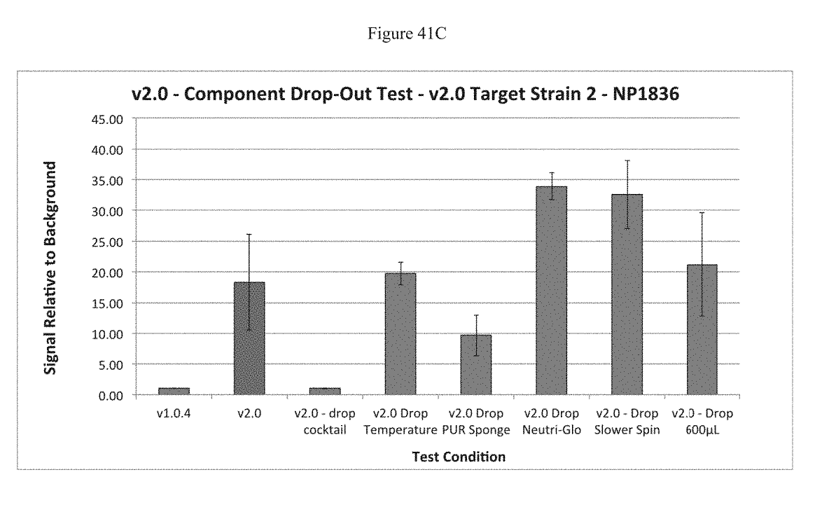

To ascertain the importance of individual components in the Listeria detection composition v2.0, individual components were of the v2.0 composition were swapped for a previous iteration of the composition (i.e. 1.0.4). In this series of experiments, the signal-to-noise ratio of the assay minus single v2.0 components were measured. The following components were substituted: v2.0 drop cocktail (v1.0.4 cocktail used), v2.0 drop temperature (30 C was used), v2.0 drop polyurethane sponge (cellulose sponge used), v2.0 drop Neutri-Glo (regular Nano-Glo used), v2.0 drop slower centrifugation speed (maximum centrifugation speed used was 13.4K RPM), v2.0 drop 600 μL (300 μL used). The results of the component substitution experiments are presented in FIGS. 41A-41C.

### Example 26: LP80 Alternate Host Characterization

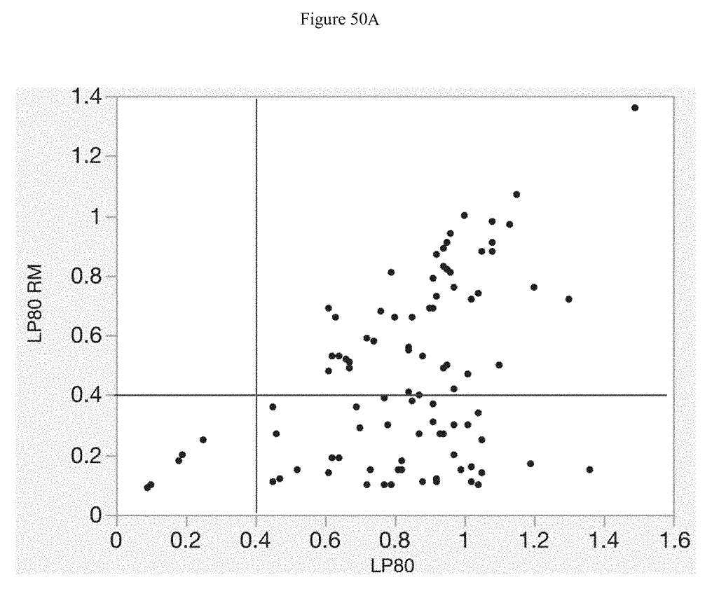

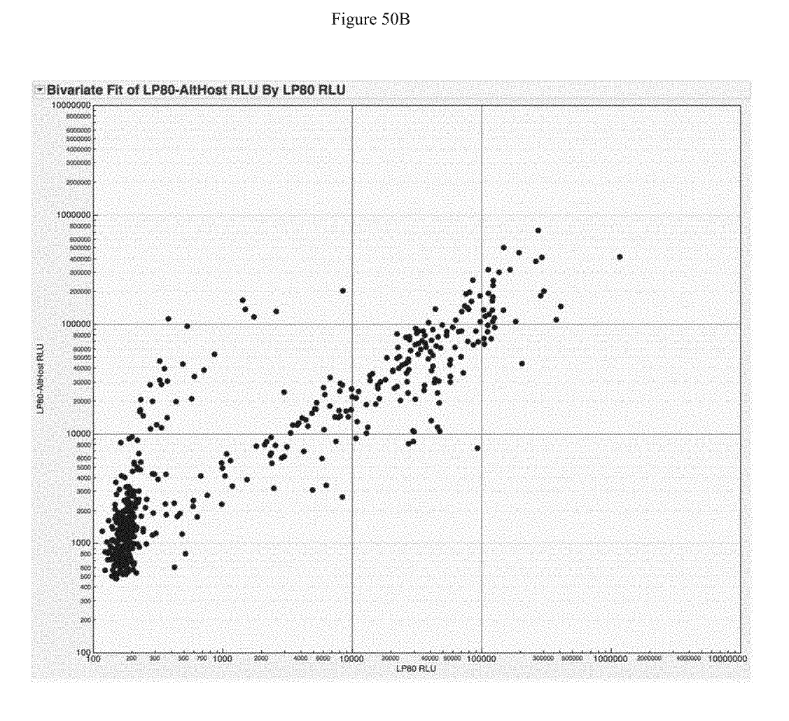

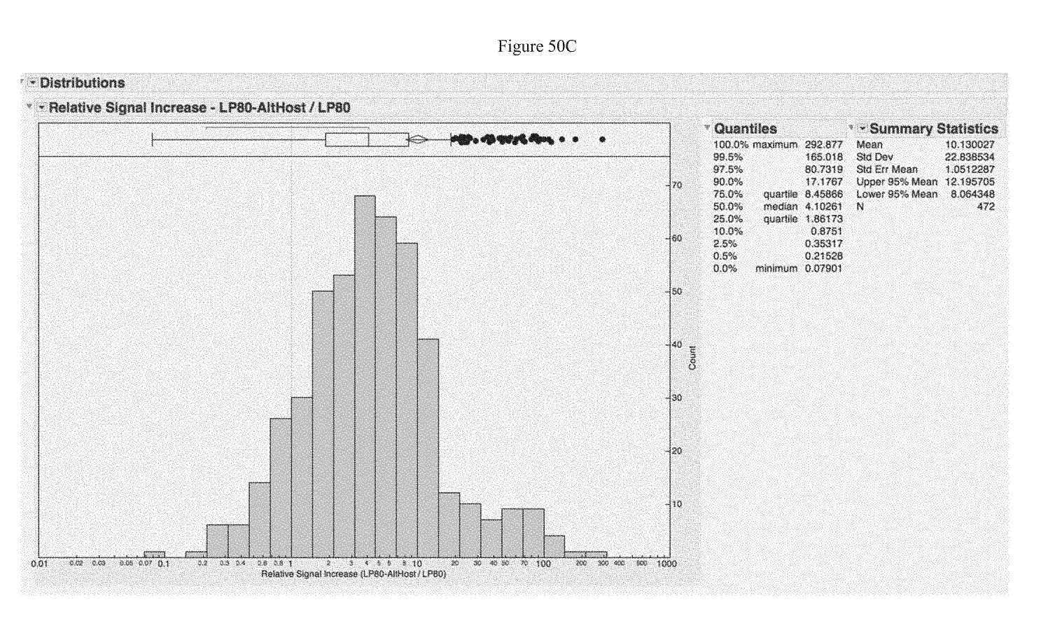

LP80:COP3 bacteriophage were used to establish a host range for said bacteriophage. For these assays a liquid-based host range analysis was performed as described in Examples 20 and 21. The tested Listeria strains comprised 96 strains that are difficult to target. See Table 36 below. The LP80 lysate generated on a typical host or an alternate host was compared. The data are depicted in FIGS. 50A-50C. The data indicate the ability of LP80 to clear Listeria cultures by the standard liquid host range clearance.

### Example 27: Supplemental Methodology

Provided below are sequences in addition to those provided above that were used for phage engineering.

The primers listed below were used to amplify COP3 and homology regions for LP80

UHR:
SO645/SO818
DHR:
SO649/SO650
COP3:
SO670/SO648
SO645 (SEQ ID 104):
TTACGCCAAGCTTGGCTGCAATATTCGCTTTGCACCACTAGC
SO818 (SEQ ID 105):
ATATTTACCTCCTCTTATTAttaggcaggtaaagtaattg
SO649 (SEQ ID 106):
CGCATTTTAGCCTAAtaaagactaagcccagcttc
SO650 (SEQ ID 107):
acgacggccagtgaattccatacctgctggcacgtct
SO670 (SEQ ID 108):
TAATAAgaggaggtaaatatatATGGTATTCACATTGGAAGA
SO648 (SEQ ID 109):
tgggcttagtattaTTAGGCTAAAATGCGCTCGC
The below primers were used to amplify COP3 and
homology regions for V18
UHR:
SO860/dbono380
DHR:
SO672/SO865
COP3:
SO670/SO671
SO860 (SEQ ID 110):
TTACGCCAAGCTTGGCTGCACCAAATCTCAATGCTTACTT
dbono380 (SEQ ID 111):
ATATTTACCTCCTCTTATTATTAGTTGCTATGAACGTTTTTTACAGG
SO672 (SEQ ID 112):
GCGAGCGCATTTTAGCCTAATAATTATAGGATAATTGAAT
SO865 (SEQ ID 113):
ACGACGGCCAGTGAATTCCCAACTACTAAATTCTGTTTGGT
SO671 (SEQ ID 114):
ATTCAATTATCCTATAATTATTAGGCTAAAATGCGCTCGC

The primers listed below were used to amplify COP3 (UTR7 Variant) and homology regions for LP22

UHR: S0645/SO646

DHR: S0649/SO650

COP3 (UTR7 Variant): S0647:S0648

Template for the COP3 (UTR7 Variant) (SEQ ID 115) was an A511 phage engineered with COP3 (UTR 7 Variant). Lowercase letters are the UTR7 sequence, Uppercase are the COP3 sequence.

COP3 (UTR7 Variant) (SEQ ID 115):
ataattttgattaactttaaaggagataaatatatATGGTATTCACATT
GGAAGATTTTGTGGGGGATTGGAGACAGACAGCTGGATATAACTTAGAC
CAAGTATTAGAACAGGGTGGAGTGTCAAGCTTATTTCAAAACTTAGGTG
TGTCAGTGACTCCAATTCAACGTATTGTGTTAAGTGGAGAAAACGGTTT
AAAAATAGACATTCATGTGATTATTCCGTACGAAGGCCTCAGTGGTGAC
CAAATGGGACAAATAGAGAAAATCTTTAAAGTAGTGTACCCTGTGGACG
ACCATCACTTTAAAGTAATCTTACACTATGGTACGTTAGTAATTGATGG
CGTAACGCCAAACATGATAGACTACTTTGGGCGTCCTTATGAAGGCATT
GCCGTGTTTGACGGCAAAAAGATCACCGTGACAGGTACTCTATGGAATG
GAAACAAAATCATTGACGAGCGTTTAATCAACCCAGACGGCTCTTTACT
ATTTCGGGTAACAATTAACGGCGTGACCGGATGGCGATTATGCGAGCGC
ATTTTAGCCTAA
SO646 (SEQ ID 116):
tatTTATTAttaggcaggtaaagtaattgtaacagaagaagc
SO647 (SEQ ID 117):
actttacctgcctaaTAATAAataattttgattaacttt
LP80 UHR seq (SEQ ID 118):
ATATTCGCTTTGCACCACTAGCTACAACATTCAATCAATTACAAGGTTC
TGTAGGGGATACAATCACTCTTCCTAACTGGAACAAAATTGGTAAAGCA
GAAGTTGTTGCAGAAGGTCAAACTTCTAATATTGACACTATTAACCAAT
CTCAAATCTCTGTAACAGTTAAAAAAGCTGTTAAAGCTGTAGCTATTTC
AGACGAACTAGAACTAGCTTCCGCAGGTAACCCTGTTAATGAAATTGTT
GACCAAATCGCAATGGCTTTAGCTCAAAAAGTAGATGATGATTTAATTG
CTATTGCTCGTTCTGCTAAAAAAGCTGTAGACCCTTCAACTGGAGAGGC
TCTTACTGTAGATGCAATCAACAAAATTCCTTTAGCTTTAGCTAACTTT
GGTGAAACTCTTTACGAAGATGCTACATATCTTTTAGTAAGCACTAACA
GTTATGCTTTGTTCGTTTCTGATGATAAGTTTGTTCCTATCATTAATCA
AGGTTCTATCATCATCAATGGTACTATCGGTACTCTTTATGGATGTACT
GTAGTTCTTTCTGATAAAGTACAAGACGGTGAGTTCTTCTTCATTAAAG
CTGGTGCTCTTGGTATTGCTCTTAAACAAGATACACGTATTCTTACTGA
ATATGATTTACTTTCTCACACTACATTAATCAGTGGTGACCGCCACTAT
GCTACATTTATGGCTGATGAAGATAAAATTGTTTATGTAGGTGCAGGCA
CTGCTGTTCCAGCGGCTCCAACTATTGCGACTCCTGCTAcaactgcttc
ttctgttacaattactttacctgcctaa
LP80 DHR seq (SEQ ID NO: 119):
TAAAGACTAAGCCCAGCTTCGGCTGGGTTATAAGTCTATTATAATCGAA
TCGGAGGAATAAATCAATGGCTGTAGAAAAGAAATATGATATAAAGAAA
AATGGTAACATTATCGAAACGTTCCATGCTTCAGGTGACTTTGTGGATA
ATGATGTATTACCAAAAACAAACTACCAATACCAAGTACGTGTGCGTGA
GGTAGACTCTGGTGTAGTAAAATCTACCTCTGAGTGGACTGCTCCTATT
AACGTCAAAACTAAGTAAGGGGAATTAGTAAATGCCAACCGTTAAGAAA
TATGATATTAAACGTAACGGCAATGTGGTAGCTACATATGTTCCCGCTG
GAAATTGGAGAGATGATAATCTTCTTCCTAATACTAACTACCAGTATTT
AGTTAGATGTCATGAAGTTACAACTACTGGTAGTGACGTCAGTATTAAA
ACAAGTGATTGGACAAACCCAATTAACGTTAAAACACTGGTATCTGATA
CTAGTATTCATCCACCTGCTCCTATTAATGTTAATGTAACAGATTTAGA
ATCTGATTCTTTCACTATTGATTGGGGTAATGGAGGATTTAATGCTAAA
ACGTATGTTGTTCGTTTAGGCATTAATGGAGATGGATTGCCAGATAATA
ATGTTTTTGTAACTACAACTAATAGCTTTACAGCTACTGAGGATGTAGT
AGTAGCATTAGCTCCATTCGCTGGTAAGAAAATTGACGTATATGTAGCA
CAATTTGCTGGAGTTTACGCGGATGCCACTTCTGCTTTAGAAAGTGCAG
AATACAATGAAGTAACTGCATGGTCTAACCTAGTTACTGTAGATGTCCC
ATCTGCAAGTCCTTCAATGTTACGTTCTTCTAGTAATAAAGAAATTGTT
AGCTTAACTGTTTCTCCTAAAACGTCTTCTAAATCTTCTTCCGGTGCAT
TAACTGGTAGCTTTACAGTTGCAGTAGACCCTACAGATGCTACAGAAGC
TTTTGAAGTTAAGGTTACAGATAAAGATGGTGCTGATTCTAGTGCTATA
AAAGCTATTATCGATGGCTTAAAAGTTAACTATACTGGAACAGACGTGC
CAGCAGGTAA
V18 UHR seq (SEQ ID NO: 120):
CCAAATCTCAATGCTTACTTGGACAGAGAATGATTTAACATTCTATAAA
GACATCGCTAAAAAACCAGCTACATCTACAGTAGCAAAATACGATGTAT
ACATGCAACATGGTAAGGTAGGTCATACTAGATTTACTCGTGAGATTGG
GGTAGCACCAGTAAGTGACCCTAACATCCGTCAAAAAACAGTAAATATG
AAATTTGCTTCCGATACTAAAAACATCAGTATCGCAGCAGGTCTAGTAA
ACAACATTCAAGACCCAATGCAAATTTTGACTGACGATGCTATCGTAAA
TATTGCTAAAACAATTGAGTGGGCTTCATTCTTTGGAGATTCTGACTTA
TCAGATAGCCCAGAACCACAAGCAGGACTAGAATTTGACGGCTTGGCTA
AACTTATTAACCAAGATAACGTTCATGATGCTCGTGGAGCTAGCTTGAC
TGAAAGCTTGTTAAACCAAGCAGCAGTAATGATTAGTAAAGGTTATGGT
ACACCTACAGATGCTTACATGCCAGTAGGGGTTCAAGCAGACTTTGTTA
ACCAACAACTTTCTAAACAAACACAACTTGTTCGCGATAACGGAAACAA
CGTAAGCGTTGGTTTCAACATCCAAGGTTTCCATTCAGCTCGTGGATTT
ATCAAACTTCACGGTTCTACAGTAATGGAAAACGAACAAATCTTAGATG
AACGTATTCTTGCTTTACCAACAGCTCCACAACCAGCTAAGGTAACTGC
AACACAAGAAGCAGGTAAAAAAGGACAATTTAGAGCAGAAGATTTAGCA
GCACATGAATATAAAGTTGTTGTAAGTTCTGACGATGCAGAGTCTATTG
CAAGTGAAGTGGCTACAGCTACAGTTACTGCAAAAGATGACGGCGTTAA
ACTAGAAATCGAATTAGCTCCAATGTATAGCTCTCGTCCACAATTCGTT
TCAATCTATAGAAAAGGTGCAGAAACAGGTTTATTCTACCTAATCGCTC
GTGTACCTGCTAGCAAAGCAGAGAACAACGTAATCACTTTCTACGACTT
AAACGACTCTATTCCTGAAACAGTAGACGTATTCGTTGGTGAAATGTCG
GCTAACGTAGTACACTTGTTTGAATTACTACCAATGATGAGATTACCTC
TAGCTCAAATTAACGCATCTGTTACATTTGCAGTTTTATGGTATGGCGC
ATTAGCTCTAAGAGCACCTAAGAAATGGGTACGTATTAGAAACGTTAAA
TATATTCCTGTAAAAAACGTTCATAGCAACTAA
V18 DHR seq
(SEQ ID NO: 121)
TAATTATAGGATAATTGAATAAAAACAGTATAGAGAGCAGATAAATACT
GCTCTCTATTTTACTAATAAGGAGGATTTAAATTGCTAAAAAATACAAA
CTTAGCTAATTATAAAAAAGTGAATACACGGTTTGGAAATCTTAGTTTT
GACGACAAAGGTATTTCTAATGACTTAACGGAAGAACAGCAAAAAGAAT
TAGGTAAGCTTAGAGGATTCGAATATATTAAGACAGAACAGAAAACGAA
AGAAGAACCTAAGAAAGAAGAACCTAAGAAAGAAGAACCTAAGAAAGAA
GAACCTAAGAAAGAAAGTACAGAAAATGAATTAGACAGCTTCTTAGCTA
AAGAGCCTTCAATCAAAGAATTAAAAGAATTTGCGAGTAAAAAAGGCAT
TAAAATTGAAAAAACTAAGAAAAATGATATAATTGAAGAACTAAAGAGA
GGGTAATGTATAATGTATGGAGGTTATGAAGGACAAGATTCTTACGAAT
ACCCTTACTCACATGGGAACCCTAAGCATGTAGAGCCAGAAAAAGTTGA
CGAATATGTTCTTTCTGATTATGGTTGGACTGCGGAAACAATTAAAGCA
TACATGTATGGTGTTCGTGTAGTAGACCCTGAAACAGGAGAGGAAATGG
GAGACACCTTCTACAATCATATTATAGAGGTTGCCGTTGATAAGGCAGA
GAAAGAACTAGATATAGCTATTCTCCCAAGACGGGAGTCAGAACACCAC
GATTATAACCAAACAGAATTTAGTAGTT
LP22 UHR seq
(SEQ ID NO: 122)
atattcgctttgCACCACTAGCTACAACATTCAATCAATTACAAGGTTC
TGTAGGGGATACAATCACTCTTCCTAACTGGAACAAAATTGGTAAAGCA
GAAGTTGTTGCAGAAGGTCAAACTTCTAATATTGACACTATTAACCAAT
CTCAAATCTCTGTAACAGTTAAAAAAGCTGTTAAAGCTGTAGCTATTTC
AGACGAACTAGAACTAGCTTCCGCAGGTAACCCTGTTAATGAAATTGTT
GACCAAATCGCAATGGCTTTAGCTCAAAAAGTAGATGATGATTTAATTG
CTATTGCTCGTTCTGCTAAAAAAGCTGTAGACCCTTCAACTGGAGAGGC
TCTTACTGTAGATGCAATCAACAAAATTCCTTTAGCTTTAGCTAACTTT
GGTGAAACTCTTTACGAAGATGCTACATATCTTTTAGTAAGCACTAACA
GTTATGCTTTGTTCGTTTCTGATGATAAGTTTGTTCCTATCATTAATCA
AGGTTCTATCATCATCAATGGTACTATCGGTACTCTTTATGGATGTACT
GTAGTTCTTTCTGATAAAGTACAAGACGGTGAGTTCTTCTTCATTAAAG
CTGGTGCTCTTGGTATTGCTCTTAAACAAGATACACGTATTCTTACTGA
ATATGATTTACTTTCTCACACTACATTAATCAGTGGTGACCGCCACTAT
GCTACATTTATGGCTGATGAAGATAAAATTGTTTATGTAGGTGCAGGCA
CTGCTGTTCCAGCGGCTCCAACTATTGCGACTCCTGCTACAACTGCTTC
TTCTGTTACAATTACTTTACCTGCCTAA
LP22 DHR seq
(SEQ ID NO: 123)
TAAAGACTAAGCCCAGCTTCGGCTGGGTTATAAGTCTATTATAATCGAA
TCGGAGGAATAAATCAATGGCTGTAGAAAAGAAATATGATATAAAGAAA
AATGGTAACATTATCGAAACGTTCCATGCTTCAGGTGACTTTGTGGATA
ATGATGTATTACCAAAAACAAACTACCAATACCAAGTACGTGTGCGTGA
GGTAGACTCTGGTGTAGTAAAATCTACCTCTGAGTGGACTGCTCCTATT
AACGTCAAAACTAAGTAAGGGGAATTAGTAAATGCCAACCGTTAAGAAA
TATGATATTAAACGTAACGGCAATGTGGTAGCTACATATGTTCCCGCTG
GAAATTGGAGAGATGATAATCTTCTTCCTAATACTAACTACCAGTATTT
AGTTAGATGTCATGAAGTTACAACTACTGGTAGTGACGTCAGTATTAAA
ACAAGTGATTGGACAAACCCAATTAACGTTAAAACACTGGTATCTGATA
CTAGTATTCATCCACCTGCTCCTATTAATGTTAATGTAACAGATTTAGA
ATCTGATTCTTTCACTATTGATTGGGGTAATGGAGGATTTAATGCTAAA
ACGTATGTTGTTCGTTTAGGCATTAATGGAGATGGATTGCCAGATAATA
ATGTTTTTGTAACTACAACTAATAGCTTTACAGCTACTGAGGATGTAGT
AGTAGCATTAGCTCCATTCGCTGGTAAGAAAATTGACGTATATGTAGCA
CAATTTGCTGGAGTTTACGCGGATGCCACTTCTGCTTTAGAAAGTGCAG
AATACAATGAAGTAACTGCATGGTCTAACCTAGTTACTGTAGATGTCCC
ATCTGCAAGTCCTTCAATGTTACGTTCTTCTAGTAATAAAGAAATTGTT
AGCTTAACTGTTTCTCCTAAAACGTCTTCTAAATCTTCTTCCGGTGCAT
TAACTGGTAGCTTTACAGTTGCAGTAGACCCTACAGATGCTACAGAAGC
TTTTGAAGTTAAGGTTACAGATAAAGATGGTGCTGATTCTAGTGCTATA
AAAGCTATTATCGATGGCTTAAAAGTTAACTATACTGgaacagacgtgc
cagcaggtaa

TABLE 36
The tested Listeria strains comprised
96 strains that are difficult to target.

| Strain # | Designation | Species |
|---|---|---|
| 1816 | FSL H6-011 | Listeria seeligeri |
| 1817 | FSL H6-169 | Listeria seeligeri |
| 1823 | FSL W3-075 | Listeria innocua |
| | | (hemolytic) |
| 1825 | FSL S4-965 | Listeria marthii |
| 1826 | FSL F6-920 | Listeria rocourtiae |
| 1828 | FSL J1-177 | Listeria monocytogenes |
| 1832 | FSL J1-049 | Listeria monocytogenes |
| 1836 | FSL W1-110 | Listeria monocytogenes |
| 1883 | FSL T1-922 | Listeria monocytogenes |
| 1886 | FSL R2-069 | Listeria monocytogenes |
| 1890 | FSL T1-041 | Listeria monocytogenes |
| 1892 | FSL R8-5247 | Listeria seeligeri |
| 1893 | FSL R8-5253 | Listeria seeligeri |
| 1894 | FSL R8-5513 | Listeria seeligeri |
| 1899 | FSL R8-6852 | Listeria seeligeri |
| 1900 | FSL R8-5085 | Listeria innocua |
| 1907 | FSL R8-5440 | Listeria innocua |
| 1909 | FSL R8-5448 | Listeria innocua |
| 1912 | FSL R8-7061 | Listeria innocua |
| 1916 | FSL R8-7548 | Listeria innocua |
| 1951 | FSL S10-320 | Listeria seeligeri |
| 1962 | FSL R8-5961 | Listeria innocua |
| 1978 | FSL R8-8163 | Listeria welshimeri |
| 1979 | FSL R8-7524 | Listeria welshimeri |
| 1981 | FSL R8-6035 | Listeria welshimeri |
| 1990 | FSL H6-027 | Listeria seeligeri |
| 1991 | FSL H6-079 | Listeria seeligeri |
| 1992 | FSL H6-185 | Listeria seeligeri |
| 1993 | FSL R8-6874 | Listeria seeligeri |
| 1994 | FSLR8-6880 | Listeria seeligeri |
| 1995 | FSL R8-7629 | Listeria seeligeri |
| 2006 | FSL S10-1311 | Listeria innocua |
| 2010 | FSL R8-5594 | Listeria innocua |
| 2011 | FSL R8-7181 | Listeria innocua |
| 2012 | FSL R2-632 | Listeria innocua |
| 2013 | FSL L3-851 | Listeria innocua |
| 2067 | FSL M1-004 | Listeria monocytogenes |
| 2071 | FSL F6-464 | Listeria monocytogenes |
| 2080 | FSL F6-352 | Listeria monocytogenes |
| 2081 | FSL H5-781 | Listeria monocytogenes |
| 2082 | FSL K2-147 | Listeria monocytogenes |
| 2085 | FSL K2-065 | Listeria monocytogenes |
| 2087 | FSL F6-184 | Listeria monocytogenes |
| 2089 | FSL H1-099 | Listeria monocytogenes |
| 2100 | FSL N1-176 | Listeria monocytogenes |
| 2101 | FSL N1-417 | Listeria monocytogenes |
| 2102 | FSL L3-051 | Listeria monocytogenes |
| 2103 | FSL T1-107 | Listeria monocytogenes |
| 2104 | FSL T1-408 | Listeria monocytogenes |
| 2105 | FSL F6-396 | Listeria monocytogenes |
| 2107 | FSL F6-551 | Listeria monocytogenes |
| 2108 | FSL F6-446 | Listeria monocytogenes |
| 2110 | FSL V1-022 | Listeria monocytogenes |
| 2112 | FSL R2-273 | Listeria monocytogenes |
| 2134 | FSL F6-449 | Listeria monocytogenes |
| 2136 | FSL S4-024 | Listeria monocytogenes |
| 2137 | FSL H1-111 | Listeria monocytogenes |
| 2138 | FSL K2-022 | Listeria monocytogenes |
| B4-G7 | Environmnetal | Listeria spp. |
| | Isolate | |
| B5-E10 | Environmnetal | Listeria spp. |
| | Isolate | |
| B6-G7 | Environmnetal | Listeria spp. |
| | Isolate | |
| B7-A10 | Environmnetal | Listeria spp. |
| | Isolate | |
| B7-F6 | Environmnetal | Listeria spp. |
| | Isolate | |
| B9-G4 | Environmnetal | Listeria spp. |
| | Isolate | |
| BG-G10 | Environmnetal | Listeria spp. |
| | Isolate | |
| 085-018-02 1 | Environmnetal | Listeria spp. |
| | Isolate | |
| 085-018-02 2 | Environmnetal | Listeria spp. |
| | Isolate | |
| 085-018-02 3 | Environmnetal | Listeria spp. |
| | Isolate | |
| 088-013 02 S1 | Environmnetal | Listeria spp. |
| | Isolate | |
| 088-013 02 S3 | Environmnetal | Listeria spp. |
| | Isolate | |
| 112-009-08 1 | Environmnetal | Listeria spp. |
| | Isolate | |
| 112-009-08 2 | Environmnetal | Listeria spp. |
| | Isolate | |
| 112-009-08 3 | Environmnetal | Listeria spp. |
| | Isolate | |
| 112-010-02 1 | Environmnetal | Listeria spp. |
| | Isolate | |
| 112-010-02 1 F | Environmnetal | Listeria spp. |
| | Isolate | |
| 112-010-02 1 L | Environmnetal | Listeria spp. |
| | Isolate | |
| 112-010-02 2 | Environmnetal | Listeria spp. |
| | Isolate | |
| 112-010-02 2 F | Environmnetal | Listeria spp. |
| | Isolate | |
| 112-010-02 2 L | Environmnetal | Listeria spp. |
| | Isolate | |
| 112-010-02 3 | Environmnetal | Listeria spp. |
| | Isolate | |
| 112-010-02 3 F | Environmnetal | Listeria spp. |
| | Isolate | |
| 112-010-02 3 L | Environmnetal | Listeria spp. |
| | Isolate | |
| 112-019-01 1 L | Environmnetal | Listeria spp. |
| | Isolate | |
| 112-019-01 2 L | Environmnetal | Listeria spp. |
| | Isolate | |
| 112-019-01 3 L | Environmnetal | Listeria spp. |
| | Isolate | |
| 113-022-01 1 | Environmnetal | Listeria spp. |
| | Isolate | |
| 113-022-01 2 | Environmnetal | Listeria spp. |
| | Isolate | |
| 113-023-02 1 L | Environmnetal | Listeria spp. |
| | Isolate | |
| 113-023-02 2 L | Environmnetal | Listeria spp. |
| | Isolate | |
| 113-023-02 3 L | Environmnetal | Listeria spp. |
| | Isolate | |

TABLE 27

| | | Growth | 10⁵ | 10¹ |
|---|---|---|---|---|
| | Growth | Temper- | negative | Listeria/10⁵ |
| Species | Medium | ature | cfu | negative cfu |
| Bacillus cereus | Nutrient Broth | 30 C. | 53 | 3756 |
| Bacillus | Nutrient Broth | 30 C. | 74 | 4814 |
| megaterium | | | | |
| Bacillus subtilis | Nutrient Broth | 30 C. | 56 | 1982 |
| Enterococcus | Brain Heart | 37 C. | 57 | 3507 |
| durans | Infusion | | | |
| Enterococcus | Brain Heart | 37 C. | 55 | 8735 |
| faceium | Infusion | | | |
| Enterococcus hirae | Brain Heart | 37 C. | 57 | 6145 |
| | Infusion | | | |
| Kocuria varians | Nutrient Broth | 30 C. | 52 | 6283 |
| Kurthia gibsonii | Brain Heart | 30 C. | 44 | 4420 |
| | Infusion | | | |
| Kurthia zopfii | Nutrient Broth | 26 C. | 54 | 7226 |
| Rhodococcus equi | Brain Heart | 37 C. | 61 | 4367 |
| | Infusion | | | |
| Staphylococcus | Tryptic Soy | 37 C. | 55 | 3575 |
| aureus | Broth | | | |
| Staphylococcus | Tryptic Soy | 37 C. | 51 | 4544 |
| epidermidis | Broth | | | |
| Staphylococcus | Nutrient Broth | 37 C. | 59 | 4434 |
| saprophyticus | | | | |
| Streptococcus equi | Brain Heart | 37 C. | 63 | 3368 |
| | Infusion | | | |
| Streptococcus | Brain Heart | 37 C. | 64 | 5287 |
| galloyticus | Infusion | | | |
| Lactobacillus casei | MRS | 37 C., | 59 | 5320 |
| | | 5% CO₂ | | |
| Lactobacillus | MRS | 37 C., | 53 | 6331 |
| buchneri | | 5% CO₃ | | |
| Lactobacillus lactus | MRS | 37 C., | 67 | 5065 |
| | | 5% CO₄ | | |
| Lactobacillus | MRS | 37 C., | 67 | 4318 |
| fermentum | | 5% CO₅ | | |
| Micrococcus lutues | Tryptic Soy | 30 C. | 79 | 3322 |
| | Broth | | | |

TABLE 28

| | | 10⁵ | 10¹ Listeria/10⁵ |
|---|---|---|---|
| Sample # | Species | negative cfu | negative cfu |
| 2501-1 | Pseudomonas protogens | 253 | 3513 |
| 250-2 | Pseudomonas florescens | 285 | 1737 |
| 251(2)-1 | Pseudomonas florescens | 236 | 2903 |
| 251(2)-2 | Aeromonas sp | 240 | 1790 |
| 261(1)-1 | Serratia liquefaciens | 318 | 6165 |
| 261(1)-2 | Serratia proteamaculans | 260 | 4614 |
| 261(2)-1 | Serratia liquefaciens | 296 | 2421 |
| 261(2)-2 | Bacillaceae bacterium | 320 | 5289 |
| 289-1 | Serratia proteamaculans | 273 | 3487 |
| 289-2 | Pseudomonas florescens | 279 | 5161 |
| 289-3 | Pseudomonas poae | 241 | 1922 |
| 290(1)-1 | Pseudomonas sp | 241 | 1965 |
| 290(1)-2 | Pseudomonas sp | 271 | 2178 |
| 290(2)-1 | Pseudomonas fragi | 223 | 3052 |
| 290(3)-1 | Pseudomonas sp | 272 | 2560 |
| 291(1)-1 | Providencia alcalifaciens | 262 | 4963 |
| 291(1)-2 | Serratia sp | 272 | 3827 |
| 291(2)-1 | Serratia grimesii | 240 | 3302 |
| 291(2)-2 | Serratia sp | 213 | 3086 |
| 291-1 | Serratia sp | 270 | 2430 |
| 293-1 | Serratia sp, Hafnia sp. | 243 | 2989 |
| 293-2 | Serratia proteamaculans | 259 | 3254 |
| 296-4 | Serratia proteamaculans | 304 | 2314 |
| 304-1 | Pseudomonas florescens | 272 | 2639 |
| 304-2 | Chryseobacterium sp. | 269 | 2911 |
| 306-1 | Pseudomonas fragi | 266 | 3212 |
| 306-2 | Enterobacteriaceae | 273 | 4358 |

TABLE 30

| | A511/P100 | A511/P100/LP124 | Signal Ratio of |
|---|---|---|---|
| Sample # | cocktail RLU | cocktail RLU | 3-phage/2-phage |
| 398 | 1164 | 2212 | 1.9 |
| 399 | 11459 | 27183 | 2.4 |
| 401 | 2100 | 3058 | 1.5 |
| 402 | 113103 | 217389 | 1.9 |
| 403 | 46219 | 58768 | 1.3 |
| 405 | 9988 | 24151 | 2.4 |
| 407 | 2732 | 5329 | 2.0 |
| 426 | 64717 | 444121 | 6.9 |
| 427 | 75896 | 613358 | 8.1 |

INFORMAL SEQUENCE LISTING

SEQ ID NO: 1 - FF luc open reading frame
ATGGAAGACGCCAAAAACATAAAGAAAGGCCCGGCGCCATTCTATCCTCTAGAGGATGGAACCGCTGGAGA
GCAACTGCATAAGGCTATGAAGAGATACGCCCTGGTTCCTGGAACAATTGCTTTTACAGATGCACATATCG
AGGTGAACATCACGTACGCGGAATACTTCGAAATGTCCGTTCGGTTGGCAGAAGCTATGAAACGATATGGG
CTGAATACAAATCACAGAATCGTCGTATGCAGTGAAAACTCTCTTCAATTCTTTATGCCGGTGTTGGGCGC
GTTATTTATCGGAGTTGCAGTTGCGCCCGCGAACGACATTTATAATGAACGTGAATTGCTCAACAGTATGA
ACATTTCGCAGCCTACCGTAGTGTTTGTTTCCAAAAAGGGGTTGCAAAAAATTTTGAACGTGCAAAAAAAA
TTACCAATAATCCAGAAAATTATTATCATGGATTCTAAAACGGATTACCAGGGATTTCAGTCGATGTACAC
GTTCGTCACATCTCATCTACCTCCCGGTTTTAATGAATACGATTTTGTACCAGAGTCCTTTGATCGTGACA
AAACAATTGCACTGATAATGAATTCCTCTGGATCTACTGGGTTACCTAAGGGTGTGGCCCTTCCGCATAGA
ACTGCCTGCGTCAGATTCTCGCATGCCAGAGATCCTATTTTTGGCAATCAAATCATTCCGGATACTGCGAT
TTTAAGTGTTGTTCCATTCCATCACGGTTTTGGAATGTTTACTACACTCGGATATTTGATATGTGGATTTC
GAGTCGTCTTAATGTATAGATTTGAAGAAGAGCTGTTTTTACGATCCCTTCAGGATTACAAAATTCAAAGT
GCGTTGCTAGTACCAACCCTATTTTCATTCTTCGCCAAAAGCACTCTGATTGACAAATACGATTTATCTAA
TTTACACGAAATTGCTTCTGGGGGCGCACCTCTTTCGAAAGAAGTCGGGGAAGCGGTTGCAAAACGCTTCC
ATCTTCCAGGGATACGACAAGGATATGGGCTCACTGAGACTACATCAGCTATTCTGATTACACCCGAGGGG
GATGATAAACCGGGCGCGGTCGGTAAAGTTGTTCCATTTTTTGAAGCGAAGGTTGTGGATCTGGATACCGG
GAAAACGCTGGGCGTTAATCAGAGAGGCGAATTATGTGTCAGAGGACCTATGATTATGTCCGGTTATGTAA
ACAATCCGGAAGCGACCAACGCCTTGATTGACAAGGATGGATGGCTACATTCTGGAGACATAGCTTACTGG
GACGAAGACGAACACTTCTTCATAGTTGACCGCTTGAAGTCTTTAATTAAATACAAAGGATATCAGGTGGC
CCCCGCTGAATTGGAATCGATATTGTTACAACACCCCAACATCTTCGACGCGGGCGTGGCAGGTCTTCCCG
ACGATGACGCCGGTGAACTTCCCGCCGCCGTTGTTGTTTTGGAGCACGGAAAGACGATGACGGAAAAAGAG
ATCGTGGATTACGTCGCCAGTCAAGTAACAACCGCGAAAAAGTTGCGCGGAGGAGTTGTGTTTGTGGACGA
AGTACCGAAAGGTCTTACCGGAAAACTCGACGCAAGAAAAATCAGAGAGATCCTCATAAAGGCCAAGAAGG
GCGGAAAGTCCAAATTGTAA
SEQ ID NO: 2 - FF luc amino acid sequence
MEDAKNIKKGPAPFYPLEDGTAGEQLHKAMKRYALVPGTIAFTDAHIEVNITYAEYFEMSVRLAEAMKRYG
LNTNHRIVVCSENSLQFFMPVLGALFIGVAVAPANDIYNERELLNSMNISQPTVVFVSKKGLQKILNVQKK
LPIIQKIIIMDSKTDYQGFQSMYTFVTSHLPPGFNEYDFVPESFDRDKTIALIMNSSGSTGLPKGVALPHR
TACVRFSHARDPIFGNQIIPDTAILSVVPFHHGFGMFTTLGYLICGFRVVLMYRFEEELFLRSLQDYKIQS
ALLVPTLFSFFAKSTLIDKYDLSNLHEIASGGAPLSKEVGEAVAKRFHLPGIRQGYGLTETTSAILITPEG
DDKPGAVGKVVPFFEAKVVDLDTGKTLGVNQRGELCVRGPMIMSGYVNNPEATNALIDKDGWLHSGDIAYW
DEDEHFFIVDRLKSLIKYKGYQVAPAELESILLQHPNIFDAGVAGLPDDDAGELPAAVVVLEHGKTMTEKE
IVDYVASQVTTAKKLRGGVVFVDEVPKGLTGKLDARKIREILIKAKKGGKSKL
SEQ ID NO: 3 - Nano luc open reading frame
ATGGTCTTCACACTCGAAGATTTCGTTGGGGACTGGCGACAGACAGCCGGCTACAACCTGGACCAAGTCCT
TGAACAGGGAGGTGTGTCCAGTTTGTTTCAGAATCTCGGGGTGTCCGTAACTCCGATCCAAAGGATTGTCC
TGAGCGGTGAAAATGGGCTGAAGATCGACATCCATGTCATCATCCCGTATGAAGGTCTGAGCGGCGACCAA
ATGGGCCAGATCGAAAAAATTTTTAAGGTGGTGTACCCTGTGGATGATCATCACTTTAAGGTGATCCTGCA
CTATGGCACACTGGTAATCGACGGGGTTACGCCGAACATGATCGACTATTTCGGACGGCCGTATGAAGGCA
TCGCCGTGTTCGACGGCAAAAAGATCACTGTAACAGGGACCCTGTGGAACGGCAACAAAATTATCGACGAG
CGCCTGATCAACCCCGACGGCTCCCTGCTGTTCCGAGTAACCATCAACGGAGTGACCGGCTGGCGGCTGTG
CGAACGCATTCTGGCGTAA
SEQ ID NO: 4 - Nano luc amino acid sequence
MVFTLEDFVGDWRQTAGYNLDQVLEQGGVSSLFQNLGVSVTPIQRIVLSGENGLKIDIHVIIPYEGLSGDQ
MGQIEKIFKVVYPVDDHHFKVILHYGTLVIDGVTPNMIDYFGRPYEGIAVEDGKKITVTGTLWNGNKIIDE
RLINPDGSLLFRVTINGVTGWRLCERILA
SEQ ID NO: 5 - LP040 Cps open reading frame
ATGCCAAAAAATAACAAAGAAGAAGAAGTTAAAGAAGTAAACCTTAATTCAGTACAAGAGGATGCGTTAAA
GTCCTTTACAACTGGTTATGGTATCACACCTGATACACAAACAGATGCAGGGGCACTAAGACGTGAGTTCC
TAGACGACCAAATCTCAATGCTTACTTGGACAGAAAATGATTTAACATTCTACAAAGACATCGCTAAAAAA
CCAGCTACATCTACAGTAGCAAAATACGATGTGTACATGCAACACGGTAAAGTAGGTCATACTAGATTTAC
TCGTGAGATTGGGGTAGCACCAGTAAGTGACCCTAACATCCGTCAAAAAACAGTAAACATGAAATTTGCTT
CTGATACTAAAAATATTAGTATCGCAGCAGGTCTAGTAAACAACATTCAAGACCCTATGCAAATTTTGACT
GATGATGCTATCGTAAATATCGCTAAAACAATTGAGTGGGCTTCATTCTTTGGAGATTCTGACTTATCAGA
TAGCCCAGAACCACAAGCAGGATTAGAATTTGATGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGCGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAA
SEQ ID NO: 6 - LP40 Cps protein
MPKNNKEEEVKEVNLNSVQEDALKSFTTGYGITPDTQTDAGALRREFLDDQISMLTWTENDLTFYKDIAKK
PATSTVAKYDVYMQHGKVGHTRFTREIGVAPVSDPNIRQKTVNMKFASDTKNISIAAGLVNNIQDPMQILT
DDAIVNIAKTIEWASFFGDSDLSDSPEPQAGLEFDGLAKLINQDNVHDARGASLTESLLNQAAVMISKGYG
TPTDAYMPVGVQADFVNQQLSKQTQLVRDNGNNVSVGFNIQGFHSARGFIKLHGSTVMENEQILDERILAL
PTAPQPAKVTATQEAGKKGQFRAEDLAAHEYKVVVSSDDAESIASEVATATVTAKDDGVKLEIELAPMYSS
RPQFVSIYRKGAETGLFYLIARVPASKAENNVITFYDLNDSIPETVDVFVGEMSANVVHLFELLPMMRLPL
AQINASVTFAVLWYGALALRAPKKWVRIRNVKYIPVKNVHSN
SEQ ID NO: 7 - LP48 Cps open reading frame
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAACATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGCGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAA
SEQ ID NO: 8 - LP48 protein
MPKNNKEEEVKEVNLNSVQEDALKSFTTGYGITPDTQTDAGALRREFLDDQISMLTWTENDLTFYKDIAKK
PATSTVAKYDVYMQHGKVGHTRFTREIGVAPVSDPNIRQKTVNMKFASDTKNISIAAGLVNNIQDPMQILT
DDAIVNIAKTIEWASFFGDSDLSDSPEPQAGLEFDGLAKLINQDNVHDARGASLTESLLNQAAVMISKGYG
TPTDAYMPVGVQADFVNQQLSKQTQLVRDNGNNVSVGFNIQGFHSARGFIKLHGSTVMENEQILDERILAL
PTAPQPAKVTATQEAGKKGQFRAEDLAAHEYKVVVSSDDAESIASEVATATVTAKDDGVKLEIELAPMYSS
RPQFVSIYRKGAETGLFYLIARVPASKAENNVITFYDLNDSIPETVDVFVGEMSANVVHLFELLPMMRLPL
AQINASVTFAVLWYGALALRAPKKWVRIRNVKYIPVKNVHSN
SEQ ID NO: 9 - LP099 Cps open reading frame
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAACATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGCGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAA
SEQ ID NO: 10 - LP099 Cps protein
MPKNNKEEEVKEVNLNSVQEDALKSFTTGYGITPDTQTDAGALRREFLDDQISMLTWTENDLTFYKDIAKK
PATSTVAKYDVYMQHGKVGHTRFTREIGVAPVSDPNIRQKTVNMKFASDTKNISIAAGLVNNIQDPMQILT
DDAIVNIAKTIEWASFFGDSDLSDSPEPQAGLEFDGLAKLINQDNVHDARGASLTESLLNQAAVMISKGYG
TPTDAYMPVGVQADFVNQQLSKQTQLVRDNGNNVSVGFNIQGFHSARGFIKLHGSTVMENEQILDERILAL
PTAPQPAKVTATQEAGKKGQFRAEDLAAHEYKVVVSSDDAESIASEVATATVTAKDDGVKLEIELAPMYSS
RPQFVSIYRKGAETGLFYLIARVPASKAENNVITFYDLNDSIPETVDVFVGEMSANVVHLFELLPMMRLPL
AQINASVTFAVLWYGALALRAPKKWVRIRNVKYIPVKNVHSN
SEQ ID NO: 11 - LP101 Cps open reading frame
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAACATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGCGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAA
SEQ ID NO: 12 - LP101 Cps protein
MPKNNKEEEVKEVNLNSVQEDALKSFTTGYGITPDTQTDAGALRREFLDDQISMLTWTENDLTFYKDIAKK
PATSTVAKYDVYMQHGKVGHTRFTREIGVAPVSDPNIRQKTVNMKFASDTKNISIAAGLVNNIQDPMQILT
DDAIVNIAKTIEWASFFGDSDLSDSPEPQAGLEFDGLAKLINQDNVHDARGASLTESLLNQAAVMISKGYG
TPTDAYMPVGVQADFVNQQLSKQTQLVRDNGNNVSVGFNIQGFHSARGFIKLHGSTVMENEQILDERILAL
PTAPQPAKVTATQEAGKKGQFRAEDLAAHEYKVVVSSDDAESIASEVATATVTAKDDGVKLEIELAPMYSS
RPQFVSIYRKGAETGLFYLIARVPASKAENNVITFYDLNDSIPETVDVFVGEMSANVVHLFELLPMMRLPL
AQINASVTFAVLWYGALALRAPKKWVRIRNVKYIPVKNVHSN
SEQ ID NO: 13 - LP124 Cps open reading frame
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAACATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGCGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAA
SEQ ID NO: 14 - LP124 Cps protein
MPKNNKEEEVKEVNLNSVQEDALKSFTTGYGITPDTQTDAGALRREFLDDQISMLTWTENDLTFYKDIAKK
PATSTVAKYDVYMQHGKVGHTRFTREIGVAPVSDPNIRQKTVNMKFASDTKNISIAAGLVNNIQDPMQILT
DDAIVNIAKTIEWASFFGDSDLSDSPEPQAGLEFDGLAKLINQDNVHDARGASLTESLLNQAAVMISKGYG
TPTDAYMPVGVQADFVNQQLSKQTQLVRDNGNNVSVGFNIQGFHSARGFIKLHGSTVMENEQILDERILAL
PTAPQPAKVTATQEAGKKGQFRAEDLAAHEYKVVVSSDDAESIASEVATATVTAKDDGVKLEIELAPMYSS
RPQFVSIYRKGAETGLFYLIARVPASKAENNVITFYDLNDSIPETVDVFVGEMSANVVHLFELLPMMRLPL
AQINASVTFAVLWYGALALRAPKKWVRIRNVKYIPVKNVHSN
SEQ ID NO: 15 - LP125 Cps open reading frame
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAACATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGTGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAA
SEQ ID NO: 16 - LP125 Cps protein
MPKNNKEEEVKEVNLNSVQEDALKSFTTGYGITPDTQTDAGALRREFLDDQISMLTWTENDLTFYKDIAKK
PATSTVAKYDVYMQHGKVGHTRFTREIGVAPVSDPNIRQKTVNMKFASDTKNISIAAGLVNNIQDPMQILT
DDAIVNIAKTIEWASFFGDSDLSDSPEPQAGLEFDGLAKLINQDNVHDARGASLTESLLNQAAVMISKGYG
TPTDAYMPVGVQADFVNQQLSKQTQLVRDNGNNVSVGFNIQGFHSARGFIKLHGSTVMENEQILDERILAL
PTAPQPAKVTATQEAGKKGQFRAEDLAAHEYKVVVSSDDAESIASEVATATVTAKDDGVKLEIELAPMYSS
RPQFVSIYRKGAETGLFYLIARVPASKAENNVITFYDLNDSIPETVDVFVGEMSANVVHLFELLPMMRLPL
AQINASVTFAVLWYGALALRAPKKWVRIRNVKYIPVKNVHSN
SEQ ID NO: 17 - LP143 Cps open reading frame
ATGCCAAAAAATAACAAAGAAGAAGTTAAAGAAGTAAACCTTAATTCAGTACAAGAGGATGCGTTAAAGTC
CTTTACGACTGGTTATGGTATCACACCTGATACACAAACAGATGCAGGAGCATTAAGACGTGAGTTCCTAG
ACGACCAAATCTCAATGCTTACTTGGACAGAGAATGATTTAACATTCTATAAAGACATCGCTAAAAAACCA
GCTACATCTACAGTAGCAAAATACGATGTATACATGCAACATGGTAAGGTAGGTCATACTAGATTTACTCG
TGAGATTGGGGTAGCACCAGTAAGTGACCCTAACATCCGTCAAAAAACAGTAAATATGAAATTTGCTTCCG
ATACTAAAAACATCAGTATCGCAGCAGGTCTAGTAAACAACATTCAAGACCCAATGCAAATTTTGACTGAC
GATGCTATCGTAAATATTGCTAAAACAATTGAGTGGGCTTCATTCTTTGGAGATTCTGACTTATCAGATAG
CCCAGAACCACAAGCAGGACTAGAATTTGACGGCTTGGCTAAACTTATTAACCAAGATAACGTTCATGATG
CTCGTGGAGCTAGCTTGACTGAAAGCTTGTTAAACCAAGCAGCAGTAATGATTAGTAAAGGTTATGGTACA
CCTACAGATGCTTACATGCCAGTAGGGGTTCAAGCAGACTTTGTTAACCAACAACTTTCTAAACAAACACA
ACTTGTTCGCGATAACGGAAACAACGTAAGCGTTGGTTTCAACATCCAAGGTTTCCATTCAGCTCGTGGAT
TTATCAAACTTCACGGTTCTACAGTAATGGAAAACGAACAAATCTTAGATGAACGTATTCTTGCTTTACCA
ACAGCTCCACAACCAGCTAAGGTAACTGCAACACAAGAAGCAGGTAAAAAAGGACAATTTAGAGCAGAAGA
TTTAGCAGCACATGAATATAAAGTTGTTGTAAGTTCTGACGATGCAGAGTCTATTGCAAGTGAAGTGGCTA
CAGCTACAGTTACTGCAAAAGATGACGGCGTTAAACTAGAAATCGAATTAGCTCCAATGTATAGCTCTCGT
CCACAATTCGTTTCAATCTATAGAAAAGGTGCAGAAACAGGTTTATTCTACCTAATCGCTCGTGTACCTGC
TAGCAAAGCAGAGAACAACGTAATCACTTTCTACGACTTAAACGACTCTATTCCTGAAACAGTAGACGTAT
TCGTTGGTGAAATGTCGGCTAACGTAGTACACTTGTTTGAATTACTACCAATGATGAGATTACCTCTAGCT
CAAATTAACGCATCTGTTACATTTGCAGTTTTATGGTATGGCGCATTAGCTCTAAGAGCACCTAAGAAATG
GGTACGTATTAGAAACGTTAAATATATTCCTGTAAAAAACGTTCATAGCAACTAA
SEQ ID NO: 18 - LP143 Cps protein
MPKNNKEEVKEVNLNSVQEDALKSFTTGYGITPDTQTDAGALRREFLDDQISMLTWTENDLTFYKDIAKKP
ATSTVAKYDVYMQHGKVGHTRFTREIGVAPVSDPNIRQKTVNMKFASDTKNISIAAGLVNNIQDPMQILTD
DAIVNIAKTIEWASFFGDSDLSDSPEPQAGLEFDGLAKLINQDNVHDARGASLTESLLNQAAVMISKGYGT
PTDAYMPVGVQADFVNQQLSKQTQLVRDNGNNVSVGFNIQGFHSARGFIKLHGSTVMENEQILDERILALP
TAPQPAKVTATQEAGKKGQFRAEDLAAHEYKVVVSSDDAESIASEVATATVTAKDDGVKLEIELAPMYSSR
PQFVSIYRKGAETGLFYLIARVPASKAENNVITFYDLNDSIPETVDVFVGEMSANVVHLFELLPMMRLPLA
QINASVTFAVLWYGALALRAPKKWVRIRNVKYIPVKNVHSN
SEQ ID NO: 19 - A511 Cps open reading frame
ATGCCAAAAAATAACAAAGAAGAAGTTAAAGAAGTAAACCTTAATTCAGTACAAGAGGATGCGTTAAAGTC
CTTTACGACTGGTTATGGTATCACACCTGATACACAAACAGATGCAGGAGCATTAAGACGTGAGTTCCTAG
ACGACCAAATCTCAATGCTTACTTGGACAGAGAATGATTTAACATTCTATAAAGACATCGCTAAAAAACCA
GCTACATCTACAGTAGCAAAATACGATGTATACATGCAACATGGTAAGGTAGGTCATACTAGATTTACTCG
TGAGATTGGGGTAGCACCAGTAAGTGACCCTAACATCCGTCAAAAAACAGTAAATATGAAATTTGCTTCCG
ATACTAAAAACATCAGTATCGCAGCAGGTCTAGTAAACAACATTCAAGACCCAATGCAAATTTTGACTGAC
GATGCTATCGTAAATATTGCTAAAACAATTGAGTGGGCTTCATTCTTTGGAGATTCTGACTTATCAGATAG
CCCAGAACCACAAGCAGGACTAGAATTTGACGGCTTGGCTAAACTTATTAACCAAGATAACGTTCATGATG
CTCGTGGAGCTAGCTTGACTGAAAGCTTGTTAAACCAAGCAGCAGTAATGATTAGTAAAGGTTATGGTACA
CCTACAGATGCTTACATGCCAGTAGGGGTTCAAGCAGACTTTGTTAACCAACAACTTTCTAAACAAACACA
ACTTGTTCGCGATAACGGAAACAACGTAAGCGTTGGTTTCAACATCCAAGGTTTCCATTCAGCTCGTGGAT
TTATCAAACTTCACGGTTCTACAGTAATGGAAAACGAACAAATCTTAGATGAACGTATTCTTGCTTTACCA
ACAGCTCCACAACCAGCTAAGGTAACTGCAACACAAGAAGCAGGTAAAAAAGGACAATTTAGAGCAGAAGA
TTTAGCAGCACATGAATATAAAGTTGTTGTAAGTTCTGACGATGCAGAGTCTATTGCAAGTGAAGTGGCTA
CAGCTACAGTTACTGCAAAAGATGACGGCGTTAAACTAGAAATCGAATTAGCTCCAATGTATAGCTCTCGT
CCACAATTCGTTTCAATCTATAGAAAAGGTGCAGAAACAGGTTTATTCTACCTAATCGCTCGTGTACCTGC
TAGCAAAGCAGAGAACAACGTAATCACTTTCTACGACTTAAACGACTCTATTCCTGAAACAGTAGACGTAT
TCGTTGGTGAAATGTCGGCTAACGTAGTACACTTGTTTGAATTACTACCAATGATGAGATTACCTCTAGCT
CAAATTAACGCATCTGTTACATTTGCAGTTTTATGGTATGGCGCATTAGCTCTAAGAGCACCTAAGAAATG
GGTACGTATTAGAAACGTTAAATATATTCCTGTAAAAAACGTTCATAGCAACTAA
SEQ ID NO: 20 - A511 Cps protein
MPKNNKEEVKEVNLNSVQEDALKSFTTGYGITPDTQTDAGALRREFLDDQISMLTWTENDLTFYKDIAKKP
ATSTVAKYDVYMQHGKVGHTRFTREIGVAPVSDPNIRQKTVNMKFASDTKNISIAAGLVNNIQDPMQILTD
DAIVNIAKTIEWASFFGDSDLSDSPEPQAGLEFDGLAKLINQDNVHDARGASLTESLLNQAAVMISKGYGT
PTDAYMPVGVQADFVNQQLSKQTQLVRDNGNNVSVGFNIQGFHSARGFIKLHGSTVMENEQILDERILALP
TAPQPAKVTATQEAGKKGQFRAEDLAAHEYKVVVSSDDAESIASEVATATVTAKDDGVKLEIELAPMYSSR
PQFVSIYRKGAETGLFYLIARVPASKAENNVITFYDLNDSIPETVDVFVGEMSANVVHLFELLPMMRLPLA
QINASVTFAVLWYGALALRAPKKWVRIRNVKYIPVKNVHSN
SEQ ID NO: 21 - P100 Cps open reading frame
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAATATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAGCTTGTTCGTGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGACTTAGCAGCACACGAATACAAAGTTGTTGTAAGTTCTGACGATGCAGAGTCTATTGCAAGTGAAGTGG
CTACAGCTACAGTTACTGCAAAAGATGACGGCGTTAAACTAGAAATCGAGTTAGCTCCAATGTACAGCTCC
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTATGACTTAAACGACTCTATTCCTGAAACAGTAGACG
TATTCGTTGGTGAAATGTCTGCTAACGTAGTACACTTGTTTGAATTACTACCAATGATGAGATTACCTCTA
GCTCAAATTAACGCATCTGTTACATTTGCAGTTTTATGGTATGGAGCATTAGCTCTAAGAGCACCTAAGAA
ATGGGTACGTATTAGAAACGTTAAATATATTCCTGTAAAAAACGTTCATAGCAACTAA
SEQ ID NO: 22 - P100 Cps protein
MPKNNKEEEVKEVNLNSVQEDALKSFTTGYGITPDTQTDAGALRREFLDDQISMLTWTENDLTFYKDIAKK
PATSTVAKYDVYMQHGKVGHTRFTREIGVAPVSDPNIRQKTVNMKFASDTKNISIAAGLVNNIQDPMQILT
DDAIVNIAKTIEWASFFGDSDLSDSPEPQAGLEFDGLAKLINQDNVHDARGASLTESLLNQAAVMISKGYG
TPTDAYMPVGVQADFVNQQLSKQTQLVRDNGNNVSVGFNIQGFHSARGFIKLHGSTVMENEQILDERILAL
PTAPQPAKVTATQEAGKKGQFRAEDLAAHEYKVVVSSDDAESIASEVATATVTAKDDGVKLEIELAPMYSS
RPQFVSIYRKGAETGLFYLIARVPASKAENNVITFYDLNDSIPETVDVFVGEMSANVVHLFELLPMMRLPL
AQINASVTFAVLWYGALALRAPKKWVRIRNVKYIPVKNVHSN
SEQ ID NO: 23 - LP48:: ffluc
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAACATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGCGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAAGAGGAGGTAAATA
TATATGGAAGACGCCAAAAACATAAAGAAAGGCCCGGCGCCATTCTATCCTCTAGAGGATGGAACCGCTGG
AGAGCAACTGCATAAGGCTATGAAGAGATACGCCCTGGTTCCTGGAACAATTGCTTTTACAGATGCACATA
TCGAGGTGAACATCACGTACGCGGAATACTTCGAAATGTCCGTTCGGTTGGCAGAAGCTATGAAACGATAT
GGGCTGAATACAAATCACAGAATCGTCGTATGCAGTGAAAACTCTCTTCAATTCTTTATGCCGGTGTTGGG
CGCGTTATTTATCGGAGTTGCAGTTGCGCCCGCGAACGACATTTATAATGAACGTGAATTGCTCAACAGTA
TGAACATTTCGCAGCCTACCGTAGTGTTTGTTTCCAAAAAGGGGTTGCAAAAAATTTTGAACGTGCAAAAA
AAATTACCAATAATCCAGAAAATTATTATCATGGATTCTAAAACGGATTACCAGGGATTTCAGTCGATGTA
CACGTTCGTCACATCTCATCTACCTCCCGGTTTTAATGAATACGATTTTGTACCAGAGTCCTTTGATCGTG
ACAAAACAATTGCACTGATAATGAATTCCTCTGGATCTACTGGGTTACCTAAGGGTGTGGCCCTTCCGCAT
AGAACTGCCTGCGTCAGATTCTCGCATGCCAGAGATCCTATTTTTGGCAATCAAATCATTCCGGATACTGC
GATTTTAAGTGTTGTTCCATTCCATCACGGTTTTGGAATGTTTACTACACTCGGATATTTGATATGTGGAT
TTCGAGTCGTCTTAATGTATAGATTTGAAGAAGAGCTGTTTTTACGATCCCTTCAGGATTACAAAATTCAA
AGTGCGTTGCTAGTACCAACCCTATTTTCATTCTTCGCCAAAAGCACTCTGATTGACAAATACGATTTATC
TAATTTACACGAAATTGCTTCTGGGGGCGCACCTCTTTCGAAAGAAGTCGGGGAAGCGGTTGCAAAACGCT
TCCATCTTCCAGGGATACGACAAGGATATGGGCTCACTGAGACTACATCAGCTATTCTGATTACACCCGAG
GGGGATGATAAACCGGGCGCGGTCGGTAAAGTTGTTCCATTTTTTGAAGCGAAGGTTGTGGATCTGGATAC
CGGGAAAACGCTGGGCGTTAATCAGAGAGGCGAATTATGTGTCAGAGGACCTATGATTATGTCCGGTTATG
TAAACAATCCGGAAGCGACCAACGCCTTGATTGACAAGGATGGATGGCTACATTCTGGAGACATAGCTTAC
TGGGACGAAGACGAACACTTCTTCATAGTTGACCGCTTGAAGTCTTTAATTAAATACAAAGGATATCAGGT
GGCCCCCGCTGAATTGGAATCGATATTGTTACAACACCCCAACATCTTCGACGCGGGCGTGGCAGGTCTTC
CCGACGATGACGCCGGTGAACTTCCAGCCGCCGTTGTTGTTTTGGAGCACGGAAAGACGATGACGGAAAAA
GAGATCGTGGATTACGTCGCCAGTCAAGTAACAACCGCGAAAAAGTTGCGCGGAGGAGTTGTGTTTGTGGA
CGAAGTACCGAAAGGTCTTACCGGAAAACTCGACGCAAGAAAAATCAGAGAGATCCTCATAAAGGCCAAGA
AGGGCGGAAAGTCCAAATTGTAATAATTATAGGATAATTGAATAAAAACAGTATAGAGAGCAGATAAATAC
TGCTCTCTATTTTACTAATAAGGAGGATTTAAATTGCTAAAAAATACAAACTTAGCTAATTATAAAAAAGT
GAATACACGGTTTGGAAATCTTAGTTTTGACGACAAAGGTATTTCTAATGACTTAACGGAAGAACAGCAAA
AAGAATTAGGTAAGCTTCGAGGATTCGAATATATTAAGACAGAACAGAAAACAAAAGAAGAACCTAAGAAA
GAAGAACCTAAGAAAGAAAGTACAGAAAATGAATTAGACAGCTTCTTAGCTAAAGAGCCTTCAATCAAAGA
ATTAAAAGAATTTGCGAGTAAAAAAGGCATTAAAATTGAAAAAACTAAGAAAAATGATATAATTGAAGAAC
TAAAGAGAGGGTAATGTATAATGTATGGAGGTTATGAAGGACAAGATTCTTACGAATACCCTTACTCACAT
GGGAACCCTAAGCATGTAGAGCCAGAAAAAGTTGACGAATATGTTCTTTCTGATTATGGTTGGACTGCGGA
AACAATTAAAGCATACATGTATGGTGTTCGTGTAGTAGACCCTGAAACAGGAGAGGAAATGGGAGACACCT
TCTACAATCATATTATAGAGGTTGCCGTTGATAAGGC
SEQ ID NO: 24 - LP99:: ffluc
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAACATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGCGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAAGAGGAGGTAAATA
TATATGGAAGACGCCAAAAACATAAAGAAAGGCCCGGCGCCATTCTATCCTCTAGAGGATGGAACCGCTGG
AGAGCAACTGCATAAGGCTATGAAGAGATACGCCCTGGTTCCTGGAACAATTGCTTTTACAGATGCACATA
TCGAGGTGAACATCACGTACGCGGAATACTTCGAAATGTCCGTTCGGTTGGCAGAAGCTATGAAACGATAT
GGGCTGAATACAAATCACAGAATCGTCGTATGCAGTGAAAACTCTCTTCAATTCTTTATGCCGGTGTTGGG
CGCGTTATTTATCGGAGTTGCAGTTGCGCCCGCGAACGACATTTATAATGAACGTGAATTGCTCAACAGTA
TGAACATTTCGCAGCCTACCGTAGTGTTTGTTTCCAAAAAGGGGTTGCAAAAAATTTTGAACGTGCAAAAA
AAATTACCAATAATCCAGAAAATTATTATCATGGATTCTAAAACGGATTACCAGGGATTTCAGTCGATGTA
CACGTTCGTCACATCTCATCTACCTCCCGGTTTTAATGAATACGATTTTGTACCAGAGTCCTTTGATCGTG
ACAAAACAATTGCACTGATAATGAATTCCTCTGGATCTACTGGGTTACCTAAGGGTGTGGCCCTTCCGCAT
AGAACTGCCTGCGTCAGATTCTCGCATGCCAGAGATCCTATTTTTGGCAATCAAATCATTCCGGATACTGC
GATTTTAAGTGTTGTTCCATTCCATCACGGTTTTGGAATGTTTACTACACTCGGATATTTGATATGTGGAT
TTCGAGTCGTCTTAATGTATAGATTTGAAGAAGAGCTGTTTTTACGATCCCTTCAGGATTACAAAATTCAA
AGTGCGTTGCTAGTACCAACCCTATTTTCATTCTTCGCCAAAAGCACTCTGATTGACAAATACGATTTATC
TAATTTACACGAAATTGCTTCTGGGGGCGCACCTCTTTCGAAAGAAGTCGGGGAAGCGGTTGCAAAACGCT
TCCATCTTCCAGGGATACGACAAGGATATGGGCTCACTGAGACTACATCAGCTATTCTGATTACACCCGAG
GGGGATGATAAACCGGGCGCGGTCGGTAAAGTTGTTCCATTTTTTGAAGCGAAGGTTGTGGATCTGGATAC
CGGGAAAACGCTGGGCGTTAATCAGAGAGGCGAATTATGTGTCAGAGGACCTATGATTATGTCCGGTTATG
TAAACAATCCGGAAGCGACCAACGCCTTGATTGACAAGGATGGATGGCTACATTCTGGAGACATAGCTTAC
TGGGACGAAGACGAACACTTCTTCATAGTTGACCGCTTGAAGTCTTTAATTAAATACAAAGGATATCAGGT
GGCCCCCGCTGAATTGGAATCGATATTGTTACAACACCCCAACATCTTCGACGCGGGCGTGGCAGGTCTTC
CCGACGATGACGCCGGTGAACTTCCCGCCGCCGTTGTTGTTTTGGAGCACGGAAAGACGATGACGGAAAAA
GAGATCGTGGATTACGTCGCCAGTCAAGTAACAACCGCGAAAAAGTTGCGCGGAGGAGTTGTGTTTGTGGA
CGAAGTACCGAAAGGTCTTACCGGAAAACTCGACGCAAGAAAAATCAGAGAGATCCTCATAAAGGCCAAGA
AGGGCGGAAAGTCCAAATTGTAATAATTATAGGATAATTGAATAAAAACAGTATAGAGAGCAGATAAATAC
TGCTCTCTATTTTACTAATAAGGAGGATTTAAATTGCTAAAAAATACAAACTTAGCTAATTATAAAAAAGT
GAATACACGGTTTGGAAATCTTAGTTTTGACGACAAAGGTATTTCTAATGACTTAACGGAAGAACAGCAAA
AAGAATTAGGTAAGCTTCGAGGATTCGAATATATTAAGACAGAACAGAAAACAAAAGAAGAACCTAAGAAA
GAAGAACCTAAGAAAGAAGAACCTAAGAAAGAAGAACCTAAGAAAGAAGAACCTAAGAAAGAAGAACCTAA
GAAAGAAAGTACAGAAAATGAATTAGACAGCTTCTTAGCTAAAGAGCCTTCAATCAAAGAATTAAAAGAAT
TTGCGAGTAAAAAAGGCATTAAAATTGAAAAAACTAAGAAAAATGATATAATTGAAGAACTAAAGAGAGGG
TAATGTATAATGTATGGAGGTTATGAAGGACAAGATTCTTACGAATACCCTTACTCACATGGGAACCCTAA
GCATGTAGAGCCAGAAAAAGTTGACGAATATGTTCTTTCTGATTATGGTTGGACTGCGGAAACAATTAAAG
CATACATGTATGGTGTTCGTGTAGTAGACCCTGAAACAGGAGAGGAAATGGGAGACACCTTCTACAATCAT
ATTATAGAGGTTGCCGTTGATAAGGC
SEQ ID NO: 25 - LP101:: ffluc
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAACATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGCGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAAGAGGAGGTAAATA
TATATGGAAGACGCCAAAAACATAAAGAAAGGCCCGGCGCCATTCTATCCTCTAGAGGATGGAACCGCTGG
AGAGCAACTGCATAAGGCTATGAAGAGATACGCCCTGGTTCCTGGAACAATTGCTTTTACAGATGCACATA
TCGAGGTGAACATCACGTACGCGGAATACTTCGAAATGTCCGTTCGGTTGGCAGAAGCTATGAAACGATAT
GGGCTGAATACAAATCACAGAATCGTCGTATGCAGTGAAAACTCTCTTCAATTCTTTATGCCGGTGTTGGG
CGCGTTATTTATCGGAGTTGCAGTTGCGCCCGCGAACGACATTTATAATGAACGTGAATTGCTCAACAGTA
TGAACATTTCGCAGCCTACCGTAGTGTTTGTTTCCAAAAAGGGGTTGCAAAAAATTTTGAACGTGCAAAAA
AAATTACCAATAATCCAGAAAATTATTATCATGGATTCTAAAACGGATTACCAGGGATTTCAGTCGATGTA
CACGTTCGTCACATCTCATCTACCTCCCGGTTTTAATGAATACGATTTTGTACCAGAGTCCTTTGATCGTG
ACAAAACAATTGCACTGATAATGAATTCCTCTGGATCTACTGGGTTACCTAAGGGTGTGGCCCTTCCGCAT
AGAACTGCCTGCGTCAGATTCTCGCATGCCAGAGATCCTATTTTTGGCAATCAAATCATTCCGGATACTGC
GATTTTAAGTGTTGTTCCATTCCATCACGGTTTTGGAATGTTTACTACACTCGGATATTTGATATGTGGAT
TTCGAGTCGTCTTAATGTATAGATTTGAAGAAGAGCTGTTTTTACGATCCCTTCAGGATTACAAAATTCAA
AGTGCGTTGCTAGTACCAACCCTATTTTCATTCTTCGCCAAAAGCACTCTGATTGACAAATACGATTTATC
TAATTTACACGAAATTGCTTCTGGGGGCGCACCTCTTTCGAAAGAAGTCGGGGAAGCGGTTGCAAAACGCT
TCCATCTTCCAGGGATACGACAAGGATATGGGCTCACTGAGACTACATCAGCTATTCTGATTACACCCGAG
GGGGATGATAAACCGGGCGCGGTCGGTAAAGTTGTTCCATTTTTTGAAGCGAAGGTTGTGGATCTGGATAC
CGGGAAAACGCTGGGCGTTAATCAGAGAGGCGAATTATGTGTCAGAGGACCTATGATTATGTCCGGTTATG
TAAACAATCCGGAAGCGACCAACGCCTTGATTGACAAGGATGGATGGCTACATTCTGGAGACATAGCTTAC
TGGGACGAAGACGAACACTTCTTCATAGTTGACCGCTTGAAGTCTTTAATTAAATACAAAGGATATCAGGT
GGCCCCCGCTGAATTGGAATCGATATTGTTACAACACCCCAACATCTTCGACGCGGGCGTGGCAGGTCTTC
CCGACGATGACGCCGGTGAACTTCCCGCCGCCGTTGTTGTTTTGGAGCACGGAAAGACGATGACGGAAAAA
GAGATCGTGGATTACGTCGCCAGTCAAGTAACAACCGCGAAAAAGTTGCGCGGAGGAGTTGTGTTTGTGGA
CGAAGTACCGAAAGGTCTTACCGGAAAACTCGACGCAAGAAAAATCAGAGAGATCCTCATAAAGGCCAAGA
AGGGCGGAAAGTCCAAATTGTAATAATTATAGGATAATTGAATAAAAACAGTATAGAGAGCAGATAAATAC
TGCTCTCTATTTTACTAATAAGGAGGATTTAAATTGCTAAAAAATACAAACTTAGCTAATTATAAAAAAGT
GAATACACGGTTTGGAAATCTTAGTTTTGACGACAAAGGTATTTCTAATGACTTAACGGAAGAACAGCAAA
AAGAATTAGGTAAGCTTCGAGGATTCGAATATATTAAGACAGAACAGAAAACAAAAGAAGAACCTAAGAAA
GAAGAACCTAAGAAAGAAGAACCTAAGAAAGAAGAACCTAAGAAAGAAGAACCTAAGAAAGAAGAACCTAA
GAAAGAAAGTACAGAAAATGAATTAGACAGCTTCTTAGCTAAAGAGCCTTCAATCAAAGAATTAAAAGAAT
TTGCGAGTAAAAAAGGCATTAAAATTGAAAAAACTAAGAAAAATGATATAATTGAAGAACTAAAGAGAGGG
TAATGTATAATGTATGGAGGTTATGAAGGACAAGATTCTTACGAATACCCTTACTCACATGGGAACCCTAA
GCATGTAGAGCCAGAAAAAGTTGACGAATATGTTCTTTCTGATTATGGTTGGACTGCGGAAACAATTAAAG
CATACATGTATGGTGTTCGTGTAGTAGACCCTGAAACAGGAGAGGAAATGGGAGACACCTTCTACAATCAT
ATTATAGAGGTTGCCGTTGATAAGGC
SEQ ID NO: 26 - LP124:: ffluc
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAACATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGCGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAAGAGGAGGTAAATA
TATATGGAAGACGCCAAAAACATAAAGAAAGGCCCGGCGCCATTCTATCCTCTAGAGGATGGAACCGCTGG
AGAGCAACTGCATAAGGCTATGAAGAGATACGCCCTGGTTCCTGGAACAATTGCTTTTACAGATGCACATA
TCGAGGTGAACATCACGTACGCGGAATACTTCGAAATGTCCGTTCGGTTGGCAGAAGCTATGAAACGATAT
GGGCTGAATACAAATCACAGAATCGTCGTATGCAGTGAAAACTCTCTTCAATTCTTTATGCCGGTGTTGGG
CGCGTTATTTATCGGAGTTGCAGTTGCGCCCGCGAACGACATTTATAATGAACGTGAATTGCTCAACAGTA
TGAACATTTCGCAGCCTACCGTAGTGTTTGTTTCCAAAAAGGGGTTGCAAAAAATTTTGAACGTGCAAAAA
AAATTACCAATAATCCAGAAAATTATTATCATGGATTCTAAAACGGATTACCAGGGATTTCAGTCGATGTA
CACGTTCGTCACATCTCATCTACCTCCCGGTTTTAATGAATACGATTTTGTACCAGAGTCCTTTGATCGTG
ACAAAACAATTGCACTGATAATGAATTCCTCTGGATCTACTGGGTTACCTAAGGGTGTGGCCCTTCCGCAT
AGAACTGCCTGCGTCAGATTCTCGCATGCCAGAGATCCTATTTTTGGCAATCAAATCATTCCGGATACTGC
GATTTTAAGTGTTGTTCCATTCCATCACGGTTTTGGAATGTTTACTACACTCGGATATTTGATATGTGGAT
TTCGAGTCGTCTTAATGTATAGATTTGAAGAAGAGCTGTTTTTACGATCCCTTCAGGATTACAAAATTCAA
AGTGCGTTGCTAGTACCAACCCTATTTTCATTCTTCGCCAAAAGCACTCTGATTGACAAATACGATTTATC
TAATTTACACGAAATTGCTTCTGGGGGCGCACCTCTTTCGAAAGAAGTCGGGGAAGCGGTTGCAAAACGCT
TCCATCTTCCAGGGATACGACAAGGATATGGGCTCACTGAGACTACATCAGCTATTCTGATTACACCCGAG
GGGGATGATAAACCGGGCGCGGTCGGTAAAGTTGTTCCATTTTTTGAAGCGAAGGTTGTGGATCTGGATAC
CGGGAAAACGCTGGGCGTTAATCAGAGAGGCGAATTATGTGTCAGAGGACCTATGATTATGTCCGGTTATG
TAAACAATCCGGAAGCGACCAACGCCTTGATTGACAAGGATGGATGGCTACATTCTGGAGACATAGCTTAC
TGGGACGAAGACGAACACTTCTTCATAGTTGACCGCTTGAAGTCTTTAATTAAATACAAAGGATATCAGGT
GGCCCCCGCTGAATTGGAATCGATATTGTTACAACACCCCAACATCTTCGACGCGGGCGTGGCAGGTCTTC
CCGACGATGACGCCGGTGAACTTCCCGCCGCCGTTGTTGTTTTGGAGCACGGAAAGACGATGACGGAAAAA
GAGATCGTGGATTACGTCGCCAGTCAAGTAACAACCGCGAAAAAGTTGCGCGGAGGAGTTGTGTTTGTGGA
CGAAGTACCGAAAGGTCTTACCGGAAAACTCGACGCAAGAAAAATCAGAGAGATCCTCATAAAGGCCAAGA
AGGGCGGAAAGTCCAAATTGTAATAATTATAGGATAATTGAATAAAAACAGTATAGAGAGCAGATAAATAC
TGCTCTCTATTTTACTAATAAGGAGGATTTAAATTGCTAAAAAATACAAACTTAGCTAATTATAAAAAAGT
GAATACACGGTTTGGAAATCTTAGTTTTGACGACAAAGGTATTTCTAATGACTTAACGGAAGAACAGCAAA
AAGAATTAGGTAAGCTTCGAGGATTCGAATATATTAAGACAGAACAGAAAACAAAAGAAGAACCTAAGAAA
GAAGAACCTAAGAAAGAAGAACCTAAGAAAGAAGAACCTAAGAAAGAAGAACCTAAGAAAGAAGAACCTAA
GAAAGAAAGTACAGAAAATGAATTAGACAGCTTCTTAGCTAAAGAGCCTTCAATCAAAGAATTAAAAGAAT
TTGCGAGTAAAAAAGGCATTAAAATTGAAAAAACTAAGAAAAATGATATAATTGAAGAACTAAAGAGAGGG
TAATGTATAATGTATGGAGGTTATGAAGGACAAGATTCTTACGAATACCCTTACTCACATGGGAACCCTAA
GCATGTAGAGCCAGAAAAAGTTGACGAATATGTTCTTTCTGATTATGGTTGGACTGCGGAAACAATTAAAG
CATACATGTATGGTGTTCGTGTAGTAGACCCTGAAACAGGAGAGGAAATGGGAGACACCTTCTACAATCAT
ATTATAGAGGTTGCCGTTGATAAGGC
SEQ ID NO: 27 - LP125:: ffluc
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAACATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGTGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAAGAGGAGGTAAATA
TATATGGAAGACGCCAAAAACATAAAGAAAGGCCCGGCGCCATTCTATCCTCTAGAGGATGGAACCGCTGG
AGAGCAACTGCATAAGGCTATGAAGAGATACGCCCTGGTTCCTGGAACAATTGCTTTTACAGATGCACATA
TCGAGGTGAACATCACGTACGCGGAATACTTCGAAATGTCCGTTCGGTTGGCAGAAGCTATGAAACGATAT
GGGCTGAATACAAATCACAGAATCGTCGTATGCAGTGAAAACTCTCTTCAATTCTTTATGCCGGTGTTGGG
CGCGTTATTTATCGGAGTTGCAGTTGCGCCCGCGAACGACATTTATAATGAACGTGAATTGCTCAACAGTA
TGAACATTTCGCAGCCTACCGTAGTGTTTGTTTCCAAAAAGGGGTTGCAAAAAATTTTGAACGTGCAAAAA
AAATTACCAATAATCCAGAAAATTATTATCATGGATTCTAAAACGGATTACCAGGGATTTCAGTCGATGTA
CACGTTCGTCACATCTCATCTACCTCCCGGTTTTAATGAATACGATTTTGTACCAGAGTCCTTTGATCGTG
ACAAAACAATTGCACTGATAATGAATTCCTCTGGATCTACTGGGTTACCTAAGGGTGTGGCCCTTCCGCAT
AGAACTGCCTGCGTCAGATTCTCGCATGCCAGAGATCCTATTTTTGGCAATCAAATCATTCCGGATACTGC
GATTTTAAGTGTTGTTCCATTCCATCACGGTTTTGGAATGTTTACTACACTCGGATATTTGATATGTGGAT
TTCGAGTCGTCTTAATGTATAGATTTGAAGAAGAGCTGTTTTTACGATCCCTTCAGGATTACAAAATTCAA
AGTGCGTTGCTAGTACCAACCCTATTTTCATTCTTCGCCAAAAGCACTCTGATTGACAAATACGATTTATC
TAATTTACACGAAATTGCTTCTGGGGGCGCACCTCTTTCGAAAGAAGTCGGGGAAGCGGTTGCAAAACGCT
TCCATCTTCCAGGGATACGACAAGGATATGGGCTCACTGAGACTACATCAGCTATTCTGATTACACCCGAG
GGGGATGATAAACCGGGCGCGGTCGGTAAAGTTGTTCCATTTTTTGAAGCGAAGGTTGTGGATCTGGATAC
CGGGAAAACGCTGGGCGTTAATCAGAGAGGCGAATTATGTGTCAGAGGACCTATGATTATGTCCGGTTATG
TAAACAATCCGGAAGCGACCAACGCCTTGATTGACAAGGATGGATGGCTACATTCTGGAGACATAGCTTAC
TGGGACGAAGACGAACACTTCTTCATAGTTGACCGCTTGAAGTCTTTAATTAAATACAAAGGATATCAGGT
GGCCCCCGCTGAATTGGAATCGATATTGTTACAACACCCCAACATCTTCGACGCGGGCGTGGCAGGTCTTC
CCGACGATGACGCCGGTGAACTTCCAGCCGCCGTTGTTGTTTTGGAGCACGGAAAGACGATGACGGAAAAA
GAGATCGTGGATTACGTCGCCAGTCAAGTAACAACCGCGAAAAAGTTGCGCGGAGGAGTTGTGTTTGTGGA
CGAAGTACCGAAAGGTCTTACCGGAAAACTCGACGCAAGAAAAATCAGAGAGATCCTCATAAAGGCCAAGA
AGGGCGGAAAGTCCAAATTGTAATAATTATAGGATAATTGAATAAAAACAGTATAGAGAGCAGATAAATAC
TGCTCTCTATTTTACTAATAAGGAGGATTTAAATTGCTAAAAAATACAAACTTAGCTAATTATAAAAAAGT
GAATACACGGTTTGGAAATCTTAGTTTTGACGACAAAGGTATTTCTAATGACTTAACGGAAGAACAGCAAA
AAGAATTAGGTAAGCTTCGAGGATTCGAATATATTAAGACAGAACAGAAAACAAAAGAAGAACCTAAGAAA
GAAGAACCTAAGAAAGAAAGTACAGAAAATGAATTAGACAGCTTCTTAGCTAAAGAGCCTTCAATCAAAGA
ATTAAAAGAATTTGCGAGTAAAAAAGGCATTAAAATTGAAAAAACTAAGAAAAATGATATAATTGAAGAAC
TAAAGAGAGGGTAATGTATAATGTATGGAGGTTATGAAGGACAAGATTCTTACGAATACCCTTACTCACAT
GGGAACCCTAAGCATGTAGAGCCAGAAAAAGTTGACGAATATGTTCTTTCTGATTATGGTTGGACTGCGGA
AACAATTAAAGCATACATGTATGGTGTTCGTGTAGTAGACCCTGAAACAGGAGAGGAAATGGGAGACACCT
TCTACAATCATATTATAGAGGTTGCCGTTGATAAGGC
SEQ ID NO: 28 - LP143:: ffluc
ATGCCAAAAAATAACAAAGAAGAAGTTAAAGAAGTAAACCTTAATTCAGTACAAGAGGATGCGTTAAAGTC
CTTTACGACTGGTTATGGTATCACACCTGATACACAAACAGATGCAGGAGCATTAAGACGTGAGTTCCTAG
ACGACCAAATCTCAATGCTTACTTGGACAGAGAATGATTTAACATTCTATAAAGACATCGCTAAAAAACCA
GCTACATCTACAGTAGCAAAATACGATGTATACATGCAACATGGTAAGGTAGGTCATACTAGATTTACTCG
TGAGATTGGGGTAGCACCAGTAAGTGACCCTAACATCCGTCAAAAAACAGTAAATATGAAATTTGCTTCCG
ATACTAAAAACATCAGTATCGCAGCAGGTCTAGTAAACAACATTCAAGACCCAATGCAAATTTTGACTGAC
GATGCTATCGTAAATATTGCTAAAACAATTGAGTGGGCTTCATTCTTTGGAGATTCTGACTTATCAGATAG
CCCAGAACCACAAGCAGGACTAGAATTTGACGGCTTGGCTAAACTTATTAACCAAGATAACGTTCATGATG
CTCGTGGAGCTAGCTTGACTGAAAGCTTGTTAAACCAAGCAGCAGTAATGATTAGTAAAGGTTATGGTACA
CCTACAGATGCTTACATGCCAGTAGGGGTTCAAGCAGACTTTGTTAACCAACAACTTTCTAAACAAACACA
ACTTGTTCGCGATAACGGAAACAACGTAAGCGTTGGTTTCAACATCCAAGGTTTCCATTCAGCTCGTGGAT
TTATCAAACTTCACGGTTCTACAGTAATGGAAAACGAACAAATCTTAGATGAACGTATTCTTGCTTTACCA
ACAGCTCCACAACCAGCTAAGGTAACTGCAACACAAGAAGCAGGTAAAAAAGGACAATTTAGAGCAGAAGA
TTTAGCAGCACATGAATATAAAGTTGTTGTAAGTTCTGACGATGCAGAGTCTATTGCAAGTGAAGTGGCTA
CAGCTACAGTTACTGCAAAAGATGACGGCGTTAAACTAGAAATCGAATTAGCTCCAATGTATAGCTCTCGT
CCACAATTCGTTTCAATCTATAGAAAAGGTGCAGAAACAGGTTTATTCTACCTAATCGCTCGTGTACCTGC
TAGCAAAGCAGAGAACAACGTAATCACTTTCTACGACTTAAACGACTCTATTCCTGAAACAGTAGACGTAT
TCGTTGGTGAAATGTCGGCTAACGTAGTACACTTGTTTGAATTACTACCAATGATGAGATTACCTCTAGCT
CAAATTAACGCATCTGTTACATTTGCAGTTTTATGGTATGGCGCATTAGCTCTAAGAGCACCTAAGAAATG
GGTACGTATTAGAAACGTTAAATATATTCCTGTAAAAAACGTTCATAGCAACTAAGAGGAGGTAAATATAT
ATGGAAGACGCCAAAAACATAAAGAAAGGCCCGGCGCCATTCTATCCTCTAGAGGATGGAACCGCTGGAGA
GCAACTGCATAAGGCTATGAAGAGATACGCCCTGGTTCCTGGAACAATTGCTTTTACAGATGCACATATCG
AGGTGAACATCACGTACGCGGAATACTTCGAAATGTCCGTTCGGTTGGCAGAAGCTATGAAACGATATGGG
CTGAATACAAATCACAGAATCGTCGTATGCAGTGAAAACTCTCTTCAATTCTTTATGCCGGTGTTGGGCGC
GTTATTTATCGGAGTTGCAGTTGCGCCCGCGAACGACATTTATAATGAACGTGAATTGCTCAACAGTATGA
ACATTTCGCAGCCTACCGTAGTGTTTGTTTCCAAAAAGGGGTTGCAAAAAATTTTGAACGTGCAAAAAAAA
TTACCAATAATCCAGAAAATTATTATCATGGATTCTAAAACGGATTACCAGGGATTTCAGTCGATGTACAC
GTTCGTCACATCTCATCTACCTCCCGGTTTTAATGAATACGATTTTGTACCAGAGTCCTTTGATCGTGACA
AAACAATTGCACTGATAATGAATTCCTCTGGATCTACTGGGTTACCTAAGGGTGTGGCCCTTCCGCATAGA
ACTGCCTGCGTCAGATTCTCGCATGCCAGAGATCCTATTTTTGGCAATCAAATCATTCCGGATACTGCGAT
TTTAAGTGTTGTTCCATTCCATCACGGTTTTGGAATGTTTACTACACTCGGATATTTGATATGTGGATTTC
GAGTCGTCTTAATGTATAGATTTGAAGAAGAGCTGTTTTTACGATCCCTTCAGGATTACAAAATTCAAAGT
GCGTTGCTAGTACCAACCCTATTTTCATTCTTCGCCAAAAGCACTCTGATTGACAAATACGATTTATCTAA
TTTACACGAAATTGCTTCTGGGGGCGCACCTCTTTCGAAAGAAGTCGGGGAAGCGGTTGCAAAACGCTTCC
ATCTTCCAGGGATACGACAAGGATATGGGCTCACTGAGACTACATCAGCTATTCTGATTACACCCGAGGGG
GATGATAAACCGGGCGCGGTCGGTAAAGTTGTTCCATTTTTTGAAGCGAAGGTTGTGGATCTGGATACCGG
GAAAACGCTGGGCGTTAATCAGAGAGGCGAATTATGTGTCAGAGGACCTATGATTATGTCCGGTTATGTAA
ACAATCCGGAAGCGACCAACGCCTTGATTGACAAGGATGGATGGCTACATTCTGGAGACATAGCTTACTGG
GACGAAGACGAACACTTCTTCATAGTTGACCGCTTGAAGTCTTTAATTAAATACAAAGGATATCAGGTGGC
CCCCGCTGAATTGGAATCGATATTGTTACAACACCCCAACATCTTCGACGCGGGCGTGGCAGGTCTTCCCG
ACGATGACGCCGGTGAACTTCCCGCCGCCGTTGTTGTTTTGGAGCACGGAAAGACGATGACGGAAAAAGAG
ATCGTGGATTACGTCGCCAGTCAAGTAACAACCGCGAAAAAGTTGCGCGGAGGAGTTGTGTTTGTGGACGA
AGTACCGAAAGGTCTTACCGGAAAACTCGACGCAAGAAAAATCAGAGAGATCCTCATAAAGGCCAAGAAGG
GCGGAAAGTCCAAATTGTAATAATTATAGGATAATTGAATAAAAACAGTATAGAGAGCAGATAAATACTGC
TCTCTATTTTACTAATAAGGAGGATTTAAATTGCTAAAAAATACAAACTTAGCTAATTATAAAAAAGTGAA
TACACGGTTTGGAAATCTTAGTTTTGACGACAAAGGTATTTCTAATGACTTAACGGAAGAACAGCAAAAAG
AATTAGGTAAGCTTCGAGGATTCGAATATATTAAGACAGAACAGAAAACAAAAGAAGAACCTAAGAAAGAA
GAACCTAAGAAAGAAGAACCTAAGAAAGAAGAACCTAAGAAAGAAGAACCTAAGAAAGAAGAACCTAAGAA
AGAAAGTACAGAAAATGAATTAGACAGCTTCTTAGCTAAAGAGCCTTCAATCAAAGAATTAAAAGAATTTG
CGAGTAAAAAAGGCATTAAAATTGAAAAAACTAAGAAAAATGATATAATTGAAGAACTAAAGAGAGGGTAA
TGTATAATGTATGGAGGTTATGAAGGACAAGATTCTTACGAATACCCTTACTCACATGGGAACCCTAAGCA
TGTAGAGCCAGAAAAAGTTGACGAATATGTTCTTTCTGATTATGGTTGGACTGCGGAAACAATTAAAGCAT
ACATGTATGGTGTTCGTGTAGTAGACCCTGAAACAGGAGAGGAAATGGGAGACACCTTCTACAATCATATT
ATAGAGGTTGCCGTTGATAAGGC
SEQ ID NO: 29 - A511:ffluc
ATGCCAAAAAATAACAAAGAAGAAGTTAAAGAAGTAAACCTTAATTCAGTACAAGAGGATGCGTTAAAGTC
CTTTACGACTGGTTATGGTATCACACCTGATACACAAACAGATGCAGGAGCATTAAGACGTGAGTTCCTAG
ACGACCAAATCTCAATGCTTACTTGGACAGAGAATGATTTAACATTCTATAAAGACATCGCTAAAAAACCA
GCTACATCTACAGTAGCAAAATACGATGTATACATGCAACATGGTAAGGTAGGTCATACTAGATTTACTCG
TGAGATTGGGGTAGCACCAGTAAGTGACCCTAACATCCGTCAAAAAACAGTAAATATGAAATTTGCTTCCG
ATACTAAAAACATCAGTATCGCAGCAGGTCTAGTAAACAACATTCAAGACCCAATGCAAATTTTGACTGAC
GATGCTATCGTAAATATTGCTAAAACAATTGAGTGGGCTTCATTCTTTGGAGATTCTGACTTATCAGATAG
CCCAGAACCACAAGCAGGACTAGAATTTGACGGCTTGGCTAAACTTATTAACCAAGATAACGTTCATGATG
CTCGTGGAGCTAGCTTGACTGAAAGCTTGTTAAACCAAGCAGCAGTAATGATTAGTAAAGGTTATGGTACA
CCTACAGATGCTTACATGCCAGTAGGGGTTCAAGCAGACTTTGTTAACCAACAACTTTCTAAACAAACACA
ACTTGTTCGCGATAACGGAAACAACGTAAGCGTTGGTTTCAACATCCAAGGTTTCCATTCAGCTCGTGGAT
TTATCAAACTTCACGGTTCTACAGTAATGGAAAACGAACAAATCTTAGATGAACGTATTCTTGCTTTACCA
ACAGCTCCACAACCAGCTAAGGTAACTGCAACACAAGAAGCAGGTAAAAAAGGACAATTTAGAGCAGAAGA
TTTAGCAGCACATGAATATAAAGTTGTTGTAAGTTCTGACGATGCAGAGTCTATTGCAAGTGAAGTGGCTA
CAGCTACAGTTACTGCAAAAGATGACGGCGTTAAACTAGAAATCGAATTAGCTCCAATGTATAGCTCTCGT
CCACAATTCGTTTCAATCTATAGAAAAGGTGCAGAAACAGGTTTATTCTACCTAATCGCTCGTGTACCTGC
TAGCAAAGCAGAGAACAACGTAATCACTTTCTACGACTTAAACGACTCTATTCCTGAAACAGTAGACGTAT
TCGTTGGTGAAATGTCGGCTAACGTAGTACACTTGTTTGAATTACTACCAATGATGAGATTACCTCTAGCT
CAAATTAACGCATCTGTTACATTTGCAGTTTTATGGTATGGCGCATTAGCTCTAAGAGCACCTAAGAAATG
GGTACGTATTAGAAACGTTAAATATATTCCTGTAAAAAACGTTCATAGCAACTAAGAGGAGGTAAATATAT
ATGGAAGACGCCAAAAACATAAAGAAAGGCCCGGCGCCATTCTATCCTCTAGAGGATGGAACCGCTGGAGA
GCAACTGCATAAGGCTATGAAGAGATACGCCCTGGTTCCTGGAACAATTGCTTTTACAGATGCACATATCG
AGGTGAACATCACGTACGCGGAATACTTCGAAATGTCCGTTCGGTTGGCAGAAGCTATGAAACGATATGGG
CTGAATACAAATCACAGAATCGTCGTATGCAGTGAAAACTCTCTTCAATTCTTTATGCCGGTGTTGGGCGC
GTTATTTATCGGAGTTGCAGTTGCGCCCGCGAACGACATTTATAATGAACGTGAATTGCTCAACAGTATGA
ACATTTCGCAGCCTACCGTAGTGTTTGTTTCCAAAAAGGGGTTGCAAAAAATTTTGAACGTGCAAAAAAAA
TTACCAATAATCCAGAAAATTATTATCATGGATTCTAAAACGGATTACCAGGGATTTCAGTCGATGTACAC
GTTCGTCACATCTCATCTACCTCCCGGTTTTAATGAATACGATTTTGTACCAGAGTCCTTTGATCGTGACA
AAACAATTGCACTGATAATGAATTCCTCTGGATCTACTGGGTTACCTAAGGGTGTGGCCCTTCCGCATAGA
ACTGCCTGCGTCAGATTCTCGCATGCCAGAGATCCTATTTTTGGCAATCAAATCATTCCGGATACTGCGAT
TTTAAGTGTTGTTCCATTCCATCACGGTTTTGGAATGTTTACTACACTCGGATATTTGATATGTGGATTTC
GAGTCGTCTTAATGTATAGATTTGAAGAAGAGCTGTTTTTACGATCCCTTCAGGATTACAAAATTCAAAGT
GCGTTGCTAGTACCAACCCTATTTTCATTCTTCGCCAAAAGCACTCTGATTGACAAATACGATTTATCTAA
TTTACACGAAATTGCTTCTGGGGGCGCACCTCTTTCGAAAGAAGTCGGGGAAGCGGTTGCAAAACGCTTCC
ATCTTCCAGGGATACGACAAGGATATGGGCTCACTGAGACTACATCAGCTATTCTGATTACACCCGAGGGG
GATGATAAACCGGGCGCGGTCGGTAAAGTTGTTCCATTTTTTGAAGCGAAGGTTGTGGATCTGGATACCGG
GAAAACGCTGGGCGTTAATCAGAGAGGCGAATTATGTGTCAGAGGACCTATGATTATGTCCGGTTATGTAA
ACAATCCGGAAGCGACCAACGCCTTGATTGACAAGGATGGATGGCTACATTCTGGAGACATAGCTTACTGG
GACGAAGACGAACACTTCTTCATAGTTGACCGCTTGAAGTCTTTAATTAAATACAAAGGATATCAGGTGGC
CCCCGCTGAATTGGAATCGATATTGTTACAACACCCCAACATCTTCGACGCGGGCGTGGCAGGTCTTCCCG
ACGATGACGCCGGTGAACTTCCCGCCGCCGTTGTTGTTTTGGAGCACGGAAAGACGATGACGGAAAAAGAG
ATCGTGGATTACGTCGCCAGTCAAGTAACAACCGCGAAAAAGTTGCGCGGAGGAGTTGTGTTTGTGGACGA
AGTACCGAAAGGTCTTACCGGAAAACTCGACGCAAGAAAAATCAGAGAGATCCTCATAAAGGCCAAGAAGG
GCGGAAAGTCCAAATTGTAATAATTATAGGATAATTGAATAAAAACAGTATAGAGAGCAGATAAATACTGC
TCTCTATTTTACTAATAAGGAGGATTTAAATTGCTAAAAAATACAAACTTAGCTAATTATAAAAAAGTGAA
TACACGGTTTGGAAATCTTAGTTTTGACGACAAAGGTATTTCTAATGACTTAACGGAAGAACAGCAAAAAG
AATTAGGTAAGCTTCGAGGATTCGAATATATTAAGACAGAACAGAAAACAAAAGAAGAACCTAAGAAAGAA
GAACCTAAGAAAGAAGAACCTAAGAAAGAAGAACCTAAGAAAGAAGAACCTAAGAAAGAAGAACCTAAGAA
AGAAAGTACAGAAAATGAATTAGACAGCTTCTTAGCTAAAGAGCCTTCAATCAAAGAATTAAAAGAATTTG
CGAGTAAAAAAGGCATTAAAATTGAAAAAACTAAGAAAAATGATATAATTGAAGAACTAAAGAGAGGGTAA
TGTATAATGTATGGAGGTTATGAAGGACAAGATTCTTACGAATACCCTTACTCACATGGGAACCCTAAGCA
TGTAGAGCCAGAAAAAGTTGACGAATATGTTCTTTCTGATTATGGTTGGACTGCGGAAACAATTAAAGCAT
ACATGTATGGTGTTCGTGTAGTAGACCCTGAAACAGGAGAGGAAATGGGAGACACCTTCTACAATCATATT
ATAGAGGTTGCCGTTGATAAGGC
SEQ ID NO: 30 - P100:ffluc
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAATATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAGCTTGTTCGTGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGACTTAGCAGCACACGAATACAAAGTTGTTGTAAGTTCTGACGATGCAGAGTCTATTGCAAGTGAAGTGG
CTACAGCTACAGTTACTGCAAAAGATGACGGCGTTAAACTAGAAATCGAGTTAGCTCCAATGTACAGCTCC
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTATGACTTAAACGACTCTATTCCTGAAACAGTAGACG
TATTCGTTGGTGAAATGTCTGCTAACGTAGTACACTTGTTTGAATTACTACCAATGATGAGATTACCTCTA
GCTCAAATTAACGCATCTGTTACATTTGCAGTTTTATGGTATGGAGCATTAGCTCTAAGAGCACCTAAGAA
ATGGGTACGTATTAGAAACGTTAAATATATTCCTGTAAAAAACGTTCATAGCAACTAAGAGGAGGTAAATA
TATATGGAAGACGCCAAAAACATAAAGAAAGGCCCGGCGCCATTCTATCCTCTAGAGGATGGAACCGCTGG
AGAGCAACTGCATAAGGCTATGAAGAGATACGCCCTGGTTCCTGGAACAATTGCTTTTACAGATGCACATA
TCGAGGTGAACATCACGTACGCGGAATACTTCGAAATGTCCGTTCGGTTGGCAGAAGCTATGAAACGATAT
GGGCTGAATACAAATCACAGAATCGTCGTATGCAGTGAAAACTCTCTTCAATTCTTTATGCCGGTGTTGGG
CGCGTTATTTATCGGAGTTGCAGTTGCGCCCGCGAACGACATTTATAATGAACGTGAATTGCTCAACAGTA
TGAACATTTCGCAGCCTACCGTAGTGTTTGTTTCCAAAAAGGGGTTGCAAAAAATTTTGAACGTGCAAAAA
AAATTACCAATAATCCAGAAAATTATTATCATGGATTCTAAAACGGATTACCAGGGATTTCAGTCGATGTA
CACGTTCGTCACATCTCATCTACCTCCCGGTTTTAATGAATACGATTTTGTACCAGAGTCCTTTGATCGTG
ACAAAACAATTGCACTGATAATGAATTCCTCTGGATCTACTGGGTTACCTAAGGGTGTGGCCCTTCCGCAT
AGAACTGCCTGCGTCAGATTCTCGCATGCCAGAGATCCTATTTTTGGCAATCAAATCATTCCGGATACTGC
GATTTTAAGTGTTGTTCCATTCCATCACGGTTTTGGAATGTTTACTACACTCGGATATTTGATATGTGGAT
TTCGAGTCGTCTTAATGTATAGATTTGAAGAAGAGCTGTTTTTACGATCCCTTCAGGATTACAAAATTCAA
AGTGCGTTGCTAGTACCAACCCTATTTTCATTCTTCGCCAAAAGCACTCTGATTGACAAATACGATTTATC
TAATTTACACGAAATTGCTTCTGGGGGCGCACCTCTTTCGAAAGAAGTCGGGGAAGCGGTTGCAAAACGCT
TCCATCTTCCAGGGATACGACAAGGATATGGGCTCACTGAGACTACATCAGCTATTCTGATTACACCCGAG
GGGGATGATAAACCGGGCGCGGTCGGTAAAGTTGTTCCATTTTTTGAAGCGAAGGTTGTGGATCTGGATAC
CGGGAAAACGCTGGGCGTTAATCAGAGAGGCGAATTATGTGTCAGAGGACCTATGATTATGTCCGGTTATG
TAAACAATCCGGAAGCGACCAACGCCTTGATTGACAAGGATGGATGGCTACATTCTGGAGACATAGCTTAC
TGGGACGAAGACGAACACTTCTTCATAGTTGACCGCTTGAAGTCTTTAATTAAATACAAAGGATATCAGGT
GGCCCCCGCTGAATTGGAATCGATATTGTTACAACACCCCAACATCTTCGACGCGGGCGTGGCAGGTCTTC
CCGACGATGACGCCGGTGAACTTCCCGCCGCCGTTGTTGTTTTGGAGCACGGAAAGACGATGACGGAAAAA
GAGATCGTGGATTACGTCGCCAGTCAAGTAACAACCGCGAAAAAGTTGCGCGGAGGAGTTGTGTTTGTGGA
CGAAGTACCGAAAGGTCTTACCGGAAAACTCGACGCAAGAAAAATCAGAGAGATCCTCATAAAGGCCAAGA
AGGGCGGAAAGTCCAAATTGTAATAATTATAGGATAATTGAATAAAAACAGTATAGAGAGCAGATAAATAC
TGCTCTCTATTTTACTAATAAGGAGGATTTAAATTGCTAAAAAATACAAACTTAGCTAATTATAAAAAAGT
GAATACACGATTTGGAAATCTTAGTTTTGATGATAAAGGTATTTCTAATGACCTAACGGAAGAGCAGCAAA
AAGAATTAGGTAAGCTTAGAGGATTCGAATATATTAAGACAGAACAGAAAACGAAAGAAGAACCTAAGAAA
GAAGAACCTAAGAAAGAAAGTACAGAAAATGAATTAGACAGCTTCTTAGCTAAAGAACCTTCAATCAAAGA
ATTAAAAGAATTTGCGAGTAAAAAAGGCATTAAAATTGAAAAAACTAAGAAAAATGATATAATTGAAGAAC
TAAAGAGAGGGTAATGTACAATGTATGGAGGTTATGAAGGACAAGATTCTTACGAATACCCTTACTCACAC
GGGAACCCTAAGCATGTAGAGCCAGAAAAAGTTGACGAATATGTTCTTTCTGATTATGGCTGGACTGCGGA
AACAATTAAAGCATACATGTATGGTGTTCGTGTAGTAGACCCTGAAACAGGAGAGGAAATGGGAGACACCT
TCTACAATCATATTATAGAGGTTGCCGTTGATAAGGC
SEQ ID NO: 31 - LP124:: nluc
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAACATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGCGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAATAATAAGAGGAGG
TAAATATATATGGTCTTCACACTCGAAGATTTCGTTGGGGACTGGCGACAGACAGCCGGCTACAACCTGGA
CCAAGTCCTTGAACAGGGAGGTGTGTCCAGTTTGTTTCAGAATCTCGGGGTGTCCGTAACTCCGATCCAAA
GGATTGTCCTGAGCGGTGAAAATGGGCTGAAGATCGACATCCATGTCATCATCCCGTATGAAGGTCTGAGC
GGCGACCAAATGGGCCAGATCGAAAAAATTTTTAAGGTGGTGTACCCTGTGGATGATCATCACTTTAAGGT
GATCCTGCACTATGGCACACTGGTAATCGACGGGGTTACGCCGAACATGATCGACTATTTCGGACGGCCGT
ATGAAGGCATCGCCGTGTTCGACGGCAAAAAGATCACTGTAACAGGGACCCTGTGGAACGGCAACAAAATT
ATCGACGAGCGCCTGATCAACCCCGACGGCTCCCTGCTGTTCCGAGTAACCATCAACGGAGTGACCGGCTG
GCGGCTGTGCGAACGCATTCTGGCGTAATAATTATAGGATAATTGAATAAAAACAGTATAGAGAGCAGATA
AATACTGCTCTCTATTTTACTAATAAGGAGGATTTAAATTGCTAAAAAATACAAACTTAGCTAATTATAAA
AAAGTGAATACACGGTTTGGAAATCTTAGTTTTGACGACAAAGGTATTTCTAATGACTTAACGGAAGAACA
GCAAAAAGAATTAGGTAAGCTTCGAGGATTCGAATATATTAAGACAGAACAGAAAACAAAAGAAGAACCTA
AGAAAGAAGAACCTAAGAAAGAAGAACCTAAGAAAGAAGAACCTAAGAAAGAAGAACCTAAGAAAGAAGAA
CCTAAGAAAGAAAGTACAGAAAATGAATTAGACAGCTTCTTAGCTAAAGAGCCTTCAATCAAAGAATTAAA
AGAATTTGCGAGTAAAAAAGGCATTAAAATTGAAAAAACTAAGAAAAATGATATAATTGAAGAACTAAAGA
GAGGGTAATGTATAATGTATGGAGGTTATGAAGGACAAGATTCTTACGAATACCCTTACTCACATGGGAAC
CCTAAGCATGTAGAGCCAGAAAAAGTTGACGAATATGTTCTTTCTGATTATGGTTGGACTGCGGAAACAAT
TAAAGCATACATGTATGGTGTTCGTGTAGTAGACCCTGAAACAGGAGAGGAAATGGGAGACACCTTCTACA
ATCATATTATAGAGGTTGCCGTTGATAAGGC
SEQ ID NO: 32 - LP125:: nluc
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAACATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGTGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAATAATAAGAGGAGG
TAAATATATATGGTCTTCACACTCGAAGATTTCGTTGGGGACTGGCGACAGACAGCCGGCTACAACCTGGA
CCAAGTCCTTGAACAGGGAGGTGTGTCCAGTTTGTTTCAGAATCTCGGGGTGTCCGTAACTCCGATCCAAA
GGATTGTCCTGAGCGGTGAAAATGGGCTGAAGATCGACATCCATGTCATCATCCCGTATGAAGGTCTGAGC
GGCGACCAAATGGGCCAGATCGAAAAAATTTTTAAGGTGGTGTACCCTGTGGATGATCATCACTTTAAGGT
GATCCTGCACTATGGCACACTGGTAATCGACGGGGTTACGCCGAACATGATCGACTATTTCGGACGGCCGT
ATGAAGGCATCGCCGTGTTCGACGGCAAAAAGATCACTGTAACAGGGACCCTGTGGAACGGCAACAAAATT
ATCGACGAGCGCCTGATCAACCCCGACGGCTCCCTGCTGTTCCGAGTAACCATCAACGGAGTGACCGGCTG
GCGGCTGTGCGAACGCATTCTGGCGTAATAATTATAGGATAATTGAATAAAAACAGTATAGAGAGCAGATA
AATACTGCTCTCTATTTTACTAATAAGGAGGATTTAAATTGCTAAAAAATACAAACTTAGCTAATTATAAA
AAAGTGAATACACGGTTTGGAAATCTTAGTTTTGACGACAAAGGTATTTCTAATGACTTAACGGAAGAACA
GCAAAAAGAATTAGGTAAGCTTCGAGGATTCGAATATATTAAGACAGAACAGAAAACAAAAGAAGAACCTA
AGAAAGAAGAACCTAAGAAAGAAAGTACAGAAAATGAATTAGACAGCTTCTTAGCTAAAGAGCCTTCAATC
AAAGAATTAAAAGAATTTGCGAGTAAAAAAGGCATTAAAATTGAAAAAACTAAGAAAAATGATATAATTGA
AGAACTAAAGAGAGGGTAATGTATAATGTATGGAGGTTATGAAGGACAAGATTCTTACGAATACCCTTACT
CACATGGGAACCCTAAGCATGTAGAGCCAGAAAAAGTTGACGAATATGTTCTTTCTGATTATGGTTGGACT
GCGGAAACAATTAAAGCATACATGTATGGTGTTCGTGTAGTAGACCCTGAAACAGGAGAGGAAATGGGAGA
CACCTTCTACAATCATATTATAGAGGTTGCCGTTGATAAGGC
SEQ ID NO: 33 - A511:: nluc
ATGCCAAAAAATAACAAAGAAGAAGTTAAAGAAGTAAACCTTAATTCAGTACAAGAGGATGCGTTAAAGTC
CTTTACGACTGGTTATGGTATCACACCTGATACACAAACAGATGCAGGAGCATTAAGACGTGAGTTCCTAG
ACGACCAAATCTCAATGCTTACTTGGACAGAGAATGATTTAACATTCTATAAAGACATCGCTAAAAAACCA
GCTACATCTACAGTAGCAAAATACGATGTATACATGCAACATGGTAAGGTAGGTCATACTAGATTTACTCG
TGAGATTGGGGTAGCACCAGTAAGTGACCCTAACATCCGTCAAAAAACAGTAAATATGAAATTTGCTTCCG
ATACTAAAAACATCAGTATCGCAGCAGGTCTAGTAAACAACATTCAAGACCCAATGCAAATTTTGACTGAC
GATGCTATCGTAAATATTGCTAAAACAATTGAGTGGGCTTCATTCTTTGGAGATTCTGACTTATCAGATAG
CCCAGAACCACAAGCAGGACTAGAATTTGACGGCTTGGCTAAACTTATTAACCAAGATAACGTTCATGATG
CTCGTGGAGCTAGCTTGACTGAAAGCTTGTTAAACCAAGCAGCAGTAATGATTAGTAAAGGTTATGGTACA
CCTACAGATGCTTACATGCCAGTAGGGGTTCAAGCAGACTTTGTTAACCAACAACTTTCTAAACAAACACA
ACTTGTTCGCGATAACGGAAACAACGTAAGCGTTGGTTTCAACATCCAAGGTTTCCATTCAGCTCGTGGAT
TTATCAAACTTCACGGTTCTACAGTAATGGAAAACGAACAAATCTTAGATGAACGTATTCTTGCTTTACCA
ACAGCTCCACAACCAGCTAAGGTAACTGCAACACAAGAAGCAGGTAAAAAAGGACAATTTAGAGCAGAAGA
TTTAGCAGCACATGAATATAAAGTTGTTGTAAGTTCTGACGATGCAGAGTCTATTGCAAGTGAAGTGGCTA
CAGCTACAGTTACTGCAAAAGATGACGGCGTTAAACTAGAAATCGAATTAGCTCCAATGTATAGCTCTCGT
CCACAATTCGTTTCAATCTATAGAAAAGGTGCAGAAACAGGTTTATTCTACCTAATCGCTCGTGTACCTGC
TAGCAAAGCAGAGAACAACGTAATCACTTTCTACGACTTAAACGACTCTATTCCTGAAACAGTAGACGTAT
TCGTTGGTGAAATGTCGGCTAACGTAGTACACTTGTTTGAATTACTACCAATGATGAGATTACCTCTAGCT
CAAATTAACGCATCTGTTACATTTGCAGTTTTATGGTATGGCGCATTAGCTCTAAGAGCACCTAAGAAATG
GGTACGTATTAGAAACGTTAAATATATTCCTGTAAAAAACGTTCATAGCAACTAAGAGGAGGTAAATATAT
ATGGTCTTCACACTCGAAGATTTCGTTGGGGACTGGCGACAGACAGCCGGCTACAACCTGGACCAAGTCCT
TGAACAGGGAGGTGTGTCCAGTTTGTTTCAGAATCTCGGGGTGTCCGTAACTCCGATCCAAAGGATTGTCC
TGAGCGGTGAAAATGGGCTGAAGATCGACATCCATGTAATCATCCCGTATGAAGGTCTGAGCGGCGACCAA
ATGGGCCAGATCGAAAAAATTTTTAAGGTGGTGTACCCTGTGGATGATCATCACTTTAAGGTGATCCTGCA
CTATGGCACACTGGTAATCGACGGGGTTACGCCGAACATGATCGACTATTTCGGACGGCCGTATGAAGGCA
TCGCCGTGTTCGACGGCAAAAAGATCACTGTAACAGGGACCCTGTGGAACGGCAACAAAATTATCGACGAG
CGCCTGATCAACCCCGACGGCTCCCTGCTGTTCCGAGTAACCATCAACGGAGTGACCGGCTGGCGGCTGTG
CGAACGCATTCTGGCGTAATAATTATAGGATAATTGAATAAAAACAGTATAGAGAGCAGATAAATACTGCT
CTCTATTTTACTAATAAGGAGGATTTAAATTGCTAAAAAATACAAACTTAGCTAATTATAAAAAAGTGAAT
ACACGGTTTGGAAATCTTAGTTTTGACGACAAAGGTATTTCTAATGACTTAACGGAAGAACAGCAAAAAGA
ATTAGGTAAGCTTCGAGGATTCGAATATATTAAGACAGAACAGAAAACAAAAGAAGAACCTAAGAAAGAAG
AACCTAAGAAAGAAGAACCTAAGAAAGAAGAACCTAAGAAAGAAGAACCTAAGAAAGAAGAACCTAAGAAA
GAAAGTACAGAAAATGAATTAGACAGCTTCTTAGCTAAAGAGCCTTCAATCAAAGAATTAAAAGAATTTGC
GAGTAAAAAAGGCATTAAAATTGAAAAAACTAAGAAAAATGATATAATTGAAGAACTAAAGAGAGGGTAAT
GTATAATGTATGGAGGTTATGAAGGACAAGATTCTTACGAATACCCTTACTCACATGGGAACCCTAAGCAT
GTAGAGCCAGAAAAAGTTGACGAATATGTTCTTTCTGATTATGGTTGGACTGCGGAAACAATTAAAGCATA
CATGTATGGTGTTCGTGTAGTAGACCCTGAAACAGGAGAGGAAATGGGAGACACCTTCTACAATCATATTA
TAGAGGTTGCCGTTGATAAGGC
SEQ ID NO: 34 - P100:: nluc
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAATATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAGCTTGTTCGTGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGACTTAGCAGCACACGAATACAAAGTTGTTGTAAGTTCTGACGATGCAGAGTCTATTGCAAGTGAAGTGG
CTACAGCTACAGTTACTGCAAAAGATGACGGCGTTAAACTAGAAATCGAGTTAGCTCCAATGTACAGCTCC
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTATGACTTAAACGACTCTATTCCTGAAACAGTAGACG
TATTCGTTGGTGAAATGTCTGCTAACGTAGTACACTTGTTTGAATTACTACCAATGATGAGATTACCTCTA
GCTCAAATTAACGCATCTGTTACATTTGCAGTTTTATGGTATGGAGCATTAGCTCTAAGAGCACCTAAGAA
ATGGGTACGTATTAGAAACGTTAAATATATTCCTGTAAAAAACGTTCATAGCAACTAAGAGGAGGTAAATA
TATATGGTCTTCACACTCGAAGATTTCGTTGGGGACTGGCGACAGACAGCCGGCTACAACCTGGACCAAGT
CCTTGAACAGGGAGGTGTGTCCAGTTTGTTTCAGAATCTCGGGGTGTCCGTAACTCCGATCCAAAGGATTG
TCCTGAGCGGTGAAAATGGGCTGAAGATCGACATCCATGTCATCATCCCGTATGAAGGTCTGAGCGGCGAC
CAAATGGGCCAGATCGAAAAAATTTTTAAGGTGGTGTACCCTGTGGATGATCATCACTTTAAGGTGATCCT
GCACTATGGCACACTGGTAATCGACGGGGTTACGCCGAACATGATCGACTATTTCGGACGGCCGTATGAAG
GCATCGCCGTGTTCGACGGCAAAAAGATCACTGTAACAGGGACCCTGTGGAACGGCAACAAAATTATCGAC
GAGCGCCTGATCAACCCCGACGGCTCCCTGCTGTTCCGAGTAACCATCAACGGAGTGACCGGCTGGCGGCT
GTGCGAACGCATTCTGGCGTAATAATTATAGGATAATTGAATAAAAACAGTATAGAGAGCAGATAAATACT
GCTCTCTATTTTACTAATAAGGAGGATTTAAATTGCTAAAAAATACAAACTTAGCTAATTATAAAAAAGTG
AATACACGATTTGGAAATCTTAGTTTTGATGATAAAGGTATTTCTAATGACCTAACGGAAGAGCAGCAAAA
AGAATTAGGTAAGCTTAGAGGATTCGAATATATTAAGACAGAACAGAAAACGAAAGAAGAACCTAAGAAAG
AAGAACCTAAGAAAGAAAGTACAGAAAATGAATTAGACAGCTTCTTAGCTAAAGAACCTTCAATCAAAGAA
TTAAAAGAATTTGCGAGTAAAAAAGGCATTAAAATTGAAAAAACTAAGAAAAATGATATAATTGAAGAACT
AAAGAGAGGGTAATGTACAATGTATGGAGGTTATGAAGGACAAGATTCTTACGAATACCCTTACTCACACG
GGAACCCTAAGCATGTAGAGCCAGAAAAAGTTGACGAATATGTTCTTTCTGATTATGGCTGGACTGCGGAA
ACAATTAAAGCATACATGTATGGTGTTCGTGTAGTAGACCCTGAAACAGGAGAGGAAATGGGAGACACCTT
CTACAATCATATTATAGAGGTTGCCGTTGATAAGGC
SEQ ID NO: 35 - LP40:: nluc
ATGCCAAAAAATAACAAAGAAGAAGAAGTTAAAGAAGTAAACCTTAATTCAGTACAAGAGGATGCGTTAAA
GTCCTTTACAACTGGTTATGGTATCACACCTGATACACAAACAGATGCAGGGGCACTAAGACGTGAGTTCC
TAGACGACCAAATCTCAATGCTTACTTGGACAGAAAATGATTTAACATTCTACAAAGACATCGCTAAAAAA
CCAGCTACATCTACAGTAGCAAAATACGATGTGTACATGCAACACGGTAAAGTAGGTCATACTAGATTTAC
TCGTGAGATTGGGGTAGCACCAGTAAGTGACCCTAACATCCGTCAAAAAACAGTAAACATGAAATTTGCTT
CTGATACTAAAAATATTAGTATCGCAGCAGGTCTAGTAAACAACATTCAAGACCCTATGCAAATTTTGACT
GATGATGCTATCGTAAATATCGCTAAAACAATTGAGTGGGCTTCATTCTTTGGAGATTCTGACTTATCAGA
TAGCCCAGAACCACAAGCAGGATTAGAATTTGATGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGCGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAATAATAAGAGGAGG
TAAATATATATGGTCTTCACACTCGAAGATTTCGTTGGGGACTGGCGACAGACAGCCGGCTACAACCTGGA
CCAAGTCCTTGAACAGGGAGGTGTGTCCAGTTTGTTTCAGAATCTCGGGGTGTCCGTAACTCCGATCCAAA
GGATTGTCCTGAGCGGTGAAAATGGGCTGAAGATCGACATCCATGTCATCATCCCGTATGAAGGTCTGAGC
GGCGACCAAATGGGCCAGATCGAAAAAATTTTTAAGGTGGTGTACCCTGTGGATGATCATCACTTTAAGGT
GATCCTGCACTATGGCACACTGGTAATCGACGGGGTTACGCCGAACATGATCGACTATTTCGGACGGCCGT
ATGAAGGCATCGCCGTGTTCGACGGCAAAAAGATCACTGTAACAGGGACCCTGTGGAACGGCAACAAAATT
ATCGACGAGCGCCTGATCAACCCCGACGGCTCCCTGCTGTTCCGAGTAACCATCAACGGAGTGACCGGCTG
GCGGCTGTGCGAACGCATTCTGGCGTAATAATTATAGGATAATTGAATAAAAACAGTATAGAGAGCAGATA
AATACTGCTCTCTATTTTACTAATAAGGAGGATTTAAATTGCTAAAAAATACAAACTTAGCTAATTATAAA
AAAGTGAATACACGGTTTGGAAATCTTAGTTTTGACGACAAAGGTATTTCTAATGACTTAACGGAAGAACA
GCAAAAAGAATTAGGTAAGCTTCGAGGATTCGAATATATTAAGACAGAACAGAAAACAAAAGAAGAACCTA
AGAAAGAAGAACCTAAGAAAGAAGAACCTAAGAAAGAAAGTACAGAAAATGAATTAGACAGCTTCTTAGCT
AAAGAGCCTTCAATCAAAGAATTAAAAGAATTTGCGAGTAAAAAAGGCATTAAAATTGAAAAAACTAAGAA
AAACGATATAATTGAAGAACTAAAGAGAGGGTAATGTATAATGTATGGAGGTTATGAAGGACAAGATTCTT
ACGAATACCCTTACTCACATGGGAACCCTAAGCATGTAGAGCCAGAAAAAGTTGACGAATATGTTCTTTCT
GATTATGGTTGGACTGCGGAAACAATTAAAGCATACATGTATGGTGTTCGTGTAGTAGACCCTGAAACAGG
AGAGGAAATGGGAGACACCTTCTACAATCATATTATAGAGGTTGCCGTTGATAAGGC
SEQ ID NO: 36 - COP2 NanoLuc
GAGGAGGTAAATATATATGGTATTCACATTAGAAGATTTTGTAGGGGATTGGCGACAAACAGCGGGATATA
ACTTAGATCAAGTTTTGGAACAGGGTGGAGTCTCAAGCCTCTTTCAAAATCTTGGAGTGAGTGTTACTCCT
ATTCAAAGAATTGTACTATCTGGTGAAAATGGCTTAAAGATTGATATACATGTTATCATTCCATACGAAGG
CTTATCGGGTGATCAAATGGGTCAAATTGAGAAAATCTTTAAAGTAGTGTATCCTGTAGACGATCATCATT
TCAAAGTTATTCTTCACTATGGTACGCTTGTGATAGACGGGGTTACACCAAATATGATTGATTACTTTGGT
CGGCCGTATGAAGGCATTGCTGTTTTTGACGGGAAAAAAATCACCGTCACTGGAACTTTATGGAATGGTAA
CAAAATCATTGATGAACGTTTGATAAATCCAGATGGATCCTTACTTTTCCGCGTGACAATCAACGGAGTAA
CGGGCTGGAGATTATGTGAACGTATTCTAGCATAA
SEQ ID NO: 37-W40_VIP_MLi178 (COP3)
ATGGTATTCACATTGGAAGATTTTGTGGGGGATTGGAGACAGACAGCTGGATATAACTTAGACCAAGTATT
AGAACAGGGTGGAGTGTCAAGCTTATTTCAAAACTTAGGTGTGTCAGTGACTCCAATTCAACGTATTGTGT
TAAGTGGAGAAAACGGTTTAAAAATAGACATTCATGTGATTATTCCGTACGAAGGCCTCAGTGGTGACCAA
ATGGGACAAATAGAGAAAATCTTTAAAGTAGTGTACCCTGTGGACGACCATCACTTTAAAGTAATCTTACA
CTATGGTACGTTAGTAATTGATGGCGTAACGCCAAACATGATAGACTACTTTGGGCGTCCTTATGAAGGCA
TTGCCGTGTTTGACGGCAAAAAGATCACCGTGACAGGTACTCTATGGAATGGAAACAAAATCATTGACGAG
CGTTTAATCAACCCAGACGGCTCTTTACTATTTCGGGTAACAATTAACGGCGTGACCGGATGGCGATTATG
CGAGCGCATTTTAGCCTAA
SEQ ID NO: 38 - A511:: COP2
ATGCCAAAAAATAACAAAGAAGAAGTTAAAGAAGTAAACCTTAATTCAGTACAAGAGGATGCGTTAAAGTC
CTTTACGACTGGTTATGGTATCACACCTGATACACAAACAGATGCAGGAGCATTAAGACGTGAGTTCCTAG
ACGACCAAATCTCAATGCTTACTTGGACAGAGAATGATTTAACATTCTATAAAGACATCGCTAAAAAACCA
GCTACATCTACAGTAGCAAAATACGATGTATACATGCAACATGGTAAGGTAGGTCATACTAGATTTACTCG
TGAGATTGGGGTAGCACCAGTAAGTGACCCTAACATCCGTCAAAAAACAGTAAATATGAAATTTGCTTCCG
ATACTAAAAACATCAGTATCGCAGCAGGTCTAGTAAACAACATTCAAGACCCAATGCAAATTTTGACTGAC
GATGCTATCGTAAATATTGCTAAAACAATTGAGTGGGCTTCATTCTTTGGAGATTCTGACTTATCAGATAG
CCCAGAACCACAAGCAGGACTAGAATTTGACGGCTTGGCTAAACTTATTAACCAAGATAACGTTCATGATG
CTCGTGGAGCTAGCTTGACTGAAAGCTTGTTAAACCAAGCAGCAGTAATGATTAGTAAAGGTTATGGTACA
CCTACAGATGCTTACATGCCAGTAGGGGTTCAAGCAGACTTTGTTAACCAACAACTTTCTAAACAAACACA
ACTTGTTCGCGATAACGGAAACAACGTAAGCGTTGGTTTCAACATCCAAGGTTTCCATTCAGCTCGTGGAT
TTATCAAACTTCACGGTTCTACAGTAATGGAAAACGAACAAATCTTAGATGAACGTATTCTTGCTTTACCA
ACAGCTCCACAACCAGCTAAGGTAACTGCAACACAAGAAGCAGGTAAAAAAGGACAATTTAGAGCAGAAGA
TTTAGCAGCACATGAATATAAAGTTGTTGTAAGTTCTGACGATGCAGAGTCTATTGCAAGTGAAGTGGCTA
CAGCTACAGTTACTGCAAAAGATGACGGCGTTAAACTAGAAATCGAATTAGCTCCAATGTATAGCTCTCGT
CCACAATTCGTTTCAATCTATAGAAAAGGTGCAGAAACAGGTTTATTCTACCTAATCGCTCGTGTACCTGC
TAGCAAAGCAGAGAACAACGTAATCACTTTCTACGACTTAAACGACTCTATTCCTGAAACAGTAGACGTAT
TCGTTGGTGAAATGTCGGCTAACGTAGTACACTTGTTTGAATTACTACCAATGATGAGATTACCTCTAGCT
CAAATTAACGCATCTGTTACATTTGCAGTTTTATGGTATGGCGCATTAGCTCTAAGAGCACCTAAGAAATG
GGTACGTATTAGAAACGTTAAATATATTCCTGTAAAAAACGTTCATAGCAACTAATAATAAGAGGAGGTAA
ATATATATGGTATTCACATTAGAAGATTTTGTAGGGGATTGGCGACAAACAGCGGGATATAACTTAGATCA
AGTTTTGGAACAGGGTGGAGTCTCAAGCCTCTTTCAAAATCTTGGAGTGAGTGTTACTCCTATTCAAAGAA
TTGTACTATCTGGTGAAAATGGCTTAAAGATTGATATACATGTTATCATTCCATACGAAGGCTTATCGGGT
GATCAAATGGGTCAAATTGAGAAAATCTTTAAAGTAGTGTATCCTGTAGACGATCATCATTTCAAAGTTAT
TCTTCACTATGGTACGCTTGTGATAGACGGGGTTACACCAAATATGATTGATTACTTTGGTCGGCCGTATG
AAGGCATTGCTGTTTTTGACGGGAAAAAAATCACCGTCACTGGAACTTTATGGAATGGTAACAAAATCATT
GATGAACGTTTGATAAATCCAGATGGATCCTTACTTTTCCGCGTGACAATCAACGGAGTAACGGGCTGGAG
ATTATGTGAACGTATTCTAGCATAATAATTATAGGATAATTGAATAAAAACAGTATAGAGAGCAGATAAAT
ACTGCTCTCTATTTTACTAATAAGGAGGATTTAAATTGCTAAAAAATACAAACTTAGCTAATTATAAAAAA
GTGAATACACGGTTTGGAAATCTTAGTTTTGACGACAAAGGTATTTCTAATGACTTAACGGAAGAACAGCA
AAAAGAATTAGGTAAGCTTCGAGGATTCGAATATATTAAGACAGAACAGAAAACAAAAGAAGAACCTAAGA
AAGAAGAACCTAAGAAAGAAGAACCTAAGAAAGAAGAACCTAAGAAAGAAGAACCTAAGAAAGAAGAACCT
AAGAAAGAAAGTACAGAAAATGAATTAGACAGCTTCTTAGCTAAAGAGCCTTCAATCAAAGAATTAAAAGA
ATTTGCGAGTAAAAAAGGCATTAAAATTGAAAAAACTAAGAAAAATGATATAATTGAAGAACTAAAGAGAG
GGTAATGTATAATGTATGGAGGTTATGA
SEQ ID NO: 39 - LP124:: COP2
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAACATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGCGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAATAATAAGAGGAGG
TAAATATATATGGTATTCACATTAGAAGATTTTGTAGGGGATTGGCGACAAACAGCGGGATATAACTTAGA
TCAAGTTTTGGAACAGGGTGGAGTCTCAAGCCTCTTTCAAAATCTTGGAGTGAGTGTTACTCCTATTCAAA
GAATTGTACTATCTGGTGAAAATGGCTTAAAGATTGATATACATGTTATCATTCCATACGAAGGCTTATCG
GGTGATCAAATGGGTCAAATTGAGAAAATCTTTAAAGTAGTGTATCCTGTAGACGATCATCATTTCAAAGT
TATTCTTCACTATGGTACGCTTGTGATAGACGGGGTTACACCAAATATGATTGATTACTTTGGTCGGCCGT
ATGAAGGCATTGCTGTTTTTGACGGGAAAAAAATCACCGTCACTGGAACTTTATGGAATGGTAACAAAATC
ATTGATGAACGTTTGATAAATCCAGATGGATCCTTACTTTTCCGCGTGACAATCAACGGAGTAACGGGCTG
GAGATTATGTGAACGTATTCTAGCATAATAATTATAGGATAATTGAATAAAAACAGTATAGAGAGCAGATA
AATACTGCTCTCTATTTTACTAATAAGGAGGATTTAAATTGCTAAAAAATACAAACTTAGCTAATTATAAA
AAAGTGAATACACGGTTTGGAAATCTTAGTTTTGACGACAAAGGTATTTCTAATGACTTAACGGAAGAACA
GCAAAAAGAATTAGGTAAGCTTCGAGGATTCGAATATATTAAGACAGAACAGAAAACAAAAGAAGAACCTA
AGAAAGAAGAACCTAAGAAAGAAGAACCTAAGAAAGAAGAACCTAAGAAAGAAGAACCTAAGAAAGAAGAA
CCTAAGAAAGAAAGTACAGAAAATGAATTAGACAGCTTCTTAGCTAAAGAGCCTTCAATCAAAGAATTAAA
AGAATTTGCGAGTAAAAAAGGCATTAAAATTGAAAAAACTAAGAAAAATGATATAATTGAAGAACTAAAGA
GAGGGTAATGTATAATGTATGGAGGTTATGA
SEQ ID NO: 40 - LP40:: COP2
ATGCCAAAAAATAACAAAGAAGAAGAAGTTAAAGAAGTAAACCTTAATTCAGTACAAGAGGATGCGTTAAA
GTCCTTTACAACTGGTTATGGTATCACACCTGATACACAAACAGATGCAGGGGCACTAAGACGTGAGTTCC
TAGACGACCAAATCTCAATGCTTACTTGGACAGAAAATGATTTAACATTCTACAAAGACATCGCTAAAAAA
CCAGCTACATCTACAGTAGCAAAATACGATGTGTACATGCAACACGGTAAAGTAGGTCATACTAGATTTAC
TCGTGAGATTGGGGTAGCACCAGTAAGTGACCCTAACATCCGTCAAAAAACAGTAAACATGAAATTTGCTT
CTGATACTAAAAATATTAGTATCGCAGCAGGTCTAGTAAACAACATTCAAGACCCTATGCAAATTTTGACT
GATGATGCTATCGTAAATATCGCTAAAACAATTGAGTGGGCTTCATTCTTTGGAGATTCTGACTTATCAGA
TAGCCCAGAACCACAAGCAGGATTAGAATTTGATGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGCGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAATAATAAGAGGAGG
TAAATATATATGGTATTCACATTAGAAGATTTTGTAGGGGATTGGCGACAAACAGCGGGATATAACTTAGA
TCAAGTTTTGGAACAGGGTGGAGTCTCAAGCCTCTTTCAAAATCTTGGAGTGAGTGTTACTCCTATTCAAA
GAATTGTACTATCTGGTGAAAATGGCTTAAAGATTGATATACATGTTATCATTCCATACGAAGGCTTATCG
GGTGATCAAATGGGTCAAATTGAGAAAATCTTTAAAGTAGTGTATCCTGTAGACGATCATCATTTCAAAGT
TATTCTTCACTATGGTACGCTTGTGATAGACGGGGTTACACCAAATATGATTGATTACTTTGGTCGGCCGT
ATGAAGGCATTGCTGTTTTTGACGGGAAAAAAATCACCGTCACTGGAACTTTATGGAATGGTAACAAAATC
ATTGATGAACGTTTGATAAATCCAGATGGATCCTTACTTTTCCGCGTGACAATCAACGGAGTAACGGGCTG
GAGATTATGTGAACGTATTCTAGCATAATAATTATAGGATAATTGAATAAAAACAGTATAGAGAGCAGATA
AATACTGCTCTCTATTTTACTAATAAGGAGGATTTAAATTGCTAAAAAATACAAACTTAGCTAATTATAAA
AAAGTGAATACACGGTTTGGAAATCTTAGTTTTGACGACAAAGGTATTTCTAATGACTTAACGGAAGAACA
GCAAAAAGAATTAGGTAAGCTTCGAGGATTCGAATATATTAAGACAGAACAGAAAACAAAAGAAGAACCTA
AGAAAGAAGAACCTAAGAAAGAAGAACCTAAGAAAGAAAGTACAGAAAATGAATTAGACAGCTTCTTAGCT
AAAGAGCCTTCAATCAAAGAATTAAAAGAATTTGCGAGTAAAAAAGGCATTAAAATTGAAAAAACTAAGAA
AAACGATATAATTGAAGAACTAAAGAGAGGGTAATGTATAATGTATGGAGGTTATGAAGGACAAGATTCTT
ACGAATACCCTTACTCACATGGGAACCCTAA
SEQ ID NO: 41 - COP2 NanoLuc
GAGGAGGTAAATATATATGGTATTCACATTAGAAGATTTTGTAGGGGATTGGCGACAAACAGCGGGATATA
ACTTAGATCAAGTTTTGGAACAGGGTGGAGTCTCAAGCCTCTTTCAAAATCTTGGAGTGAGTGTTACTCCT
ATTCAAAGAATTGTACTATCTGGTGAAAATGGCTTAAAGATTGATATACATGTTATCATTCCATACGAAGG
CTTATCGGGTGATCAAATGGGTCAAATTGAGAAAATCTTTAAAGTAGTGTATCCTGTAGACGATCATCATT
TCAAAGTTATTCTTCACTATGGTACGCTTGTGATAGACGGGGTTACACCAAATATGATTGATTACTTTGGT
CGGCCGTATGAAGGCATTGCTGTTTTTGACGGGAAAAAAATCACCGTCACTGGAACTTTATGGAATGGTAA
CAAAATCATTGATGAACGTTTGATAAATCCAGATGGATCCTTACTTTTCCGCGTGACAATCAACGGAGTAA
CGGGCTGGAGATTATGTGAACGTATTCTAGCATAA
SEQ ID NO: 42-A511:: COP3
ATGCCAAAAAATAACAAAGAAGAAGTTAAAGAAGTAAACCTTAATTCAGTACAAGAGGATGCGTTAAAGTC
CTTTACGACTGGTTATGGTATCACACCTGATACACAAACAGATGCAGGAGCATTAAGACGTGAGTTCCTAG
ACGACCAAATCTCAATGCTTACTTGGACAGAGAATGATTTAACATTCTATAAAGACATCGCTAAAAAACCA
GCTACATCTACAGTAGCAAAATACGATGTATACATGCAACATGGTAAGGTAGGTCATACTAGATTTACTCG
TGAGATTGGGGTAGCACCAGTAAGTGACCCTAACATCCGTCAAAAAACAGTAAATATGAAATTTGCTTCCG
ATACTAAAAACATCAGTATCGCAGCAGGTCTAGTAAACAACATTCAAGACCCAATGCAAATTTTGACTGAC
GATGCTATCGTAAATATTGCTAAAACAATTGAGTGGGCTTCATTCTTTGGAGATTCTGACTTATCAGATAG
CCCAGAACCACAAGCAGGACTAGAATTTGACGGCTTGGCTAAACTTATTAACCAAGATAACGTTCATGATG
CTCGTGGAGCTAGCTTGACTGAAAGCTTGTTAAACCAAGCAGCAGTAATGATTAGTAAAGGTTATGGTACA
CCTACAGATGCTTACATGCCAGTAGGGGTTCAAGCAGACTTTGTTAACCAACAACTTTCTAAACAAACACA
ACTTGTTCGCGATAACGGAAACAACGTAAGCGTTGGTTTCAACATCCAAGGTTTCCATTCAGCTCGTGGAT
TTATCAAACTTCACGGTTCTACAGTAATGGAAAACGAACAAATCTTAGATGAACGTATTCTTGCTTTACCA
ACAGCTCCACAACCAGCTAAGGTAACTGCAACACAAGAAGCAGGTAAAAAAGGACAATTTAGAGCAGAAGA
TTTAGCAGCACATGAATATAAAGTTGTTGTAAGTTCTGACGATGCAGAGTCTATTGCAAGTGAAGTGGCTA
CAGCTACAGTTACTGCAAAAGATGACGGCGTTAAACTAGAAATCGAATTAGCTCCAATGTATAGCTCTCGT
CCACAATTCGTTTCAATCTATAGAAAAGGTGCAGAAACAGGTTTATTCTACCTAATCGCTCGTGTACCTGC
TAGCAAAGCAGAGAACAACGTAATCACTTTCTACGACTTAAACGACTCTATTCCTGAAACAGTAGACGTAT
TCGTTGGTGAAATGTCGGCTAACGTAGTACACTTGTTTGAATTACTACCAATGATGAGATTACCTCTAGCT
CAAATTAACGCATCTGTTACATTTGCAGTTTTATGGTATGGCGCATTAGCTCTAAGAGCACCTAAGAAATG
GGTACGTATTAGAAACGTTAAATATATTCCTGTAAAAAACGTTCATAGCAACTAATAATAAGAGGAGGTAA
ATATATATGGTATTCACATTGGAAGATTTTGTGGGGGATTGGAGACAGACAGCTGGATATAACTTAGACCA
AGTATTAGAACAGGGTGGAGTGTCAAGCTTATTTCAAAACTTAGGTGTGTCAGTGACTCCAATTCAACGTA
TTGTGTTAAGTGGAGAAAACGGTTTAAAAATAGACATTCATGTGATTATTCCGTACGAAGGCCTCAGTGGT
GACCAAATGGGACAAATAGAGAAAATCTTTAAAGTAGTGTACCCTGTGGACGACCATCACTTTAAAGTAAT
CTTACACTATGGTACGTTAGTAATTGATGGCGTAACGCCAAACATGATAGACTACTTTGGGCGTCCTTATG
AAGGCATTGCCGTGTTTGACGGCAAAAAGATCACCGTGACAGGTACTCTATGGAATGGAAACAAAATCATT
GACGAGCGTTTAATCAACCCAGACGGCTCTTTACTATTTCGGGTAACAATTAACGGCGTGACCGGATGGCG
ATTATGCGAGCGCATTTTAGCCTAATAATTATAGGATAATTGAATAAAAACAGTATAGAGAGCAGATAAAT
ACTGCTCTCTATTTTACTAATAAGGAGGATTTAAATTGCTAAAAAATACAAACTTAGCTAATTATAAAAAA
GTGAATACACGGTTTGGAAATCTTAGTTTTGACGACAAAGGTATTTCTAATGACTTAACGGAAGAACAGCA
AAAAGAATTAGGTAAGCTTCGAGGATTCGAATATATTAAGACAGAACAGAAAACAAAAGAAGAACCTAAGA
AAGAAGAACCTAAGAAAGAAGAACCTAAGAAAGAAGAACCTAAGAAAGAAGAACCTAAGAAAGAAGAACCT
AAGAAAGAAAGTACAGAAAATGAATTAGACAGCTTCTTAGCTAAAGAGCCTTCAATCAAAGAATTAAAAGA
ATTTGCGAGTAAAAAAGGCATTAAAATTGAAAAAACTAAGAAAAATGATATAATTGAAGAACTAAAGAGAG
GGTAATGTATAATGTATGGAGGTTATGA
SEQ ID NO: 43-LP124:: COP3
ATGCCAAAAAATAACAAAGAAGAAGAAGTTAAAGAAGTAAACCTTAATTCAGTACAAGAGGACGCGTTAAA
GTCCTTTACAACTGGTTATGGTATCACACCTGATACACAAACAGATGCAGGAGCATTAAGACGTGAGTTCC
TAGACGACCAAATCTCAATGCTTACTTGGACAGAGAATGATTTAACATTCTATAAAGACATCGCTAAAAAA
CCAGCTACATCTACAGTAGCAAAATACGATGTATACATGCAACATGGTAAGGTAGGTCATACTAGATTTAC
TCGTGAGATTGGGGTAGCACCAGTAAGTGACCCTAACATCCGTCAAAAAACAGTAAACATGAAATTTGCTT
CCGATACTAAAAACATCAGTATCGCAGCAGGTCTAGTAAACAACATTCAAGACCCAATGCAAATTTTGACT
GACGATGCTATCGTAAATATTGCTAAAACAATTGAGTGGGCTTCATTCTTTGGAGATTCTGACTTATCAGA
TAGCCCAGAACCACAAGCAGGACTAGAATTTGACGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGCGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAATAATAAGAGGAGG
TAAATATATATGGTATTCACATTGGAAGATTTTGTGGGGGATTGGAGACAGACAGCTGGATATAACTTAGA
CCAAGTATTAGAACAGGGTGGAGTGTCAAGCTTATTTCAAAACTTAGGTGTGTCAGTGACTCCAATTCAAC
GTATTGTGTTAAGTGGAGAAAACGGTTTAAAAATAGACATTCATGTGATTATTCCGTACGAAGGCCTCAGT
GGTGACCAAATGGGACAAATAGAGAAAATCTTTAAAGTAGTGTACCCTGTGGACGACCATCACTTTAAAGT
AATCTTACACTATGGTACGTTAGTAATTGATGGCGTAACGCCAAACATGATAGACTACTTTGGGCGTCCTT
ATGAAGGCATTGCCGTGTTTGACGGCAAAAAGATCACCGTGACAGGTACTCTATGGAATGGAAACAAAATC
ATTGACGAGCGTTTAATCAACCCAGACGGCTCTTTACTATTTCGGGTAACAATTAACGGCGTGACCGGATG
GCGATTATGCGAGCGCATTTTAGCCTAATAATTATAGGATAATTGAATAAAAACAGTATAGAGAGCAGATA
AATACTGCTCTCTATTTTACTAATAAGGAGGATTTAAATTGCTAAAAAATACAAACTTAGCTAATTATAAA
AAAGTGAATACACGGTTTGGAAATCTTAGTTTTGACGACAAAGGTATTTCTAATGACTTAACGGAAGAACA
GCAAAAAGAATTAGGTAAGCTTCGAGGATTCGAATATATTAAGACAGAACAGAAAACAAAAGAAGAACCTA
AGAAAGAAGAACCTAAGAAAGAAGAACCTAAGAAAGAAGAACCTAAGAAAGAAGAACCTAAGAAAGAAGAA
CCTAAGAAAGAAAGTACAGAAAATGAATTAGACAGCTTCTTAGCTAAAGAGCCTTCAATCAAAGAATTAAA
AGAATTTGCGAGTAAAAAAGGCATTAAAATTGAAAAAACTAAGAAAAATGATATAATTGAAGAACTAAAGA
GAGGGTAATGTATAATGTATGGAGGTTATGA
SEQ ID NO: 44 - LP40:: COP3
ATGCCAAAAAATAACAAAGAAGAAGAAGTTAAAGAAGTAAACCTTAATTCAGTACAAGAGGATGCGTTAAA
GTCCTTTACAACTGGTTATGGTATCACACCTGATACACAAACAGATGCAGGGGCACTAAGACGTGAGTTCC
TAGACGACCAAATCTCAATGCTTACTTGGACAGAAAATGATTTAACATTCTACAAAGACATCGCTAAAAAA
CCAGCTACATCTACAGTAGCAAAATACGATGTGTACATGCAACACGGTAAAGTAGGTCATACTAGATTTAC
TCGTGAGATTGGGGTAGCACCAGTAAGTGACCCTAACATCCGTCAAAAAACAGTAAACATGAAATTTGCTT
CTGATACTAAAAATATTAGTATCGCAGCAGGTCTAGTAAACAACATTCAAGACCCTATGCAAATTTTGACT
GATGATGCTATCGTAAATATCGCTAAAACAATTGAGTGGGCTTCATTCTTTGGAGATTCTGACTTATCAGA
TAGCCCAGAACCACAAGCAGGATTAGAATTTGATGGCTTGGCTAAACTTATTAACCAAGATAACGTTCATG
ATGCTCGTGGAGCTAGCTTGACTGAAAGCTTGTTAAACCAAGCAGCAGTAATGATTAGTAAAGGTTATGGT
ACACCTACAGATGCTTACATGCCAGTAGGGGTTCAAGCAGACTTTGTTAACCAACAACTTTCTAAACAAAC
ACAACTTGTTCGCGATAACGGAAACAACGTAAGCGTTGGTTTCAACATCCAAGGTTTCCATTCAGCTCGTG
GATTTATCAAACTTCACGGTTCTACAGTAATGGAAAACGAACAAATCTTAGATGAACGTATTCTTGCTTTA
CCAACAGCTCCACAACCAGCTAAGGTAACTGCAACACAAGAAGCAGGTAAAAAAGGACAATTTAGAGCAGA
AGATTTAGCAGCACATGAATATAAAGTTGTTGTAAGTTCTGACGATGCAGAGTCTATTGCAAGTGAAGTGG
CTACAGCTACAGTTACTGCAAAAGATGACGGCGTTAAACTAGAAATCGAATTAGCTCCAATGTATAGCTCT
CGTCCACAATTCGTTTCAATCTATAGAAAAGGTGCAGAAACAGGTTTATTCTACCTAATCGCTCGTGTACC
TGCTAGCAAAGCAGAGAACAACGTAATCACTTTCTACGACTTAAACGACTCTATTCCTGAAACAGTAGACG
TATTCGTTGGTGAAATGTCGGCTAACGTAGTACACTTGTTTGAATTACTACCAATGATGAGATTACCTCTA
GCTCAAATTAACGCATCTGTTACATTTGCAGTTTTATGGTATGGCGCATTAGCTCTAAGAGCACCTAAGAA
ATGGGTACGTATTAGAAACGTTAAATATATTCCTGTAAAAAACGTTCATAGCAACTAATAATAAGAGGAGG
TAAATATATATGGTATTCACATTGGAAGATTTTGTGGGGGATTGGAGACAGACAGCTGGATATAACTTAGA
CCAAGTATTAGAACAGGGTGGAGTGTCAAGCTTATTTCAAAACTTAGGTGTGTCAGTGACTCCAATTCAAC
GTATTGTGTTAAGTGGAGAAAACGGTTTAAAAATAGACATTCATGTGATTATTCCGTACGAAGGCCTCAGT
GGTGACCAAATGGGACAAATAGAGAAAATCTTTAAAGTAGTGTACCCTGTGGACGACCATCACTTTAAAGT
AATCTTACACTATGGTACGTTAGTAATTGATGGCGTAACGCCAAACATGATAGACTACTTTGGGCGTCCTT
ATGAAGGCATTGCCGTGTTTGACGGCAAAAAGATCACCGTGACAGGTACTCTATGGAATGGAAACAAAATC
ATTGACGAGCGTTTAATCAACCCAGACGGCTCTTTACTATTTCGGGTAACAATTAACGGCGTGACCGGATG
GCGATTATGCGAGCGCATTTTAGCCTAATAATTATAGGATAATTGAATAAAAACAGTATAGAGAGCAGATA
AATACTGCTCTCTATTTTACTAATAAGGAGGATTTAAATTGCTAAAAAATACAAACTTAGCTAATTATAAA
AAAGTGAATACACGGTTTGGAAATCTTAGTTTTGACGACAAAGGTATTTCTAATGACTTAACGGAAGAACA
GCAAAAAGAATTAGGTAAGCTTCGAGGATTCGAATATATTAAGACAGAACAGAAAACAAAAGAAGAACCTA
AGAAAGAAGAACCTAAGAAAGAAGAACCTAAGAAAGAAAGTACAGAAAATGAATTAGACAGCTTCTTAGCT
AAAGAGCCTTCAATCAAAGAATTAAAAGAATTTGCGAGTAAAAAAGGCATTAAAATTGAAAAAACTAAGAA
AAACGATATAATTGAAGAACTAAAGAGAGGGTAATGTATAATGTATGGAGGTTATGAAGGACAAGATTCTT
ACGAATACCCTTACTCACATGGGAACCCTAA
SEQ ID NO: 45 UTR1 (original)
GAGGAGGTAAATATAT
SEQ ID NO: 46 UTR2
ATAATTTTGATTAACTTTAGAGGAGGTAAATATAT
SEQ ID NO: 47 UTR3
AAGGAGATAAATATAT
SEQ ID NO: 48 UTR4
GAGGAGGTAAATA
SEQ ID NO: 49 UTR5
AAGGAGATAAATA
SEQ ID NO: 50 UTR6
ATAATTTTGATTAACTTTAGAGGAGGTAAATA
SEQ ID NO: 51 UTR7
ATAATTTTGATTAACTTTAAAGGAGATAAATATAT
SEQ ID NO: 52 UTR8
ATAATTTTGATTAACTTTAAAGGAGATAAATA
SEQ ID NO: 53 COPD12
ATGGTTTTTACACTAGAGGATTTTGTCGGGGATTGGCGTCAAACTGCCGGATACAACTTAGATCAAGTGTT
AGAACAGGGTGGAGTAAGTAGTCTTTTCCAAAACTTAGGTGTGTCAGTAACTCCTATTCAACGGATTGTTT
TATCTGGAGAGAACGGTTTGAAAATTGATATTCACGTGATAATTCCGTACGAAGGATTAAGCGGAGATCAG
ATGGGGCAAATTGAGAAAATCTTTAAAGTAGTATACCCAGTTGATGACCATCATTTCAAAGTGATTTTACA
TTACGGAACTCTAGTAATTGACGGTGTGACCCCAAATATGATTGACTATTTTGGCCGTCCATACGAAGGAA
TAGCTGTCTTTGACGGTAAAAAAATTACAGTAACTGGAACATTATGGAACGGAAACAAAATCATTGACGAG
CGTTTAATCAATCCGGATGGCTCTTTACTCTTTCGCGTGACGATTAACGGAGTGACAGGTTGGCGTTTGTG
TGAGCGTATTCTTGCCTAATGA
SEQ ID NO: 54 - Ribosome binding site
TAATAAGAGGAGGTAAATATAT
SEQ ID NO: 55 - pMAK upf
TTACGCCAAGCTTGGCTGCAACGTGAGTTCCTAGACGACC
SEQ ID NO: 56 - dbono380
ATATTTACCTCCTCTTATTATTAGTTGCTATGAACGTTTTTTACAGG
SEQ ID NO: 57 - SO472
ATAAGAGGAGGTAAATATATATGGTATTCACATTAGAAGATTTTG
SEQ ID NO: 58 - SO473
ATTCAATTATCCTATAATTATTATGCTAGAATACGTTCACATAA
SEQ ID NO: 59 - SO474
GTGAACGTATTCTAGCATAATAATTATAGGATAATTGAATAAAAAC
SEQ ID NO: 60 - dbono382
ACGACGGCCAGTGAATTCCCTCGTGGTGTTCTGACTCCCG
SEQ ID NO: 64 - SO670
TAATAAGAGGAGGTAAATATATATGGTATTCACATTGGAAGA
SEQ ID NO: 65 - SO671
ATTCAATTATCCTATAATTATTAGGCTAAAATGCGCTCGC
SEQ ID NO: 66 - SO672
GCGAGCGCATTTTAGCCTAATAATTATAGGATAATTGAAT

### EQUIVALENTS

The details of one or more embodiments of the invention are set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but by the claims appended hereto.

1. A composition comprising at least one recombinant phage capable of infecting a Listeria target microbe, said phage comprising at least a capsid protein sequence, a ribosome binding site, and a codon-optimized luciferase marker.

2. The composition of claim 1, further comprising at least two, three, four, five, or six recombinant phages capable of infecting a target microbe, each of said phage comprising at least a capsid protein sequence, a ribosome binding site, and a codon-optimized marker.

3-4. (canceled)

5. The composition of claim 1, wherein the ribosome binding site of each phage is SEQ ID NO: 54

6. The composition of claim 1, wherein the codon-optimized luciferase marker is selected from the group consisting of SEQ ID NO:36 (COP2), SEQ ID NO:37 (COP3), and SEQ ID NO:115 (UTR7 variant).

7-9. (canceled)

10. The composition of claim 1, wherein the at least one recombinant phage is selected from the group consisting of LP173, LP80, V18, LP22, LP143, A511, LP101, LP124, LP99, LP48, LP125, P100, and LP40.

11. The composition of claim 10, wherein the at least one recombinant phage is selected from the group consisting of LP80, V18, LP22, A511, LP40 and LP124.

12. The composition of claim 11, wherein the composition comprises LP80, V18, LP22, A511, LP40, and LP124.

13-19. (canceled)

20. The composition of claim 1, wherein the target microbe is Listeria selected from the group consisting of Listeria innocua, Listeria monocytogenes, Listeria seeligeri, Listeria ivanovii, Listeria grayi, Listeria marthii, Listeria rocourti, Listeria welshimeri, Listeria floridensis, Listeria aquatic, Listeria cornellensis, Listeria riparia, Listeria weihenstephanensis, Listeria flieschmannii, Listeria neworkensis, and Listeria grandensis.

21. The composition of claim 20, wherein the target microbe is Listeria monocytogenes.

22. The composition of claim 1, further comprising an aqueous solution, wherein the aqueous solution comprises:
a) at least one nutrient;
b) at least one selective agent suitable to inhibit growth of at least one non-target microbe in an environmental sample or an agricultural sample;
c) at least one vitamin;
d) at least one divalent metal; and
e) at least one buffering agent capable of maintaining the composition at pH 7.0-7.5.

23. The composition of claim 22, further comprising at least one agent to prevent the decomposition of luciferin, wherein the agent is selected from the group consisting of non-ionic detergents, oxygen scavengers, and emulsifiers.

24-28. (canceled)

29. The composition of claim 23, wherein the at least one agent to prevent the decomposition of luciferin is selected from the group consisting of sodium metabisulfite, sodium thiosulfate, Tween-80, HEPES, and lecithin.

30. The composition of claim 22, further comprising at least one agent suitable to neutralize a sanitizer present in an environmental sample, wherein the at least one agent suitable to neutralize a sanitizer is selected from the group consisting of sodium metabisulfite, sodium pyruvate, sodium thiosulfate, Tween-80, HEPES, and lecithin.

31-33. (canceled)

34. The composition of claim 22, wherein the at least one nutrient is selected from a culture medium, alcohol, sugar, sugar derivatives, and combinations thereof, wherein the at least one selective agent suitable to inhibit growth of a non-target microbe is selected from LiCl, acriflavine, nalidixic acid, cycloheximide, and combinations thereof, wherein the at least one vitamin comprises yeast extract, wherein the at least one divalent metal is selected from CaCl2, MgSO4, and combinations thereof, and wherein the at least one buffering agent comprises HEPES buffer.

35-39. (canceled)

40. The composition of claim 34, wherein the aqueous solution comprises Tryptic Soy Broth, LiCl, nalidixic acid, yeast extract, glucose, MgSO4, pyruvate, and HEPES, and optionally may further comprise Tween-80, lecithin, and potassium phosphate.

41. (canceled)

42. The composition of claim 1, further comprising a substrate for luciferase.

43. The composition of claim 42, wherein the substrate is luciferin.

44-47. (canceled)

48. A method of determining a presence or absence of a target microbe in an environmental sample, an agricultural sample or both, comprising:
forming a reaction mixture by contacting an environmental sample, an agricultural sample, or both with a composition comprising at least one recombinant phage capable of infecting a Listeria target microbe, said phage comprising at least a capsid protein sequence, a ribosome binding site, and a codon-optimized luciferase marker; and
detecting, using a luciferase substrate present in the reaction mixture, a presence or absence of light generated in the reaction mixture, whereby
the presence or the absence, respectively, of the target microbe in the environmental sample and/or the agricultural sample is determined.

49. The method of claim 48, further comprising the step of incubating the reaction mixture at a temperature between 30° C. and 35° C., inclusive of the endpoints, prior to the detecting step.

50-51. (canceled)

52. The method of claim 48, further comprising the step of centrifuging the test sample prior to the detecting step.

53-56. (canceled)

57. The method of claim 48, wherein the reaction mixture has a volume of at least 300 μl to 600 μl at the time the presence or absence of light is detected.

58-59. (canceled)

60. The method of claim 48, further comprising confirming a positive result of the detecting step by contacting the detected reaction mixture or a portion thereof, with a confirmation composition comprising an organic solvent, and wherein a decrease in an abundance or intensity of light confirms that the positive result is a true result.

61-62. (canceled)

63. The method of claim 60, wherein the organic solvent comprises acetone or ethanol.

64-65. (canceled)

66. The method of claim 48, further comprising collecting the environmental sample, the agricultural sample, or both prior to the contacting step, and wherein collecting comprises contacting a sponge to a portion or a surface of the environmental sample and/or the agricultural sample to form a test sponge and subsequently contacting the test sponge to the composition.

67-68. (canceled)

69. The method of claim 48, wherein the environmental sample is selected from the group consisting of an agricultural production facility, a food production facility, a container, a machine, a processing plant, a storage facility, a health care facility, an educational institution, a loading dock, a cargo hold, a sink, a vehicle, an airport, and a customs facility.

70-75. (canceled)

76. The method of 48, wherein the environmental sample, the agricultural sample, or both is liquid or solid, and is plant or animal.

77. The method of 76, wherein the sample is a dairy product, a fruit product, a grain product, a sweet, a vegetable product, a meat product, or a combination thereof.

78-87. (canceled)

88. A kit comprising at least one recombinant phage capable of infecting a Listeria target microbe, said phage comprising at least a capsid protein sequence, a ribosome binding site, and a codon-optimized luciferase marker.

89. The kit of claim 88, further comprising an organic solvent as a confirmation composition, or a polyurethane sponge, or both.

90-96. (canceled)

97. The kit of claim 88, further comprising:
an aqueous solution composition comprising Tryptic Soy Broth, LiCl, nalidixic acid, yeast extract, glucose, MgSO4, pyruvate, HEPES, Tween-80, lecithin, and potassium phosphate;
a luciferase substrate; and
a buffer comprising Tween 80, lecithin, and HK2PO4.

98. The kit of claim 97, further comprising
an organic solvent as a confirmation solution, or a sponge, or both.

99-105. (canceled)

106. A method of making a recombinant phage capable of infecting a target microbe, said phage comprising at least a capsid protein sequence, a ribosome binding site, and a codon-optimized marker, comprising:
inserting into a phage targeting vector (PTV), a nucleic acid sequence encoding the capsid protein sequence, a nucleic acid sequence encoding a ribosome binding site, and a nucleic acid sequence encoding a codon-optimized marker;
transforming the PTV into a phage host cell; and
incubating the phage host cell with a starting phage, thereby generating a recombinant phage capable of infecting a target microbe.

107. The method of claim 106, wherein at least one of the nucleic acid sequence encoding the capsid protein sequence, the nucleic acid sequence encoding a ribosome binding site, and the nucleic acid sequence encoding a codon-optimized marker are a heterologous nucleic acid sequence.

108. (canceled)

109. The method of claim 106, wherein a contiguous nucleic acid molecule comprises the nucleic acid sequence encoding the capsid protein sequence, the nucleic acid sequence encoding a ribosome binding site, and the nucleic acid sequence encoding a codon-optimized marker.

110. The method of claim 106, wherein the host cell is isolated or derived from a strain selected from the group consisting of 1816, 1817, 1823, 1825, 1826, 1828, 1832, 1836, 1883, 1886, 1890, 1892, 1893, 1894, 1899, 1900, 1907, 1909, 1912, 1916, 1951, 1962, 1978, 1979, 1981, 1990, 1991, 1992, 1993, 1994, 1995, 2006, 2010, 2011, 2012, 2013, 2067, 2071, 2080, 2081, 2082, 2085, 2087, 2089, 2100, 2101, 2102, 2103, 2104, 2105, 2107, 2108, 2110, 2112, 2134, 2136, 2137, 2138, B4-G7, B5-E10, B6-G7, B7-A10, B7-F6, B9-G4, BG-G10, 085-018-02 1, 085-018-02 2, 085-018-02 3, 088-013 02 S1, 088-013 02 S3, 112-009-08 1, 112-009-08 2, 112-009-08 3, 112-010-02 1, 112-010-02 1 F, 112-010-02 1 L, 112-010-02 2, 112-010-02 2 F, 112-010-02 2 L, 112-010-02 3, 112-010-02 3 F, 112-010-02 3 L, 112-019-01 1 L, 112-019-01 2 L, 112-019-01 3 L, 113-022-01 1, 113-022-01 2, 113-023-02 1 L, 113-023-02 2 L and 113-023-02 3 L.
